(19) United States
(12) Patent Application Publication
(10) Pub. No.: US 2020/0277373 A1
(43) Pub. Date: Sep. 3, 2020

(54) ANTI-TREM2 ANTIBODIES AND METHODS OF USE THEREOF

(71) Applicant: Denali Therapeutics Inc., South San Francisco, CA (US)

(72) Inventors: Hang Chen, South San Francisco, CA (US); Gilbert Di Paolo, South San Francisco, CA (US); Rui Hao, South San Francisco, CA (US); Joseph W. Lewcock, South San Francisco, CA (US); Nathan Moerke, South San Francisco, CA (US); Alicia A. Nugent, South San Francisco, CA (US); Rishi Rakhit, South San Francisco, CA (US); Ju Shi, South San Francisco, CA (US); Rinkan Shukla, South San Francisco, CA (US); Ankita Srivastava, South San Francisco, CA (US); Bettina Van Lengerich, South San Francisco, CA (US); Yin Zhang, South San Francisco, CA (US)

(73) Assignee: Denali Therapeutics Inc., South San Francisco, CA (US)

(21) Appl. No.: 16/646,536

(22) PCT Filed: Sep. 14, 2018
(86) PCT No.: PCT/US2018/051166

§ 371 Date: Mar. 11, 2020

Related U.S. Application Data
(60) Provisional application No. 62/621,380, filed on Jan. 24, 2018.
(60) Provisional application No. 62/583,379, filed on Nov. 8, 2017.
(60) Provisional application No. 62/558,803, filed on Sep. 14, 2017.

(51) Int. Cl. C07K 16/28; A61P 25/28; C07K 16/40

(52) U.S. Cl. CPC C07K 16/2803; A61P 25/28; C07K 16/40; C07K 2317/33; C07K 2317/34; A61K 2039/505; C07K 2317/75; C07K 2317/76; C07K 2317/92; C12Y 207/10002; C07K 2317/526

(57) ABSTRACT

In one aspect, antibodies that specifically bind to a human triggering receptor expressed on myeloid cells 2 (TREM2) protein are provided. In some embodiments, the antibody increases levels of soluble TREM2 (sTREM2). In some embodiments, the antibody decreases levels of sTREM2. In some embodiments, the antibody enhances TREM2 activity. In some embodiments, the antibody inhibits TREM2 activity.

### CROSS-REFERENCE TO RELATED APPLICATIONS

This application claims priority to U.S. Provisional Patent Application Nos. 62/558,803, filed Sep. 14, 2017, 62/583,379, filed Nov. 8, 2017, and 62/621,380, filed Jan. 24, 2018, the content of each of which is incorporated by reference in its entirety.

### BACKGROUND

Triggering receptor expressed on myeloid cells-2 (TREM2) is a transmembrane receptor that is expressed on microglia and is believed to function in regulating phagocytosis, cell survival, and the production of pro-inflammatory cytokines. Mutations in TREM2 have been identified in neurodegenerative diseases including Alzheimer's disease, Nasu-Hakola disease, Parkinson's disease, amyotrophic lateral sclerosis, and frontotemporal dementia. Additionally, altered levels of soluble TREM2 (sTREM2) have been reported in the cerebrospinal fluid of patients having Alzheimer's disease or frontotemporal dementia who have a mutation in TREM2.

There remains a need for therapeutic agents that modulate TREM2 activity or levels of sTREM2.

### BRIEF SUMMARY

In one aspect, isolated antibodies or antigen-binding portions thereof that specifically bind to a human triggering receptor expressed on myeloid cells 2 (TREM2) protein are provided. In some embodiments, the antibody or antigen-binding portion thereof modulates (e.g., decreases or increases) levels of sTREM2. In some embodiments, the antibody or antigen-binding portion thereof modulates (e.g., decreases or increases) levels of sTREM2 and further modulates (e.g., enhances or inhibits) one or more TREM2 activities. In some embodiments, the antibody or antigen-binding portion thereof modulates (e.g., enhances or inhibits) spleen tyrosine kinase (Syk) phosphorylation. In some embodiments, the antibody or antigen-binding portion thereof modulates (e.g., enhances or inhibits) phagocytosis. In some embodiments, the antibody or antigen-binding portion thereof modulates (e.g., enhances or inhibits) migration of myeloid cells, macrophages, microglia, or disease-associated microglia. In some embodiments, the antibody or antigen-binding portion thereof modulates (e.g., enhances or inhibits) differentiation of myeloid cells, macrophages, microglia, or disease-associated microglia. In some embodiments, the antibody or antigen-binding portion thereof modulates (e.g., enhances or inhibits) survival of myeloid cells, macrophages, microglia, or disease-associated microglia. In some embodiments, the antibody or antigen-binding portion thereof modulates (e.g., enhances or inhibits) one or more TREM2 activities without blocking binding of a native TREM2 ligand. In some embodiments, the antibody or antigen-binding portion thereof modulates (e.g., enhances or inhibits) one or more TREM2 activities that is induced by a TREM2 ligand. In some embodiments, the antibody or antigen-binding portion thereof modulates (e.g., selectively enhances or selectively inhibits) one or more TREM2 activities that is induced by a TREM2 ligand. In some embodiments, the antibody or antigen-binding portion thereof modulates (e.g., enhances or inhibits) one or more TREM2 activities that is induced by a TREM2 ligand but does not modulate (e.g., enhance or inhibit) TREM2 activity in the absence of the TREM2 ligand. In some embodiments, the antibody or antigen-binding portion thereof prevents activation of TREM2 by a TREM2 ligand. In some embodiments, the antibody or antigen-binding portion thereof blocks binding of a TREM2 ligand to TREM2. In some embodiments, the TREM2 ligand is selected from the group consisting of 1-palmitoyl-2-(5′-oxo-valeroyl)-sn-glycero-3-phosphocholine (POVPC), 2-Arachidonoylglycerol (2-AG), 7-ketocholesterol (7-KC), 24(S)hydroxycholesterol (240HC), 25(S)hydroxycholesterol (25OHC), 27-hydroxycholesterol (270HC), Acyl Carnitine (AC), alkylacylglycerophosphocholine (PAF), α-galactosylceramide (KRN7000), Bis(monoacylglycero)phosphate (BMP), Cardiolipin (CL), Ceramide, Ceramide-1-phosphate (C1P), Cholesteryl ester (CE), Cholesterol phosphate (CP), Diacylglycerol 34:1 (DG 34:1), Diacylglycerol 38:4 (DG 38:4), Diacylglycerol pyrophosphate (DGPP), Dihyrdoceramide (DhCer), Dihydrosphingomyelin (DhSM), Ether phosphatidylcholine (PCe), Free cholesterol (FC), Galactosylceramide (GalCer), Galactosylsphingosine (GalSo), Ganglioside GM1, Ganglioside GM3, Glucosylsphingosine (GlcSo), Hank's Balanced Salt Solution (HBSS), Kdo2-Lipid A (KLA), Lactosylceramide (LacCer), lysoalkylacylglycerophosphocholine (LPAF), Lysophosphatidic acid (LPA), Lysophosphatidylcholine (LPC), Lysophosphatidylethanolamine (LPE), Lysophosphatidylglycerol (LPG), Lysophosphatidylinositol (LPI), Lysosphingomyelin (LSM), Lysophosphatidylserine (LPS), N-Acyl-phosphatidylethanolamine (NAPE), N-Acyl-Serine (NSer), Oxidized phosphatidylcholine (oxPC), Palmitic-acid-9-hydroxy-stearic-acid (PAHSA), Phosphatidylethanolamine (PE), Phosphatidylethanol (PEtOH), Phosphatidic acid (PA), Phosphatidylcholine (PC), Phosphatidylglycerol (PG), Phosphatidylinositol (PI), Phosphatidylserine (PS), Sphinganine, Sphinganine-1-phosphate (Sa1P), Sphingomyelin (SM), Sphingosine, Sphingosine-1-phosphate (So1P), and Sulfatide.

In another aspect, isolated antibodies or antigen-binding portions thereof that specifically bind to human TREM2 and that recognize an epitope of human TREM2 that is the same or substantially the same as an epitope recognized by an antibody clone as described herein. In some embodiments, the antibody or antigen-binding portion recognizes an epitope of human TREM2 that is the same or substantially the same as an epitope recognized by an antibody clone selected from the group consisting of 2G4.B1, 3D3.A1, 7B10.A2, 8A111.B1, 13B11.A1, 14D5.F1, 14H11.A1, 19F10.F3, 21D4.D1, 21D6.G2, 21D11, 22B8.B1, 22G9.D1, 24B4.A1, 26D2.D1, 26D5.A1, 26D11.B1, 26E2.A3, 30A8.A1, 30F2.A2, 38E9.E5, 39H10.A1, 40H3.A4, 42E8.H1, 43E9.H1, 44E2.H1, 44E3.B1, 49H11.B1, 51D4, 52H9.D1, 53H11.D3, 54C2.A1, 55B9.A1, 57D7.A1, 59C6.F1, 60A4.B1, RS9.E2, RS9.F6, RS9.F10, RS11.1F5, RS11.1G6, RS11.1A10, RS11.1D11, RS11.4A5, RS11.4H6, RS11.4D7, RS11.4D9, RS11.4F11, RS12.1C6, RS12.1C10, RS12.2D1, RS12.2D4, RS12.2E1, RS12.2F2, RS12.2G1, RS12.2H1, and RS12.3C10. In some embodiments, the antibody or antigen-binding portion recognizes an epitope of human TREM2 that is the same or substantially the same as the epitope recognized by RS9.F6. In some embodiments, the antibody or antigen-binding portion binds to an epitope on human TREM2 that comprises amino acid residues 140-144. In some embodiments, the antibody or antigen-binding portion makes direct contact with one or more of residues Asp140, Leu141, Trp142, Phe143, and Pro144. In some embodiments, the antibody or antigen-binding portion makes direct contact with residue Trp142. In some embodiments, the antibody or antigen-binding portion makes direct contact with each of residues Asp140, Leu141, Trp142, Phe143, and Pro144. In some embodiments, the antibody or antigen-binding portion binds a TREM2 fragment that comprises or consists of amino acid residues 140-148.

In some embodiments, an antibody or antigen-binding portion thereof having one or more TREM2-associated activities as described herein also recognizes an epitope of human TREM2 that is the same or substantially the same as an epitope recognized by an antibody clone as described herein (e.g., an antibody clone selected from the group consisting of 2G4.B1, 3D3.A1, 7B10.A2, 8A11.B1, 13B11.A1, 14D5.F1, 14H11.A1, 19F10.F3, 21D4.D1, 21D6.G2, 21D11, 22B8.B1, 22G9.D1, 24B4.A1, 26D2.D1, 26D5.A1, 26D11.B1, 26E2.A3, 30A8.A1, 30F2.A2, 38E9.E5, 39H10.A1, 40H3.A4, 42E8.H1, 43E9.H1, 44E2.H1, 44E3.B1, 49H11.B1, 51D4, 52H9.D1, 53H11.D3, 54C2.A1, 55B9.A1, 57D7.A1, 59C6.F1, 60A4.B1, RS9.E2, RS9.F6, RS9.F10, RS11.1F5, RS11.1G6, RS11.1A10, RS11.1D11, RS11.4A5, RS11.4H6, RS11.4D7, RS11.4D9, RS11.4F11, RS12.1C6, RS12.1C10, RS12.2D1, RS12.2D4, RS12.2E1, RS12.2F2, RS12.2G1, RS12.2H1, and RS12.3C10). In some embodiments, the antibody or antigen-binding portion recognizes an epitope of human TREM2 that is the same or substantially the same as the epitope recognized by RS9.F6. In some embodiments, the antibody or antigen-binding portion binds to an epitope on human TREM2 that comprises amino acid residues 140-144. In some embodiments, the antibody or antigen-binding portion makes direct contact with one or more of residues Asp140, Leu141, Trp142, Phe143, and Pro144. In some embodiments, the antibody or antigen-binding portion makes direct contact with residue Trp142. In some embodiments, the antibody or antigen-binding portion makes direct contact with each of residues Asp140, Leu141, Trp142, Phe143, and Pro144. In some embodiments, the antibody or antigen-binding portion binds a TREM2 fragment that comprises or consists of amino acid residues 140-148.

In another aspect, antibodies or antigen-binding portion thereof having one or more CDR, heavy chain variable region, and/or light chain variable region sequences of an antibody described herein are provided. In some embodiments, the antibody or antigen-binding portion comprises one or more complementarity determining regions (CDRs) (e.g., all CDRs) having at least 90%, 91%, 92%, 93%, 94%, 95%, 96%, 97%, 98%, or 99% sequence identity to a CDR of an antibody clone selected from the group consisting of 2G4.B1, 3D3.A1, 7B10.A2, 8A11.B1, 13B11.A1, 14D5.F1, 14H11.A1, 19F10.F3, 21D4.D1, 21D6.G2, 21D11, 22B8.B1, 22G9.D1, 24B4.A1, 26D2.D1, 26D5.A1, 26D11.B1, 26E2.A3, 30A8.A1, 30F2.A2, 38E9.E5, 39H10.A1, 40H3.A4, 42E8.H1, 43E9.H1, 44E2.H1, 44E3.B1, 49H11.B1, 51D4, 52H9.D1, 53H11.D3, 54C2.A1, 55B9.A1, 57D7.A1, 59C6.F1, 60A4.B1, RS9.E2, RS9.F6, RS9.F10, RS11.1F5, RS11.1G6, RS11.1A10, RS11.1D11, RS11.4A5, RS11.4H6, RS11.4D7, RS11.4D9, RS11.4F11, RS12.1C6, RS12.1C10, RS12.2D1, RS12.2D4, RS12.2E1, RS12.2F2, RS12.2G1, RS12.2H1, and RS12.3C10. In some embodiments, the antibody or antigen-binding portion thereof comprises one or more CDRs (e.g., all CDRs) that has up to two amino acid substitutions relative to a CDR of an antibody clone selected from the group consisting of 2G4.B1, 3D3.A1, 7B10.A2, 8A11.B1, 13B11.A1, 14D5.F1, 14H11.A1, 19F10.F3, 21D4.D1, 21D6.G2, 21D11, 22B8.B1, 22G9.D1, 24B4.A1, 26D2.D1, 26D5.A1, 26D11.B1, 26E2.A3, 30A8.A1, 30F2.A2, 38E9.E5, 39H10.A1, 40H3.A4, 42E8.H1, 43E9.H1, 44E2.H1, 44E3.B1, 49H11.B1, 51D4, 52H9.D1, 53H11.D3, 54C2.A1, 55B9.A1, 57D7.A1, 59C6.F1, 60A4.B1, RS9.E2, RS9.F6, RS9.F10, RS11.1F5, RS11.1G6, RS11.1A10, RS11.1D11, RS11.4A5, RS11.4H6, RS11.4D7, RS11.4D9, RS11.4F11, RS12.1C6, RS12.1C10, RS12.2D1, RS12.2D4, RS12.2E1, RS12.2F2, RS12.2G1, RS12.2H1, and RS12.3C10. In some embodiments, the antibody or antigen-binding portion thereof comprises each of a heavy chain CDR1 (CDR-H1), a heavy chain CDR2 (CDR-H2), a heavy chain CDR3 (CDR-H3), a light chain CDR1 (CDR-L1), a light chain CDR2 (CDR-L2), and a light chain CDR3 (CDR-L3) that is identical to a CDR-H1, CDR-H2, CDR-H3, CDR-L1, CDR-L2, and CDR-L3 of an antibody clone selected from the group consisting of 2G4.B1, 3D3.A1, 7B10.A2, 8A11.B1, 13B11.A1, 14D5.F1, 14H11.A1, 19F10.F3, 21D4.D1, 21D6.G2, 21D11, 22B8.B1, 22G9.D1, 24B4.A1, 26D2.D1, 26D5.A1, 26D11.B1, 26E2.A3, 30A8.A1, 30F2.A2, 38E9.E5, 39H10.A1, 40H3.A4, 42E8.H1, 43E9.H1, 44E2.H1, 44E3.B1, 49H11.B1, 51D4, 52H9.D1, 53H11.D3, 54C2.A1, 55B9.A1, 57D7.A1, 59C6.F1, 60A4.B1, RS9.E2, RS9.F6, RS9.F10, RS11.1F5, RS11.1G6, RS11.1A10, RS11.1D11, RS11.4A5, RS11.4H6, RS11.4D7, RS11.4D9, RS11.4F11, RS12.1C6, RS12.1C10, RS12.2D1, RS12.2D4, RS12.2E1, RS12.2F2, RS12.2G1, RS12.2H1, and RS12.3C10.

In some embodiments, the antibody or antigen-binding portion thereof comprises a heavy chain variable region comprising an amino acid sequence that has at least 90%, 91%, 92%, 93%, 94%, 95%, 96%, 97%, 98%, or 99% sequence identity to the heavy chain variable region of an antibody clone selected from the group consisting of 2G4.B1, 3D3.A1, 7B10.A2, 8A11.B1, 13B11.A1, 14D5.F1, 14H11.A1, 19F10.F3, 21D4.D1, 21D6.G2, 21D11, 22B8.B1, 22G9.D1, 24B4.A1, 26D2.D1, 26D5.A1, 26D11.B1, 26E2.A3, 30A8.A1, 30F2.A2, 38E9.E5, 39H10.A1, 40H3.A4, 42E8.H1, 43E9.H1, 44E2.H1, 44E3.B1, 49H11.B1, 51D4, 52H9.D1, 53H11.D3, 54C2.A1, 55B9.A1, 57D7.A1, 59C6.F1, 60A4.B1, RS9.E2, RS9.F6, RS9.F10, RS11.1F5, RS11.1G6, RS11.1A10, RS11.1D11, RS11.4A5, RS11.4H6, RS11.4D7, RS11.4D9, RS11.4F11, RS12.1C6, RS12.1C10, RS12.2D1, RS12.2D4, RS12.2E1, RS12.2F2, RS12.2G1, RS12.2H1, and RS12.3C10. In some embodiments, the antibody or antigen-binding portion thereof comprises a light chain variable region comprising an amino acid sequence that has at least 90%, 91%, 92%, 93%, 94%, 95%, 96%, 97%, 98%, or 99% sequence identity to the light chain variable region of an antibody clone selected from the group consisting of 2G4.B1, 3D3.A1, 7B10.A2, 8A11.B1, 13B11.A1, 14D5.F1, 14H11.A1, 19F10.F3, 21D4.D1, 21D6.G2, 21D11, 22B8.B1, 22G9.D1, 24B4.A1, 26D2.D1, 26D5.A1, 26D11.B1, 26E2.A3, 30A8.A1, 30F2.A2, 38E9.E5, 39H10.A1, 40H3.A4, 42E8.H1, 43E9.H1, 44E2.H1, 44E3.B1, 49H11.B1, 51D4, 52H9.D1, 53H11.D3, 54C2.A1, 55B9.A1, 57D7.A1, 59C6.F1, 60A4.B1, RS9.E2, RS9.F6, RS9.F10, RS11.1F5, RS11.1G6, RS11.1A10, RS11.1D11, RS11.4A5, RS11.4H6, RS11.4D7, RS11.4D9, RS11.4F11, RS12.1C6, RS12.1C10, RS12.2D1, RS12.2D4, RS12.2E1, RS12.2F2, RS12.2G1, RS12.2H1, and RS12.3C10. In some embodiments, the antibody or antigen-binding portion thereof comprises a heavy chain variable region comprising an amino acid sequence that has at least 90%, 91%, 92%, 93%, 94%, 95%, 96%, 97%, 98%, or 99% sequence identity to the heavy chain variable region of an antibody clone selected from the group consisting of 2G4.B1, 3D3.A1, 7B10.A2, 8A11.B1, 13B11.A1, 14D5.F1, 14H11.A1, 19F10.F3, 21D4.D1, 21D6.G2, 21D11, 22B8.B1, 22G9.D1, 24B4.A1, 26D2.D1, 26D5.A1, 26D11.B1, 26E2.A3, 30A8.A1, 30F2.A2, 38E9.E5, 39H10.A1, 40H3.A4, 42E8.H1, 43E9.H1, 44E2.H1, 44E3.B1, 49H11.B1, 51D4, 52H9.D1, 53H11.D3, 54C2.A1, 55B9.A1, 57D7.A1, 59C6.F1, 60A4.B1, RS9.E2, RS9.F6, RS9.F10, RS11.1F5, RS11.1G6, RS11.1A10, RS11.1D11, RS11.4A5, RS11.4H6, RS11.4D7, RS11.4D9, RS11.4F11, RS12.1C6, RS12.1C10, RS12.2D1, RS12.2D4, RS12.2E1, RS12.2F2, RS12.2G1, RS12.2H1, and RS12.3C10, and comprises a light chain variable region comprising an amino acid sequence that has at least 90%, 91%, 92%, 93%, 94%, 95%, 96%, 97%, 98%, or 99% sequence identity to the light chain variable region of an antibody clone selected from the group consisting of 2G4.B1, 3D3.A1, 7B10.A2, 8A11.B1, 13B11.A1, 14D5.F1, 14H11.A1, 19F10.F3, 21D4.D1, 21D6.G2, 21D11, 22B8.B1, 22G9.D1, 24B4.A1, 26D2.D1, 26D5.A1, 26D11.B1, 26E2.A3, 30A8.A1, 30F2.A2, 38E9.E5, 39H10.A1, 40H3.A4, 42E8.H1, 43E9.H1, 44E2.H1, 44E3.B1, 49H11.B1, 51D4, 52H9.D1, 53H11.D3, 54C2.A1, 55B9.A1, 57D7.A1, 59C6.F1, 60A4.B1, RS9.E2, RS9.F6, RS9.F10, RS11.1F5, RS11.1G6, RS11.1A10, RS11.1D11, RS11.4A5, RS11.4H6, RS11.4D7, RS11.4D9, RS11.4F11, RS12.1C6, RS12.1C10, RS12.2D1, RS12.2D4, RS12.2E1, RS12.2F2, RS12.2G1, RS12.2H1, and RS12.3C10.

In some embodiments, the antibody or antigen-binding portion thereof comprises a heavy chain variable region comprising an amino acid sequence that (i) has at least 75%, 80%, 85%, 90%, 91%, 92%, 93%, 94%, 95%, 96%, 97%, 98%, or 99% sequence identity to the heavy chain variable region of an antibody clone selected from the group consisting of 2G4.B1, 3D3.A1, 7B10.A2, 8A11.B1, 13B11.A1, 14D5.F1, 14H11.A1, 19F10.F3, 21D4.D1, 21D6.G2, 21D11, 22B8.B1, 22G9.D1, 24B4.A1, 26D2.D1, 26D5.A1, 26D11.B1, 26E2.A3, 30A8.A1, 30F2.A2, 38E9.E5, 39H10.A1, 40H3.A4, 42E8.H1, 43E9.H1, 44E2.H1, 44E3.B1, 49H11.B1, 51D4, 52H9.D1, 53H11.D3, 54C2.A1, 55B9.A1, 57D7.A1, 59C6.F1, 60A4.B1, RS9.E2, RS9.F6, RS9.F10, RS11.1F5, RS11.1G6, RS11.1A10, RS11.1D11, RS11.4A5, RS11.4H6, RS11.4D7, RS11.4D9, RS11.4F11, RS12.1C6, RS12.1C10, RS12.2D1, RS12.2D4, RS12.2E1, RS12.2F2, RS12.2G1, RS12.2H1, and RS12.3C10 and (ii) comprises a CDR-H1, CDR-H2, and CDR-H3 that is identical to the CDR-H1, CDR-H2, and CDR-H3 of the antibody clone. In some embodiments, the antibody or antigen-binding portion thereof comprises a light chain variable region comprising an amino acid sequence that (i) has at least 75%, 80%, 85%, 90%, 91%, 92%, 93%, 94%, 95%, 96%, 97%, 98%, or 99% sequence identity to the light chain variable region of an antibody clone selected from the group consisting of 2G4.B1, 3D3.A1, 7B10.A2, 8A11.B1, 13B11.A1, 14D5.F1, 14H11.A1, 19F10.F3, 21D4.D1, 21D6.G2, 21D11, 22B8.B1, 22G9.D1, 24B4.A1, 26D2.D1, 26D5.A1, 26D11.B1, 26E2.A3, 30A8.A1, 30F2.A2, 38E9.E5, 39H10.A1, 40H3.A4, 42E8.H1, 43E9.H1, 44E2.H1, 44E3.B1, 49H11.B1, 51D4, 52H9.D1, 53H11.D3, 54C2.A1, 55B9.A1, 57D7.A1, 59C6.F1, 60A4.B1, RS9.E2, RS9.F6, RS9.F10, RS11.1F5, RS11.1G6, RS11.1A10, RS11.1D11, RS11.4A5, RS11.4H6, RS11.4D7, RS11.4D9, RS11.4F11, RS12.1C6, RS12.1C10, RS12.2D1, RS12.2D4, RS12.2E1, RS12.2F2, RS12.2G1, RS12.2H1, and RS12.3C10 and (ii) comprises a CDR-L1, CDR-L2, and CDR-L3 that is identical to the CDR-L1, CDR-L2, and CDR-L3 of the antibody clone.

In some embodiments, the antibody or antigen-binding portion thereof comprises: a heavy chain variable region comprising an amino acid sequence that (i) has at least 75%, 80%, 85%, 90%, 91%, 92%, 93%, 94%, 95%, 96%, 97%, 98%, or 99% sequence identity to the heavy chain variable region of an antibody clone selected from the group consisting of 2G4.B1, 3D3.A1, 7B10.A2, 8A11.B1, 13B11.A1, 14D5.F1, 14H11.A1, 19F10.F3, 21D4.D1, 21D6.G2, 21D11, 22B8.B1, 22G9.D1, 24B4.A1, 26D2.D1, 26D5.A1, 26D11.B1, 26E2.A3, 30A8.A1, 30F2.A2, 38E9.E5, 39H10.A1, 40H3.A4, 42E8.H1, 43E9.H1, 44E2.H1, 44E3.B1, 49H11.B1, 51D4, 52H9.D1, 53H11.D3, 54C2.A1, 55B9.A1, 57D7.A1, 59C6.F1, 60A4.B1, RS9.E2, RS9.F6, RS9.F10, RS11.1F5, RS11.1G6, RS11.1A10, RS11.1D11, RS11.4A5, RS11.4H6, RS11.4D7, RS11.4D9, RS11.4F11, RS12.1C6, RS12.1C10, RS12.2D1, RS12.2D4, RS12.2E1, RS12.2F2, RS12.2G1, RS12.2H1, and RS12.3C10 and (ii) comprises a CDR-H1, CDR-H2, and CDR-H3 that is identical to the CDR-H1, CDR-H2, and CDR-H3 of the antibody clone; and a light chain variable region comprising an amino acid sequence that (i) has at least 75%, 80%, 85%, 90%, 91%, 92%, 93%, 94%, 95%, 96%, 97%, 98%, or 99% sequence identity to the light chain variable region of an antibody clone selected from the group consisting of 2G4.B1, 3D3.A1, 7B10.A2, 8A11.B1, 13B11.A1, 14D5.F1, 14H11.A1, 19F10.F3, 21D4.D1, 21D6.G2, 21D11, 22B8.B1, 22G9.D1, 24B4.A1, 26D2.D1, 26D5.A1, 26D11.B1, 26E2.A3, 30A8.A1, 30F2.A2, 38E9.E5, 39H10.A1, 40H3.A4, 42E8.H1, 43E9.H1, 44E2.H1, 44E3.B1, 49H11.B1, 51D4, 52H9.D1, 53H11.D3, 54C2.A1, 55B9.A1, 57D7.A1, 59C6.F1, 60A4.B1, RS9.E2, RS9.F6, RS9.F10, RS11.1F5, RS11.1G6, RS11.1A10, RS11.1D11, RS11.4A5, RS11.4H6, RS11.4D7, RS11.4D9, RS11.4F11, RS12.1C6, RS12.1C10, RS12.2D1, RS12.2D4, RS12.2E1, RS12.2F2, RS12.2G1, RS12.2H1, and RS12.3C10 and (ii) comprises a CDR-L1, CDR-L2, and CDR-L3 that is identical to the CDR-L1, CDR-L2, and CDR-L3 of the antibody clone.

In some embodiments, an antibody or antigen-binding portion thereof having one or more TREM2-associated activities as described herein also comprises one or more CDR, heavy chain variable region, and/or light chain variable region sequences of an antibody described herein (e.g., an antibody clone selected from the group consisting of 2G4.B1, 3D3.A1, 7B10.A2, 8A11.B1, 13B11.A1, 14D5.F1, 14H11.A1, 19F10.F3, 21D4.D1, 21D6.G2, 21D11, 22B8.B1, 22G9.D1, 24B4.A1, 26D2.D1, 26D5.A1, 26D11.B1, 26E2.A3, 30A8.A1, 30F2.A2, 38E9.E5, 39H10.A1, 40H3.A4, 42E8.H1, 43E9.H1, 44E2.H1, 44E3.B1, 49H11.B1, 51D4, 52H9.D1, 53H11.D3, 54C2.A1, 55B9.A1, 57D7.A1, 59C6.F1, 60A4.B1, RS9.E2, RS9.F6, RS9.F10, RS11.1F5, RS11.1G6, RS11.1A10, RS11.1D11, RS11.4A5, RS11.4H6, RS11.4D7, RS11.4D9, RS11.4F11, RS12.1C6, RS12.1C10, RS12.2D1, RS12.2D4, RS12.2E1, RS12.2F2, RS12.2G1, RS12.2H1, and RS12.3C10).

In some embodiments, an antibody or antigen-binding portion thereof that specifically bind to human TREM2 comprises one or more (e.g., one, two, three, four, five, or all six) CDRs selected from the group consisting of:
(a) a heavy chain CDR1 sequence having at least 90% sequence identity to the amino acid sequence of any one of SEQ ID NOs:8, 36, 39, 45, 51, 57, 62, 68, 74, 81, 85, 307, and 315 or having up to two amino acid substitutions relative to the amino acid sequence of any one of SEQ ID NOs:8, 36, 39, 45, 51, 57, 62, 68, 74, 81, 85, 307, and 315;
(b) a heavy chain CDR2 sequence having at least 90% sequence identity to the amino acid sequence of any one of SEQ ID NOs:9, 37, 40, 46, 52, 58, 63, 75, 79, 82, 86, 308, and 316 or having up to two amino acid substitutions relative to the amino acid sequence of any one of SEQ ID NOs:9, 37, 40, 46, 52, 58, 63, 75, 79, 82, 86, 308, and 316;
(c) a heavy chain CDR3 sequence having at least 90% sequence identity to the amino acid sequence of any one of SEQ ID NOs:10, 41, 47, 53, 59, 64, 70, 76, 83, 87, 309, and 317 or having up to two amino acid substitutions relative to the amino acid sequence of any one of SEQ ID NOs:10, 41, 47, 53, 59, 64, 70, 76, 83, 87, 309, and 317;
(d) a light chain CDR1 sequence having at least 90% sequence identity to the amino acid sequence of any one of SEQ ID NOs: 11, 42, 48, 54, 60, 65, 71, 77, 88, and 311 or having up to two amino acid substitutions relative to the amino acid sequence of any one of SEQ ID NOs:11, 42, 48, 54, 60, 65, 71, 77, 88, and 311;
(e) a light chain CDR2 sequence having at least 90% sequence identity to the amino acid sequence of any one of SEQ ID NOs:12, 38, 43, 49, 55, 66, 72, 312, and 319 or having up to two amino acid substitutions relative to the amino acid sequence of any one of SEQ ID NOs: 12, 38, 43, 49, 55, 66, 72, 312, and 319; and
(f) a light chain CDR3 sequence having at least 90% sequence identity to the amino acid sequence of any one of SEQ ID NOs: 13, 44, 50, 56, 61, 67, 73, 78, 80, 84, 89, and 313 or having up to two amino acid substitutions relative to the amino acid sequence of any one of SEQ ID NOs:13, 44, 50, 56, 61, 67, 73, 78, 80, 84, 89, and 313.

In some embodiments, the antibody or antigen-binding portion thereof comprises comprises one or more (e.g., one, two, three, four, five, or all six) CDRs selected from the group consisting of:
(a) a heavy chain CDR1 sequence comprising the amino acid sequence of any one of SEQ ID NOs:8, 36, 39, 45, 51, 57, 62, 68, 74, 81, 85, 307, and 315;
(b) a heavy chain CDR2 sequence comprising the amino acid sequence of any one of SEQ ID NOs:9, 37, 40, 46, 52, 58, 63, 75, 79, 82, 86, 308, and 316;
(c) a heavy chain CDR3 sequence comprising the amino acid sequence of any one of SEQ ID NOs: 10, 41, 47, 53, 59, 64, 70, 76, 83, 87, 309, and 317;
(d) a light chain CDR1 sequence comprising the amino acid sequence of any one of SEQ ID NOs:11, 42, 48, 54, 60, 65, 71, 77, 88, and 311;
(e) a light chain CDR2 sequence comprising the amino acid sequence of any one of SEQ ID NOs: 12, 38, 43, 49, 55, 66, 72, 312, and 319; and
(f) a light chain CDR3 sequence comprising the amino acid sequence of any one of SEQ ID NOs: 13, 44, 50, 56, 61, 67, 73, 78, 80, 84, 89, and 313.

In some embodiments, the antibody or antigen-binding portion thereof comprises:
(a) a heavy chain CDR1 sequence comprising the amino acid sequence of SEQ ID NO:8, a heavy chain CDR2 sequence comprising the amino acid sequence of SEQ ID NO:9, a heavy chain CDR3 sequence comprising the amino acid sequence of SEQ ID NO: 10, a light chain CDR1 sequence comprising the amino acid sequence of SEQ ID NO: 11, a light chain CDR2 sequence comprising the amino acid sequence of SEQ ID NO: 12, and a light chain CDR3 sequence comprising the amino acid sequence of SEQ ID NO: 13; or
(b) a heavy chain CDR1 sequence comprising the amino acid sequence of SEQ ID NO:36, a heavy chain CDR2 sequence comprising the amino acid sequence of SEQ ID NO:37, a heavy chain CDR3 sequence comprising the amino acid sequence of SEQ ID NO: 10, a light chain CDR1 sequence comprising the amino acid sequence of SEQ ID NO: 11, a light chain CDR2 sequence comprising the amino acid sequence of SEQ ID NO:38, and a light chain CDR3 sequence comprising the amino acid sequence of SEQ ID NO:13; or
(c) a heavy chain CDR1 sequence comprising the amino acid sequence of SEQ ID NO:39, a heavy chain CDR2 sequence comprising the amino acid sequence of SEQ ID NO:40, a heavy chain CDR3 sequence comprising the amino acid sequence of SEQ ID NO:41, a light chain CDR1 sequence comprising the amino acid sequence of SEQ ID NO:42, a light chain CDR2 sequence comprising the amino acid sequence of SEQ ID NO:43, and a light chain CDR3 sequence comprising the amino acid sequence of SEQ ID NO:44; or
(d) a heavy chain CDR1 sequence comprising the amino acid sequence of SEQ ID NO:45, a heavy chain CDR2 sequence comprising the amino acid sequence of SEQ ID NO:46, a heavy chain CDR3 sequence comprising the amino acid sequence of SEQ ID NO:47, a light chain CDR1 sequence comprising the amino acid sequence of SEQ ID NO:48, a light chain CDR2 sequence comprising the amino acid sequence of SEQ ID NO:49, and a light chain CDR3 sequence comprising the amino acid sequence of SEQ ID NO:50; or
(e) a heavy chain CDR1 sequence comprising the amino acid sequence of SEQ ID NO:51, a heavy chain CDR2 sequence comprising the amino acid sequence of SEQ ID NO:52, a heavy chain CDR3 sequence comprising the amino acid sequence of SEQ ID NO:53, a light chain CDR1 sequence comprising the amino acid sequence of SEQ ID NO:54, a light chain CDR2 sequence comprising the amino acid sequence of SEQ ID NO:55, and a light chain CDR3 sequence comprising the amino acid sequence of SEQ ID NO:56; or
(f) a heavy chain CDR1 sequence comprising the amino acid sequence of SEQ ID NO:57, a heavy chain CDR2 sequence comprising the amino acid sequence of SEQ ID NO:58, a heavy chain CDR3 sequence comprising the amino acid sequence of SEQ ID NO:59, a light chain CDR1 sequence comprising the amino acid sequence of SEQ ID NO:60, a light chain CDR2 sequence comprising the amino acid sequence of SEQ ID NO:38, and a light chain CDR3 sequence comprising the amino acid sequence of SEQ ID NO:61; or
(g) a heavy chain CDR1 sequence comprising the amino acid sequence of SEQ ID NO:62, a heavy chain CDR2 sequence comprising the amino acid sequence of SEQ ID NO:63, a heavy chain CDR3 sequence comprising the amino acid sequence of SEQ ID NO:64, a light chain CDR1 sequence comprising the amino acid sequence of SEQ ID NO:65, a light chain CDR2 sequence comprising the amino acid sequence of SEQ ID NO:66, and a light chain CDR3 sequence comprising the amino acid sequence of SEQ ID NO:67; or
(h) a heavy chain CDR1 sequence comprising the amino acid sequence of SEQ ID NO:68, a heavy chain CDR2 sequence comprising the amino acid sequence of SEQ ID NO:69, a heavy chain CDR3 sequence comprising the amino acid sequence of SEQ ID NO:70, a light chain CDR1 sequence comprising the amino acid sequence of SEQ ID NO:71, a light chain CDR2 sequence comprising the amino acid sequence of SEQ ID NO:72, and a light chain CDR3 sequence comprising the amino acid sequence of SEQ ID NO:73; or
(i) a heavy chain CDR1 sequence comprising the amino acid sequence of SEQ ID NO:74, a heavy chain CDR2 sequence comprising the amino acid sequence of SEQ ID NO:75, a heavy chain CDR3 sequence comprising the amino acid sequence of SEQ ID NO:76, a light chain CDR1 sequence comprising the amino acid sequence of SEQ ID NO:77, a light chain CDR2 sequence comprising the amino acid sequence of SEQ ID NO:38, and a light chain CDR3 sequence comprising the amino acid sequence of SEQ ID NO:78; or
(j) a heavy chain CDR1 sequence comprising the amino acid sequence of SEQ ID NO:74, a heavy chain CDR2 sequence comprising the amino acid sequence of SEQ ID NO:79, a heavy chain CDR3 sequence comprising the amino acid sequence of SEQ ID NO:76, a light chain CDR1 sequence comprising the amino acid sequence of SEQ ID NO:77, a light chain CDR2 sequence comprising the amino acid sequence of SEQ ID NO:38, and a light chain CDR3 sequence comprising the amino acid sequence of SEQ ID NO:80; or
(k) a heavy chain CDR1 sequence comprising the amino acid sequence of SEQ ID NO:81, a heavy chain CDR2 sequence comprising the amino acid sequence of SEQ ID NO:82, a heavy chain CDR3 sequence comprising the amino acid sequence of SEQ ID NO:83, a light chain CDR1 sequence comprising the amino acid sequence of SEQ ID NO:60, a light chain CDR2 sequence comprising the amino acid sequence of SEQ ID NO:38, and a light chain CDR3 sequence comprising the amino acid sequence of SEQ ID NO:84; or
(l) a heavy chain CDR1 sequence comprising the amino acid sequence of SEQ ID NO:85, a heavy chain CDR2 sequence comprising the amino acid sequence of SEQ ID NO:86, a heavy chain CDR3 sequence comprising the amino acid sequence of SEQ ID NO:87, a light chain CDR1 sequence comprising the amino acid sequence of SEQ ID NO:88, a light chain CDR2 sequence comprising the amino acid sequence of SEQ ID NO:38, and a light chain CDR3 sequence comprising the amino acid sequence of SEQ ID NO:89; or
(m) a heavy chain CDR1 sequence comprising the amino acid sequence of SEQ ID NO:307, a heavy chain CDR2 sequence comprising the amino acid sequence of SEQ ID NO:308, a heavy chain CDR3 sequence comprising the amino acid sequence of SEQ ID NO:309, a light chain CDR1 sequence comprising the amino acid sequence of SEQ ID NO:311, a light chain CDR2 sequence comprising the amino acid sequence of SEQ ID NO:312, and a light chain CDR3 sequence comprising the amino acid sequence of SEQ ID NO:313; or
(n) a heavy chain CDR1 sequence comprising the amino acid sequence of SEQ ID NO:315, a heavy chain CDR2 sequence comprising the amino acid sequence of SEQ ID NO:316, a heavy chain CDR3 sequence comprising the amino acid sequence of SEQ ID NO:317, a light chain CDR1 sequence comprising the amino acid sequence of SEQ ID NO:48, a light chain CDR2 sequence comprising the amino acid sequence of SEQ ID NO:319, and a light chain CDR3 sequence comprising the amino acid sequence of SEQ ID NO:50.

In some embodiments, the antibody or antigen-binding portion thereof comprises:
a heavy chain variable region comprising (i) an amino acid sequence that has at least 75% sequence identity to SEQ ID NO:6 and (ii) the CDR-H1, CDR-H2, and CDR-H3 of SEQ ID NOs:8, 9, and 10, respectively; and/or
a light chain variable region comprising (i) an amino acid sequence that has at least 75% sequence identity to SEQ ID NO:7 and (ii) the CDR-L1, CDR-L2, and CDR-L3 of SEQ ID NOs:11, 12, and 13, respectively.

In some embodiments, the antibody or antigen-binding portion thereof comprises a heavy chain variable region comprising an amino acid sequence that has at least 90% sequence identity to any one of SEQ ID NOs:6, 14, 15, 16, 17, 18, 19, 20, 21, 22, 23, 24, 306, and 314. In some embodiments, the antibody or antigen-binding portion thereof comprises a light chain variable region comprising an amino acid sequence that has at least 90% sequence identity to any one of SEQ ID NOs:7, 25, 26, 27, 28, 29, 30, 31, 32, 33, 34, 35, 310, and 318.

In some embodiments, the antibody or antigen-binding portion thereof comprises a heavy chain variable region comprising an amino acid sequence that has at least 90% sequence identity to any one of SEQ ID NOs:6, 14, 15, 16, 17, 18, 19, 20, 21, 22, 23, 24, 306, and 314 and comprises a light chain variable region comprising an amino acid sequence that has at least 90% sequence identity to any one of SEQ ID NOs:7, 25, 26, 27, 28, 29, 30, 31, 32, 33, 34, 35, 310, and 318.

In some embodiments, the antibody or antigen-binding portion thereof comprises:
(a) a heavy chain variable region comprising an amino acid sequence that has at least 90% sequence identity to SEQ ID NO:6; and a light chain variable region comprising an amino acid sequence that has at least 90% sequence identity to SEQ ID NO:7; or
(b) a heavy chain variable region comprising an amino acid sequence that has at least 90% sequence identity to SEQ ID NO:14; and a light chain variable region comprising an amino acid sequence that has at least 90% sequence identity to SEQ ID NO:25; or
(c) a heavy chain variable region comprising an amino acid sequence that has at least 90% sequence identity to SEQ ID NO:15; and a light chain variable region comprising an amino acid sequence that has at least 90% sequence identity to SEQ ID NO:26; or
(d) a heavy chain variable region comprising an amino acid sequence that has at least 90% sequence identity to SEQ ID NO:16; and a light chain variable region comprising an amino acid sequence that has at least 90% sequence identity to SEQ ID NO:27; or
(e) a heavy chain variable region comprising an amino acid sequence that has at least 90% sequence identity to SEQ ID NO:17; and a light chain variable region comprising an amino acid sequence that has at least 90% sequence identity to SEQ ID NO:28; or
(f) a heavy chain variable region comprising an amino acid sequence that has at least 90% sequence identity to SEQ ID NO:18; and a light chain variable region comprising an amino acid sequence that has at least 90% sequence identity to SEQ ID NO:29; or
(g) a heavy chain variable region comprising an amino acid sequence that has at least 90% sequence identity to SEQ ID NO:19; and a light chain variable region comprising an amino acid sequence that has at least 90% sequence identity to SEQ ID NO:30; or
(h) a heavy chain variable region comprising an amino acid sequence that has at least 90% sequence identity to SEQ ID N020; and a light chain variable region comprising an amino acid sequence that has at least 90% sequence identity to SEQ ID NO:31; or
(i) a heavy chain variable region comprising an amino acid sequence that has at least 90% sequence identity to SEQ ID NO:21; and a light chain variable region comprising an amino acid sequence that has at least 90% sequence identity to SEQ ID NO:32; or
(j) a heavy chain variable region comprising an amino acid sequence that has at least 90% sequence identity to SEQ ID NO:22; and a light chain variable region comprising an amino acid sequence that has at least 90% sequence identity to SEQ ID NO:33; or
(k) a heavy chain variable region comprising an amino acid sequence that has at least 90% sequence identity to SEQ ID NO:23; and a light chain variable region comprising an amino acid sequence that has at least 90% sequence identity to SEQ ID NO:34; or
(l) a heavy chain variable region comprising an amino acid sequence that has at least 90% sequence identity to SEQ ID NO:24; and a light chain variable region comprising an amino acid sequence that has at least 90% sequence identity to SEQ ID NO:35; or
(m) a heavy chain variable region comprising an amino acid sequence that has at least 90% sequence identity to SEQ ID NO:306, and a light chain variable region comprising an amino acid sequence that has at least 90% sequence identity to SEQ ID NO:310; or
(n) a heavy chain variable region comprising an amino acid sequence that has at least 90% sequence identity to SEQ ID NO:314, and a light chain variable region comprising an amino acid sequence that has at least 90% sequence identity to SEQ ID NO:318.

In some embodiments, in any of the anti-TREM2 antibodies described herein, the antibody comprises a first Fc polypeptide and optionally a second Fc polypeptide. In some embodiments, the antibody comprises a first Fc polypeptide and a second Fc polypeptide. In some embodiments, the first Fc polypeptide is a modified Fc polypeptide and/or the second Fc polypeptide is a modified Fc polypeptide.

In some embodiments, in any of the anti-TREM2 antibodies described herein, the antibody comprises:
(a) a first antigen-binding portion comprising a first variable region that specifically binds to a TREM2 protein (e.g., human TREM2), wherein the first antigen-binding portion comprises (i) a first heavy chain comprising a first Fc polypeptide and (ii) a first light chain; and
(b) a second antigen-binding portion comprising a second variable region that specifically binds to the TREM2 protein (e.g., human TREM2), wherein the second antigen-binding portion comprises (i) a second heavy chain comprising a first Fc polypeptide and (ii) a second light chain;
wherein the first Fc polypeptide and the second Fc polypeptide form an Fc dimer.

In some embodiments, the first Fc polypeptide is a modified Fc polypeptide and/or the second Fc polypeptide is a modified Fc polypeptide.

In some embodiments, the first variable region and the second variable region recognize the same epitope in the TREM2 protein. In some embodiments, the first variable region and the second variable region recognize different epitopes in the TREM2 protein.

In some embodiments, the first Fc polypeptide and the second Fc polypeptide each contain modifications that promote heterodimerization. In some embodiments, one of the Fc polypeptides has a T366W substitution and the other Fc polypeptide has T366S, L368A, and Y407V substitutions, according to EU numbering.

In some embodiments, the first Fc polypeptide and/or the second Fc polypeptide comprises a native FcRn binding site. In some embodiments, the first Fc polypeptide and/or the second Fc polypeptide comprises a modification that alters FcRn binding.

In some embodiments, the first Fc polypeptide and the second Fc polypeptide do not have effector function. In some embodiments, the first Fc polypeptide and/or the second Fc polypeptide comprises a modification that reduces effector function. In some embodiments, the modification that reduces effector function comprises substitutions of Ala at position 234 and Ala at position 235, according to EU numbering.

In some embodiments, the first Fc polypeptide and/or the second Fc polypeptide comprises amino acid changes relative to the native Fc sequence that extend serum half-life. In some embodiments, the amino acid changes comprise substitutions of Tyr at position 252, Thr at position 254, and Glu at position 256, according to EU numbering. Alternatively, in other embodiments, the amino acid changes comprise substitutions of Leu at position 428 and Ser at position 434, according to EU numbering. Alternatively, in further embodiments, the amino acid changes comprise a substitution of Ser or Ala at position 434, according to EU numbering.

In some embodiments, the first Fc polypeptide and/or the second Fc polypeptide specifically binds to the transferrin receptor. In some embodiments, the first Fc polypeptide and/or the second Fc polypeptide comprises at least two substitutions at positions selected from the group consisting of 384, 386, 387, 388, 389, 390, 413, 416, and 421, according to EU numbering. In some embodiments, the first Fc polypeptide and/or the second Fc polypeptide comprises substitutions at at least three, four, five, six, seven, eight, or nine of the positions.

In some embodiments, the first Fc polypeptide and/or the second Fc polypeptide further comprises one, two, three, or four substitutions at positions comprising 380, 391, 392, and 415, according to EU numbering. In some embodiments, the first Fc polypeptide and/or the second Fc polypeptide further comprises one, two, or three substitutions at positions comprising 414, 424, and 426, according to EU numbering.

In some embodiments, the first Fc polypeptide and/or the second Fc polypeptide comprises Trp at position 388. In some embodiments, the first Fc polypeptide and/or the second Fc polypeptide comprises an aromatic amino acid at position 421. In some embodiments, the aromatic amino acid at position 421 is Trp or Phe.

In some embodiments, the first Fc polypeptide and/or the second Fc polypeptide comprises at least one position selected from the following: position 380 is Trp, Leu, or Glu; position 384 is Tyr or Phe; position 386 is Thr; position 387 is Glu; position 388 is Trp; position 389 is Ser, Ala, Val, or Asn; position 390 is Ser or Asn; position 413 is Thr or Ser; position 415 is Glu or Ser; position 416 is Glu; and position 421 is Phe.

In some embodiments, the first Fc polypeptide and/or the second Fc polypeptide comprises 2, 3, 4, 5, 6, 7, 8, 9, 10, or 11 positions selected from the following: position 380 is Trp, Leu, or Glu; position 384 is Tyr or Phe; position 386 is Thr; position 387 is Glu; position 388 is Trp; position 389 is Ser, Ala, Val, or Asn; position 390 is Ser or Asn; position 413 is Thr or Ser; position 415 is Glu or Ser; position 416 is Glu; and position 421 is Phe.

In some embodiments, the first Fc polypeptide and/or the second Fc polypeptide comprises 11 positions as follows: position 380 is Trp, Leu, or Glu; position 384 is Tyr or Phe; position 386 is Thr; position 387 is Glu; position 388 is Trp; position 389 is Ser, Ala, Val, or Asn; position 390 is Ser or Asn; position 413 is Thr or Ser; position 415 is Glu or Ser; position 416 is Glu; and position 421 is Phe.

In some embodiments, the first Fc polypeptide and/or the second Fc polypeptide has a CH3 domain with at least 85% identity, at least 90% identity, or at least 95% identity to amino acids 111-217 of any one of SEQ ID NOs:100-185, 219-298, and 337-460. In some embodiments, the first Fc polypeptide and/or the second Fc polypeptide comprises the amino acid sequence of any one of SEQ ID NOs:100-185, 219-298, and 337-460. In some embodiments, the residues for at least 5, 6, 7, 8, 9, 10, 11, 12, 13, 14, 15, or 16 of the positions corresponding to EU index positions 380, 384, 386, 387, 388, 389, 390, 391, 392, 413, 414, 415, 416, 421, 424 and 426 of any one of SEQ ID NOs:100-185, 219-298, and 337-460 are not deleted or substituted.

In some embodiments, in any of the anti-TREM2 antibodies described herein, the antibody comprises an Fc polypeptide selected from the group consisting of SEQ ID NOs:219-298 and 351-460. In some embodiments, the antibody comprises a first Fc polypeptide selected from the group consisting of SEQ ID NOs:219, 220, 221, 222, 227, 228, 229, 230, 231, 232, 239, 240, 241, 242, 243, 244, 251, 252, 253, 254, 255, 256, 263, 264, 265, 266, 267, 268, 275, 276, 277, 278, 279, 280, 287, 288, 289, 290, 291, 292, 351, 352, 355, 356, 357, 358, 359, 360, 367, 368, 369, 370, 371, 372, 379, 380, 381, 382, 383, 384, 392, 393, 394, 399, 400, 401, 406, 407, 408, 413, 414, 415, 420, 421, 422, 427, 428, 429, 434, 435, 436, 441, 442, 443, 448, 449, 450, 455, 456, and 457, and a second Fc polypeptide selected from the group consisting of SEQ ID NOs:223, 224, 225, 226, 233, 234, 235, 236, 237, 238, 245, 246, 247, 248, 249, 250, 257, 258, 259, 260, 261, 262, 269, 270, 271, 272, 273, 274, 281, 282, 283, 284, 285, 286, 293, 294, 295, 296, 297, 298, 353, 354, 361, 362, 363, 364, 365, 366, 373, 374, 375, 376, 377, 378, 385, 386, 387, 388, 389, 390, 395, 396, 397, 402, 403, 404, 409, 410, 411, 416, 417, 418, 423, 424, 425, 430, 431, 432, 437, 438, 439, 444, 445, 446, 451, 452, 453, 458, 459, and 460. In some embodiments, the antibody comprises a first Fc polypeptide selected from the group consisting of SEQ ID NOs:219, 220, 221, 222, 351, 352, 406, 407, and 408 and a second Fc polypeptide selected from the group consisting of SEQ ID NOs:223, 224, 225, 226, 353, 354, 409, 410, and 411. In some embodiments, the antibody comprises a first Fc polypeptide selected from the group consisting of SEQ ID NOs:227, 228, 229, 230, 231, 232, 392, 393, and 394 and a second Fc polypeptide selected from the group consisting of SEQ ID NOs:233, 234, 235, 236, 237, 238, 395, 396, and 397. In some embodiments, the antibody comprises a first Fc polypeptide selected from the group consisting of SEQ ID NOs:239, 240, 241, 242, 243, 244, 434, 435, and 436 and a second Fc polypeptide selected from the group consisting of SEQ ID NOs:245, 246, 247, 248, 249, 250, 437, 438, and 439. In some embodiments, the antibody comprises a first Fc polypeptide selected from the group consisting of SEQ ID NOs:251, 252, 253, 254, 255, 256, 448, 449, and 450 and a second Fc polypeptide selected from the group consisting of SEQ ID NOs:257, 258, 259, 260, 261, 262, 451, 452, and 453. In some embodiments, the antibody comprises a first Fc polypeptide selected from the group consisting of SEQ ID NOs:263, 264, 265, 266, 267, 268, 455, 456, and 457 and a second Fc polypeptide selected from the group consisting of SEQ ID NOs:269, 270, 271, 272, 273, 274, 458, 459, and 460. In some embodiments, the antibody comprises a first Fc polypeptide selected from the group consisting of SEQ ID NOs:275, 276, 277, 278, 279, 280, 413, 414, and 415 and a second Fc polypeptide selected from the group consisting of SEQ ID NOs:281, 282, 283, 284, 285, 286, 416, 417, and 418. In some embodiments, the antibody comprises a first Fc polypeptide selected from the group consisting of SEQ ID NOs:287, 288, 289, 290, 291, 292, 420, 421, and 422 and a second Fc polypeptide selected from the group consisting of SEQ ID NOs:293, 294, 295, 296, 297, 298, 423, 424, and 425. In some embodiments, the antibody comprises a first Fc polypeptide selected from the group consisting of SEQ ID NOs:355, 356, 357, 358, 360, 399, 400, and 401 and a second Fc polypeptide selected from the group consisting of SEQ ID NOs:361, 362, 363, 364, 365, 366, 402, 403, and 404. In some embodiments, the antibody comprises a first Fc polypeptide selected from the group consisting of SEQ ID NOs:367, 368, 369, 370, 371, 372, 441, 442, and 443 and a second Fc polypeptide selected from the group consisting of SEQ ID NOs:373, 374, 375, 376, 377, 378, 444, 445, and 446. In some embodiments, the antibody comprises a first Fc polypeptide selected from the group consisting of SEQ ID NOs:379, 380, 381, 382, 383, 384, 427, 428, and 429 and a second Fc polypeptide selected from the group consisting of SEQ ID NOs:385, 386, 387, 388, 389, 390, 430, 431, and 432.

In some embodiments, the first Fc polypeptide and/or the second Fc polypeptide binds to the apical domain of the transferrin receptor. In some embodiments, the binding of the bispecific antibody to the transferrin receptor does not substantially inhibit binding of transferrin to the transferrin receptor.

In some embodiments, the first Fc polypeptide and/or the second Fc polypeptide has an amino acid sequence identity of at least 75%, or at least 80%, 90%, 92%, or 95%, as compared to the corresponding wild-type Fc polypeptide (e.g., a wild-type Fc polypeptide that is a human IgG1, IgG2, IgG3, or IgG4 Fc polypeptide).

In some embodiments, uptake of the antibody or antigen-binding portion thereof into the brain is greater than the uptake of the antibody or antigen-binding portion thereof without the modifications in the first Fc polypeptide and/or the second Fc polypeptide that result in transferrin receptor binding. In some embodiments, uptake of the antibody or antigen-binding portion thereof into the brain is at least 2, 3, 4, 5, 6, 7, 8, 9, 10, 15, 20, 25, 30, 35, 40, 45, 50, 55, 60, 65, 70, 75, 80, 85, 90, 95, or 100-fold greater as compared to the uptake of the antibody or antigen-binding portion thereof without the modifications in the first Fc polypeptide and/or the second Fc polypeptide that result in transferrin receptor binding.

In some embodiments, one of the Fc polypeptides is not modified to bind to a blood-brain barrier receptor and the other Fc polypeptide is modified to specifically bind to a transferrin receptor.

In some embodiments, the antibody or antigen-binding portion thereof exhibits cross-reactivity with a mouse TREM2 protein. In some embodiments, the antibody is a chimeric antibody. In some embodiments, the antibody is a humanized antibody. In some embodiments, the antibody is a fully human antibody. In some embodiments, the antigen-binding portion is a Fab, a F(ab′)2, a scFv, or a bivalent scFv.

In another aspect, antigen-binding fragments that specifically bind to a TREM2 protein (e.g., human TREM2) are provided. In some embodiments, the antigen-binding fragment further comprises an Fc polypeptide. In some embodiments, the Fc polypeptide is a modified Fc polypeptide. In some embodiments, the Fc polypeptide contains one or more of the modifications described herein, e.g., to promote heterodimerization, reduce effector function, extend serum half-life, and/or bind to a transferrin receptor. As a non-limiting example, the antigen-binding fragment may include a Fab fragment that further comprises an Fc polypeptide, e.g., an Fab-Fc fusion. In other embodiments, the antigen-binding fragment further comprises a first Fc polypeptide and a second Fc polypeptide. In some embodiments, the first Fc polypeptide is a modified Fc polypeptide and/or the second Fc polypeptide is a modified Fc polypeptide. In some embodiments, the first Fc polypeptide and/or the second Fc polypeptide contains one or more of the modifications described herein, e.g., to promote heterodimerization, reduce effector function, extend serum half-life, and/or bind to a transferrin receptor. As a non-limiting example, the antigen-binding fragment may include a F(ab′)2 fragment that further comprises a first Fc polypeptide and a second Fc polypeptide, e.g., an F(ab′)2-Fc fusion.

In some embodiments, the antibody or antigen-binding portion thereof is a multispecific antibody. In some embodiments, the multispecific antibody is a bispecific antibody. In some embodiments, the bispecific antibody recognizes two different TREM2 epitopes. In some embodiments, the bispecific antibody is capable of inducing TREM2 clustering at the surface of a cell. In some embodiments, the bispecific antibody has an EC₅₀ that is at least 2-fold (e.g., at least 5-fold, 10-fold, 20-fold, 30-fold, 50-fold, 100-fold, 200-fold, 500-fold, or 1000-fold) lower than a bivalent monospecific antibody comprising the same sequence as a single arm of the bispecific antibody. In some embodiments, each of the two arms of the bispecific antibody is selected from an antibody clone selected from the group consisting of 2G4.B1, 3D3.A1, 7B10.A2, 8A11.B1, 13B11.A1, 14D5.F1, 14H11.A1, 19F10.F3, 21D4.D1, 21D6.G2, 21D11, 22B8.B1, 22G9.D1, 24B4.A1, 26D2.D1, 26D5.A1, 26D11.B1, 26E2.A3, 30A8.A1, 30F2.A2, 38E9.E5, 39H10.A1, 40H3.A4, 42E8.H1, 43E9.H1, 44E2.H1, 44E3.B1, 49H11.B1, 51D4, 52H9.D1, 53H11.D3, 54C2.A1, 55B9.A1, 57D7.A1, 59C6.F1, 60A4.B1, RS9.E2, RS9.F6, RS9.F10, RS11.1F5, RS11.1G6, RS11.1A10, RS11.1D11, RS11.4A5, RS11.4H6, RS11.4D7, RS11.4D9, RS11.4F11, RS12.1C6, RS12.1C10, RS12.2D1, RS12.2D4, RS12.2E1, RS12.2F2, RS12.2G1, RS12.2H1, and RS12.3C10, wherein the two arms are in different epitope bins.

In another aspect, pharmaceutical compositions are provided. In some embodiments, the pharmaceutical composition comprises an antibody or antigen-binding portion thereof that specifically binds to human TREM2. In some embodiments, the pharmaceutical composition comprises an antibody or antigen-binding portion thereof that has one or more TREM2-associated activities as described herein, recognizes an epitope of human TREM2 that is the same or substantially the same as an epitope recognized by an antibody clone as described herein, and/or comprises one or more CDR, heavy chain, and/or light chain sequences of an antibody clone as described herein.

In another aspect, isolated polynucleotides are provided. In some embodiments, the isolated polynucleotide comprises a nucleotide sequence encoding an isolated antibody or antigen-binding portion thereof that specifically binds to human TREM2 as described herein. In another aspect, vectors and host cells comprising such an isolated polynucleotide are provided.

In still another aspect, antibodies are provided that compete with an antibody clone as described herein (e.g., an antibody clone selected from the group consisting of 2G4.B1, 3D3.A1, 7B10.A2, 8A11.B1, 13B11.A1, 14D5.F1, 14H11.A1, 19F10.F3, 21D4.D1, 21D6.G2, 21D11, 22B8.B1, 22G9.D1, 24B4.A1, 26D2.D1, 26D5.A1, 26D11.B1, 26E2.A3, 30A8.A1, 30F2.A2, 38E9.E5, 39H10.A1, 40H3.A4, 42E8.H1, 43E9.H1, 44E2.H1, 44E3.B1, 49H11.B1, 51D4, 52H9.D1, 53H11.D3, 54C2.A1, 55B9.A1, 57D7.A1, 59C6.F1, 60A4.B1, RS9.E2, RS9.F6, RS9.F10, RS11.1F5, RS11.1G6, RS11.1A10, RS11.1D11, RS11.4A5, RS11.4H6, RS11.4D7, RS11.4D9, RS11.4F11, RS12.1C6, RS12.1C10, RS12.2D1, RS12.2D4, RS12.2E1, RS12.2F2, RS12.2G1, RS12.2H1, and RS12.3C10) for specific binding to human TREM2.

In yet another aspect, kits are provided for therapeutic and prognostic use as described herein. In some embodiments, the kit comprises an isolated antibody or antigen-binding portion thereof that specifically binds to human TREM2 as described herein, and further comprises instructions for therapeutic or prognostic use.

In another aspect, methods of treating a neurodegenerative disease are provided. In some embodiments, the method comprises administering to a subject having a neurodegenerative disease an antibody or pharmaceutical composition as described herein. In some embodiments, the neurodegenerative disease is selected from the group consisting of Alzheimer's disease, primary age-related tauopathy, progressive supranuclear palsy (PSP), frontotemporal dementia, frontotemporal dementia with parkinsonism linked to chromosome 17, argyrophilic grain dementia, amyotrophic lateral sclerosis, amyotrophic lateral sclerosis/parkinsonism-dementia complex of Guam (ALS-PDC), corticobasal degeneration, chronic traumatic encephalopathy, Creutzfeldt-Jakob disease, dementia pugilistica, diffuse neurofibrillary tangles with calcification, Down's syndrome, familial British dementia, familial Danish dementia, Gerstmann-Straussler-Scheinker disease, globular glial tauopathy, Guadeloupean parkinsonism with dementia, Guadelopean PSP, Hallevorden-Spatz disease, hereditary diffuse leukoencephalopathy with spheroids (HDLS), Huntington's disease, inclusion-body myositis, multiple system atrophy, myotonic dystrophy, Nasu-Hakola disease, neurofibrillary tangle-predominant dementia, Niemann-Pick disease type C, pallido-ponto-nigral degeneration, Parkinson's disease, Pick's disease, postencephalitic parkinsonism, prion protein cerebral amyloid angiopathy, progressive subcortical gliosis, subacute sclerosing panencephalitis, and tangle only dementia. In some embodiments, the neurodegenerative disease is Alzheimer's disease.

In still another aspect, methods of decreasing levels of sTREM2 in a subject having a neurodegenerative disease are provided. In some embodiments, the method comprises administering to the subject an antibody or pharmaceutical composition as described herein.

In yet another aspect, methods of increasing levels of sTREM2 in a subject having a neurodegenerative disease are provided. In some embodiments, the method comprises administering to the subject an antibody or pharmaceutical composition as described herein.

In yet another aspect, methods of enhancing TREM2 activity in a subject having a neurodegenerative disease are provided. In some embodiments, the method comprises administering to the subject an antibody or pharmaceutical composition as described herein. In some embodiments, the antibody is an antibody that enhances TREM2 activity in the presence of a TREM2 ligand. In some embodiments, the antibody is an antibody that enhances TREM2 activity in the presence but not the absence of a TREM2 ligand.

In still another aspect, methods of inhibiting TREM2 activity in a subject having a neurodegenerative disease are provided. In some embodiments, the method comprises administering to the subject an antibody or pharmaceutical composition as described herein. In some embodiments, the antibody is an antibody that inhibits TREM2 activity in the presence of a TREM2 ligand. In some embodiments, the antibody is an antibody that inhibits TREM2 activity in the presence but not the absence of a TREM2 ligand. In some embodiments, the antibody is an antibody that inhibits TREM2 activity in the absence of a TREM2 ligand and inhibits ligand activation of TREM2.

In still another aspect, methods of reducing plaque accumulation in a subject having a neurodegenerative disease are provided. In some embodiments, the method comprises administering to the subject an antibody or pharmaceutical composition as described herein. In some embodiments, the subject has Alzheimer's disease.

In yet another aspect, methods of identifying a subject having a neurodegenerative disease as a candidate for treatment with an anti-TREM2 antibody are provided.

In some embodiments, the method comprises:
measuring the level of sTREM2 in a sample from the subject;
comparing the level of sTREM2 in the sample from the subject to a control value, wherein a level of sTREM2 in the sample from the subject that is elevated relative to the control value identifies the subject as a candidate for treatment; and
for a subject identified as a candidate for treatment, administering to the subject an isolated antibody or an antigen-binding portion thereof that specifically binds to a human TREM2 protein (e.g., any antibody described herein). In some embodiments, the isolated antibody or antigen-binding portion thereof is an antibody that decreases levels of sTREM2.

In some embodiments, the method comprises:
measuring the level of sTREM2 in a sample from the subject;
comparing the level of sTREM2 in the sample from the subject to a control value, wherein a level of sTREM2 in the sample from the subject that is reduced relative to the control value identifies the subject as a candidate for treatment; and
for a subject identified as a candidate for treatment, administering to the subject an isolated antibody or an antigen-binding portion thereof that specifically binds to a human TREM2 protein (e.g., any antibody described herein). In some embodiments, the isolated antibody or antigen-binding portion thereof is an antibody that increases levels of sTREM2.

In another aspect, methods of treating a subject having a neurodegenerative disease that has been identified as a candidate for treatment with an anti-TREM2 antibody are provided. In some embodiments, the method comprises:
administering to the subject an isolated antibody or an antigen-binding portion thereof that specifically binds to a human TREM2 protein (e.g., any antibody described herein), wherein the subject has been identified as having an increased level of sTREM2, relative to a control value. In some embodiments, the isolated antibody or antigen-binding portion thereof is an antibody that decreases levels of sTREM2.

In some embodiments, the method comprises:
administering to the subject an isolated antibody or an antigen-binding portion thereof that specifically binds to a human TREM2 protein (e.g., any antibody described herein), wherein the subject has been identified as having a reduced level of sTREM2, relative to a control value. In some embodiments, the isolated antibody or antigen-binding portion thereof is an antibody that increases levels of sTREM2.

In still another aspect, methods of monitoring the efficacy of treatment with an anti-TREM2 antibody for a subject having a neurodegenerative disease are provided. In some embodiments, the method comprises:
measuring the level of sTREM2 in a first sample from the subject taken prior to an administration of an anti-TREM2 antibody (e.g., the first administration to the subject);
treating the subject with an isolated antibody or an antigen-binding portion thereof that specifically binds to a human TREM2 protein (e.g., any antibody described herein); and
measuring the level of sTREM2 in a second sample from the subject taken subsequent to the administration of the anti-TREM2 antibody;
wherein a change in sTREM2 level in the second sample from the subject, as compared to the first sample from the subject, indicates that the subject is responding to treatment with the anti-TREM2 antibody.

In some embodiments, a decrease in sTREM2 level in the second sample from the subject, as compared to the first sample from the subject, indicates that the subject is responding to treatment with the anti-TREM2 antibody. In some embodiments, an increase in sTREM2 level in the second sample from the subject, as compared to the first sample from the subject, indicates that the subject is responding to treatment with the anti-TREM2 antibody.

### BRIEF DESCRIPTION OF THE DRAWINGS

FIGS. 1A-1D. Anti-TREM2 antibodies bind surface TREM2 on cells. (A) Anti-TREM2 antibodies were screened for binding to HEK cells expressing human TREM2 (unlabeled) and mouse TREM2 HEK expressing cells (labeled with NucBlue). Antibody binding was detected with a secondary anti-mouse multiple adsorption antibody conjugated to APC. RS9.F6 demonstrates binding to both mouse and human TREM2 cells. 57D7.A1 binds specifically to human TREM2 cells. (B) Parental HEK-GFP cells were used as a negative control to rule out non-specific binding. (C) Anti-TREM2 antibody surface binding dose response in hTREM2-HEK cells. Anti-TREM2 antibodies were titrated for binding to HEK cells expressing human TREM2. Antibody binding was detected with a secondary anti-mouse multiple adsorption antibody conjugated to APC. RS9.F6 and RS9.F10 demonstrates <2 nM EC50 binding to human TREM2 cells. (D) Anti-TREM2 antibody surface binding dose response in mTREM2-293F cells. Mouse TREM2 HEK cells were titrated for binding as in (C). RS9.F6 and RS9.F10 demonstrates <8 nM EC50 binding to mouse TREM2 HEK cells.

FIG. 2. Anti-TREM2 antibody binding to primary human macrophages. Anti-TREM2 antibodies were screened for binding to primary human monocyte derived macrophages by FACS. Antibody binding was detected with a secondary anti-mouse multiple adsorption antibody conjugated to APC. Gray line=secondary antibody alone. Black line=TREM2 antibody.

FIGS. 3A-3C. P-Syk activation by anti-TREM2 antibodies. (A) Anti-TREM2 antibodies were added at 30 nM to human TREM2 expressing HEK cells induce pSyk as calculated by fold over buffer control. (B-C) P-Syk activation dose response was determined for anti-TREM2 antibodies. Anti-TREM2 antibodies were dose titrated on human TREM2 expressing HEK cells and pSyk induction was calculated by fold over buffer control. EC50 values show nM potency and fold induction of pSyk for each antibody.

FIG. 4. Modulation of soluble TREM2 by anti-TREM2 antibodies. Human TREM2-expressing cells were treated overnight (18 hours) with antibody in solution at the indicated concentration. Soluble TREM2 concentrations were measured by ELISA after denaturation in SDS. Absolute quantities of sTREM2 were determined based on a standard curve. Data was fit with a four-parameter logistic equation.

FIGS. 5A-5B. Anti-TREM2 antibodies induce survival of human macrophages. Human monocytes isolated from peripheral blood were incubated with 5 ng/mL M-CSF (A) or no M-CSF (B) in the presence of titrated concentrations of plate coated anti-TREM2 antibodies or isotype controls. On day 6 cell viability was determined by CellTiter Glo viability assay. Anti-TREM2 antibodies increased survival of human macrophages cultured in restricted or no M-CSF.

FIGS. 6A-6D. Anti-TREM2 antibody induced signaling pathways. (A-D) Human macrophages were stimulated with 30 nM antibody or control for 15 minutes. Cell lysates were measured for p-Y525/526-SYK (A), p-T202/Y204-ERK1/2 (B), p-S9-GSK3-beta (C), and p-S473-AKT (D) by Alpha-LISA. Anti-TREM2 antibodies induced Syk phosphorylation, ERK phosphorylation, GSK3-beta phosphorylation, and AKT phosphorylation.

FIG. 7. Anti-TREM2 antibody epitope bins. Anti-TREM2 antibody epitopes were characterized by competitive binning and demonstrate numerous epitope bins. Cross-competition was assessed using biotinylated detection antibodies and binding was measured with streptavidin-conjugated reagents in an ELISA format. Distance of connecting lines indicate similarity of bin. Circular lines note self-competition which serves as a positive control validating the method.

FIGS. 8A-8C. TREM2 p-Syk induction by novel lipid ligands. (A-B) HEK293 cells stably overexpressing human TREM2 (black bars) and DAP12 (gray bars) (A), or mutant TREM2 R47H (black bars) and DAP12 (gray bars) (B) were stimulated with liposomes containing 30% of the indicated lipids and 70% phosphatidylcholine (PC) at 0.5 mg/mL, except Kdo2-Lipid A (KLA) which contains 10% KLA and 90% PC. pSyk was measured by AlphaLISA, and data are shown as fold change over buffer control (HBSS). Bars represent mean±standard deviation from 1-2 independent experiments. (C) Human macrophages were stimulated with liposomes containing 30% of the indicated lipids and 70% phosphatidylcholine (PC) at 0.5 mg/mL, except Kdo2-Lipid A (KLA) which contains 10% KLA and 90% PC. pSyk was measured by AlphaLISA and data is graphed as fold change over buffer control. Data points represent average value of 2-3 technical replicates from 3 independent human donors. Bars represent mean+standard deviation. ***p<0.001, **p<0.01, *p<0.05 by one-way ANOVA comparison to 100% PC liposomes with Dunnett's posthoc test. See Table 9 for key of lipid abbreviations.

FIGS. 9A-9G. Characterization of anti-TREM2 antibodies' interaction with lipid ligand to activate or block p-Syk. (A) Antibody induction of pSyk in the presence of TREM2 lipid ligand. Anti-TREM2 antibodies dosed at 30 nM on human TREM2 expressing HEK cells either with or without EC20 (0.046 mg/mL), EC50 (0.212 mg/mL), or EC80 (0.967 mg/mL) concentrations of liposomes containing 30% phosphatidylserine (PS)/70% phosphatidylcholine (PC). pSyk activation was calculated by fold over buffer alone control. 21D6.G2 and 3D3.A1 define a class of TREM2 antibody that are additive with lipid TREM2 activators. (B) Antibody induction of pSyk in the presence of TREM2 lipid ligand. Anti-TREM2 antibodies dosed at 10 nM on human macrophages either with or without EC20 (0.046 mg/mL), EC50 (0.212 mg/mL), or EC80 (0.967 mg/mL) concentrations of liposomes containing 30% phosphatidylserine (PS)/70% phosphatidylcholine (PC). The signal due to liposomes alone was subtracted at each value to determine if the antibodies have a synergistic, neutral, or inhibitor effect on lipid ligand driven pSyk activation. (C) Anti-TREM2 antibodies (30 nM) or buffer was incubated with human macrophages for 30 minutes at 37° C., antibody was removed, then liposomes or buffer was added for 5 minutes at 37° C. Detection of pSyk by AlphaLISA on lysed cells was used to determine antibodies that synergize with liposome ligand or block liposome ligand. (D) Percentage of inhibition or synergy was determined by quantifying the extent to which pre-incubating the cells with antibody resulted in blocking or enhanced liposome mediated pSyk signaling. 100% inhibition was defined as entirely blocking the liposome mediated increase in pSyk. (E) Antibody inhibition of pSyk in the presence of TREM2 lipid ligand. 21D4 significantly reduced TREM2 activation by liposomes as compared to controls. (F-G) Antibody inhibition by antibodies 21D4 (F) and 21D11 (G) at increasing antibody concentrations.

FIGS. 10A-10C. ATV-Anti-TREM2 Biacore analysis for TREM2 and hTfR binding. (A) TREM2 binding of RS9.F6/3C35.21.17_LALAPG. (B) TREM2 binding of RS9.F6. (C) Human TfR binding of RS9.F6/3C35.21.17_LALAPG.

FIGS. 11A-11D. Differential heat map comparing time-course hydrogen/deuterium exchange of TREM2 alone to time-course hydrogen/deuterium exchange of TREM2 and F6 Fab mixture for peptides derived from TREM2. (A) Differential heat map over the length of the TREM2 protein of SEQ ID NO:465. The residues of the TREM2 protein are indicated at the top of the heat map. Residues 1-37, which were not covered by the TREM2-derived peptides, are not shown. (B-D) Heat maps showing portions of the differential heat map shown in (A). (B) Differential heat map showing residues 1-68 of the TREM2 protein. (C) Differential heat map showing residues 69-144 of the TREM2 protein. (D) Differential heat map showing residues 145-193 of the TREM2 protein.

FIGS. 12A-12B. F6 Fab-TREM2 peptide co-complex structures. (A) Cartoon representation of the Fab:peptide complex. Shown are the Fab light chain (VL, left), the Fab heavy chain (VH, right), and the TREM2 peptide (center). (B) Interactions at the binding site. Shown are the TREM2 peptide (center), the light chain (left), and the heavy chain (right). Sequential numbering of Fab residues.

FIGS. 13A-13B. F6-TREM2 complex interface residues. (A) Amino acid sequences of the F6 Fab light chain variable domain (residues 1-112 of SEQ ID NO: 112) and heavy chain variable domain (SEQ ID NO:24) with Chothia numbering. CDRs in Kabat definition are underscored. Residues in direct contact with TREM2 peptide are in red. (B) Direct contacts between TREM2 peptide and Fab. Peptide residues are in circles, and Fab residues are in boxes. Sequential numbering of Fab residues.

### DETAILED DESCRIPTION

### I. Introduction

Triggering receptor expressed on myeloid cells-2 (TREM2) is a transmembrane receptor that is expressed on the cell surface of microglia, dendritic cells, macrophages, and osteoclasts. Without being bound to a particular theory, it is believed that upon ligand binding, TREM2 forms a signaling complex with a transmembrane adapter protein, DNAX-activating protein 12 (DAP12), which in turn is tyrosine phosphorylated by the protein kinase SRC. It is believed that the activated TREM2/DAP12 signaling complex mediates intracellular signaling by recruiting and phosphorylating kinases such as Syk kinase.

TREM2/DAP12 signaling modulates activities such as phagocytosis, cell growth and survival, pro-inflammatory cytokine secretion, and the migration of cells such as microglia and macrophages.

TREM2 undergoes regulated intramembrane proteolysis, in which the membrane-associated full-length TREM2 is cleaved by the metalloprotease ADAM10 into a sTREM2 portion that is shed from the cell and a membrane-retained C-terminal fragment that is further degraded by a gamma-secretase. Altered levels of sTREM2 have been reported in patients having Alzheimer's disease or frontotemporal dementia and having a mutation in TREM2. Additionally, mutations in TREM2 are associated with altered functions such as impaired phagocytosis and reduced microglial function.

As detailed in the Examples section below, antibodies have been generated that specifically bind to human TREM2 and that modulate one or more downstream functions of the TREM2/DAP12 signaling complex, such as phosphorylation of Syk kinase. Accordingly, in one aspect, the present disclosure provides anti-TREM2 antibodies and antigen-binding portions thereof.

In some embodiments, the anti-TREM2 antibodies enhance TREM2 activity. Thus, in another aspect, methods of enhancing TREM2 activity, for example in a subject having a neurodegenerative disease, are provided.

In some embodiments, the anti-TREM2 antibodies inhibit TREM2 activity. Thus, in another aspect, methods of inhibiting TREM2 activity, for example in a subject having a neurodegenerative disease, are provided.

In some embodiments, the anti-TREM2 antibodies of the disclosure reduce shedding of sTREM2. Thus, in yet another aspect, methods of decreasing levels of sTREM2, for example in a subject having a neurodegenerative disease, are provided.

In some embodiments, the anti-TREM2 antibodies of the disclosure increase shedding of sTREM2. Accordingly, in still another aspect, methods of increasing levels of sTREM2, for example in a subject having a neurodegenerative disease, are provided.

### II. Definitions

As used herein, the singular forms “a,” “an” and “the” include plural referents unless the content clearly dictates otherwise. Thus, for example, reference to “an antibody” optionally includes a combination of two or more such molecules, and the like.

As used herein, the terms “about” and “approximately,” when used to modify an amount specified in a numeric value or range indicate that the numeric value as well as reasonable deviations from the value known to the skilled person in the art, for example ±20%, ±10%, or ±5%, are within the intended meaning of the recited value.

As used herein, the term “TREM2 protein” refers to a triggering receptor expressed on myeloid cells 2 protein that is encoded by the gene Trem2. As used herein, a “TREM2 protein” refers to a native (i.e., wild-type) TREM2 protein of any vertebrate, such as but not limited to human, non-human primates (e.g., cynomolgus monkey), rodents (e.g., mice, rat), and other mammals. In some embodiments, a TREM2 protein is a human TREM2 protein having the sequence identified in UniprotKB accession number Q9NZC2 (SEQ ID NO:96).

As used herein, the term “anti-TREM2 antibody” refers to an antibody that specifically binds to a TREM2 protein (e.g., human TREM2).

As used herein, the term “antibody” refers to a protein with an immunoglobulin fold that specifically binds to an antigen via its variable regions. The term encompasses intact polyclonal antibodies, intact monoclonal antibodies, single chain antibodies, multispecific antibodies such as bispecific antibodies, monospecific antibodies, monovalent antibodies, chimeric antibodies, humanized antibodies, and human antibodies. The term “antibody,” as used herein, also includes antibody fragments that retain binding specificity, including but not limited to Fab, F(ab′)₂, Fv, scFv, and bivalent scFv. Antibodies can contain light chains that are classified as either kappa or lambda. Antibodies can contain heavy chains that are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms “variable light chain” (VL) and “variable heavy chain” (VH) refer to these light and heavy chains, respectively.

The term “variable region” or “variable domain” refers to a domain in an antibody heavy chain or light chain that is derived from a germline Variable (V) gene, Diversity (D) gene, or Joining (J) gene (and not derived from a Constant (Cμ and Cδ) gene segment), and that gives an antibody its specificity for binding to an antigen. Typically, an antibody variable region comprises four conserved “framework” regions interspersed with three hypervariable “complementarity determining regions.”

The term “complementarity determining region” or “CDR” refers to the three hypervariable regions in each chain that interrupt the four framework regions established by the light and heavy chain variable regions. The CDRs are primarily responsible for antibody binding to an epitope of an antigen. The CDRs of each chain are typically referred to as CDR1, CDR2, and CDR3, numbered sequentially starting from the N-terminus, and are also typically identified by the chain in which the particular CDR is located. Thus, a VH CDR3 or CDR-H3 is located in the variable region of the heavy chain of the antibody in which it is found, whereas a VL CDR1 or CDR-L1 is the CDR1 from the variable region of the light chain of the antibody in which it is found.

The “framework regions” or “FRs” of different light or heavy chains are relatively conserved within a species. The framework region of an antibody, that is the combined framework regions of the constituent light and heavy chains, serves to position and align the CDRs in three-dimensional space. Framework sequences can be obtained from public DNA databases or published references that include germline antibody gene sequences. For example, germline DNA sequences for human heavy and light chain variable region genes can be found in the “VBASE2” germline variable gene sequence database for human and mouse sequences.

The amino acid sequences of the CDRs and framework regions can be determined using various well-known definitions in the art, e.g., Kabat, Chothia, international ImMunoGeneTics database (IMGT), AbM, and observed antigen contacts (“Contact”). In some embodiments, CDRs are determined according to the Contact definition. See, MacCallum et al., J. Mol. Biol., 262:732-745 (1996). In some embodiments, CDRs are determined by a combination of Kabat, Chothia, and/or Contact CDR definitions.

The terms “antigen-binding portion” and “antigen-binding fragment” are used interchangeably herein and refer to one or more fragments of an antibody that retains the ability to specifically bind to an antigen (e.g., a TREM2 protein) via its variable region. Examples of antigen-binding fragments include, but are not limited to, a Fab fragment (a monovalent fragment consisting of the VL, VH, CL and CH1 domains), F(ab′)₂ fragment (a bivalent fragment comprising two Fab fragments linked by a disulfide bridge at the hinge region), single chain Fv (scFv), disulfide-linked Fv (dsFv), complementarity determining regions (CDRs), a VL (light chain variable region), and a VH (heavy chain variable region).

The term “epitope” refers to the area or region of an antigen to which the CDRs of an antibody specifically binds and can include a few amino acids or portions of a few amino acids, e.g., 5 or 6, or more, e.g., 20 or more amino acids, or portions of those amino acids. For example, where the target is a protein, the epitope can be comprised of consecutive amino acids (e.g., a linear epitope), or amino acids from different parts of the protein that are brought into proximity by protein folding (e.g., a discontinuous or conformational epitope). In some embodiments, the epitope is phosphorylated at one amino acid (e.g., at a serine or threonine residue).

As used herein, the phrase “recognizes an epitope,” as used with reference to an anti-TREM2 antibody, means that the antibody CDRs interact with or specifically bind to the antigen (i.e., the TREM2 protein) at that epitope or a portion of the antigen containing that epitope.

As used herein, the term “multispecific antibody” refers to an antibody that comprises two or more different antigen-binding portions, in which each antigen-binding portion comprises a different variable region that recognizes a different antigen, or a fragment or portion of the antibody that binds to the two or more different antigens via its variable regions. As used herein, the term “bispecific antibody” refers to an antibody that comprises two different antigen-binding portions, in which each antigen-binding portion comprises a different variable region that recognizes a different antigen, or a fragment or portion of the antibody that binds to the two different antigens via its variable regions.

A “monoclonal antibody” refers to antibodies produced by a single clone of cells or a single cell line and consisting of or consisting essentially of antibody molecules that are identical in their primary amino acid sequence.

A “polyclonal antibody” refers to an antibody obtained from a heterogeneous population of antibodies in which different antibodies in the population bind to different epitopes of an antigen.

A “chimeric antibody” refers to an antibody molecule in which the constant region, or a portion thereof, is altered, replaced or exchanged so that the antigen-binding site (i.e., variable region, CDR, or portion thereof) is linked to a constant region of a different or altered class, effector function and/or species, or in which the variable region, or a portion thereof, is altered, replaced or exchanged with a variable region having a different or altered antigen specificity (e.g., CDR and framework regions from different species). In some embodiments, a chimeric antibody is a monoclonal antibody comprising a variable region from one source or species (e.g., mouse) and a constant region derived from a second source or species (e.g., human). Methods for producing chimeric antibodies are described in the art.

A “humanized antibody” is a chimeric antibody derived from a non-human source (e.g., murine) that contains minimal sequences derived from the non-human immunoglobulin outside the CDRs. In general, a humanized antibody will comprise at least one (e.g., two) antigen-binding variable domain(s), in which the CDR regions substantially correspond to those of the non-human immunoglobulin and the framework regions substantially correspond to those of a human immunoglobulin sequence. In some instances, certain framework region residues of a human immunoglobulin can be replaced with the corresponding residues from a non-human species to, e.g., improve specificity, affinity, and/or serum half-life. The humanized antibody can also comprise at least a portion of an immunoglobulin constant region (Fc), typically that of a human immunoglobulin sequence. Methods of antibody humanization are known in the art.

A “human antibody” or a “fully human antibody” is an antibody having human heavy chain and light chain sequences, typically derived from human germline genes. In some embodiments, the antibody is produced by a human cell, by a non-human animal that utilizes human antibody repertoires (e.g., transgenic mice that are genetically engineered to express human antibody sequences), or by phage display platforms.

The term “specifically binds” refers to a molecule (e.g., an antibody (or an antigen-binding portion thereof) or a modified Fc polypeptide (or a target-binding portion thereof)) that binds to an epitope or target with greater affinity, greater avidity, and/or greater duration to that epitope or target in a sample than it binds to another epitope or non-target compound (e.g., a structurally different antigen). In some embodiments, an antibody (or an antigen-binding portion thereof) or a modified Fc polypeptide (or a target-binding portion thereof) that specifically binds to an epitope or target is an antibody (or an antigen-binding portion thereof) or a modified Fc polypeptide (or a target-binding portion thereof) that binds to the epitope or target with at least 5-fold greater affinity than other epitopes or non-target compounds, e.g., at least 6-fold, 7-fold, 8-fold, 9-fold, 10-fold, 20-fold, 25-fold, 50-fold, 100-fold, 1,000-fold, 10,000-fold, or greater affinity. In some embodiments, an antibody that specifically binds to a TREM2 protein (e.g., human TREM2) binds to the TREM2 protein with at least a 5-fold greater affinity than to a non-TREM2 protein (e.g., at least 10-fold, 50-fold, 100-fold, 1,000-fold, 10,000-fold or greater affinity). The term “specific binding,” “specifically binds to,” or “is specific for” a particular epitope or target, as used herein, can be exhibited, for example, by a molecule having an equilibrium dissociation constant K_{D} for the epitope or target to which it binds of, e.g., 10⁻⁴ M or smaller, e.g., 10⁻⁵ M, 10⁻⁶ M, 10⁻⁷ M, 10⁻⁸ M, 10⁻⁹ M, 10⁻¹⁰ M, 10⁻¹¹ M, or 10⁻¹² M. It will be recognized by one of skill that an antibody that specifically binds to a TREM2 protein from one species may also specifically bind to orthologs of the TREM2 protein.

The term “binding affinity” is used herein to refer to the strength of a non-covalent interaction between two molecules, e.g., between an antibody (or an antigen-binding portion thereof) and an antigen, or between a modified Fc polypeptide (or a target-binding portion thereof) and a target. Thus, for example, the term may refer to 1:1 interactions between an antibody (or an antigen-binding portion thereof) and an antigen or between a modified Fc polypeptide (or a target-binding portion thereof) and a target, unless otherwise indicated or clear from context. Binding affinity may be quantified by measuring an equilibrium dissociation constant (K_{D}), which refers to the dissociation rate constant (k_{d}, time⁻¹) divided by the association rate constant (kₐ, time⁻¹ M⁻¹). K_{D} can be determined by measurement of the kinetics of complex formation and dissociation, e.g., using Surface Plasmon Resonance (SPR) methods, e.g., a Biacore™ system; kinetic exclusion assays such as KinExA®; and BioLayer interferometry (e.g., using the ForteBio® Octet platform). As used herein, “binding affinity” includes not only formal binding affinities, such as those reflecting 1:1 interactions between an antibody (or an antigen-binding portion thereof) and an antigen or between a modified Fc polypeptide (or a target-binding portion thereof) and a target, but also apparent affinities for which K_{DS} are calculated that may reflect avid binding.

A “transferrin receptor” or “TfR,” as used herein, refers to transferrin receptor protein 1. The human transferrin receptor 1 polypeptide sequence is set forth in SEQ ID NO:97. Transferrin receptor protein 1 sequences from other species are also known (e.g., chimpanzee, accession number XP_003310238.1; rhesus monkey, NP_001244232.1; dog, NP_001003111.1; cattle, NP_001193506.1; mouse, NP_035768.1; rat, NP_073203.1; and chicken, NP_990587.1). The term “transferrin receptor” also encompasses allelic variants of exemplary reference sequences, e.g., human sequences, that are encoded by a gene at a transferrin receptor protein 1 chromosomal locus. Full-length transferrin receptor protein includes a short N-terminal intracellular region, a transmembrane region, and a large extracellular domain. The extracellular domain is characterized by three domains: a protease-like domain, a helical domain, and an apical domain.

As used herein, the term “Fc polypeptide” refers to the C-terminal region of a naturally occurring immunoglobulin heavy chain polypeptide that is characterized by an Ig fold as a structural domain. An Fc polypeptide contains constant region sequences including at least the CH2 domain and/or the CH3 domain and may contain at least part of the hinge region, but does not contain a variable region.

A “modified Fc polypeptide” refers to an Fc polypeptide that has at least one mutation, e.g., a substitution, deletion or insertion, as compared to a wild-type immunoglobulin heavy chain Fc polypeptide sequence, but retains the overall Ig fold or structure of the native Fc polypeptide.

As used herein, the term “FcRn” refers to the neonatal Fc receptor. Binding of Fc polypeptides to FcRn reduces clearance and increases serum half-life of the Fc polypeptide. The human FcRn protein is a heterodimer that is composed of a protein of about 50 kDa in size that is similar to a major histocompatibility (MHC) class I protein and a β2-microglobulin of about 15 kDa in size.

As used herein, an “FcRn binding site” refers to the region of an Fc polypeptide that binds to FcRn. In human IgG, the FcRn binding site, as numbered using the EU index, includes L251, M252, I253, S254, R255, T256, M428, H433, N434, H435, and Y436. These positions correspond to positions 21 to 26, 198, and 203 to 206 of SEQ ID NO:98.

As used herein, a “native FcRn binding site” refers to a region of an Fc polypeptide that binds to FcRn and that has the same amino acid sequence as the region of a naturally occurring Fc polypeptide that binds to FcRn.

As used herein, the terms “CH3 domain” and “CH2 domain” refer to immunoglobulin constant region domain polypeptides. For purposes of this application, a CH3 domain polypeptide refers to the segment of amino acids from about position 341 to about position 447 as numbered according to the EU numbering scheme, and a CH2 domain polypeptide refers to the segment of amino acids from about position 231 to about position 340 as numbered according to the EU numbering scheme and does not include hinge region sequences. CH2 and CH3 domain polypeptides may also be numbered by the IMGT (ImMunoGeneTics) numbering scheme in which the CH2 domain numbering is 1-110 and the CH3 domain numbering is 1-107, according to the IMGT Scientific chart numbering (IMGT website). CH2 and CH3 domains are part of the Fc region of an immunoglobulin. An Fc region refers to the segment of amino acids from about position 231 to about position 447 as numbered according to the EU numbering scheme, but as used herein, can include at least a part of a hinge region of an antibody. An illustrative hinge region sequence is the human IgG1 hinge sequence EPKSCDKTHTCPPCP (SEQ ID NO:99).

The terms “wild-type,” “native,” and “naturally occurring,” as used with reference to a CH3 or CH2 domain, refer to a domain that has a sequence that occurs in nature.

As used herein, the term “mutant,” as used with reference to a mutant polypeptide or mutant polynucleotide is used interchangeably with “variant.” A variant with respect to a given wild-type CH3 or CH2 domain reference sequence can include naturally occurring allelic variants. A “non-naturally” occurring CH3 or CH2 domain refers to a variant or mutant domain that is not present in a cell in nature and that is produced by genetic modification, e.g., using genetic engineering technology or mutagenesis techniques, of a native CH3 domain or CH2 domain polynucleotide or polypeptide. A “variant” includes any domain comprising at least one amino acid mutation with respect to wild-type. Mutations may include substitutions, insertions, and deletions.

The term “cross-reacts,” as used herein, refers to the ability of an antibody to bind to an antigen other than the antigen against which the antibody was raised. In some embodiments, cross-reactivity refers to the ability of an antibody to bind to an antigen from another species than the antigen against which the antibody was raised. As a non-limiting example, an anti-TREM2 antibody as described herein that is raised against a human TREM2 peptide can exhibit cross-reactivity with a TREM2 peptide or protein from a different species (e.g., monkey or mouse).

The term “isolated,” as used with reference to a nucleic acid or protein (e.g., antibody), denotes that the nucleic acid or protein is essentially free of other cellular components with which it is associated in the natural state. Purity and homogeneity are typically determined using analytical chemistry techniques such as electrophoresis (e.g., polyacrylamide gel electrophoresis) or chromatography (e.g., high performance liquid chromatography). In some embodiments, an isolated nucleic acid or protein (e.g., antibody) is at least 85% pure, at least 90% pure, at least 95% pure, or at least 99% pure.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, y-carboxyglutamate, and O-phosphoserine. “Amino acid analogs” refer to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid. Amino acids may be referred to herein by either their commonly known three letter symbols or by the one-letter symbols recommended by the IUPAC-IUB Biochemical Nomenclature Commission.

The terms “polypeptide” and “peptide,” are used interchangeably herein to refer to a polymer of amino acid residues in a single chain. The terms apply to amino acid polymers in which one or more amino acid residue is an artificial chemical mimetic of a corresponding naturally occurring amino acid, as well as to naturally occurring amino acid polymers and non-naturally occurring amino acid polymers. Amino acid polymers may comprise entirely L-amino acids, entirely D-amino acids, or a mixture of L and D amino acids.

The term “protein” as used herein refers to either a polypeptide or a dimer (i.e, two) or multimer (i.e., three or more) of single chain polypeptides. The single chain polypeptides of a protein may be joined by a covalent bond, e.g., a disulfide bond, or non-covalent interactions.

The terms “polynucleotide” and “nucleic acid” interchangeably refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. Examples of polynucleotides contemplated herein include single- and double-stranded DNA, single- and double-stranded RNA, and hybrid molecules having mixtures of single- and double-stranded DNA and RNA.

The terms “conservative substitution” and “conservative mutation” refer to an alteration that results in the substitution of an amino acid with another amino acid that can be categorized as having a similar feature. Examples of categories of conservative amino acid groups defined in this manner can include: a “charged/polar group” including Glu (Glutamic acid or E), Asp (Aspartic acid or D), Asn (Asparagine or N), Gln (Glutamine or Q), Lys (Lysine or K), Arg (Arginine or R), and His (Histidine or H); an “aromatic group” including Phe (Phenylalanine or F), Tyr (Tyrosine or Y), Trp (Tryptophan or W), and (Histidine or H); and an “aliphatic group” including Gly (Glycine or G), Ala (Alanine or A), Val (Valine or V), Leu (Leucine or L), Ile (Isoleucine or I), Met (Methionine or M), Ser (Serine or S), Thr (Threonine or T), and Cys (Cysteine or C). Within each group, subgroups can also be identified. For example, the group of charged or polar amino acids can be sub-divided into sub-groups including: a “positively-charged sub-group” comprising Lys, Arg and His; a “negatively-charged sub-group” comprising Glu and Asp; and a “polar sub-group” comprising Asn and Gln. In another example, the aromatic or cyclic group can be sub-divided into sub-groups including: a “nitrogen ring sub-group” comprising Pro, His and Trp; and a “phenyl sub-group” comprising Phe and Tyr. In another further example, the aliphatic group can be sub-divided into sub-groups, e.g., an “aliphatic non-polar sub-group” comprising Val, Leu, Gly, and Ala; and an “aliphatic slightly-polar sub-group” comprising Met, Ser, Thr, and Cys. Examples of categories of conservative mutations include amino acid substitutions of amino acids within the sub-groups above, such as, but not limited to: Lys for Arg or vice versa, such that a positive charge can be maintained; Glu for Asp or vice versa, such that a negative charge can be maintained; Ser for Thr or vice versa, such that a free —OH can be maintained; and Gln for Asn or vice versa, such that a free —NH₂ can be maintained. In some embodiments, hydrophobic amino acids are substituted for naturally occurring hydrophobic amino acid, e.g., in the active site, to preserve hydrophobicity.

The terms “identical” or percent “identity,” in the context of two or more polypeptide sequences, refer to two or more sequences or subsequences that are the same or have a specified percentage of amino acid residues, e.g., at least 60% identity, at least 65%, at least 70%, at least 75%, at least 80%, at least 85%, at least 90%, or at least 95% or greater, that are identical over a specified region when compared and aligned for maximum correspondence over a comparison window, or designated region as measured using a sequence comparison algorithm or by manual alignment and visual inspection.

For sequence comparison of polypeptides, typically one amino acid sequence acts as a reference sequence, to which a candidate sequence is compared. Alignment can be performed using various methods available to one of skill in the art, e.g., visual alignment or using publicly available software using known algorithms to achieve maximal alignment. Such programs include the BLAST programs, ALIGN, ALIGN-2 (Genentech, South San Francisco, Calif.) or Megalign (DNASTAR). The parameters employed for an alignment to achieve maximal alignment can be determined by one of skill in the art. For sequence comparison of polypeptide sequences for purposes of this application, the BLASTP algorithm standard protein BLAST for aligning two proteins sequence with the default parameters is used.

The terms “corresponding to,” “determined with reference to,” or “numbered with reference to” when used in the context of the identification of a given amino acid residue in a polypeptide sequence, refers to the position of the residue of a specified reference sequence when the given amino acid sequence is maximally aligned and compared to the reference sequence. Thus, for example, an amino acid residue in a modified Fc polypeptide “corresponds to” an amino acid in SEQ ID NO:98, when the residue aligns with the amino acid in SEQ ID NO:98 when optimally aligned to SEQ ID NO:98. The polypeptide that is aligned to the reference sequence need not be the same length as the reference sequence.

The terms “subject,” “individual,” and “patient,” as used interchangeably herein, refer to a mammal, including but not limited to humans, non-human primates, rodents (e.g., rats, mice, and guinea pigs), rabbits, cows, pigs, horses, and other mammalian species. In one embodiment, the subject, individual, or patient is a human.

The terms “treating,” “treatment,” and the like are used herein to generally mean obtaining a desired pharmacologic and/or physiologic effect. “Treating” or “treatment” may refer to any indicia of success in the treatment or amelioration of a neurodegenerative disease (e.g., Alzheimer's disease or another neurodegenerative disease described herein), including any objective or subjective parameter such as abatement, remission, improvement in patient survival, increase in survival time or rate, diminishing of symptoms or making the disease more tolerable to the patient, slowing in the rate of degeneration or decline, or improving a patient's physical or mental well-being. The treatment or amelioration of symptoms can be based on objective or subjective parameters. The effect of treatment can be compared to an individual or pool of individuals not receiving the treatment, or to the same patient prior to treatment or at a different time during treatment.

The term “pharmaceutically acceptable excipient” refers to a non-active pharmaceutical ingredient that is biologically or pharmacologically compatible for use in humans or animals, such as, but not limited to a buffer, carrier, or preservative.

As used herein, a “therapeutic amount” or “therapeutically effective amount” of an agent (e.g., an antibody as described herein) is an amount of the agent that treats, alleviates, abates, or reduces the severity of symptoms of a disease in a subject. A “therapeutic amount” of an agent (e.g., an antibody as described herein) may improve patient survival, increase survival time or rate, diminish symptoms, make an injury, disease, or condition (e.g., a neurodegenerative disease) more tolerable, slow the rate of degeneration or decline, or improve a patient's physical or mental well-being.

The term “administer” refers to a method of delivering agents, compounds, or compositions to the desired site of biological action. These methods include, but are not limited to, topical delivery, parenteral delivery, intravenous delivery, intradermal delivery, intramuscular delivery, intrathecal delivery, colonic delivery, rectal delivery, or intraperitoneal delivery. In one embodiment, an antibody as described herein is administered intravenously.

The term “selectively enhances,” as used in the context of a TREM2 antibody enhancing activity that is induced by a TREM2 ligand, means that the antibody enhances the activity of the TREM2 ligand to a greater extent (e.g., at least 1.5-fold, 2-fold, 2.5-fold, 3-fold, 4-fold, 5-fold, 8-fold, 10-fold, 15-fold, 20-fold, 30-fold, or 50-fold) as compared to an appropriate reference, for example, its enhancement of a reference TREM2 ligand (e.g., any described herein) or as compared to the average enhance of a group of TREM2 ligands (e.g., any or all described herein.

The term “control” or “control value” refers to a reference value or baseline value. Appropriate controls can be determined by one skilled in the art. In some instances, control values can be determined relative to a baseline within the same subject or experiment, e.g., a measurement of sTREM2 taken prior to treatment with an anti-TREM2 antibody can be a control value for a post-treatment measurement of sTREM2 levels in the same subject. In other instances, the control value can be determined relative to a control subject (e.g., a healthy control or a disease control) or an average value in a population of control subjects (e.g., healthy controls or disease controls, e.g., a population of 10, 20, 50, 100, 200, 500, 1000 control subjects or more), e.g, a measurement of a subject's level of sTREM2 either at baseline or after treatment can be compared to a healthy control value.

### III. Anti-Trem2 Antibodies

In one aspect, antibodies and antigen-binding portions thereof that specifically bind to a TREM2 protein are provided. In some embodiments, the antibody specifically binds to a human TREM2 protein. In some embodiments, an anti-TREM2 antibody is selective for TREM2 over other TREM-like receptors (e.g., TREM1).

In some embodiments, an antibody that specifically binds to TREM2 is an antibody having one or more TREM2 activities as described herein, e.g., is an antibody that modulates recruitment or phosphorylation of a kinase that interacts with a TREM2/DAP12 signaling complex (e.g., Syk kinase), modulates phagocytosis, modulates cell migration, and/or modulates cell differentiation; modulates levels of sTREM2; modulates ligand activation of TREM2; recognizes an epitope that is the same or substantially the same as the epitope recognized by antibody clone as described herein (e.g., 2G4.B1, 3D3.A1, 7B10.A2, 8A11.B1, 13B11.A1, 14D5.F1, 14H11.A1, 19F10.F3, 21D4.D1, 21D6.G2, 21D11, 22B8.B1, 22G9.D1, 24B4.A1, 26D2.D1, 26D5.A1, 26D11.B1, 26E2.A3, 30A8.A1, 30F2.A2, 38E9.E5, 39H10.A1, 40H3.A4, 42E8.H1, 43E9.H1, 44E2.H1, 44E3.B1, 49H11.B1, 51D4, 52H9.D1, 53H11.D3, 54C2.A1, 55B9.A1, 57D7.A1, 59C6.F1, 60A4.B1, RS9.E2, RS9.F6, RS9.F10, RS11.1F5, RS11.1G6, RS11.1A10, RS11.1D11, RS11.4A5, RS11.4H6, RS11.4D7, RS11.4D9, RS11.4F11, RS12.1C6, RS12.1C10, RS12.2D1, RS12.2D4, RS12.2E1, RS12.2F2, RS12.2G1, RS12.2H1, or RS12.3C10); and/or has one or more CDR, heavy chain variable region, and/or light chain variable region sequences as an antibody clone described herein (e.g., 2G4.B1, 3D3.A1, 7B10.A2, 8A11.B1, 13B11.A1, 14D5.F1, 14H11.A1, 19F10.F3, 21D4.D1, 21D6.G2, 21D11, 22B8.B1, 22G9.D1, 24B4.A1, 26D2.D1, 26D5.A1, 26D11.B1, 26E2.A3, 30A8.A1, 30F2.A2, 38E9.E5, 39H10.A1, 40H3.A4, 42E8.H1, 43E9.H1, 44E2.H1, 44E3.B1, 49H11.B1, 51D4, 52H9.D1, 53H11.D3, 54C2.A1, 55B9.A1, 57D7.A1, 59C6.F1, 60A4.B1, RS9.E2, RS9.F6, RS9.F10, RS11.1F5, RS11.1G6, RS11.1A10, RS11.1D11, RS11.4A5, RS11.4H6, RS11.4D7, RS11.4D9, RS11.4F11, RS12.1C6, RS12.1C10, RS12.2D1, RS12.2D4, RS12.2E1, RS12.2F2, RS12.2G1, RS12.2H1, or RS12.3C10).

Anti-TREM2 Antibodies that Modulate sTREM2 Shedding

In some embodiments, an anti-TREM2 antibody alters levels of sTREM2 protein in a sample, e.g., levels of sTREM2 that are shed from the cell surface into an extracellular sample. In some embodiments, an anti-TREM2 antibody decreases levels of sTREM2. In some embodiments, an anti-TREM2 antibody increases levels of sTREM2.

In some embodiments, an anti-TREM2 antibody decreases levels of sTREM2 if the amount of sTREM2 in a treated sample is decreased by at least 10%, at least 20%, at least 30%, at least 40%, at least 50%, at least 60%, at least 70%, at least 80%, at least 90% or more as compared to a control value. In some embodiments, an anti-TREM2 antibody decreases levels of sTREM2 if the amount of sTREM2 in a treated sample is decreased by at least 2-fold, 3-fold, 4-fold, 5-fold, 6-fold, 7-fold, 8-fold, 9-fold, 10-fold or more as compared to a control value. In some embodiments, the control value is the amount of sTREM2 in an untreated sample (e.g., a supernatant from a TREM2-expressing cell that has not been treated with an anti-TREM2 antibody, or a sample from a subject that has not been treated with an anti-TREM2 antibody) or a sample treated with an appropriate non-TREM2-binding antibody.

In some embodiments, an anti-TREM2 antibody increases levels of sTREM2 if the amount of sTREM2 in a treated sample is increased by at least 10%, at least 20%, at least 30%, at least 40%, at least 50%, at least 60%, at least 70%, at least 80%, at least 90% or more as compared to a control value. In some embodiments, an anti-TREM2 antibody increases levels of sTREM2 if the amount of sTREM2 in a treated sample is increased by at least 2-fold, 3-fold, 4-fold, 5-fold, 6-fold, 7-fold, 8-fold, 9-fold, 10-fold or more as compared to a control value. In some embodiments, the control value is the amount of sTREM2 in an untreated sample (e.g., a supernatant from a TREM2-expressing cell that has not been treated with an anti-TREM2 antibody, or a sample from a subject that has not been treated with an anti-TREM2 antibody) or a sample treated with an appropriate non-TREM2-binding antibody.

In some embodiments, sTREM2 shedding is measured using a sample that comprises a fluid, e.g., blood, plasma, serum, urine, or cerebrospinal fluid. In some embodiments, the sample comprises cerebrospinal fluid. In some embodiments, the sample comprises supernatant from cell cultures (e.g., supernatant from a primary cell or cell line that endogenously expresses TREM2, such as human macrophages, or a primary cell or cell line that has been engineered to express TREM2, e.g., as described in the Examples section below).

In some embodiments, the level of sTREM2 in a sample is measured using an immunoassay. Immunoassays are known in the art and include, but are not limited to, enzyme immunoassays (EIA) such as enzyme multiplied immunoassay (EMIA), enzyme-linked immunosorbent assay (ELISA), microparticle enzyme immunoassay (MEIA), immunohistochemistry (IHC), immunocytochemistry, capillary electrophoresis immunoassays (CEIA), radioimmunoassays (RIA), immunofluorescence, chemiluminescence immunoassays (CL), and electrochemiluminescence immunoassays (ECL). In some embodiments, sTREM2 levels are measuring using an ELISA assay. In some embodiments, sTREM2 levels are measured using an ELISA assay as described in the Examples section below.

In some embodiments, an anti-TREM2 antibody that decreases levels of sTREM2 also modulates one or more TREM2 activities as described below. In some embodiments, an anti-TREM2 antibody that increases levels of sTREM2 also modulates one or more TREM2 activities as described below.

Anti-TREM2 Antibodies that Modulate TREM2 Activities

In some embodiments, an anti-TREM2 antibody modulates one or more TREM2 activities. For example, in some embodiments, an anti-TREM2 antibody modulates the recruitment or phosphorylation of a kinase that interacts with the TREM2/DAP12 signaling complex. In some embodiments, the anti-TREM2 antibody modulates one or more downstream activities such as phagocytosis, cell growth, cell survival, cell differentiation, cytokine secretion, or cell migration.

In some embodiments, an anti-TREM2 antibody enhances one or more TREM2 activities, including but not limited to inducing phosphorylation of a kinase that interacts with the TREM2/DAP12 signaling complex, enhancing phagocytosis (e.g., phagocytosis of cell debris, amyloid beta particles, etc.), enhancing cell migration (e.g., migration of microglia or macrophages), enhancing cell function (e.g., for myeloid cells, microglia, including disease associated microglia, and macrophages), and/or enhancing cell survival or cell differentiation (e.g., for myeloid cells, microglia, including disease associated microglia, and macrophages).

In some embodiments, an anti-TREM2 antibody enhances one or more TREM2 activities (e.g., those described above) that are induced by a ligand. An anti-TREM2 antibody that enhances one or more TREM2 activities that is induced by a ligand is referred to herein as a “positive allosteric modulator” (“PAM”). In some embodiments, an anti-TREM2 antibody enhances one or more TREM2 activities without blocking binding of a native TREM2 ligand. In some embodiments, an anti-TREM2 antibody blocks binding of a TREM2 ligand to TREM2. In some embodiments, an anti-TREM2 antibody enhances one or more TREM2 activities that is induced by a ligand but does not enhance TREM2 activity in the absence of a ligand. In some embodiments, an anti-TREM2 antibody selectively enhances activity of a TREM2 ligand. In some embodiments, an anti-TREM2 antibody prevents activation of TREM2 by a TREM2 ligand.

In some embodiments, an anti-TREM2 antibody inhibits one or more TREM2 activities, including but not limited to decreasing or inhibiting phosphorylation of a kinase that interacts with the TREM2/DAP12 signaling complex, reducing or inhibiting phagocytosis (e.g., phagocytosis of cell debris, amyloid beta particles, etc.), decreasing or inhibiting cell migration (e.g., migration of microglia or macrophages), and/or decreasing or inhibiting cell survival or cell differentiation (e.g., for myeloid cells, microglia, including disease associated microglia, and macrophages). In some embodiments, an anti-TREM2 antibody inhibits one or more TREM2 activities that are induced by a ligand. An anti-TREM2 antibody that inhibits one or more TREM2 activities that is induced by a ligand is referred to herein as a “negative allosteric modulator” (“NAM”). In some embodiments, an anti-TREM2 antibody inhibits one or more TREM2 activities that is induced by a ligand but does not inhibit TREM2 activity in the absence of a ligand. In some embodiments, an anti-TREM2 antibody inhibits TREM2 activity in the absence of a TREM2 ligand and inhibits one or more TREM2 activities that is induced by a ligand. In some embodiments, an anti-TREM2 antibody prevents activation of TREM2 by a TREM2 ligand. In some embodiments, an anti-TREM2 antibody binds TREM2 at the ligand-binding site. In some embodiments, an anti-TREM2 antibody blocks binding of a TREM2 ligand to TREM2.

Kinase Phosphorylation

In some embodiments, an anti-TREM2 antibody induces phosphorylation of a kinase that interacts with the TREM2/DAP12 signaling complex (such as, but not limited to, Syk, ZAP70, PI3K, Erk, AKT, or GSK3b). In some embodiments, an anti-TREM2 antibody induces phosphorylation of a kinase that interacts with the TREM2/DAP12 signaling complex without blocking binding of a native TREM2 ligand. In some embodiments, an anti-TREM2 antibody induces phosphorylation of Syk. In some embodiments, an anti-TREM2 antibody induces phosphorylation of Syk if the level of Syk phosphorylation in a sample treated with the anti-TREM2 antibody is increased by at least 10%, at least 20%, at least 30%, at least 40%, at least 50%, at least 60%, at least 70%, at least 80%, at least 90% or more as compared to a control value. In some embodiments, an anti-TREM2 antibody induces phosphorylation of Syk if the level of Syk phosphorylation in a sample treated with the anti-TREM2 antibody is increased by at least 2-fold, 3-fold, 4-fold, 5-fold, 6-fold, 7-fold, 8-fold, 9-fold, 10-fold, or more as compared to a control value. In some embodiments, the control value is the level of Syk phosphorylation in an untreated sample (e.g., a sample comprising a TREM2-expressing cell that has not been treated with an anti-TREM2 antibody, or a sample from a subject that has not been treated with an anti-TREM2 antibody) or a sample treated with an appropriate non-TREM2-binding antibody.

In some embodiments, an anti-TREM2 antibody induces phosphorylation of a kinase that interacts with the TREM2/DAP12 signaling complex (such as, but not limited to, Syk, ZAP70, PI3K, Erk, AKT, or GSK3b) in the presence of a ligand. In some embodiments, an anti-TREM2 antibody induces phosphorylation of Syk in the presence of a ligand. In some embodiments, an anti-TREM2 antibody induces phosphorylation of Syk in the presence of a ligand if the level of Syk phosphorylation in a sample treated with a TREM2 ligand and the anti-TREM2 antibody is increased by at least 10%, at least 20%, at least 30%, at least 40%, at least 50%, at least 60%, at least 70%, at least 80%, at least 90% or more as compared to a control value. In some embodiments, an anti-TREM2 antibody induces phosphorylation of Syk in the presence of a ligand if the level of Syk phosphorylation in a sample treated with a TREM2 ligand and the anti-TREM2 antibody is increased by at least 2-fold, 3-fold, 4-fold, 5-fold, 6-fold, 7-fold, 8-fold, 9-fold, 10-fold, 15-fold, 20-fold, or more as compared to a control value (e.g., the level in the presence of the ligand but in the absence of the antibody). In some embodiments, the control value is the level of Syk phosphorylation in an untreated sample (e.g., a sample comprising a TREM2-expressing cell that has not been treated with a TREM2 ligand or an anti-TREM2 antibody, or a sample from a subject that has not been treated with a TREM2 ligand or an anti-TREM2 antibody) or a sample treated with an appropriate non-TREM2-binding antibody.

In some embodiments, an anti-TREM2 antibody induces phosphorylation of a kinase that interacts with the TREM2/DAP12 signaling complex (such as, but not limited to, Syk, ZAP70, PI3K, Erk, AKT, or GSK3b) in the presence of a ligand but not in the absence of a ligand. In some embodiments, an anti-TREM2 antibody induces phosphorylation of Syk in the presence but not the absence of a ligand. In some embodiments, an anti-TREM2 antibody induces phosphorylation of Syk in the presence but not the absence of a ligand if the level of Syk phosphorylation in a sample treated with a TREM2 ligand and the anti-TREM2 antibody is increased by at least 10%, at least 20%, at least 30%, at least 40%, at least 50%, at least 60%, at least 70%, at least 80%, at least 90% or more as compared to a control value, and if the level of Syk phosphorylation in a sample treated with the anti-TREM2 antibody but not the TREM2 ligand is not substantially increased as compared to a control value (e.g., is not increased more than 10% or more than 5% as compared to the control value). In some embodiments, an anti-TREM2 antibody induces phosphorylation of Syk in the presence but not the absence of a ligand if the level of Syk phosphorylation in a sample treated with a TREM2 ligand and the anti-TREM2 antibody is increased by at least 2-fold, 3-fold, 4-fold, 5-fold, 6-fold, 7-fold, 8-fold, 9-fold, 10-fold, 15-fold, 20-fold, 30-fold, or more as compared to a control value, and if the level of Syk phosphorylation in a sample treated with the anti-TREM2 antibody but not the TREM2 ligand is not substantially increased as compared to a control value (e.g., is not increased more than 1.5-fold or more than 1-fold as compared to the control value). In some embodiments, the control value is the level of Syk phosphorylation in an untreated sample (e.g., a sample comprising a TREM2-expressing cell that has not been treated with a TREM2 ligand or an anti-TREM2 antibody, or a sample from a subject that has not been treated with a TREM2 ligand or an anti-TREM2 antibody) or a sample treated with an appropriate non-TREM2-binding antibody.

In some embodiments, an anti-TREM2 antibody inhibits phosphorylation of a kinase that interacts with the TREM2/DAP12 signaling complex (such as, but not limited to, Syk, ZAP70, PI3K, Erk, AKT, or GSK3b). In some embodiments, an anti-TREM2 antibody inhibits phosphorylation of Syk. In some embodiments, an anti-TREM2 antibody inhibits phosphorylation of Syk if the level of Syk phosphorylation in a sample treated with the anti-TREM2 antibody is decreased by at least 10%, at least 20%, at least 30%, at least 40%, at least 50%, at least 60%, at least 70%, at least 80%, at least 90% or more as compared to a control value. In some embodiments, an anti-TREM2 antibody inhibits phosphorylation of Syk if the level of Syk phosphorylation in a sample treated with the anti-TREM2 antibody is decreased by at least 2-fold, 3-fold, 4-fold, 5-fold, 6-fold, 7-fold, 8-fold, 9-fold, 10-fold, or more as compared to a control value. In some embodiments, the control value is the level of Syk phosphorylation in an untreated sample (e.g., a sample comprising a TREM2-expressing cell that has not been treated with an anti-TREM2 antibody, or a sample from a subject that has not been treated with an anti-TREM2 antibody) or a sample treated with an appropriate non-TREM2-binding antibody.

In some embodiments, an anti-TREM2 antibody inhibits phosphorylation of a kinase that interacts with the TREM2/DAP12 signaling complex (such as, but not limited to, Syk, ZAP70, PI3K, Erk, AKT, or GSK3b) in the presence of a ligand. In some embodiments, an anti-TREM2 antibody inhibits phosphorylation of Syk in the presence of a ligand. In some embodiments, an anti-TREM2 antibody inhibits phosphorylation of Syk in the presence of a ligand if the level of Syk phosphorylation in a sample treated with a TREM2 ligand and the anti-TREM2 antibody is decreased by at least 10%, at least 20%, at least 30%, at least 40%, at least 50%, at least 60%, at least 70%, at least 80%, at least 90% or more as compared to a control value. In some embodiments, an anti-TREM2 antibody inhibits phosphorylation of Syk in the presence of a ligand if the level of Syk phosphorylation in a sample treated with a TREM2 ligand and the anti-TREM2 antibody is decreased by at least 2-fold, 3-fold, 4-fold, 5-fold, 6-fold, 7-fold, 8-fold, 9-fold, 10-fold, or more as compared to a control value. In some embodiments, the control value is the level of Syk phosphorylation in an untreated sample (e.g., a sample comprising a TREM2-expressing cell that has not been treated with a TREM2 ligand or an anti-TREM2 antibody, or a sample from a subject that has not been treated with a TREM2 ligand or an anti-TREM2 antibody) or a sample treated with an appropriate non-TREM2-binding antibody.

In some embodiments, an anti-TREM2 antibody inhibits phosphorylation of a kinase that interacts with the TREM2/DAP12 signaling complex (such as, but not limited to, Syk, ZAP70, PI3K, Erk, AKT, or GSK3b) in the presence of a ligand but not in the absence of a ligand. In some embodiments, an anti-TREM2 antibody inhibits phosphorylation of Syk in the presence but not the absence of a ligand. In some embodiments, an anti-TREM2 antibody inhibits phosphorylation of Syk in the presence but not the absence of a ligand if the level of Syk phosphorylation in a sample treated with a TREM2 ligand and the anti-TREM2 antibody is decreased by at least 10%, at least 20%, at least 30%, at least 40%, at least 50%, at least 60%, at least 70%, at least 80%, at least 90% or more as compared to a control value, and if the level of Syk phosphorylation in a sample treated with the anti-TREM2 antibody but not the TREM2 ligand is not substantially decreased as compared to a control value (e.g., is not decreased more than 10% or more than 5% as compared to the control value). In some embodiments, an anti-TREM2 antibody inhibits phosphorylation of Syk in the presence but not the absence of a ligand if the level of Syk phosphorylation in a sample treated with a TREM2 ligand and the anti-TREM2 antibody is decreased by at least 2-fold, 3-fold, 4-fold, 5-fold, 6-fold, 7-fold, 8-fold, 9-fold, 10-fold, or more as compared to a control value, and if the level of Syk phosphorylation in a sample treated with the anti-TREM2 antibody but not the TREM2 ligand is not substantially decreased as compared to a control value (e.g., is not decreased more than 1.5-fold or more than 1-fold as compared to the control value). In some embodiments, the control value is the level of Syk phosphorylation in an untreated sample (e.g., a sample comprising a TREM2-expressing cell that has not been treated with a TREM2 ligand or an anti-TREM2 antibody, or a sample from a subject that has not been treated with a TREM2 ligand or an anti-TREM2 antibody) or a sample treated with an appropriate non-TREM2-binding antibody.

In some embodiments, an anti-TREM2 antibody inhibits phosphorylation of a kinase that interacts with the TREM2/DAP12 signaling complex (such as, but not limited to, Syk, ZAP70, PI3K, Erk, AKT, or GSK3b) in the absence of a ligand and inhibits phosphorylation of a kinase that interacts with the TREM2/DAP12 signaling complex that is induced by a ligand. In some embodiments, an anti-TREM2 antibody inhibits phosphorylation of Syk in the absence of a ligand and inhibits phosphorylation of Syk that is induced by a ligand. In some embodiments, an anti-TREM2 antibody inhibits phosphorylation of Syk in the absence of a ligand and inhibits phosphorylation of Syk that is induced by a ligand if the level of Syk phosphorylation in a sample treated with the anti-TREM2 antibody in the absence of a TREM2 ligand is decreased by at least 10%, at least 20%, at least 30%, at least 40%, at least 50%, at least 60%, at least 70%, at least 80%, at least 90% or more as compared to a control value, and if the level of Syk phosphorylation in a sample treated with the anti-TREM2 antibody and a TREM2 ligand is decreased by at least 10%, at least 20%, at least 30%, at least 40%, at least 50%, at least 60%, at least 70%, at least 80%, at least 90% or more as compared to a control value. In some embodiments, an anti-TREM2 antibody inhibits phosphorylation of Syk in the absence of a ligand and inhibits phosphorylation of Syk that is induced by a ligand if the level of Syk phosphorylation in a sample treated with the anti-TREM2 antibody in the absence of a TREM2 ligand is decreased by at least 2-fold, 3-fold, 4-fold, 5-fold, 6-fold, 7-fold, 8-fold, 9-fold, 10-fold, or more as compared to a control value, and if the level of Syk phosphorylation in a sample treated with the anti-TREM2 antibody and a TREM2 ligand is decreased by at least 2-fold, 3-fold, 4-fold, 5-fold, 6-fold, 7-fold, 8-fold, 9-fold, 10-fold, or more as compared to a control value. In some embodiments, the control value is the level of Syk phosphorylation in an untreated sample (e.g., a sample comprising a TREM2-expressing cell that has not been treated with a TREM2 ligand or an anti-TREM2 antibody, or a sample from a subject that has not been treated with a TREM2 ligand or an anti-TREM2 antibody) or a sample treated with an appropriate non-TREM2-binding antibody.

For detecting and/or quantifying phosphorylation (e.g., Syk phosphorylation) in a sample, in some embodiments, an immunoassay is used. In some embodiments, the immunoassay is an enzyme immunoassay (EIA), enzyme multiplied immunoassay (EMIA), enzyme-linked immunosorbent assay (ELISA), microparticle enzyme immunoassay (MEIA), immunohistochemistry (IHC), immunocytochemistry, capillary electrophoresis immunoassay (CEIA), radioimmunoassay (RIA), immunofluorescence, chemiluminescence immunoassay (CL), or electrochemiluminescence immunoassay (ECL). In some embodiments, phosphorylation is detected and/or quantified using an immunoassay that utilizes an amplified luminescent proximity homogenous assay (AlphaLISA®, PerkinElmer Inc.).

In some embodiments, phosphorylation is measured using a sample that comprises one or more cells, e.g., one or more TREM2-expressing cells (e.g., a primary cell or cell line that endogenously expresses TREM2, such as human macrophages, or a primary cell or cell line that has been engineered to express TREM2, e.g., as described in the Examples section below). In some embodiments, the sample comprises a fluid, e.g., blood, plasma, serum, urine, or cerebrospinal fluid. In some embodiments, the sample comprises tissue (e.g., lung, brain, kidney, spleen, nervous tissue, or skeletal muscle) or cells from such tissue. In some embodiments, the sample comprises endogenous fluid, tissue, or cells (e.g., from a human or non-human subject).

Phagocytosis

In some embodiments, an anti-TREM2 antibody enhances phagocytosis of dead cell debris, tissue debris, amyloid beta particles, or foreign material. In some embodiments, an anti-TREM2 antibody enhances phagocytosis without blocking binding of a native TREM2 ligand. In some embodiments, an anti-TREM2 antibody enhances phagocytosis if the level of phagocytosis in a sample treated with the anti-TREM2 antibody is increased by at least 10%, at least 20%, at least 30%, at least 40%, at least 50%, at least 60%, at least 70%, at least 80%, at least 90% or more as compared to a control value. In some embodiments, an anti-TREM2 antibody enhances phagocytosis if the level of phagocytosis in a sample treated with the anti-TREM2 antibody is increased by at least 2-fold, 3-fold, 4-fold, 5-fold, 6-fold, 7-fold, 8-fold, 9-fold, 10-fold, or more as compared to a control value. In some embodiments, the control value is the level of phagocytosis in an untreated sample or a sample treated with an appropriate non-TREM2-binding antibody.

In some embodiments, an anti-TREM2 antibody enhances phagocytosis in the presence of a ligand. In some embodiments, an anti-TREM2 antibody enhances phagocytosis in the presence of a ligand if the level of phagocytosis in a sample treated with a TREM2 ligand and the anti-TREM2 antibody is increased by at least 10%, at least 20%, at least 30%, at least 40%, at least 50%, at least 60%, at least 70%, at least 80%, at least 90% or more as compared to a control value. In some embodiments, an anti-TREM2 antibody enhances phagocytosis in the presence of a ligand if the level of phagocytosis in a sample treated with a TREM2 ligand and the anti-TREM2 antibody is increased by at least 2-fold, 3-fold, 4-fold, 5-fold, 6-fold, 7-fold, 8-fold, 9-fold, 10-fold, or more as compared to a control value. In some embodiments, the control value is the level of phagocytosis in an untreated sample or a sample treated with an appropriate non-TREM2-binding antibody.

In some embodiments, an anti-TREM2 antibody enhances phagocytosis in the presence of a ligand but not in the absence of a ligand. In some embodiments, an anti-TREM2 antibody enhances phagocytosis in the presence but not the absence of a ligand if the level of phagocytosis in a sample treated with a TREM2 ligand and the anti-TREM2 antibody is increased by at least 10%, at least 20%, at least 30%, at least 40%, at least 50%, at least 60%, at least 70%, at least 80%, at least 90% or more as compared to a control value, and if the level of phagocytosis in a sample treated with the anti-TREM2 antibody but not the TREM2 ligand is not substantially increased as compared to a control value (e.g., is not increased more than 10% or more than 5% as compared to the control value). In some embodiments, an anti-TREM2 antibody enhances phagocytosis in the presence but not the absence of a ligand if the level of phagocytosis in a sample treated with a TREM2 ligand and the anti-TREM2 antibody is increased by at least 2-fold, 3-fold, 4-fold, 5-fold, 6-fold, 7-fold, 8-fold, 9-fold, 10-fold, or more as compared to a control value, and if the level of phagocytosis in a sample treated with the anti-TREM2 antibody but not the TREM2 ligand is not substantially increased as compared to a control value (e.g., is not increased more than 1.5-fold or more than 1-fold as compared to the control value). In some embodiments, the control value is the level of phagocytosis in an untreated sample or a sample treated with an appropriate non-TREM2-binding antibody.

In some embodiments, an anti-TREM2 antibody reduces phagocytosis. In some embodiments, an anti-TREM2 antibody reduces phagocytosis if the level of phagocytosis in a sample treated with the anti-TREM2 antibody is decreased by at least 10%, at least 20%, at least 30%, at least 40%, at least 50%, at least 60%, at least 70%, at least 80%, at least 90% or more as compared to a control value. In some embodiments, an anti-TREM2 antibody reduces phagocytosis if the level of phagocytosis in a sample treated with the anti-TREM2 antibody is decreased by at least 2-fold, 3-fold, 4-fold, 5-fold, 6-fold, 7-fold, 8-fold, 9-fold, 10-fold, or more as compared to a control value. In some embodiments, the control value is the level of phagocytosis in an untreated sample or a sample treated with an appropriate non-TREM2-binding antibody.

In some embodiments, an anti-TREM2 antibody reduces phagocytosis in the presence of a ligand. In some embodiments, an anti-TREM2 antibody reduces phagocytosis in the presence of a ligand if the level of phagocytosis in a sample treated with a TREM2 ligand and the anti-TREM2 antibody is decreased by at least 10%, at least 20%, at least 30%, at least 40%, at least 50%, at least 60%, at least 70%, at least 80%, at least 90% or more as compared to a control value. In some embodiments, an anti-TREM2 antibody reduces phagocytosis in the presence of a ligand if the level of phagocytosis in a sample treated with a TREM2 ligand and the anti-TREM2 antibody is decreased by at least 2-fold, 3-fold, 4-fold, 5-fold, 6-fold, 7-fold, 8-fold, 9-fold, 10-fold, or more as compared to a control value. In some embodiments, the control value is the level of phagocytosis in an untreated sample or a sample treated with an appropriate non-TREM2-binding antibody.

In some embodiments, an anti-TREM2 antibody reduces phagocytosis in the presence of a ligand but not in the absence of a ligand. In some embodiments, an anti-TREM2 antibody reduces phagocytosis in the presence but not the absence of a ligand if the level of phagocytosis in a sample treated with a TREM2 ligand and the anti-TREM2 antibody is decreased by at least 10%, at least 20%, at least 30%, at least 40%, at least 50%, at least 60%, at least 70%, at least 80%, at least 90% or more as compared to a control value, and if the level of phagocytosis in a sample treated with the anti-TREM2 antibody but not the TREM2 ligand is not substantially decreased as compared to a control value (e.g., is not decreased more than 10% or more than 5% as compared to the control value). In some embodiments, an anti-TREM2 antibody reduces phagocytosis in the presence but not the absence of a ligand if the level of phagocytosis in a sample treated with a TREM2 ligand and the anti-TREM2 antibody is decreased by at least 2-fold, 3-fold, 4-fold, 5-fold, 6-fold, 7-fold, 8-fold, 9-fold, 10-fold, or more as compared to a control value, and if the level of phagocytosis in a sample treated with the anti-TREM2 antibody but not the TREM2 ligand is not substantially decreased as compared to a control value (e.g., is not decreased more than 1.5-fold or more than 1-fold as compared to the control value). In some embodiments, the control value is the level of phagocytosis in an untreated sample or a sample treated with an appropriate non-TREM2-binding antibody.

In some embodiments, phagocytosis is measured using a phagocytosis assay with a labeled substrate. Phagocytosis assays are known in the art. In some embodiments, the phagocytosis assay is performed on a sample comprising cells that endogenously express TREM2, such as human macrophages or microglia. In some embodiments, the phagocytosis assay is performed on a sample comprising cells that have been engineered to express TREM2. In some embodiments, cell migration is measured using a human macrophage phagocytosis assay as described in the Examples section below.

Cell Migration, Survival, Function, and Differentiation

In some embodiments, an anti-TREM2 antibody enhances cell migration, cell survival, or cell differentiation (e.g., for myeloid cells, macrophages, and microglia, including disease-associated microglia). Disease-associated microglia and methods of detecting disease-associated microglia are described in Keren-Shaul et al., Cell, 2017, 169:1276-1290. In some embodiments, an anti-TREM2 antibody enhances cell migration of one or more cell types (e.g., myeloid cells, macrophages, or microglia). In some embodiments, an anti-TREM2 antibody enhances cell survival of one or more cell types (e.g., myeloid cells, macrophages, or microglia). In some embodiments, an anti-TREM2 antibody enhances cell differentiation of one or more cell types (e.g., myeloid cells, macrophages, or microglia). In some embodiments, an anti-TREM2 antibody enhances the migration, survival, and/or differentiation of myeloid cells. In some embodiments, an anti-TREM2 antibody enhances the migration, survival, and/or differentiation of macrophages. In some embodiments, an anti-TREM2 antibody enhances the migration, survival, and/or differentiation of microglia. In some embodiments, an anti-TREM2 antibody enhances microglia activation. In some embodiments, an anti-TREM2 antibody enhances the migration, survival, and/or differentiation of disease-associated microglia. In some embodiments, an anti-TREM2 antibody enhances cell migration, cell survival, or cell differentiation without blocking binding of a native TREM2 ligand.

In some embodiments, an anti-TREM2 antibody enhances cell migration, cell survival, or cell differentiation if the level of activity (e.g., migration, survival, or differentiation) in a sample treated with the anti-TREM2 antibody is increased by at least 10%, at least 20%, at least 30%, at least 40%, at least 50%, at least 60%, at least 70%, at least 80%, at least 90% or more as compared to a control value. In some embodiments, an anti-TREM2 antibody enhances cell migration, cell survival, or cell differentiation if the level of activity (e.g., migration, survival, or differentiation) in a sample treated with the anti-TREM2 antibody is increased by at least 2-fold, 3-fold, 4-fold, 5-fold, 6-fold, 7-fold, 8-fold, 9-fold, 10-fold, or more as compared to a control value. In some embodiments, the control value is the level of activity (e.g., migration, survival, or differentiation) in an untreated sample (e.g., a sample that has not been treated with an anti-TREM2 antibody) or a sample treated with an appropriate non-TREM2-binding antibody.

In some embodiments, an anti-TREM2 antibody enhances cell migration, cell survival, or cell differentiation in the presence of a ligand. In some embodiments, an anti-TREM2 antibody enhances cell migration, cell survival, or cell differentiation in the presence of a ligand if the level of activity (e.g., migration, survival, or differentiation) in a sample treated with a TREM2 ligand and the anti-TREM2 antibody is increased by at least 10%, at least 20%, at least 30%, at least 40%, at least 50%, at least 60%, at least 70%, at least 80%, at least 90% or more as compared to a control value. In some embodiments, an anti-TREM2 antibody enhances cell migration, cell survival, or cell differentiation in the presence of a ligand if the level of activity (e.g., migration, survival, or differentiation) in a sample treated with a TREM2 ligand and the anti-TREM2 antibody is increased by at least 2-fold, 3-fold, 4-fold, 5-fold, 6-fold, 7-fold, 8-fold, 9-fold, 10-fold, or more as compared to a control value. In some embodiments, the control value is the level of activity (e.g., migration, survival, or differentiation) in an untreated sample (e.g., a sample that has not been treated with a TREM2 ligand or an anti-TREM2 antibody) or a sample treated with an appropriate non-TREM2-binding antibody.

In some embodiments, an anti-TREM2 antibody enhances cell migration, cell survival, or cell differentiation in the presence of a ligand but not in the absence of a ligand. In some embodiments, an anti-TREM2 antibody enhances cell migration, cell survival, or cell differentiation in the presence but not the absence of a ligand if the level of activity (e.g., migration, survival, or differentiation) in a sample treated with a TREM2 ligand and the anti-TREM2 antibody is increased by at least 10%, at least 20%, at least 30%, at least 40%, at least 50%, at least 60%, at least 70%, at least 80%, at least 90% or more as compared to a control value, and if the level of activity in a sample treated with the anti-TREM2 antibody but not the TREM2 ligand is not substantially increased as compared to a control value (e.g., is not increased more than 10% or more than 5% as compared to the control value). In some embodiments, an anti-TREM2 antibody enhances cell migration, cell survival, or cell differentiation in the presence but not the absence of a ligand if the level of activity (e.g., migration, survival, or differentiation) in a sample treated with a TREM2 ligand and the anti-TREM2 antibody is increased by at least 2-fold, 3-fold, 4-fold, 5-fold, 6-fold, 7-fold, 8-fold, 9-fold, 10-fold, or more as compared to a control value, and if the level of activity in a sample treated with the anti-TREM2 antibody but not the TREM2 ligand is not substantially increased as compared to a control value (e.g., is not increased more than 1.5-fold or more than 1-fold as compared to the control value). In some embodiments, the control value is the level of activity (e.g., migration, survival, or differentiation) in an untreated sample (e.g., a sample that has not been treated with a TREM2 ligand or an anti-TREM2 antibody) or a sample treated with an appropriate non-TREM2-binding antibody.

In some embodiments, an anti-TREM2 antibody inhibits cell migration, cell survival, or cell differentiation (e.g., for myeloid cells, macrophages, and microglia, including disease-associated microglia). In some embodiments, an anti-TREM2 antibody inhibits cell migration of one or more cell types (e.g., myeloid cells, macrophages, or microglia). In some embodiments, an anti-TREM2 antibody inhibits cell survival of one or more cell types (e.g., myeloid cells, macrophages, or microglia). In some embodiments, an anti-TREM2 antibody inhibits cell differentiation of one or more cell types (e.g., myeloid cells, macrophages, or microglia). In some embodiments, an anti-TREM2 antibody inhibits the migration, survival, and/or differentiation of myeloid cells. In some embodiments, an anti-TREM2 antibody inhibits the migration, survival, and/or differentiation of macrophages. In some embodiments, an anti-TREM2 antibody inhibits the migration, survival, and/or differentiation of microglia. In some embodiments, an anti-TREM2 antibody inhibits microglia activation. In some embodiments, an anti-TREM2 antibody inhibits the migration, survival, and/or differentiation of disease-associated microglia.

In some embodiments, an anti-TREM2 antibody inhibits cell migration, cell survival, or cell differentiation if the level of activity (e.g., migration, survival, or differentiation) in a sample treated with the anti-TREM2 antibody is decreased by at least 10%, at least 20%, at least 30%, at least 40%, at least 50%, at least 60%, at least 70%, at least 80%, at least 90% or more as compared to a control value. In some embodiments, an anti-TREM2 antibody inhibits cell migration, cell survival, or cell differentiation if the level of activity (e.g., migration, survival, or differentiation) in a sample treated with the anti-TREM2 antibody is decreased by at least 2-fold, 3-fold, 4-fold, 5-fold, 6-fold, 7-fold, 8-fold, 9-fold, 10-fold or more as compared to a control value. In some embodiments, the control value is the level of Syk phosphorylation in an untreated sample (e.g., a sample that has not been treated with an anti-TREM2 antibody) or a sample treated with an appropriate non-TREM2-binding antibody.

In some embodiments, an anti-TREM2 antibody inhibits cell migration, cell survival, or cell differentiation in the presence of a ligand. In some embodiments, an anti-TREM2 antibody inhibits cell migration, cell survival, or cell differentiation in the presence of a ligand if the level of activity (e.g., migration, survival, or differentiation) in a sample treated with a TREM2 ligand and the anti-TREM2 antibody is decreased by at least 10%, at least 20%, at least 30%, at least 40%, at least 50%, at least 60%, at least 70%, at least 80%, at least 90% or more as compared to a control value. In some embodiments, an anti-TREM2 antibody inhibits cell migration, cell survival, or cell differentiation in the presence of a ligand if the level of activity (e.g., migration, survival, or differentiation) in a sample treated with a TREM2 ligand and the anti-TREM2 antibody is decreased by at least 2-fold, 3-fold, 4-fold, 5-fold, 6-fold, 7-fold, 8-fold, 9-fold, 10-fold or more as compared to a control value. In some embodiments, the control value is the level of activity (e.g., migration, survival, or differentiation) in an untreated sample (e.g., a sample that has not been treated with a TREM2 ligand or an anti-TREM2 antibody) or a sample treated with an appropriate non-TREM2-binding antibody.

In some embodiments, an anti-TREM2 antibody inhibits cell migration, cell survival, or cell differentiation in the presence of a ligand but not in the absence of a ligand. In some embodiments, an anti-TREM2 antibody inhibits cell migration, cell survival, or cell differentiation in the presence but not the absence of a ligand if the level of activity (e.g., migration, survival, or differentiation) in a sample treated with a TREM2 ligand and the anti-TREM2 antibody is decreased by at least 10%, at least 20%, at least 30%, at least 40%, at least 50%, at least 60%, at least 70%, at least 80%, at least 90% or more as compared to a control value, and if the level of activity in a sample treated with the anti-TREM2 antibody but not the TREM2 ligand is not substantially decreased as compared to a control value (e.g., is not decreased more than 10% or more than 5% as compared to the control value). In some embodiments, an anti-TREM2 antibody inhibits cell migration, cell survival, or cell differentiation in the presence but not the absence of a ligand if the level of activity (e.g., migration, survival, or differentiation) in a sample treated with a TREM2 ligand and the anti-TREM2 antibody is decreased by at least 2-fold, 3-fold, 4-fold, 5-fold, 6-fold, 7-fold, 8-fold, 9-fold, 10-fold or more as compared to a control value, and if the level of activity in a sample treated with the anti-TREM2 antibody but not the TREM2 ligand is not substantially decreased as compared to a control value (e.g., is not decreased more than 1.5-fold or more than 1-fold as compared to the control value). In some embodiments, the control value is the level of activity (e.g., migration, survival, or differentiation) in an untreated sample (e.g., a sample that has not been treated with a TREM2 ligand or an anti-TREM2 antibody).

In some embodiments, cell migration is measured using a chemotaxis assay. Chemotaxis assays are known in the art. In some embodiments, the cell migration assay (e.g., chemotaxis assay) is performed on a sample comprising cells that endogenously express TREM2, such as human macrophages. In some embodiments, the cell migration assay (e.g., chemotaxis assay) is performed on a sample comprising cells that have been engineered to express TREM2. In some embodiments, cell migration is measured using a human macrophage chemotaxis assay as described in the Examples section below.

In some embodiments, cell survival is measured using a cell viability assay. Cell viability assays are known in the art. In some embodiments, the cell survival assay (e.g., cell viability assay) is performed on a sample comprising cells that endogenously express TREM2, such as human macrophages. In some embodiments, the cell survival assay (e.g., cell viability assay) is performed on a sample comprising cells that have been engineered to express TREM2. In some embodiments, cell survival is measured using a human macrophage viability assay as described in the Examples section below.

In some embodiments, cell differentiation is measured by evaluating the ability of cells that endogenously express TREM2 to differentiate. For example, in some embodiments, cell differentiation is measured by evaluating the ability of macrophages to differentiate from monocytes, e.g., as described in the Examples section below.

In some embodiments, activation of microglia is measured in vivo. In some embodiments, microglia activation is measured using TSPO-PET imaging. TSPO-PET imaging methods are known in the art.

In some embodiments, an anti-TREM2 antibody enhances microglia function without increasing neuroinflammation. Levels of neuroinflammation can be determined by measuring levels of cytokines (e.g., inflammatory cytokines), such as but not limited to TNF-α, IL-1β, IL-6, IL-1ra, TGFβ, IL-15, or IFN-γ. In some embodiments, cytokine levels are measured using immunoassays, for example, an enzyme immunoassay (EIA), enzyme multiplied immunoassay (EMIA), enzyme-linked immunosorbent assay (ELISA), microparticle enzyme immunoassay (MEIA), immunohistochemistry (IHC), immunocytochemistry, capillary electrophoresis immunoassay (CEIA), radioimmunoassay (RIA), immunofluorescence, chemiluminescence immunoassay (CL), or electrochemiluminescence immunoassay (ECL).

TREM2 Ligands

In some embodiments, a TREM2 ligand is a lipid ligand as described herein, e.g., as described in Example 4 below. In some embodiments, the TREM2 ligand is selected from the group consisting of 1-palmitoyl-2-(5′-oxo-valeroyl)-sn-glycero-3-phosphocholine (POVPC), 2-Arachidonoylglycerol (2-AG), 7-ketocholesterol (7-KC), 24(S)hydroxycholesterol (240HC), 25 (S)hydroxycholesterol (25OHC), 27-hydroxycholesterol (270HC), Acyl Carnitine (AC), alkylacylglycerophosphocholine (PAF), α-galactosylceramide (KRN7000), Bis(monoacylglycero)phosphate (BMP), Cardiolipin (CL), Ceramide, Ceramide-1-phosphate (C1P), Cholesteryl ester (CE), Cholesterol phosphate (CP), Diacylglycerol 34:1 (DG 34:1), Diacylglycerol 38:4 (DG 38:4), Diacylglycerol pyrophosphate (DGPP), Dihyrdoceramide (DhCer), Dihydrosphingomyelin (DhSM), Ether phosphatidylcholine (PCe), Free cholesterol (FC), Galactosylceramide (GalCer), Galactosylsphingosine (GalSo), Ganglioside GM1, Ganglioside GM3, Glucosylsphingosine (GlcSo), Hank's Balanced Salt Solution (HBSS), Kdo2-Lipid A (KLA), Lactosylceramide (LacCer), lysoalkylacylglycerophosphocholine (LPAF), Lysophosphatidic acid (LPA), Lysophosphatidylcholine (LPC), Lysophosphatidylethanolamine (LPE), Lysophosphatidylglycerol (LPG), Lysophosphatidylinositol (LPI), Lysosphingomyelin (LSM), Lysophosphatidylserine (LPS), N-Acyl-phosphatidylethanolamine (NAPE), N-Acyl-Serine (NSer), Oxidized phosphatidylcholine (oxPC), Palmitic-acid-9-hydroxy-stearic-acid (PAHSA), Phosphatidylethanolamine (PE), Phosphatidylethanol (PEtOH), Phosphatidic acid (PA), Phosphatidylcholine (PC), Phosphatidylglycerol (PG), Phosphatidylinositol (PI), Phosphatidylserine (PS), Sphinganine, Sphinganine-1-phosphate (Sa1P), Sphingomyelin (SM), Sphingosine, Sphingosine-1-phosphate (So1P), and Sulfatide.

In some embodiments, an anti-TREM2 antibody as described herein interacts with one or more lipid ligands (e.g., a lipid ligand as described herein) to modulate TREM2 activity. In some embodiments, an anti-TREM2 antibody as described herein interacts with one or more lipid ligands to modulate a TREM2 signaling pathway involving a kinase that interacts with the TREM2/DAP12 signaling complex, such as, but not limited to, Syk, ZAP70, PI3K, Erk, AKT, or GSK3b. In some embodiments, the anti-TREM2 antibody interacts with the lipid ligand to activate Syk, ZAP70, PI3K, Erk, AKT, or GSK3b. In some embodiments, the anti-TREM2 antibody exhibits an additive effect with the lipid ligand on the activation of the signaling pathway (e.g., Syk, ZAP70, PI3K, Erk, AKT, or GSK3b). In some embodiments, the anti-TREM2 antibody exhibits an additive effect with the lipid ligand on the activation of p-Syk. In some embodiments, the anti-TREM2 antibody exhibits a blocking effect on the lipid ligand activation of the signaling pathway (e.g., Syk, ZAP70, PI3K, Erk, AKT, or GSK3b). In some embodiments, the anti-TREM2 antibody exhibits a blocking effect on the lipid ligand activation of p-Syk.

In some embodiments, an anti-TREM2 antibody exhibits an additive effect with the lipid ligand on the activation of a TREM2 signaling pathway component (e.g., p-Syk) and recognizes an epitope that is the same or substantially the same as the epitope recognized by antibody clone RS9.F6, 22B8.B1, 3D3.A1, 42E8.H1, 43E9.H1, 21D6.G2, 59C6.F1, 53H11.D3, 60A4.B1, 26E2.A3, 54C2.A1, 44E2.H1, 22G9.D1, 49H11.B1, 14D5.F1, 26D11.B1, 52H9.D1, or 7B10.A2. In some embodiments, an anti-TREM2 antibody exhibits a blocking effect with the lipid ligand on the activation of a TREM2 signaling pathway component (e.g., p-Syk) and recognizes an epitope that is the same or substantially the same as the epitope recognized by antibody clone RS9.F10, 13B11.A1, 21D4.D1, 30A8.A1, 57D7.A1, 24B4.A1, 39H10.A1, 55B9.A1, 14H11.B1, 40H3.A4, 30F2.A1, 51D4.A1, 26D2.D1, 21D11.B1, 44E3.B1, 26D5.A1, 38E9.E5, RS9.E2, or 2G4.B1.

### Binding Characteristics of Anti-TREM2 Antibodies

In some embodiments, an antibody that specifically binds to a TREM2 protein as described herein binds to TREM2 that is expressed on a cell (e.g., a primary cell or cell line that endogenously expresses TREM2, such as human macrophages, or a primary cell or cell line that has been engineered to express TREM2, e.g., as described in the Examples section below). In some embodiments, an antibody that specifically binds to a TREM2 protein as described herein binds to purified or recombinant TREM2 protein or to a chimeric protein comprising TREM2 or a portion thereof (e.g., an Fc-fusion protein comprising TREM2 or an Fc-fusion protein comprising the ecto-domain of TREM2).

In some embodiments, an antibody that specifically binds to human TREM2 protein exhibits cross-reactivity with one or more other TREM2 proteins of another species. In some embodiments, an antibody that specifically binds to human TREM2 protein exhibits cross-reactivity with a mouse TREM2 protein. In some embodiments, an antibody that specifically binds to human TREM2 protein exhibits cross-reactivity with a cynomolgus monkey (“cyno”) TREM2 protein. In some embodiments, an antibody that specifically binds to human TREM2 protein exhibits cross-reactivity with a rat TREM2 protein. In some embodiments, an antibody that specifically binds to human TREM2 protein exhibits cross-reactivity with one, two, or all three of mouse TREM2, cyno TREM2, and rat TREM2. In some embodiments, an anti-TREM2 antibody exhibits cross-reactivity with human TREM2, cyno TREM2, and mouse TREM2.

Methods for analyzing binding affinity, binding kinetics, and cross-reactivity are known in the art. These methods include, but are not limited to, solid-phase binding assays (e.g., ELISA assay), immunoprecipitation, surface plasmon resonance (e.g., Biacore™ (GE Healthcare, Piscataway, N.J.)), kinetic exclusion assays (e.g., KinExA®), flow cytometry, fluorescence-activated cell sorting (FACS), BioLayer interferometry (e.g., Octet™ (FortéBio, Inc., Menlo Park, Calif.)), and western blot analysis. In some embodiments, ELISA is used to determine binding affinity and/or cross-reactivity. Methods for performing ELISA assays are known in the art, and are also described in the Examples section below. In some embodiments, surface plasmon resonance (SPR) is used to determine binding affinity, binding kinetics, and/or cross-reactivity. In some embodiments, kinetic exclusion assays are used to determine binding affinity, binding kinetics, and/or cross-reactivity. In some embodiments, BioLayer interferometry assays are used to determine binding affinity, binding kinetics, and/or cross-reactivity.

### Epitopes Recognized by Anti-TREM2 Antibodies

In some embodiments, an anti-TREM2 antibody recognizes an epitope of human TREM2 that is the same or substantially the same as the epitope recognized by an antibody clone as described herein. As used herein, the term “substantially the same,” as used with reference to an epitope recognized by an antibody clone as described herein, means that the anti-TREM2 antibody recognizes an epitope that is identical, within, or nearly identical to (e.g., has at least 90% sequence identity to, or has one, two, or three amino acid substitutions, e.g., conservative substitutions, relative to), or has substantial overlap with (e.g., at least 50%, 60%, 70%, 80%, 90%, or 95% overlap with) the epitope recognized by the antibody clone as described herein.

In some embodiments, the anti-TREM2 antibody recognizes an epitope of human TREM2 that is the same or substantially the same as the epitope recognized by an antibody clone selected from the group consisting of 2G4.B1, 3D3.A1, 7B10.A2, 8A11.B1, 13B11.A1, 14D5.F1, 14H11.A1, 19F10.F3, 21D4.D1, 21D6.G2, 21D11, 22B8.B1, 22G9.D1, 24B4.A1, 26D2.D1, 26D5.A1, 26D11.B1, 26E2.A3, 30A8.A1, 30F2.A2, 38E9.E5, 39H10.A1, 40H3.A4, 42E8.H1, 43E9.H1, 44E2.H1, 44E3.B1, 49H11.B1, 51D4, 52H9.D1, 53H11.D3, 54C2.A1, 55B9.A1, 57D7.A1, 59C6.F1, 60A4.B1, RS9.E2, RS9.F6, RS9.F10. RS11.1F5, RS11.1G6, RS11.1A10, RS11.1D11, RS11.4A5, RS11.4H6, RS11.4D7, RS11.4D9, RS11.4F11, RS12.1C6, RS12.1C10, RS12.2D1, RS12.2D4, RS12.2E1, RS12.2F2, RS12.2G1, RS12.2H1, and RS12.3C10.

In some embodiments, the anti-TREM2 antibody recognizes an epitope of human TREM2 that is identical to the epitope recognized by an antibody clone selected from the group consisting of 2G4.B1, 3D3.A1, 7B10.A2, 8A11.B1, 13B11.A1, 14D5.F1, 14H11.A1, 19F10.F3, 21D4.D1, 21D6.G2, 21D11, 22B8.B1, 22G9.D1, 24B4.A1, 26D2.D1, 26D5.A1, 26D11.B1, 26E2.A3, 30A8.A1, 30F2.A2, 38E9.E5, 39H10.A1, 40H3.A4, 42E8.H1, 43E9.H1, 44E2.H1, 44E3.B1, 49H11.B1, 51D4, 52H9.D1, 53H11.D3, 54C2.A1, 55B9.A1, 57D7.A1, 59C6.F1, 60A4.B1, RS9.E2, RS9.F6, RS9.F10, RS11.1F5, RS11.1G6, RS11.1A10, RS11.1D11, RS11.4A5, RS11.4H6, RS11.4D7, RS11.4D9, RS11.4F11, RS12.1C6, RS12.1C10, RS12.2D1, RS12.2D4, RS12.2E1, RS12.2F2, RS12.2G1, RS12.2H1, and RS12.3C10.

In some embodiments, the anti-TREM2 antibody recognizes an epitope of human TREM2 that is within the epitope recognized by an antibody clone selected from the group consisting of 2G4.B1, 3D3.A1, 7B10.A2, 8A11.B1, 13B11.A1, 14D5.F1, 14H11.A1, 19F10.F3, 21D4.D1, 21D6.G2, 21D11, 22B8.B1, 22G9.D1, 24B4.A1, 26D2.D1, 26D5.A1, 26D11.B1, 26E2.A3, 30A8.A1, 30F2.A2, 38E9.E5, 39H10.A1, 40H3.A4, 42E8.H1, 43E9.H1, 44E2.H1, 44E3.B1, 49H11.B1, 51D4, 52H9.D1, 53H11.D3, 54C2.A1, 55B9.A1, 57D7.A1, 59C6.F1, 60A4.B1, RS9.E2, RS9.F6, RS9.F10, RS11.1F5, RS11.1G6, RS11.1A10, RS11.1D11, RS11.4A5, RS11.4H6, RS11.4D7, RS11.4D9, RS11.4F11, RS12.1C6, RS12.1C10, RS12.2D1, RS12.2D4, RS12.2E1, RS12.2F2, RS12.2G1, RS12.2H1, and RS12.3C10.

In some embodiments, the anti-TREM2 antibody recognizes an epitope of human TREM2 that has at least 90% identity (e.g., at least 91%, 92%, 93%, 94%, 95%, 96%, 97%, 98%, or 99%) to the epitope recognized by an antibody clone selected from the group consisting of 2G4.B1, 3D3.A1, 7B10.A2, 8A11.B1, 13B11.A1, 14D5.F1, 14H11.A1, 19F10.F3, 21D4.D1, 21D6.G2, 21D11, 22B8.B1, 22G9.D1, 24B4.A1, 26D2.D1, 26D5.A1, 26D11.B1, 26E2.A3, 30A8.A1, 30F2.A2, 38E9.E5, 39H10.A1, 40H3.A4, 42E8.H1, 43E9.H1, 44E2.H1, 44E3.B1, 49H11.B1, 51D4, 52H9.D1, 53H11.D3, 54C2.A1, 55B9.A1, 57D7.A1, 59C6.F1, 60A4.B1, RS9.E2, RS9.F6, RS9.F10, RS11.1F5, RS11.1G6, RS11.1A10, RS11.1D11, RS11.4A5, RS11.4H6, RS11.4D7, RS11.4D9, RS11.4F11, RS12.1C6, RS12.1C10, RS12.2D1, RS12.2D4, RS12.2E1, RS12.2F2, RS12.2G1, RS12.2H1, and RS12.3C10.

In some embodiments, the anti-TREM2 antibody recognizes an epitope of human TREM2 that has one, two, or three amino acid substitutions (e.g., conservative substitutions) relative to the epitope recognized by an antibody clone selected from the group consisting of 2G4.B1, 3D3.A1, 7B10.A2, 8A11.B1, 13B11.A1, 14D5.F1, 14H11.A1, 19F10.F3, 21D4.D1, 21D6.G2, 21D11, 22B8.B1, 22G9.D1, 24B4.A1, 26D2.D1, 26D5.A1, 26D11.B1, 26E2.A3, 30A8.A1, 30F2.A2, 38E9.E5, 39H10.A1, 40H3.A4, 42E8.H1, 43E9.H1, 44E2.H1, 44E3.B1, 49H11.B1, 51D4, 52H9.D1, 53H11.D3, 54C2.A1, 55B9.A1, 57D7.A1, 59C6.F1, 60A4.B1, RS9.E2, RS9.F6, RS9.F10, RS11.1F5, RS11.1G6, RS11.1A10, RS11.1D11, RS11.4A5, RS11.4H6, RS11.4D7, RS11.4D9, RS11.4F11, RS12.1C6, RS12.1C10, RS12.2D1, RS12.2D4, RS12.2E1, RS12.2F2, RS12.2G1, RS12.2H1, and RS12.3C10.

In some embodiments, an anti-TREM2 antibody recognizes an epitope of human TREM2 that substantially overlaps (e.g., has at least 50%, 60%, 70%, 80%, 90%, or 95% overlap) with the epitope recognized by an antibody clone selected from the group consisting of 2G4.B1, 3D3.A1, 7B10.A2, 8A11.B1, 13B11.A1, 14D5.F1, 14H11.A1, 19F10.F3, 21D4.D1, 21D6.G2, 21D11, 22B8.B1, 22G9.D1, 24B4.A1, 26D2.D1, 26D5.A1, 26D11.B1, 26E2.A3, 30A8.A1, 30F2.A2, 38E9.E5, 39H10.A1, 40H3.A4, 42E8.H1, 43E9.H1, 44E2.H1, 44E3.B1, 49H11.B1, 51D4, 52H9.D1, 53H11.D3, 54C2.A1, 55B9.A1, 57D7.A1, 59C6.F1, 60A4.B1, RS9.E2, RS9.F6, RS9.F10, RS11.1F5, RS11.1G6, RS11.1A10, RS11.1D11, RS11.4A5, RS11.4H6, RS11.4D7, RS11.4D9, RS11.4F11, RS12.1C6, RS12.1C10, RS12.2D1, RS12.2D4, RS12.2E1, RS12.2F2, RS12.2G1, RS12.2H1, and RS12.3C10.

In some embodiments, an anti-TREM2 antibody decreases levels of sTREM2 and recognizes an epitope that is the same or substantially the same as the epitope recognized by antibody clone 42E8.H1. In some embodiments, the anti-TREM2 antibody decreases levels of sTREM2 and recognizes an epitope of human TREM2 that is identical to the epitope recognized by antibody clone 42E8.H1. In some embodiments, the anti-TREM2 antibody decreases levels of sTREM2 and recognizes an epitope of human TREM2 that is within the epitope recognized by antibody clone 42E8.H1. In some embodiments, the anti-TREM2 antibody decreases levels of sTREM2 and recognizes an epitope of human TREM2 that has at least 90% identity to the epitope recognized by antibody clone 42E8.H1. In some embodiments, the anti-TREM2 antibody decreases levels of sTREM2 and recognizes an epitope of human TREM2 that has one, two, or three amino acid substitutions (e.g., conservative substitutions) relative to the epitope recognized by antibody clone 42E8.H1.

In some embodiments, an anti-TREM2 antibody increases levels of sTREM2 and recognizes an epitope that is the same or substantially the same as the epitope recognized by antibody clone 21D4.D1. In some embodiments, the anti-TREM2 antibody increases levels of sTREM2 and recognizes an epitope of human TREM2 that is identical to the epitope recognized by antibody clone 21D4.D1. In some embodiments, the anti-TREM2 antibody increases levels of sTREM2 and recognizes an epitope of human TREM2 that is within the epitope recognized by antibody clone 21D4.D1. In some embodiments, the anti-TREM2 antibody increases levels of sTREM2 and recognizes an epitope of human TREM2 that has at least 90% identity to the epitope recognized by antibody clone 21D4.D1. In some embodiments, the anti-TREM2 antibody increases levels of sTREM2 and recognizes an epitope of human TREM2 that has one, two, or three amino acid substitutions (e.g., conservative substitutions) relative to the epitope recognized by antibody clone 21D4.D1.

In some embodiments, an anti-TREM antibody enhances TREM2 activity (e.g., induces kinase phosphorylation, enhances phagocytosis, and/or enhances cell migration, differentiation, or survival) and recognizes an epitope that is the same or substantially the same as the epitope recognized by antibody clone 2G4.B1, 3D3.A1, 7B10.A2, 13B11.A1, 14H11.A1, 21D6.G2, 22G9.D1, 24B4.A1, 26D2.D1, 26E2.A3, 30A8.A1, 38E9.E5, 39H10.A1, 42E8.H1, 43E9.H1, 44E2.H1, 49H11.B1, 52H9.D1, 53H11.D3, 54C2.A1, 55B9.A1, 57D7.A1, 60A4.B1, RS9.E2, RS9.F6, or RS9.F10. In some embodiments, the anti-TREM2 antibody enhances TREM2 activity and recognizes an epitope of human TREM2 that is identical to the epitope recognized by an antibody clone selected from the group consisting of 2G4.B1, 3D3.A1, 7B10.A2, 13B11.A1, 14H11.A1, 21D6.G2, 22G9.D1, 24B4.A1, 26D2.D1, 26E2.A3, 30A8.A1, 38E9.E5, 39H10.A1, 42E8.H1, 43E9.H1, 44E2.H1, 49H11.B1, 52H9.D1, 53H11.D3, 54C2.A1, 55B9.A1, 57D7.A1, 60A4.B1, RS9.E2, RS9.F6, and RS9.F10. In some embodiments, the anti-TREM2 antibody enhances TREM2 activity and recognizes an epitope of human TREM2 that is within the epitope recognized by an antibody clone selected from the group consisting of 2G4.B1, 3D3.A1, 7B10.A2, 13B11.A1, 14H11.A1, 21D6.G2, 22G9.D1, 24B4.A1, 26D2.D1, 26E2.A3, 30A8.A1, 38E9.E5, 39H10.A1, 42E8.H1, 43E9.H1, 44E2.H1, 49H11.B1, 52H9.D1, 53H11.D3, 54C2.A1, 55B9.A1, 57D7.A1, 60A4.B1, RS9.E2, RS9.F6, and RS9.F10. In some embodiments, the anti-TREM2 antibody enhances TREM2 activity and recognizes an epitope of human TREM2 that has at least 90% identity to the epitope recognized by an antibody clone selected from the group consisting of 2G4.B1, 3D3.A1, 7B10.A2, 13B11.A1, 14H11.A1, 21D6.G2, 22G9.D1, 24B4.A1, 26D2.D1, 26E2.A3, 30A8.A1, 38E9.E5, 39H10.A1, 42E8.H1, 43E9.H1, 44E2.H1, 49H11.B1, 52H9.D1, 53H11.D3, 54C2.A1, 55B9.A1, 57D7.A1, 60A4.B1, RS9.E2, RS9.F6, and RS9.F10. In some embodiments, the anti-TREM2 antibody enhances TREM2 activity and recognizes an epitope of human TREM2 that has one, two, or three amino acid substitutions (e.g., conservative substitutions) relative to the epitope recognized by an antibody clone selected from the group consisting of 2G4.B1, 3D3.A1, 7B10.A2, 13B11.A1, 14H11.A1, 21D6.G2, 22G9.D1, 24B4.A1, 26D2.D1, 26E2.A3, 30A8.A1, 38E9.E5, 39H10.A1, 42E8.H1, 43E9.H1, 44E2.H1, 49H11.B1, 52H9.D1, 53H11.D3, 54C2.A1, 55B9.A1, 57D7.A1, 60A4.B1, RS9.E2, RS9.F6, and RS9.F10.

In some embodiments, an anti-TREM antibody inhibits TREM2 activity (e.g., inhibits kinase phosphorylation, inhibits phagocytosis, and/or inhibits cell migration, differentiation, or survival) and recognizes an epitope that is the same or substantially the same as the epitope recognized by antibody clone 21D4.D1 or 21D11. In some embodiments, the anti-TREM2 antibody inhibits TREM2 activity and recognizes an epitope of human TREM2 that is identical to the epitope recognized by antibody clone 21D4.D1 or 21D11. In some embodiments, the anti-TREM2 antibody inhibits TREM2 activity and recognizes an epitope of human TREM2 that is within the epitope recognized by antibody clone 21D4.D1 or 21D11. In some embodiments, the anti-TREM2 antibody inhibits TREM2 activity and recognizes an epitope of human TREM2 that has at least 90% identity to the epitope recognized by antibody clone 21D4.D1 or 21D11. In some embodiments, the anti-TREM2 antibody inhibits TREM2 activity and recognizes an epitope of human TREM2 that has one, two, or three amino acid substitutions (e.g., conservative substitutions) relative to the epitope recognized by antibody clone 21D4.D1 or 21D11.

In some embodiments, an anti-TREM2 antibody enhances TREM2 activity (e.g., induces kinase phosphorylation, enhances phagocytosis, and/or enhances cell migration, differentiation, or survival) that is induced by a ligand and recognizes an epitope that is the same or substantially the same as the epitope recognized by antibody clone 3D3.A1, 8A11.B1, 14D5.F1, 19F10.F3, 21D6.G2, 22B8.B1, 22G9.D1, 26D11.B1, 26E.2.A3, 30A8.A1, 42E8.H1, 43E9.H1, 44E2.H1, 49H11.B1, 52H9.D1, 53H11.D3, 54C2.A1, 59C6.F1, 60A4.B1, RS9.F6, or RS9.F10. In some embodiments, the anti-TREM2 antibody enhances TREM2 activity that is induced by a ligand and recognizes an epitope of human TREM2 that is identical to the epitope recognized by an antibody clone selected from the group consisting of 3D3.A1, 8A11.B1, 14D5.F1, 19F10.F3, 21D6.G2, 22B8.B1, 22G9.D1, 26D11.B1, 26E.2.A3, 30A8.A1, 42E8.H1, 43E9.H1, 44E2.H1, 49H11.B1, 52H9.D1, 53H11.D3, 54C2.A1, 59C6.F1, 60A4.B1, RS9.F6, and RS9.F10. In some embodiments, the anti-TREM2 antibody enhances TREM2 activity that is induced by a ligand and recognizes an epitope of human TREM2 that is within the epitope recognized by an antibody clone selected from the group consisting of 3D3.A1, 8A11.B1, 14D5.F1, 19F10.F3, 21D6.G2, 22B8.B1, 22G9.D1, 26D11.B1, 26E.2.A3, 30A8.A1, 42E8.H1, 43E9.H1, 44E2.H1, 49H11.B1, 52H9.D1, 53H11.D3, 54C2.A1, 59C6.F1, 60A4.B1, RS9.F6, and RS9.F10. In some embodiments, the anti-TREM2 antibody enhances TREM2 activity that is induced by a ligand and recognizes an epitope of human TREM2 that has at least 90% identity to the epitope recognized by an antibody clone selected from the group consisting of 3D3.A1, 8A11.B1, 14D5.F1, 19F10.F3, 21D6.G2, 22B8.B1, 22G9.D1, 26D11.B1, 26E.2.A3, 30A8.A1, 42E8.H1, 43E9.H1, 44E2.H1, 49H11.B1, 52H9.D1, 53H11.D3, 54C2.A1, 59C6.F1, 60A4.B1, RS9.F6, and RS9.F10. In some embodiments, the anti-TREM2 antibody enhances TREM2 activity that is induced by a ligand and recognizes an epitope of human TREM2 that has one, two, or three amino acid substitutions (e.g., conservative substitutions) relative to the epitope recognized by an antibody clone selected from the group consisting of 3D3.A1, 8A11.B1, 14D5.F1, 19F10.F3, 21D6.G2, 22B8.B1, 22G9.D1, 26D11.B1, 26E.2.A3, 30A8.A1, 42E8.H1, 43E9.H1, 44E2.H1, 49H11.B1, 52H9.D1, 53H11.D3, 54C2.A1, 59C6.F1, 60A4.B1, RS9.F6, and RS9.F10.

In some embodiments, an anti-TREM2 antibody inhibits TREM2 activity (e.g., induces kinase phosphorylation, enhances phagocytosis, and/or enhances cell migration, differentiation, or survival) that is induced by a ligand and recognizes an epitope that is the same or substantially the same as the epitope recognized by antibody clone 2G4.B1, 13B11.A, 14H11.A1, 21D4.D1, 21D11.B1, 24B4.A1, 26D2.D1, 26D5.A1, 30A8.A1, 30F2.A2, 38E9.E5, 39H10.A1, 40H3.A4, 44E3.B1, 51D4.A1, 55B9.A1, 57D7.A1, or RS9.E2. In some embodiments, the anti-TREM2 antibody inhibits TREM2 activity that is induced by a ligand and recognizes an epitope of human TREM2 that is identical to the epitope recognized by an antibody clone selected from the group consisting of 2G4.B1, 13B11.A, 14H11.A1, 21D4.D1, 21D11.B1, 24B4.A1, 26D2.D1, 26D5.A1, 30A8.A1, 30F2.A2, 38E9.E5, 39H10.A1, 40H3.A4, 44E3.B1, 51D4.A1, 55B9.A1, 57D7.A1, and RS9.E2. In some embodiments, the anti-TREM2 antibody inhibits TREM2 activity that is induced by a ligand and recognizes an epitope of human TREM2 that is within the epitope recognized by an antibody clone selected from the group consisting of 2G4.B1, 13B11.A, 14H11.A1, 21D4.D1, 21D11.B1, 24B4.A1, 26D2.D1, 26D5.A1, 30A8.A1, 30F2.A2, 38E9.E5, 39H10.A1, 40H3.A4, 44E3.B1, 51D4.A1, 55B9.A1, 57D7.A1, and RS9.E2. In some embodiments, the anti-TREM2 antibody inhibits TREM2 activity that is induced by a ligand and recognizes an epitope of human TREM2 that has at least 90% identity to the epitope recognized by an antibody clone selected from the group consisting of 2G4.B1, 13B11.A, 14H11.A1, 21D4.D1, 21D11.B1, 24B4.A1, 26D2.D1, 26D5.A1, 30A8.A1, 30F2.A2, 38E9.E5, 39H10.A1, 40H3.A4, 44E3.B1, 51D4.A1, 55B9.A1, 57D7.A1, and RS9.E2. In some embodiments, the anti-TREM2 antibody inhibits TREM2 activity that is induced by a ligand and recognizes an epitope of human TREM2 that has one, two, or three amino acid substitutions (e.g., conservative substitutions) relative to the epitope recognized by an antibody clone selected from the group consisting of 2G4.B1, 13B11.A, 14H11.A1, 21D4.D1, 21D11.B1, 24B4.A1, 26D2.D1, 26D5.A1, 30A8.A1, 30F2.A2, 38E9.E5, 39H10.A1, 40H3.A4, 44E3.B1, 51D4.A1, 55B9.A1, 57D7.A1, and RS9.E2.

In some embodiments, an anti-TREM2 antibody recognizes an epitope of human TREM2 comprising, within, or consisting of residues 24-43, 44-58, 64-78, 89-103, 94-108, 124-153, 140-144, or 159-174 of SEQ ID NO:96. In some embodiments, an anti-TREM2 antibody recognizes an epitope comprising, within, or consisting of residues 24-43 of SEQ ID NO:96. In some embodiments, an anti-TREM2 antibody recognizes an epitope of human TREM2 comprising, within, or consisting of residues 44-58 of SEQ ID NO:96. In some embodiments, an anti-TREM2 antibody recognizes an epitope of human TREM2 comprising, within, or consisting of residues 64-78 of SEQ ID NO:96. In some embodiments, an anti-TREM2 antibody recognizes an epitope of human TREM2 comprising, within, or consisting of residues 89-103 of SEQ ID NO:96. In some embodiments, an anti-TREM2 antibody recognizes an epitope of human TREM2 comprising, within, or consisting of residues 94-108 of SEQ ID NO:96. In some embodiments, an anti-TREM2 antibody recognizes an epitope of human TREM2 comprising, within, or consisting of residues 124-153 of SEQ ID NO:96. In some embodiments, an anti-TREM2 antibody recognizes an epitope of human TREM2 comprising, within, or consisting of residues 140-148 of SEQ ID NO:96. In some embodiments, an anti-TREM2 antibody recognizes an epitope of human TREM2 comprising, within, or consisting of residues 140-144 of SEQ ID NO:96. In some embodiments, an anti-TREM2 antibody recognizes an epitope of human TREM2 comprising, within, or consisting of residues 140-144 of SEQ ID NO:96. In some embodiments, an anti-TREM2 antibody recognizes an epitope of human TREM2 comprising, within, or consisting of residues 159-174 of SEQ ID NO:96.

In some embodiments, an anti-TREM2 antibody that recognizes an epitope of human TREM2 comprising, within, or consisting of residues 140-148 of SEQ ID NO:96 (e.g., that recognizes an epitope of human TREM2 comprising, within, or consisting of residues 140-144 of SEQ ID NO:96) makes direct contact with one or more of residues Asp140, Leu141, Trp142, Phe143, and Pro144. In some embodiments, an anti-TREM2 antibody makes direct contact with residue Trp142. In some embodiments, an anti-TREM2 antibody makes direct contact with each of residues Asp140, Leu141, Trp142, Phe143, and Pro144.

### Anti-TREM2 Antibody Sequences

In some embodiments, an anti-TREM2 antibody comprises one or more complementarity determining region (CDR), heavy chain variable region, and/or light chain variable region sequences of the antibodies described herein. In some embodiments, an anti-TREM2 antibody comprises one or more CDR, heavy chain variable region, and/or light chain variable region sequences of the antibodies described herein and further comprises one or more functional characteristics as described herein (e.g., altering levels of sTREM2 and/or modulating one or more TREM2 activities).

CDR Sequences

In some embodiments, an anti-TREM2 antibody comprises one or more complementarity determining regions (CDRs) having at least 90%, 91%, 92%, 93%, 94%, 95%, 96%, 97%, 98%, or 99% sequence identity to a CDR of an antibody clone selected from the group consisting of 2G4.B1, 3D3.A1, 7B10.A2, 13B11.A1, 14D5.F1, 14H11.A1, 21D4.D1, 21D6.G2, 21D11, 22B8.B1, 22G9.D1, 24B4.A1, 24G7, 26D2, 26D11.B1, 26E2.A3, 30A8.A1, 38E9.E5, 39H10.A1, 40H3.A4, 42E8.H1, 43E9.H1, 44E2.H1, 44E3.B1, 49H11.B1, 51D4, 53H11.D3, 54C2.A1, 55B9.A1, 57D7.A1, 59C6.F1, 60A4.B1, RS9.E2, RS9.F6, RS9.F10, RS11.1F5, RS11.1G6, RS11.1A10, RS11.1D11, RS11.4A5, RS11.4H6, RS11.4D7, RS11.4D9, RS11.4F11, RS12.1C6, RS12.1C10, RS12.2D1, RS12.2D4, RS12.2E1, RS12.2F2, RS12.2G1, RS12.2H1, and RS12.3C10; or comprises one or more CDRs having up to two amino acid substitutions (i.e., zero, one, or two amino acid substitutions) relative to a CDR of an antibody clone selected from the group consisting of 2G4.B1, 3D3.A1, 7B10.A2, 13B11.A1, 14D5.F1, 14H11.A1, 21D4.D1, 21D6.G2, 21D11, 22B8.B1, 22G9.D1, 24B4.A1, 24G7, 26D2, 26D11.B1, 26E2.A3, 30A8.A1, 38E9.E5, 39H10.A1, 40H3.A4, 42E8.H1, 43E9.H1, 44E2.H1, 44E3.B1, 49H11.B1, 51D4, 53H11.D3, 54C2.A1, 55B9.A1, 57D7.A1, 59C6.F1, 60A4.B1, RS9.E2, RS9.F6, RS9.F10, RS11.1F5, RS11.1G6, RS11.1A10, RS11.1D11, RS11.4A5, RS11.4H6, RS11.4D7, RS11.4D9, RS11.4F11, RS12.1C6, RS12.1C10, RS12.2D1, RS12.2D4, RS12.2E1, RS12.2F2, RS12.2G1, RS12.2H1, and RS12.3C10.

In some embodiments, an anti-TREM2 antibody comprises one or more of a heavy chain CDR1 (CDR-H1), a heavy chain CDR2 (CDR-H2), a heavy chain CDR3 (CDR-H3), a light chain CDR1 (CDR-L1), a light chain CDR2 (CDR-L2), and a light chain CDR3 (CDR-L3) that is identical to a CDR-H1, CDR-H2, CDR-H3, CDR-L1, CDR-L2, and CDR-L3 of an antibody clone selected from the group consisting of 2G4.B1, 3D3.A1, 7B10.A2, 13B11.A1, 14D5.F1, 14H11.A1, 21D4.D1, 21D6.G2, 21D11, 22B8.B1, 22G9.D1, 24B4.A1, 24G7, 26D2, 26D11.B1, 26E2.A3, 30A8.A1, 38E9.E5, 39H10.A1, 40H3.A4, 42E8.H1, 43E9.H1, 44E2.H1, 44E3.B1, 49H11.B1, 51D4, 53H11.D3, 54C2.A1, 55B9.A1, 57D7.A1, 59C6.F1, 60A4.B1, RS9.E2, RS9.F6, RS9.F10, RS11.1F5, RS11.1G6, RS11.1A10, RS11.1D11, RS11.4A5, RS11.4H6, RS11.4D7, RS11.4D9, RS11.4F11, RS12.1C6, RS12.1C10, RS12.2D1, RS12.2D4, RS12.2E1, RS12.2F2, RS12.2G1, RS12.2H1, and RS12.3C10. In some embodiments, an anti-TREM2 antibody comprises two, three, four, five, or all six of CDR-H1, CDR-H2, CDR-H3, CDR-L1, CDR-L2, and CDR-L3 of an antibody clone selected from the group consisting of 2G4.B1, 3D3.A1, 7B10.A2, 13B11.A1, 14D5.F1, 14H11.A1, 21D4.D1, 21D6.G2, 21D11, 22B8.B1, 22G9.D1, 24B4.A1, 24G7, 26D2, 26D11.B1, 26E2.A3, 30A8.A1, 38E9.E5, 39H10.A1, 40H3.A4, 42E8.H1, 43E9.H1, 44E2.H1, 44E3.B1, 49H11.B1, 51D4, 53H11.D3, 54C2.A1, 55B9.A1, 57D7.A1, 59C6.F1, 60A4.B1, RS9.E2, RS9.F6, RS9.F10, RS11.1F5, RS11.1G6, RS11.1A10, RS11.1D11, RS11.4A5, RS11.4H6, RS11.4D7, RS11.4D9, RS11.4F11, RS12.1C6, RS12.1C10, RS12.2D1, RS12.2D4, RS12.2E1, RS12.2F2, RS12.2G1, RS12.2H1, and RS12.3C10.

In some embodiments, an anti-TREM2 antibody comprises the CDR-H1, CDR-H2, and CDR-H3 of an antibody clone selected from the group consisting of 2G4.B1, 3D3.A1, 7B10.A2, 13B11.A1, 14D5.F1, 14H11.A1, 21D4.D1, 21D6.G2, 21D11, 22B8.B1, 22G9.D1, 24B4.A1, 24G7, 26D2, 26D11.B1, 26E2.A3, 30A8.A1, 38E9.E5, 39H10.A1, 40H3.A4, 42E8.H1, 43E9.H1, 44E2.H1, 44E3.B1, 49H11.B1, 51D4, 53H11.D3, 54C2.A1, 55B9.A1, 57D7.A1, 59C6.F1, 60A4.B1, RS9.E2, RS9.F6, RS9.F10, RS11.1F5, RS11.1G6, RS11.1A10, RS11.1D11, RS11.4A5, RS11.4H6, RS11.4D7, RS11.4D9, RS11.4F11, RS12.1C6, RS12.1C10, RS12.2D1, RS12.2D4, RS12.2E1, RS12.2F2, RS12.2G1, RS12.2H1, and RS12.3C10. In some embodiments, an anti-TREM2 antibody comprises the CDR-L1, CDR-L2, and CDR-L3 of an antibody clone selected from the group consisting of 2G4.B1, 3D3.A1, 7B10.A2, 13B11.A1, 14D5.F1, 14H11.A1, 21D4.D1, 21D6.G2, 21D11, 22B8.B1, 22G9.D1, 24B4.A1, 24G7, 26D2, 26D11.B1, 26E2.A3, 30A8.A1, 38E9.E5, 39H10.A1, 40H3.A4, 42E8.H1, 43E9.H1, 44E2.H1, 44E3.B1, 49H11.B1, 51D4, 53H11.D3, 54C2.A1, 55B9.A1, 57D7.A1, 59C6.F1, 60A4.B1, RS9.E2, RS9.F6, RS9.F10, RS11.1F5, RS11.1G6, RS11.1A10, RS11.1D11, RS11.4A5, RS11.4H6, RS11.4D7, RS11.4D9, RS11.4F11, RS12.1C6, RS12.1C10, RS12.2D1, RS12.2D4, RS12.2E1, RS12.2F2, RS12.2G1, RS12.2H1, and RS12.3C10.

In some embodiments, an anti-TREM2 antibody comprises one or more CDRs selected from the group consisting of:
(a) a heavy chain CDR1 sequence having at least 90% sequence identity to the amino acid sequence of any one of SEQ ID NOs:8, 36, 39, 45, 51, 57, 62, 68, 74, 81, 85, 307, or 315 or having up to two amino acid substitutions relative to the amino acid sequence of any one of SEQ ID NOs:8, 36, 39, 45, 51, 57, 62, 68, 74, 81, 85, 307, or 315;
(b) a heavy chain CDR2 sequence having at least 90% sequence identity to the amino acid sequence of any one of SEQ ID NOs:9, 37, 40, 46, 52, 58, 63, 69, 75, 79, 82, 86, 308, or 316 or having up to two amino acid substitutions relative to the amino acid sequence of any one of SEQ ID NOs:9, 37, 40, 46, 52, 58, 63, 69, 75, 79, 82, 86, 308, or 316;
(c) a heavy chain CDR3 sequence having at least 90% sequence identity to the amino acid sequence of any one of SEQ ID NOs:10, 41, 47, 53, 59, 64, 70, 76, 83, 87, 309, or 317 or having up to two amino acid substitutions relative to the amino acid sequence of any one of SEQ ID NOs:10, 41, 47, 53, 59, 64, 70, 76, 83, 87, 309, or 317;
(d) a light chain CDR1 sequence having at least 90% sequence identity to the amino acid sequence of any one of SEQ ID NOs: 11, 42, 48, 54, 60, 65, 71, 77, 88, or 311 or having up to two amino acid substitutions relative to the amino acid sequence of any one of SEQ ID NOs:11, 42, 48, 54, 60, 65, 71, 77, 88, or 311;
(e) a light chain CDR2 sequence having at least 90% sequence identity to the amino acid sequence of any one of SEQ ID NOs: 12, 38, 43, 49, 55, 66, 72, 312, or 319 or having up to two amino acid substitutions relative to the amino acid sequence of any one of SEQ ID NOs: 12, 38, 43, 49, 55, 66, 72, 312, or 319; and
(f) a light chain CDR3 sequence having at least 90% sequence identity to the amino acid sequence of any one of SEQ ID NOs: 13, 44, 50, 56, 61, 67, 73, 78, 80, 84, 89, or 313 or having up to two amino acid substitutions relative to the amino acid sequence of any one of SEQ ID NOs:13, 44, 50, 56, 61, 67, 73, 78, 80, 84, 89, or 313.

In some embodiments, an anti-TREM2 antibody comprises two, three, four, five, or all six of (a)-(f). In some embodiments, an anti-TREM2 antibody comprises the heavy chain CDR1 of (a), the heavy chain CDR2 of (b), and the heavy chain CDR3 of (c). In some embodiments, an anti-TREM2 antibody comprises the light chain CDR1 of (d), the light chain CDR2 of (e), and the light chain CDR3 of (f). In some embodiments, a CDR having up to two amino acid substitutions has one amino acid substitution relative to the reference sequence. In some embodiments, a CDR having up to two amino acid substitutions has two amino acid substitutions relative to the reference sequence. In some embodiments, the up to two amino acid substitutions are conservative substitutions.

In some embodiments, an anti-TREM2 antibody comprises one or more CDRs selected from the group consisting of:
(a) a heavy chain CDR1 sequence comprising the amino acid sequence of any one of SEQ ID NOs:8, 36, 39, 45, 51, 57, 62, 68, 74, 81, 85, 307, or 315;
(b) a heavy chain CDR2 sequence comprising the amino acid sequence of any one of SEQ ID NOs:9, 37, 40, 46, 52, 58, 63, 69, 75, 79, 82, 86, 308, or 316;
(c) a heavy chain CDR3 sequence comprising the amino acid sequence of any one of SEQ ID NOs: 10, 41, 47, 53, 59, 64, 70, 76, 83, 87, 309, or 317;
(d) a light chain CDR1 sequence comprising the amino acid sequence of any one of SEQ ID NOs:11, 42, 48, 54, 60, 65, 71, 77, 88, or 311;
(e) a light chain CDR2 sequence comprising the amino acid sequence of any one of SEQ ID NOs: 12, 38, 43, 49, 55, 66, 72, 312, or 319; and
(f) a light chain CDR3 sequence comprising the amino acid sequence of any one of SEQ ID NOs: 13, 44, 50, 56, 61, 67, 73, 78, 80, 84, or 89.

In some embodiments, an anti-TREM2 antibody comprises two, three, four, five, or all six of (a)-(f). In some embodiments, an anti-TREM2 antibody comprises the heavy chain CDR1 of (a), the heavy chain CDR2 of (b), and the heavy chain CDR3 of (c). In some embodiments, an anti-TREM2 antibody comprises the light chain CDR1 of (d), the light chain CDR2 of (e), and the light chain CDR3 of (f).

In some embodiments, an anti-TREM2 antibody comprises one or more sequences that are variants of one or more consensus sequences. As a non-limiting example, consensus sequences can be identified by aligning heavy chain or light chain sequences (e.g., CDRs) for antibodies that are from the same (or similar) germlines. In some embodiments, consensus sequences may be generated from antibodies that contain sequences that are of the same (or similar) length and/or have at least one highly similar CDR (e.g., highly similar CDR3). In some embodiments, such sequences in these antibodies may be aligned and compared to identify conserved amino acids or motifs (i.e., where alteration in sequences may alter protein function) and/or regions where variation occurs the sequences (i.e., where variation of sequence is not likely to significantly affect protein function). Alternatively, consensus sequences can be identified by aligning heavy chain or light chain sequences (e.g., CDRs) for antibodies that bind to the same or similar (e.g., overlapping) epitopes to determine conserved amino acids or motifs (i.e., where alteration in sequences may alter protein function) and regions where variation occurs in alignment of sequences (i.e., where variation of sequence is not likely to significantly affect protein function). In some embodiments, one or more consensus sequences can be identified for antibodies that recognize the same or similar epitope as an anti-TREM2 antibody as disclosed herein (e.g., 3D3.A1, 7B10.A2, 21D4.D1, 21D11, 24B4.A1, 26D2.D1, 26E2.A3, 40H3.A4, 42E8.H1, 49H11.B1, 51D4, 54C2.A1, 57D7.A1, RS9.F6, or RS9.F10). Exemplary consensus sequences include SEQ ID NOs:320-332. In the consensus sequences of SEQ ID NOs:320-332, the capitalized letter represents an amino acid residue that is absolutely conserved among the aligned sequences (e.g., aligned CDR sequences), while “X” represents an amino acid residue that is not absolutely conserved among the aligned sequences. It will be appreciated that when selecting an amino acid to insert at a position marked by an “X” that in some embodiments, the amino acid is selected from those amino acids found at the corresponding position in the aligned sequences.

In some embodiments, the antibody comprises a heavy chain CDR1 (CDR-H1) consensus sequence comprising the formula GX₂X₃X₄X₅X₆X₇XX₉X₁₀X₁₁ (I) (SEQ ID NO:320), wherein X₂ is Y or F; X₃ is T, N, or S; X₄ is F, L, or I; X₅ is T, S, or K; X₆ is D, S, or E; X₇ is D or absent; X₈ is H, Y, or T; X₉ is A, N, G, V, W, T, or Y; X₁₀ is M, I, or W; and X₁₁ is H, Q, or N. In some embodiments, the CDR-H1 consensus sequence comprises the sequence GYTFTSYWMH (SEQ ID NO:36), GYTFTSYWIQ (SEQ ID NO:39), GYTFTDHAMH (SEQ ID NO:45), GYTFTSYVMH (SEQ ID NO:51), GYTLSEYTMH (SEQ ID NO:62), GFNIKDTYMH (SEQ ID NO:68), GYSITSDYAWN (SEQ ID NO:74), GYTFTDYNMH (SEQ ID NO:307), or GYTFTDYGMH (SEQ ID NO:315).

In some embodiments, the amino acids of formula I are further defined as follows: X₂ is Y; X₃ is T or S; X₅ is T or S; X₈ is H or Y; and X₉ is A, N, G, V, W, or T. In some embodiments, X₇ is absent. In some embodiments, X₁₀ is W and X₁₁ is N.

In some embodiments, the amino acids of formula I are further defined as follows: X₃ is T or N; X₇ is absent; X₁₀ is M or I, and X₁₁ is H or Q.

In some embodiments, the antibody comprises a CDR-H1 consensus sequence comprising the formula GYX₃X₄X₅X₆X₇XX₉X₁₀X₁₁ (II) (SEQ ID NO:321), wherein X₃ is T or S; X₄ is F, L, or I; X₅ is T or S; X₆ is D, S, or E; X₇ is D or absent; X₈ is H or Y; X₉ is A, N, G, V, W, T, or A; X₁₀ is M, I, or W; and X₁₁ is H, Q, or N.

In some embodiments, the antibody comprises a CDR-H1 consensus sequence comprising the formula GX₂X₃X₄X₅X₆XX₉X₁₀X₁₁ (III) (SEQ ID NO:322), wherein X₂ is Y or F; X₃ is T or N; X₄ is F, L, or I; X₅ is T, S, or K; X₆ is D, S, or E; X₈ is H, Y, or T; X₉ is A, N, G, V, W, T, Y, or A; X₁₀ is M or I; and X₁₁ is H or Q.

In some embodiments, the antibody comprises a CDR-H1 consensus sequence comprising the formula GYTX₄X₅X₆XsX₉X₁₀X₁₁ (IV) (SEQ ID NO:323), wherein X₄ is F or L; X₅ is T or S; X₆ is D, S, or E; X₈ is H, Y; X₉ is A, N, G, V, W, T; X₁₀ is M or I; and X₁₁ is H or Q.

In some embodiments, in the CDR-H1 consensus sequence of any one of formulas I, II, III, or IV, X₄ is F. In some embodiments, in the CDR-H1 consensus sequence of any one of formulas I, II, III, or IV, X₅ is T. In some embodiments, in the CDR-H1 consensus sequence of any one of formulas I, II, III, or IV, X₄ and X₅ are F and T, respectively. In some embodiments, in the CDR-H1 consensus sequence of any one of formulas I, II, III, or IV, X₆ is D or S. In some embodiments, in the CDR-H1 consensus sequence of any one of formulas I, II, III, or IV, X₈ is Y. In some embodiments, in the CDR-H1 consensus sequence of any one of formulas I, II, III, or IV, X₁₀ is M. In some embodiments, in the CDR-H1 consensus sequence of any one of formulas I, II, III, or IV, X₁₁ is H. In some embodiments, in the CDR-H1 consensus sequence of any one of formulas I, II, III, or IV, X₁₀ and X₁₁ are M and H, respectively. In some embodiments, in the CDR-H1 consensus sequence of any one of formulas I, II, III, or IV, X₁₀ and X₁₁ are I and Q, respectively.

In some embodiments, the antibody comprises a heavy chain CDR2 (CDR-H2) consensus sequence comprising the formula X₁X₂X₃X₄X₅X₆X₇X₈X₉X₁₀YX₁₂X₁₃X₁₄X₁₅X₁₆X₁₇ (V) (SEQ ID NO:324), wherein X₁ is D, V, Y, R, G, or T; X₂ is I, S, or V; X₃ is L, S, N, D, I, or Y; X₄ is P, T, or absent; X₅ is S, Y, N, T, A, G, or F; X₆ is I, S, N, T, or D; X₇ is G or D; X₈ is G, D, N, R, or S; X₉ is R, T, or A; X₁₀ is I, G, S, K, T, N, or R; X₁₂ is G, N, D, or T; X₁₃ is V, Q, E, or P; X₁₄ is K or S; X₁₅ is F, Y or L; X₁₆ is K, R, Q, or is absent; and X₁₇ is G, T, D, S, or is absent. In some embodiments, the CDR-H1 consensus sequence comprises the sequence RSDPTTGGTNYNEKFKT (SEQ ID NO:37), TIYPGDGDARYTQKFKG (SEQ ID NO:40), VISTYSGDTGYNQKFKG (SEQ ID NO:46), YINPYTDGTKYNEKFKG (SEQ ID NO:52), DILPSIGGRIYGVKF (SEQ ID NO:58), GVIPNSGGTSYNQKFRD (SEQ ID NO:63), RIDPANGNTKYDPKFQG (SEQ ID NO:69), YINYSGRTIYNPSLKS (SEQ ID NO:75), YISFSGSTSYNPSLKS (SEQ ID NO:79), YINPNNGGTTYNQKFKG (SEQ ID NO:308), or VISTYNGNTSYNQKYKG (SEQ ID NO:316).

In some embodiments, the amino acids of formula V are further defined as follows: X₁ is V, Y, R, G, or T; X₃ is S, N, D, I, or Y; X₅ is Y, N, T, A, G, or F; X₆ is S, N, T, or D; X₉ is T or A; X₁₂ is N, D, or T; X₁₃ is Q, E, or P; X₁₆ is K, R, or Q; X₁₇ is G, T, D, or S. In some embodiments, the amino acids of formula V are further defined as follows: X₄ is P or T; X₅ is Y, N, T, A, or G; X₈ is G, D, or N; X₁₀ is G, S, K, T, N, or R; X₁₄ is K; X₁₅ is F or Y; and X₁₇ is G, T, or D.

In some embodiments, the antibody comprises a CDR-H2 consensus sequence comprising the formula X₁X₂X₃X₄X₅X₆X₇X₈X₉X₁₀YX₁₂X₁₃X₁₄X₁₅X₁₆X₁₇ (VI) (SEQ ID NO:325), wherein X₁ is V, Y, R, G, or T; X₂ is I, S, or V; X₃ is S, N, D, I, or Y; X₄ is P, T, or absent; X₅ is Y, N, T, A, G, or F; X₆ is S, N, T, or D; X₇ is G or D; X₈ is G, D, N, R, or S; X₉ is T, or A; X₁₀ is I, G, S, K, T, N, or R; X₁₂ is N, D, or T; X₁₃ is Q, E, or P; X₁₄ is K or S; X₁₅ is F, Y or L; X₁₆ is K, R, or Q; and X₁₇ is G, T, D, or S.

In some embodiments, the antibody comprises a CDR-H2 consensus sequence comprising the formula X₁X₂X₃X₄X₅X₆X₇XX₉X₁₁YX₁₂X₁₃KX₁₅X₁₆X₁₇ (VII) (SEQ ID NO:326), wherein X₁ is V, Y, R, G, or T; X₂ is I, S, or V; X₃ is S, N, D, I, or Y; X₄ is P or T; X₅ is Y, N, T, A, or G; X₆ is S, N, T, or D; X₇ is G or D; X₈ is G, D, or N; X₉ is T, or A; X₁₀ is G, S, K, T, N, or R; X₁₂ is N, D, or T; X₁₃ is Q, E, or P; X₁₅ is F or Y; X₁₆ is K, R, or Q; and X₁₇ is G, T, or D.

In some embodiments, the antibody comprises a heavy chain CDR3 (CDR-H3) consensus sequence comprising the formula ARX₃X₄X₅X₆X₇XX₉X₁₀YAX₁₃DY (VIII) (SEQ ID NO:327), wherein X₃ is G or N; X₄ is D or G; X₅ is D or I; X₆ is S or T; X₇ is Y or T; X₈ is R or A; X₉ is R or G; X₁₀ is G or Y; and X₁₃ is L or M. In some embodiments, the CDR-H3 consensus sequence comprises the sequence ARNGITTAGYYAMDY (SEQ ID NO:41) or ARGDDSYRRGYALDY (SEQ ID NO:64).

In some embodiments, the antibody comprises a light chain CDR1 (CDR-L1) consensus sequence comprising the formula X₁SSX₄SLX₇XsX₉X₁₀X₁₁X₁₂X₁₃X₁₄X₁₅LX₁₇ (IX) (SEQ ID NO:328), wherein X₁ is R or K; X₄ is Q or K; X₇ is V or L; X₈ is H, D, or Y; X₉ is I, N, or S; X₁₀ is S or absent; X₁₁ is D or N; X₁₂ is G or Q; X₁₃ is N, I, or K; X₁₄ is T or S; X₁₅ is Y or F; and X₁₇ is Q, H, Y, N, or A. In some embodiments, the CDR-L1 consensus sequence comprises the sequence RSSQSLVHNNGNTFLH (SEQ ID NO:11), KSSQSLLDSDGKTYLN (SEQ ID NO:48), RSSQSLVHINGNTYLQ (SEQ ID NO:60), KSSQSLLYSSNQKSYLA (SEQ ID NO:65), RSSKSLLHSNGITYLY (SEQ ID NO:71), or RSSQSLVHINGNTYLH (SEQ ID NO:77).

In some embodiments, X₄ of formula IX is Q. In some embodiments, X₈ of formula IX is H. In some embodiments, X₉ of formula IX is I or S. In some embodiments, X₁₀ of formula IX is absent. In some embodiments, X₁₁ of formula IX is N. In some embodiments, X₁₂ of formula IX is G. In some embodiments, X₁₃ of formula IX is N or K. In some embodiments, X₁₄ of formula IX is T. In some embodiments, X₁₅ of formula IX is Y.

In some embodiments, the antibody comprises a CDR-L1 consensus sequence comprising the formula X₁ASX₄X₅IX₇X₈X₉LX₁₁ (X) (SEQ ID NO:329), wherein X₁ is R, K, or S; X₄ is E or Q; X₅ is N, D, or G; X₇ is Y or S; X₈ is S or N; X₉ is N, R, or Y; and X₁₁ is A or N. In some embodiments, the CDR-L1 consensus sequence comprises the sequence RASENIYSNLA (SEQ ID NO:42), KASEDIYNRLA (SEQ ID NO:54), or SASQGISNYLN (SEQ ID NO:311).

In some embodiments, the antibody comprises a light chain CDR2 (CDR-L2) consensus sequence comprising the formula X₁X₂SX₄X₅X₆S (XI) (SEQ ID NO:330), wherein X₁ is K, Q, Y, V, or L; X₂ is V, M, or T; X₄ is N, K, or Y; X₅ is R or L; and X₆ is F, A, H, or D. In some embodiments, the CDR-L2 consensus sequence comprises the sequence KVSNRFS (SEQ ID NO:38), VVSKLDS (SEQ ID NO:49), QMSNLAS (SEQ ID NO:72), YTSNLHS (SEQ ID NO:312), or LVSYLDS (SEQ ID NO:319).

In some embodiments, X₂ of formula XI is V. In some embodiments, X₄ of formula XI is N. In some embodiments, X₅ of formula XI is R. In some embodiments, X₅ of formula XI is L.

In some embodiments, the antibody comprises a light chain CDR3 (CDR-L3) consensus sequence comprising the formula X₁X₂X₃X₄X₅X₆X₇X₈T (XII) (SEQ ID NO:331), wherein X₁ is S, W, or Q; X₂ is Q or H; X₃ is S, T, G, Y, or F; X₄ is T, F, W, or S; X₅ is H, S, G, or N; X₆ is V, A, F, Y, T, or L; X₇ is P, T, or L; and X₈ is Y, F, P, or W. In some embodiments, the CDR-L2 consensus sequence comprises the sequence SQTTHVPPT (SEQ ID NO:13), QHFWGTPYT (SEQ ID NO:44), WQGTHFPYT (SEQ ID NO:50), QQYWSTPWT (SEQ ID NO:56), SQSTHVPYT (SEQ ID NO:61), QQYFSYPPT (SEQ ID NO:67), SQTTHALFT (SEQ ID NO:78), SQSTHVTFT (SEQ ID NO:80), or QQYSNLPYT (SEQ ID NO:313).

In some embodiments, the amino acids of formula XII are further defined as follows: X₁ is Q; X₃ is Y or F; X₄ is F, W, or S; X₅ is S, G, or N; X₆ is Y, T, or L; X₇ is P; X₈ is P, Y, or W. In some embodiments, X₂ of formula XII is Q. In some embodiments, X₄ of formula XII is T. In some embodiments, X₅ is H. In some embodiments, the amino acids of formula XII are further defined as follows: X₁ is S or W; X₂ is Q; X₃ is S, T, or G; X₄ is T; X₅ is H; X₆ is V, A, or F; X₇ is P, T, or L; and X₈ is Y, F, or P. In some embodiments, X₆ of formula XII is V or F.

In some embodiments, the antibody comprises a CDR-L3 consensus sequence comprising the formula QX₂X₃X₄X₅X₆PX₈T (XIII) (SEQ ID NO:332), wherein X₂ is Q or H; X₃ is Y or F; X₄ is F, W, or S; X₅ is S, G, or N; X₆ is Y, T, or L; and X₈ is P, Y, or W.

Variable Region Sequences

In some embodiments, an anti-TREM2 antibody comprises a heavy chain variable region comprising an amino acid sequence that has at least 90%, 91%, 92%, 93%, 94%, 95%, 96%, 97%, 98%, or 99% sequence identity to the heavy chain variable region of an antibody clone selected from the group consisting of 2G4.B1, 3D3.A1, 7B10.A2, 8A11.B1, 13B11.A1, 14D5.F1, 14H11.A1, 19F10.F3, 21D4.D1, 21D6.G2, 21D11, 22B8.B1, 22G9.D1, 24B4.A1, 26D2.D1, 26D5.A1, 26D11.B1, 26E2.A3, 30A8.A1, 30F2.A2, 38E9.E5, 39H10.A1, 40H3.A4, 42E8.H1, 43E9.H1, 44E2.H1, 44E3.B1, 49H11.B1, 51D4, 52H9.D1, 53H11.D3, 54C2.A1, 55B9.A1, 57D7.A1, 59C6.F1, 60A4.B1, RS9.E2, RS9.F6, RS9.F10, RS11.1F5, RS11.1G6, RS11.1A10, RS11.1D11, RS11.4A5, RS11.4H6, RS11.4D7, RS11.4D9, RS11.4F11, RS12.1C6, RS12.1C10, RS12.2D1, RS12.2D4, RS12.2E1, RS12.2F2, RS12.2G1, RS12.2H1, and RS12.3C10.

In some embodiments, an anti-TREM2 antibody comprises a light chain variable region comprising an amino acid sequence that has at least 90%, 91%, 92%, 93%, 94%, 95%, 96%, 97%, 98%, or 99% sequence identity to the light chain variable region of an antibody clone selected from the group consisting of 2G4.B1, 3D3.A1, 7B10.A2, 8A11.B1, 13B11.A1, 14D5.F1, 14H11.A1, 19F10.F3, 21D4.D1, 21D6.G2, 21D11, 22B8.B1, 22G9.D1, 24B4.A1, 26D2.D1, 26D5.A1, 26D11.B1, 26E2.A3, 30A8.A1, 30F2.A2, 38E9.E5, 39H10.A1, 40H3.A4, 42E8.H1, 43E9.H1, 44E2.H1, 44E3.B1, 49H11.B1, 51D4, 52H9.D1, 53H11.D3, 54C2.A1, 55B9.A1, 57D7.A1, 59C6.F1, 60A4.B1, RS9.E2, RS9.F6, RS9.F10, RS11.1F5, RS11.1G6, RS11.1A10, RS11.1D11, RS11.4A5, RS11.4H6, RS11.4D7, RS11.4D9, RS11.4F11, RS12.1C6, RS12.1C10, RS12.2D1, RS12.2D4, RS12.2E1, RS12.2F2, RS12.2G1, RS12.2H1, and RS12.3C10.

In some embodiments, an anti-TREM2 antibody comprises a heavy chain variable region comprising an amino acid sequence that has at least 90%, 91%, 92%, 93%, 94%, 95%, 96%, 97%, 98%, or 99% sequence identity to the heavy chain variable region of an antibody clone selected from the group consisting of 2G4.B1, 3D3.A1, 7B10.A2, 8A11.B1, 13B11.A1, 14D5.F1, 14H11.A1, 19F10.F3, 21D4.D1, 21D6.G2, 21D11, 22B8.B1, 22G9.D1, 24B4.A1, 26D2.D1, 26D5.A1, 26D11.B1, 26E2.A3, 30A8.A1, 30F2.A2, 38E9.E5, 39H10.A1, 40H3.A4, 42E8.H1, 43E9.H1, 44E2.H1, 44E3.B1, 49H11.B1, 51D4, 52H9.D1, 53H11.D3, 54C2.A1, 55B9.A1, 57D7.A1, 59C6.F1, 60A4.B1, RS9.E2, RS9.F6, RS9.F10, RS11.1F5, RS11.1G6, RS11.1A10, RS11.1D11, RS11.4A5, RS11.4H6, RS11.4D7, RS11.4D9, RS11.4F11, RS12.1C6, RS12.1C10, RS12.2D1, RS12.2D4, RS12.2E1, RS12.2F2, RS12.2G1, RS12.2H1, and RS12.3C10 and comprises a light chain variable region comprising an amino acid sequence that has at least 90%, 91%, 92%, 93%, 94%, 95%, 96%, 97%, 98%, or 99% sequence identity to the light chain variable region of an antibody clone selected from the group consisting of 2G4.B1, 3D3.A1, 7B10.A2, 8A11.B1, 13B11.A1, 14D5.F1, 14H11.A1, 19F10.F3, 21D4.D1, 21D6.G2, 21D11, 22B8.B1, 22G9.D1, 24B4.A1, 26D2.D1, 26D5.A1, 26D11.B1, 26E2.A3, 30A8.A1, 30F2.A2, 38E9.E5, 39H10.A1, 40H3.A4, 42E8.H1, 43E9.H1, 44E2.H1, 44E3.B1, 49H11.B1, 51D4, 52H9.D1, 53H11.D3, 54C2.A1, 55B9.A1, 57D7.A1, 59C6.F1, 60A4.B1, RS9.E2, RS9.F6, RS9.F10, RS11.1F5, RS11.1G6, RS11.1A10, RS11.1D11, RS11.4A5, RS11.4H6, RS11.4D7, RS11.4D9, RS11.4F11, RS12.1C6, RS12.1C10, RS12.2D1, RS12.2D4, RS12.2E1, RS12.2F2, RS12.2G1, RS12.2H1, and RS12.3C10.

In some embodiments, an anti-TREM2 comprises a heavy chain variable region comprising an amino acid sequence that has at least 90% sequence identity (e.g., at least 91%, at least 92%, at least 93%, at least 94%, at least 95%, at least 96%, at least 97%, at least 98%, or at least 99% sequence identity) to any one of SEQ ID NOs:6, 14, 15, 16, 17, 18, 19, 20, 21, 22, 23, 24, 306, or 314. In some embodiments, an anti-TREM2 comprises a heavy chain variable region comprising the amino acid sequence of any one of SEQ ID NOs:6, 14, 15, 16, 17, 18, 19, 20, 21, 22, 23, 24, 306, or 314.

In some embodiments, an anti-TREM2 comprises a light chain variable region comprising an amino acid sequence that has at least 90% sequence identity (e.g., at least 91%, at least 92%, at least 93%, at least 94%, at least 95%, at least 96%, at least 97%, at least 98%, or at least 99% sequence identity) to any one of SEQ ID NOs:7, 25, 26, 27, 28, 29, 30, 31, 32, 33, 34, 35, 310, or 318. In some embodiments, an anti-TREM2 comprises a light chain variable region comprising the amino acid sequence of any one of SEQ ID NOs:7, 25, 26, 27, 28, 29, 30, 31, 32, 33, 34, 35, 310, or 318.

CDR and Variable Region Sequences

In some embodiments, an anti-TREM2 antibody comprises a heavy chain variable region comprising an amino acid sequence that (i) has at least 75%, 80%, 85%, 90%, 91%, 92%, 93%, 94%, 95%, 96%, 97%, 98%, or 99% sequence identity to the heavy chain variable region of an antibody clone selected from the group consisting of 2G4.B1, 3D3.A1, 7B10.A2, 8A11.B1, 13B11.A1, 14D5.F1, 14H11.A1, 19F10.F3, 21D4.D1, 21D6.G2, 21D11, 22B8.B1, 22G9.D1, 24B4.A1, 26D2.D1, 26D5.A1, 26D11.B1, 26E2.A3, 30A8.A1, 30F2.A2, 38E9.E5, 39H10.A1, 40H3.A4, 42E8.H1, 43E9.H1, 44E2.H1, 44E3.B1, 49H11.B1, 51D4, 52H9.D1, 53H11.D3, 54C2.A1, 55B9.A1, 57D7.A1, 59C6.F1, 60A4.B1, RS9.E2, RS9.F6, RS9.F10, RS11.1F5, RS11.1G6, RS11.1A10, RS11.1D11, RS11.4A5, RS11.4H6, RS11.4D7, RS11.4D9, RS11.4F11, RS12.1C6, RS12.1C10, RS12.2D1, RS12.2D4, RS12.2E1, RS12.2F2, RS12.2G1, RS12.2H1, and RS12.3C10 and (ii) comprises a CDR-H1, CDR-H2, and CDR-H3 that is identical to the CDR-H1, CDR-H2, and CDR-H3 of the antibody clone.

In some embodiments, an anti-TREM2 antibody comprises a light chain variable region comprising an amino acid sequence that (i) has at least 75%, 80%, 85%, 90%, 91%, 92%, 93%, 94%, 95%, 96%, 97%, 98%, or 99% sequence identity to the light chain variable region of an antibody clone selected from the group consisting of 2G4.B1, 3D3.A1, 7B10.A2, 8A11.B1, 13B11.A1, 14D5.F1, 14H11.A1, 19F10.F3, 21D4.D1, 21D6.G2, 21D11, 22B8.B1, 22G9.D1, 24B4.A1, 26D2.D1, 26D5.A1, 26D11.B1, 26E2.A3, 30A8.A1, 30F2.A2, 38E9.E5, 39H10.A1, 40H3.A4, 42E8.H1, 43E9.H1, 44E2.H1, 44E3.B1, 49H11.B1, 51D4, 52H9.D1, 53H11.D3, 54C2.A1, 55B9.A1, 57D7.A1, 59C6.F1, 60A4.B1, RS9.E2, RS9.F6, RS9.F10, RS11.1F5, RS11.1G6, RS11.1A10, RS11.1D11, RS11.4A5, RS11.4H6, RS11.4D7, RS11.4D9, RS11.4F11, RS12.1C6, RS12.1C10, RS12.2D1, RS12.2D4, RS12.2E1, RS12.2F2, RS12.2G1, RS12.2H1, and RS12.3C10 and (ii) comprises a CDR-L1, CDR-L2, and CDR-L3 that is identical to the CDR-L1, CDR-L2, and CDR-L3 of the antibody clone.

In some embodiments, an anti-TREM2 antibody comprises:
(a) a heavy chain variable region comprising an amino acid sequence that (i) has at least 75%, 80%, 85%, 90%, 91%, 92%, 93%, 94%, 95%, 96%, 97%, 98%, or 99% sequence identity to the heavy chain variable region of an antibody clone selected from the group consisting of 2G4.B1, 3D3.A1, 7B10.A2, 8A11.B1, 13B11.A1, 14D5.F1, 14H11.A1, 19F10.F3, 21D4.D1, 21D6.G2, 21D11, 22B8.B1, 22G9.D1, 24B4.A1, 26D2.D1, 26D5.A1, 26D11.B1, 26E2.A3, 30A8.A1, 30F2.A2, 38E9.E5, 39H10.A1, 40H3.A4, 42E8.H1, 43E9.H1, 44E2.H1, 44E3.B1, 49H11.B1, 51D4, 52H9.D1, 53H11.D3, 54C2.A1, 55B9.A1, 57D7.A1, 59C6.F1, 60A4.B1, RS9.E2, RS9.F6, RS9.F10, RS11.1F5, RS11.1G6, RS11.1A10, RS11.1D11, RS11.4A5, RS11.4H6, RS11.4D7, RS11.4D9, RS11.4F11, RS12.1C6, RS12.1C10, RS12.2D1, RS12.2D4, RS12.2E1, RS12.2F2, RS12.2G1, RS12.2H1, and RS12.3C10 and (ii) comprises a CDR-H1, CDR-H2, and CDR-H3 that is identical to the CDR-H1, CDR-H2, and CDR-H3 of the antibody clone; and
(b) a light chain variable region comprising an amino acid sequence that (i) has at least 75%, 80%, 85%, 90%, 91%, 92%, 93%, 94%, 95%, 96%, 97%, 98%, or 99% sequence identity to the light chain variable region of an antibody clone selected from the group consisting of 2G4.B1, 3D3.A1, 7B10.A2, 8A11.B1, 13B11.A1, 14D5.F1, 14H11.A1, 19F10.F3, 21D4.D1, 21D6.G2, 21D11, 22B8.B1, 22G9.D1, 24B4.A1, 26D2.D1, 26D5.A1, 26D11.B1, 26E2.A3, 30A8.A1, 30F2.A2, 38E9.E5, 39H10.A1, 40H3.A4, 42E8.H1, 43E9.H1, 44E2.H1, 44E3.B1, 49H11.B1, 51D4, 52H9.D1, 53H11.D3, 54C2.A1, 55B9.A1, 57D7.A1, 59C6.F1, 60A4.B1, RS9.E2, RS9.F6, RS9.F10, RS11.1F5, RS11.1G6, RS11.1A10, RS11.1D11, RS11.4A5, RS11.4H6, RS11.4D7, RS11.4D9, RS11.4F11, RS12.1C6, RS12.1C10, RS12.2D1, RS12.2D4, RS12.2E1, RS12.2F2, RS12.2G1, RS12.2H1, and RS12.3C10 and (ii) comprises a CDR-L1, CDR-L2, and CDR-L3 that is identical to the CDR-L1, CDR-L2, and CDR-L3 of the antibody clone.

In some embodiments, an anti-TREM2 antibody is an antibody that competes for binding with an antibody as described herein (e.g., an antibody clone selected from the group consisting of 2G4.B1, 3D3.A1, 7B10.A2, 8A11.B1, 13B11.A1, 14D5.F1, 14H11.A1, 19F10.F3, 21D4.D1, 21D6.G2, 21D11, 22B8.B1, 22G9.D1, 24B4.A1, 26D2.D1, 26D5.A1, 26D11.B1, 26E2.A3, 30A8.A1, 30F2.A2, 38E9.E5, 39H10.A1, 40H3.A4, 42E8.H1, 43E9.H1, 44E2.H1, 44E3.B1, 49H11.B1, 51D4, 52H9.D1, 53H11.D3, 54C2.A1, 55B9.A1, 57D7.A1, 59C6.F1, 60A4.B1, RS9.E2, RS9.F6, RS9.F10, RS11.1F5, RS11.1G6, RS11.1A10, RS11.1D11, RS11.4A5, RS11.4H6, RS11.4D7, RS11.4D9, RS11.4F11, RS12.1C6, RS12.1C10, RS12.2D1, RS12.2D4, RS12.2E1, RS12.2F2, RS12.2G1, RS12.2H1, and RS12.3C10).

RS9.F6 and RS.F10

In some embodiments, an anti-TREM2 antibody comprises a heavy chain CDR1 sequence comprising the amino acid sequence of SEQ ID NO:8 or SEQ ID NO:36, a heavy chain CDR2 sequence comprising the amino acid sequence of SEQ ID NO:9 or SEQ ID NO:37, and a heavy chain CDR3 sequence comprising the amino acid sequence of SEQ ID NO:10. In some embodiments, an anti-TREM2 antibody comprises a light chain CDR1 sequence comprising the amino acid sequence of SEQ ID NO:11, a light chain CDR2 sequence comprising the amino acid sequence of SEQ ID NO:12 or SEQ ID NO:38, and a light chain CDR3 sequence comprising the amino acid sequence of SEQ ID NO: 13. In some embodiments, an anti-TREM2 antibody comprises a heavy chain CDR1 sequence comprising the amino acid sequence of SEQ ID NO:8 or SEQ ID NO:36, a heavy chain CDR2 sequence comprising the amino acid sequence of SEQ ID NO:9 or SEQ ID NO:37, a heavy chain CDR3 sequence comprising the amino acid sequence of SEQ ID NO: 10, a light chain CDR1 sequence comprising the amino acid sequence of SEQ ID NO:11, a light chain CDR2 sequence comprising the amino acid sequence of SEQ ID NO:12 or SEQ ID NO:38, and a light chain CDR3 sequence comprising the amino acid sequence of SEQ ID NO:13.

In some embodiments, an anti-TREM2 antibody comprises a heavy chain CDR1 sequence comprising the amino acid sequence of SEQ ID NO:8, a heavy chain CDR2 sequence comprising the amino acid sequence of SEQ ID NO:9, and a heavy chain CDR3 sequence comprising the amino acid sequence of SEQ ID NO: 10. In some embodiments, an anti-TREM2 antibody comprises a light chain CDR1 sequence comprising the amino acid sequence of SEQ ID NO:11, a light chain CDR2 sequence comprising the amino acid sequence of SEQ ID NO:12, and a light chain CDR3 sequence comprising the amino acid sequence of SEQ ID NO:13. In some embodiments, an anti-TREM2 antibody comprises a heavy chain CDR1-3 and a light chain CDR1-3 comprising the amino acid sequences of SEQ ID NOs:8, 9, 10, 11, 12, and 13, respectively.

In some embodiments, an anti-TREM2 antibody comprises a heavy chain CDR1 sequence comprising the amino acid sequence of SEQ ID NO:36, a heavy chain CDR2 sequence comprising the amino acid sequence of SEQ ID NO:37, and a heavy chain CDR3 sequence comprising the amino acid sequence of SEQ ID NO: 10. In some embodiments, an anti-TREM2 antibody comprises a light chain CDR1 sequence comprising the amino acid sequence of SEQ ID NO: 11, a light chain CDR2 sequence comprising the amino acid sequence of SEQ ID NO:38, and a light chain CDR3 sequence comprising the amino acid sequence of SEQ ID NO:13. In some embodiments, an anti-TREM2 antibody comprises a heavy chain CDR1-3 and a light chain CDR1-3 comprising the amino acid sequences of SEQ ID NOs:36, 37, 10, 11, 38, and 13, respectively.

In some embodiments, an anti-TREM2 antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:6 or SEQ ID NO:24. In some embodiments, an anti-TREM2 antibody comprises a heavy chain variable region comprising the amino acid sequence of SEQ ID NO:6 or SEQ ID NO:24. In some embodiments, an anti-TREM2 antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:6. In some embodiments, an anti-TREM2 antibody comprises a heavy chain variable region comprising the amino acid sequence of SEQ ID NO:6. In some embodiments, an anti-TREM2 antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:24. In some embodiments, an anti-TREM2 antibody comprises a heavy chain variable region comprising the amino acid sequence of SEQ ID NO:24.

In some embodiments, an anti-TREM2 antibody comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:7 or SEQ ID NO:35. In some embodiments, an anti-TREM2 antibody comprises a light chain variable region comprising the amino acid sequence of SEQ ID NO:7 or SEQ ID NO:35. In some embodiments, an anti-TREM2 antibody comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:7. In some embodiments, an anti-TREM2 antibody comprises a light chain variable region comprising the amino acid sequence of SEQ ID NO:7. In some embodiments, an anti-TREM2 antibody comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:35. In some embodiments, an anti-TREM2 antibody comprises a light chain variable region comprising the amino acid sequence of SEQ ID NO:35.

In some embodiments, an anti-TREM2 antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:6 and further comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:7. In some embodiments, an anti-TREM2 antibody comprises a heavy chain variable region comprising the amino acid sequence of SEQ ID NO:6 and further comprises a light chain variable region comprising the amino acid sequence of SEQ ID NO:7.

In some embodiments, an anti-TREM2 antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:24 and further comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:35. In some embodiments, an anti-TREM2 antibody comprises a heavy chain variable region comprising the amino acid sequence of SEQ ID NO:24 and further comprises a light chain variable region comprising the amino acid sequence of SEQ ID NO:35.

In some embodiments, an anti-TREM2 antibody comprises a heavy chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO:6 and comprises a heavy chain CDR1-3 comprising the amino acid sequences of SEQ ID NOs:8, 9, and 10, respectively.

In some embodiments, an anti-TREM2 antibody comprises a light chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO:6 and comprises a light chain CDR1-3 comprising the amino acid sequences of SEQ ID NOs: 11, 12, and 13, respectively.

In some embodiments, an anti-TREM2 antibody comprises a heavy chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO:6 and comprises a heavy chain CDR1-3 comprising the amino acid sequences of SEQ ID NOs:8, 9, and 10, respectively, and comprises a light chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO:6 and comprises a light chain CDR1-3 comprising the amino acid sequences of SEQ ID NOs: 11, 12, and 13, respectively.

In some embodiments, an anti-TREM2 antibody comprises a heavy chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO:24 and comprises a heavy chain CDR1-3 comprising the amino acid sequences of SEQ ID NOs:36, 37, and 10, respectively.

In some embodiments, an anti-TREM2 antibody comprises a light chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO:35 and comprises a light chain CDR1-3 comprising the amino acid sequences of SEQ ID NOs: 11, 38, and 13, respectively.

In some embodiments, an anti-TREM2 antibody comprises a heavy chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO:24 and comprises a heavy chain CDR1-3 comprising the amino acid sequences of SEQ ID NOs:36, 37, and 10, respectively, and comprises a light chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO:35 and comprises a light chain CDR1-3 comprising the amino acid sequences of SEQ ID NOs: 11, 38, and 13, respectively.

In some embodiments, an anti-TREM2 antibody is an antibody that competes for binding with an antibody as described herein (e.g., an antibody comprising a heavy chain CDR1-3 and a light chain CDR1-3 comprising the amino acid sequences of SEQ ID NOs:8, 9, 10, 11, 12, and 13, respectively, an antibody comprising a heavy chain variable region comprising the amino acid sequence of SEQ ID NO:6 and further comprising a light chain variable region comprising the amino acid sequence of SEQ ID NO:7, an antibody comprising a heavy chain CDR1-3 and a light chain CDR1-3 comprising the amino acid sequences of SEQ ID NOs:36, 37, 10, 11, 38, and 13, respectively, or an antibody comprising a heavy chain variable region comprising the amino acid sequence of SEQ ID NO:24 and further comprising a light chain variable region comprising the amino acid sequence of SEQ ID NO:35).

21D11

In some embodiments, an anti-TREM2 antibody comprises a heavy chain CDR1 sequence comprising the amino acid sequence of SEQ ID NO:39, a heavy chain CDR2 sequence comprising the amino acid sequence of SEQ ID NO:40, and a heavy chain CDR3 sequence comprising the amino acid sequence of SEQ ID NO:41. In some embodiments, an anti-TREM2 antibody comprises a light chain CDR1 sequence comprising the amino acid sequence of SEQ ID NO:42, a light chain CDR2 sequence comprising the amino acid sequence of SEQ ID NO:43, and a light chain CDR3 sequence comprising the amino acid sequence of SEQ ID NO:44. In some embodiments, an anti-TREM2 antibody comprises a heavy chain CDR1-3 and a light chain CDR1-3 comprising the amino acid sequences of SEQ ID NOs:39, 40, 41, 42, 43, and 44, respectively.

In some embodiments, an anti-TREM2 antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:14. In some embodiments, an anti-TREM2 antibody comprises a heavy chain variable region comprising the amino acid sequence of SEQ ID NO: 14.

In some embodiments, an anti-TREM2 antibody comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:25. In some embodiments, an anti-TREM2 antibody comprises a light chain variable region comprising the amino acid sequence of SEQ ID NO:25.

In some embodiments, an anti-TREM2 antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:14 and further comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:25. In some embodiments, an anti-TREM2 antibody comprises a heavy chain variable region comprising the amino acid sequence of SEQ ID NO: 14 and further comprises a light chain variable region comprising the amino acid sequence of SEQ ID NO:25.

In some embodiments, an anti-TREM2 antibody comprises a heavy chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO: 14 and comprises a heavy chain CDR1-3 comprising the amino acid sequences of SEQ ID NOs:39, 40, and 41, respectively.

In some embodiments, an anti-TREM2 antibody comprises a light chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO:25 and comprises a light chain CDR1-3 comprising the amino acid sequences of SEQ ID NOs:42, 43, and 44, respectively.

In some embodiments, an anti-TREM2 antibody comprises a heavy chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO: 14 and comprises a heavy chain CDR1-3 comprising the amino acid sequences of SEQ ID NOs:39, 40, and 41 respectively, and comprises a light chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO:25 and comprises a light chain CDR1-3 comprising the amino acid sequences of SEQ ID NOs:42, 43, and 44, respectively.

In some embodiments, an anti-TREM2 antibody is an antibody that competes for binding with an antibody as described herein (e.g., an antibody comprising a heavy chain CDR1-3 and a light chain CDR1-3 comprising the amino acid sequences of SEQ ID NOs:39, 40, 41, 42, 43, and 44, respectively, or an antibody comprising a heavy chain variable region comprising the amino acid sequence of SEQ ID NO: 14 and further comprising a light chain variable region comprising the amino acid sequence of SEQ ID NO:25). 21D4.D1

In some embodiments, an anti-TREM2 antibody comprises a heavy chain CDR1 sequence comprising the amino acid sequence of SEQ ID NO:45, a heavy chain CDR2 sequence comprising the amino acid sequence of SEQ ID NO:46, and a heavy chain CDR3 sequence comprising the amino acid sequence of SEQ ID NO:47. In some embodiments, an anti-TREM2 antibody comprises a light chain CDR1 sequence comprising the amino acid sequence of SEQ ID NO:48, a light chain CDR2 sequence comprising the amino acid sequence of SEQ ID NO:49, and a light chain CDR3 sequence comprising the amino acid sequence of SEQ ID NO:50. In some embodiments, an anti-TREM2 antibody comprises a heavy chain CDR1-3 and a light chain CDR1-3 comprising the amino acid sequences of SEQ ID NOs:45, 46, 47, 48, 49, and 50, respectively.

In some embodiments, an anti-TREM2 antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:15. In some embodiments, an anti-TREM2 antibody comprises a heavy chain variable region comprising the amino acid sequence of SEQ ID NO:15.

In some embodiments, an anti-TREM2 antibody comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:26. In some embodiments, an anti-TREM2 antibody comprises a light chain variable region comprising the amino acid sequence of SEQ ID NO:26.

In some embodiments, an anti-TREM2 antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:15 and further comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:26. In some embodiments, an anti-TREM2 antibody comprises a heavy chain variable region comprising the amino acid sequence of SEQ ID NO:15 and further comprises a light chain variable region comprising the amino acid sequence of SEQ ID NO:26.

In some embodiments, an anti-TREM2 antibody comprises a heavy chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO:15 and comprises a heavy chain CDR1-3 comprising the amino acid sequences of SEQ ID NOs:45, 46, and 47, respectively.

In some embodiments, an anti-TREM2 antibody comprises a light chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO:26 and comprises a light chain CDR1-3 comprising the amino acid sequences of SEQ ID NOs:48, 49, and 50, respectively.

In some embodiments, an anti-TREM2 antibody comprises a heavy chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO:15 and comprises a heavy chain CDR1-3 comprising the amino acid sequences of SEQ ID NOs:45, 46, and 47, respectively, and comprises a light chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO:26 and comprises a light chain CDR1-3 comprising the amino acid sequences of SEQ ID NOs:48, 49, and 50, respectively.

In some embodiments, an anti-TREM2 antibody is an antibody that competes for binding with an antibody as described herein (e.g., an antibody comprising a heavy chain CDR1-3 and a light chain CDR1-3 comprising the amino acid sequences of SEQ ID NOs:45, 46, 47, 48, 49, and 50, respectively, or an antibody comprising a heavy chain variable region comprising the amino acid sequence of SEQ ID NO:15 and further comprising a light chain variable region comprising the amino acid sequence of SEQ ID NO:26). 26D2

In some embodiments, an anti-TREM2 antibody comprises a heavy chain CDR1 sequence comprising the amino acid sequence of SEQ ID NO:51, a heavy chain CDR2 sequence comprising the amino acid sequence of SEQ ID NO:52, and a heavy chain CDR3 sequence comprising the amino acid sequence of SEQ ID NO:53. In some embodiments, an anti-TREM2 antibody comprises a light chain CDR1 sequence comprising the amino acid sequence of SEQ ID NO:54, a light chain CDR2 sequence comprising the amino acid sequence of SEQ ID NO:55, and a light chain CDR3 sequence comprising the amino acid sequence of SEQ ID NO:56. In some embodiments, an anti-TREM2 antibody comprises a heavy chain CDR1-3 and a light chain CDR1-3 comprising the amino acid sequences of SEQ ID NOs:51, 52, 53, 54, 55, and 56, respectively.

In some embodiments, an anti-TREM2 antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:16. In some embodiments, an anti-TREM2 antibody comprises a heavy chain variable region comprising the amino acid sequence of SEQ ID NO: 16.

In some embodiments, an anti-TREM2 antibody comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:27. In some embodiments, an anti-TREM2 antibody comprises a light chain variable region comprising the amino acid sequence of SEQ ID NO:27.

In some embodiments, an anti-TREM2 antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:16 and further comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:27. In some embodiments, an anti-TREM2 antibody comprises a heavy chain variable region comprising the amino acid sequence of SEQ ID NO:16 and further comprises a light chain variable region comprising the amino acid sequence of SEQ ID NO:27.

In some embodiments, an anti-TREM2 antibody comprises a heavy chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO: 16 and comprises a heavy chain CDR1-3 comprising the amino acid sequences of SEQ ID NOs:51, 52, and 53, respectively.

In some embodiments, an anti-TREM2 antibody comprises a light chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO:27 and comprises a light chain CDR1-3 comprising the amino acid sequences of SEQ ID NOs:54, 55, and 56, respectively.

In some embodiments, an anti-TREM2 antibody comprises a heavy chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO: 16 and comprises a heavy chain CDR1-3 comprising the amino acid sequences of SEQ ID NOs:51, 52, and 53, respectively, and comprises a light chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO:27 and comprises a light chain CDR1-3 comprising the amino acid sequences of SEQ ID NOs:54, 55, and 56, respectively.

In some embodiments, an anti-TREM2 antibody is an antibody that competes for binding with an antibody as described herein (e.g., an antibody comprising a heavy chain CDR1-3 and a light chain CDR1-3 comprising the amino acid sequences of SEQ ID NOs:51, 52, 53, 54, 55, and 56, respectively, or an antibody comprising a heavy chain variable region comprising the amino acid sequence of SEQ ID NO: 16 and further comprising a light chain variable region comprising the amino acid sequence of SEQ ID NO:27).

26E2.A3 and 24B4.A1

In some embodiments, an anti-TREM2 antibody comprises a heavy chain CDR1 sequence comprising the amino acid sequence of SEQ ID NO:57, a heavy chain CDR2 sequence comprising the amino acid sequence of SEQ ID NO:58, and a heavy chain CDR3 sequence comprising the amino acid sequence of SEQ ID NO:59. In some embodiments, an anti-TREM2 antibody comprises a light chain CDR1 sequence comprising the amino acid sequence of SEQ ID NO:60, a light chain CDR2 sequence comprising the amino acid sequence of SEQ ID NO:38, and a light chain CDR3 sequence comprising the amino acid sequence of SEQ ID NO:61. In some embodiments, an anti-TREM2 antibody comprises a heavy chain CDR1-3 and a light chain CDR1-3 comprising the amino acid sequences of SEQ ID NOs:57, 58, 59, 60, 38, and 61, respectively.

In some embodiments, an anti-TREM2 antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:17. In some embodiments, an anti-TREM2 antibody comprises a heavy chain variable region comprising the amino acid sequence of SEQ ID NO: 17.

In some embodiments, an anti-TREM2 antibody comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:28. In some embodiments, an anti-TREM2 antibody comprises a light chain variable region comprising the amino acid sequence of SEQ ID NO:28.

In some embodiments, an anti-TREM2 antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:17 and further comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:28. In some embodiments, an anti-TREM2 antibody comprises a heavy chain variable region comprising the amino acid sequence of SEQ ID NO:17 and further comprises a light chain variable region comprising the amino acid sequence of SEQ ID NO:28.

In some embodiments, an anti-TREM2 antibody comprises a heavy chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO: 17 and comprises a heavy chain CDR1-3 comprising the amino acid sequences of SEQ ID NOs:57, 58, and 59, respectively.

In some embodiments, an anti-TREM2 antibody comprises a light chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO:28 and comprises a light chain CDR1-3 comprising the amino acid sequences of SEQ ID NOs:60, 38, and 61, respectively.

In some embodiments, an anti-TREM2 antibody comprises a heavy chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO: 17 and comprises a heavy chain CDR1-3 comprising the amino acid sequences of SEQ ID NOs:57, 58, and 59, respectively, and comprises a light chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO:28 and comprises a light chain CDR1-3 comprising the amino acid sequences of SEQ ID NOs:60, 38, and 61, respectively.

In some embodiments, an anti-TREM2 antibody is an antibody that competes for binding with an antibody as described herein (e.g., an antibody comprising a heavy chain CDR1-3 and a light chain CDR1-3 comprising the amino acid sequences of SEQ ID NOs:57, 58, 59, 60, 38, and 61, respectively, or an antibody comprising a heavy chain variable region comprising the amino acid sequence of SEQ ID NO: 17 and further comprising a light chain variable region comprising the amino acid sequence of SEQ ID NO:28). 3D3.A1

In some embodiments, an anti-TREM2 antibody comprises a heavy chain CDR1 sequence comprising the amino acid sequence of SEQ ID NO:62, a heavy chain CDR2 sequence comprising the amino acid sequence of SEQ ID NO:63, and a heavy chain CDR3 sequence comprising the amino acid sequence of SEQ ID NO:64. In some embodiments, an anti-TREM2 antibody comprises a light chain CDR1 sequence comprising the amino acid sequence of SEQ ID NO:65, a light chain CDR2 sequence comprising the amino acid sequence of SEQ ID NO:66, and a light chain CDR3 sequence comprising the amino acid sequence of SEQ ID NO:67. In some embodiments, an anti-TREM2 antibody comprises a heavy chain CDR1-3 and a light chain CDR1-3 comprising the amino acid sequences of SEQ ID NOs:62, 63, 64, 65, 66, and 67, respectively.

In some embodiments, an anti-TREM2 antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:18. In some embodiments, an anti-TREM2 antibody comprises a heavy chain variable region comprising the amino acid sequence of SEQ ID NO:18.

In some embodiments, an anti-TREM2 antibody comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:29. In some embodiments, an anti-TREM2 antibody comprises a light chain variable region comprising the amino acid sequence of SEQ ID NO:29.

In some embodiments, an anti-TREM2 antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:18 and further comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:29. In some embodiments, an anti-TREM2 antibody comprises a heavy chain variable region comprising the amino acid sequence of SEQ ID NO:18 and further comprises a light chain variable region comprising the amino acid sequence of SEQ ID NO:29.

In some embodiments, an anti-TREM2 antibody comprises a heavy chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO:18 and comprises a heavy chain CDR1-3 comprising the amino acid sequences of SEQ ID NOs:62, 63, and 64, respectively.

In some embodiments, an anti-TREM2 antibody comprises a light chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO:29 and comprises a light chain CDR1-3 comprising the amino acid sequences of SEQ ID NOs:65, 66, and 67, respectively.

In some embodiments, an anti-TREM2 antibody comprises a heavy chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO:18 and comprises a heavy chain CDR1-3 comprising the amino acid sequences of SEQ ID NOs:62, 63, and 64, respectively, and comprises a light chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO:29 and comprises a light chain CDR1-3 comprising the amino acid sequences of SEQ ID NOs:65, 66, and 67, respectively.

In some embodiments, an anti-TREM2 antibody is an antibody that competes for binding with an antibody as described herein (e.g., an antibody comprising a heavy chain CDR1-3 and a light chain CDR1-3 comprising the amino acid sequences of SEQ ID NOs:62, 63, 64, 65, 66, and 67, respectively, or an antibody comprising a heavy chain variable region comprising the amino acid sequence of SEQ ID NO:18 and further comprising a light chain variable region comprising the amino acid sequence of SEQ ID NO:29).

40H3.A4

In some embodiments, an anti-TREM2 antibody comprises a heavy chain CDR1 sequence comprising the amino acid sequence of SEQ ID NO:68, a heavy chain CDR2 sequence comprising the amino acid sequence of SEQ ID NO:69, and a heavy chain CDR3 sequence comprising the amino acid sequence of SEQ ID NO:70. In some embodiments, an anti-TREM2 antibody comprises a light chain CDR1 sequence comprising the amino acid sequence of SEQ ID NO:71, a light chain CDR2 sequence comprising the amino acid sequence of SEQ ID NO:72, and a light chain CDR3 sequence comprising the amino acid sequence of SEQ ID NO:73. In some embodiments, an anti-TREM2 antibody comprises a heavy chain CDR1-3 and a light chain CDR1-3 comprising the amino acid sequences of SEQ ID NOs:68, 69, 70, 71, 72, and 73, respectively.

In some embodiments, an anti-TREM2 antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:19. In some embodiments, an anti-TREM2 antibody comprises a heavy chain variable region comprising the amino acid sequence of SEQ ID NO: 19.

In some embodiments, an anti-TREM2 antibody comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:30. In some embodiments, an anti-TREM2 antibody comprises a light chain variable region comprising the amino acid sequence of SEQ ID NO:30.

In some embodiments, an anti-TREM2 antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:19 and further comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:30. In some embodiments, an anti-TREM2 antibody comprises a heavy chain variable region comprising the amino acid sequence of SEQ ID NO:19 and further comprises a light chain variable region comprising the amino acid sequence of SEQ ID NO:30.

In some embodiments, an anti-TREM2 antibody comprises a heavy chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO:19 and comprises a heavy chain CDR1-3 comprising the amino acid sequences of SEQ ID NOs:68, 69, and 70, respectively.

In some embodiments, an anti-TREM2 antibody comprises a light chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO:30 and comprises a light chain CDR1-3 comprising the amino acid sequences of SEQ ID NOs:71, 72, and 73, respectively.

In some embodiments, an anti-TREM2 antibody comprises a heavy chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO:19 and comprises a heavy chain CDR1-3 comprising the amino acid sequences of SEQ ID NOs:68, 69, and 70, respectively, and comprises a light chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO:30 and comprises a light chain CDR1-3 comprising the amino acid sequences of SEQ ID NOs:71, 72, and 73, respectively.

In some embodiments, an anti-TREM2 antibody is an antibody that competes for binding with an antibody as described herein (e.g., an antibody comprising a heavy chain CDR1-3 and a light chain CDR1-3 comprising the amino acid sequences of SEQ ID NOs:68, 69, 70, 71, 72, and 73, respectively, or an antibody comprising a heavy chain variable region comprising the amino acid sequence of SEQ ID NO: 19 and further comprising a light chain variable region comprising the amino acid sequence of SEQ ID NO:30). 42E8.H1

In some embodiments, an anti-TREM2 antibody comprises a heavy chain CDR1 sequence comprising the amino acid sequence of SEQ ID NO:74, a heavy chain CDR2 sequence comprising the amino acid sequence of SEQ ID NO:75, and a heavy chain CDR3 sequence comprising the amino acid sequence of SEQ ID NO:76. In some embodiments, an anti-TREM2 antibody comprises a light chain CDR1 sequence comprising the amino acid sequence of SEQ ID NO:77, a light chain CDR2 sequence comprising the amino acid sequence of SEQ ID NO:38, and a light chain CDR3 sequence comprising the amino acid sequence of SEQ ID NO:78. In some embodiments, an anti-TREM2 antibody comprises a heavy chain CDR1-3 and a light chain CDR1-3 comprising the amino acid sequences of SEQ ID NOs:74, 75, 76, 77, 38, and 78, respectively.

In some embodiments, an anti-TREM2 antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:20. In some embodiments, an anti-TREM2 antibody comprises a heavy chain variable region comprising the amino acid sequence of SEQ ID NO:20.

In some embodiments, an anti-TREM2 antibody comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:31. In some embodiments, an anti-TREM2 antibody comprises a light chain variable region comprising the amino acid sequence of SEQ ID NO:31.

In some embodiments, an anti-TREM2 antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:20 and further comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:31. In some embodiments, an anti-TREM2 antibody comprises a heavy chain variable region comprising the amino acid sequence of SEQ ID NO:20 and further comprises a light chain variable region comprising the amino acid sequence of SEQ ID NO:31.

In some embodiments, an anti-TREM2 antibody comprises a heavy chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO:20 and comprises a heavy chain CDR1-3 comprising the amino acid sequences of SEQ ID NOs:74, 75, and 76, respectively.

In some embodiments, an anti-TREM2 antibody comprises a light chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO:31 and comprises a light chain CDR1-3 comprising the amino acid sequences of SEQ ID NOs:77, 38, and 78, respectively.

In some embodiments, an anti-TREM2 antibody comprises a heavy chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO:20 and comprises a heavy chain CDR1-3 comprising the amino acid sequences of SEQ ID NOs:74, 75, and 76, respectively, and comprises a light chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO:31 and comprises a light chain CDR1-3 comprising the amino acid sequences of SEQ ID NOs:77, 38, and 78, respectively.

In some embodiments, an anti-TREM2 antibody is an antibody that competes for binding with an antibody as described herein (e.g., an antibody comprising a heavy chain CDR1-3 and a light chain CDR1-3 comprising the amino acid sequences of SEQ ID NOs:74, 75, 76, 77, 38, and 78, respectively, or an antibody comprising a heavy chain variable region comprising the amino acid sequence of SEQ ID NO:20 and further comprising a light chain variable region comprising the amino acid sequence of SEQ ID NO:31).

49H11.B1

In some embodiments, an anti-TREM2 antibody comprises a heavy chain CDR1 sequence comprising the amino acid sequence of SEQ ID NO:74, a heavy chain CDR2 sequence comprising the amino acid sequence of SEQ ID NO:79, and a heavy chain CDR3 sequence comprising the amino acid sequence of SEQ ID NO:76. In some embodiments, an anti-TREM2 antibody comprises a light chain CDR1 sequence comprising the amino acid sequence of SEQ ID NO:77, a light chain CDR2 sequence comprising the amino acid sequence of SEQ ID NO:38, and a light chain CDR3 sequence comprising the amino acid sequence of SEQ ID NO:80. In some embodiments, an anti-TREM2 antibody comprises a heavy chain CDR1-3 and a light chain CDR1-3 comprising the amino acid sequences of SEQ ID NOs:74, 79, 76, 77, 38, and 80, respectively.

In some embodiments, an anti-TREM2 antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:21. In some embodiments, an anti-TREM2 antibody comprises a heavy chain variable region comprising the amino acid sequence of SEQ ID NO:21.

In some embodiments, an anti-TREM2 antibody comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:32. In some embodiments, an anti-TREM2 antibody comprises a light chain variable region comprising the amino acid sequence of SEQ ID NO:32.

In some embodiments, an anti-TREM2 antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:21 and further comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:32. In some embodiments, an anti-TREM2 antibody comprises a heavy chain variable region comprising the amino acid sequence of SEQ ID NO:21 and further comprises a light chain variable region comprising the amino acid sequence of SEQ ID NO:32.

In some embodiments, an anti-TREM2 antibody comprises a heavy chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO:21 and comprises a heavy chain CDR1-3 comprising the amino acid sequences of SEQ ID NOs:74, 79, and 76, respectively.

In some embodiments, an anti-TREM2 antibody comprises a light chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO:32 and comprises a light chain CDR1-3 comprising the amino acid sequences of SEQ ID NOs:77, 38, and 80, respectively.

In some embodiments, an anti-TREM2 antibody comprises a heavy chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO:21 and comprises a heavy chain CDR1-3 comprising the amino acid sequences of SEQ ID NOs:74, 79, and 76, respectively, and comprises a light chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO:32 and comprises a light chain CDR1-3 comprising the amino acid sequences of SEQ ID NOs:77, 38, and 80, respectively.

In some embodiments, an anti-TREM2 antibody is an antibody that competes for binding with an antibody as described herein (e.g., an antibody comprising a heavy chain CDR1-3 and a light chain CDR1-3 comprising the amino acid sequences of SEQ ID NOs:74, 79, 76, 77, 38, and 80, respectively, or an antibody comprising a heavy chain variable region comprising the amino acid sequence of SEQ ID NO:21 and further comprising a light chain variable region comprising the amino acid sequence of SEQ ID NO:32).

54C2.A1

In some embodiments, an anti-TREM2 antibody comprises a heavy chain CDR1 sequence comprising the amino acid sequence of SEQ ID NO:81, a heavy chain CDR2 sequence comprising the amino acid sequence of SEQ ID NO:82, and a heavy chain CDR3 sequence comprising the amino acid sequence of SEQ ID NO:83. In some embodiments, an anti-TREM2 antibody comprises a light chain CDR1 sequence comprising the amino acid sequence of SEQ ID NO:60, a light chain CDR2 sequence comprising the amino acid sequence of SEQ ID NO:38, and a light chain CDR3 sequence comprising the amino acid sequence of SEQ ID NO:84. In some embodiments, an anti-TREM2 antibody comprises a heavy chain CDR1-3 and a light chain CDR1-3 comprising the amino acid sequences of SEQ ID NOs:81, 82, 83, 60, 38, and 84, respectively.

In some embodiments, an anti-TREM2 antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:22. In some embodiments, an anti-TREM2 antibody comprises a heavy chain variable region comprising the amino acid sequence of SEQ ID NO:22.

In some embodiments, an anti-TREM2 antibody comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:33. In some embodiments, an anti-TREM2 antibody comprises a light chain variable region comprising the amino acid sequence of SEQ ID NO:33.

In some embodiments, an anti-TREM2 antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:22 and further comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:33. In some embodiments, an anti-TREM2 antibody comprises a heavy chain variable region comprising the amino acid sequence of SEQ ID NO:22 and further comprises a light chain variable region comprising the amino acid sequence of SEQ ID NO:33.

In some embodiments, an anti-TREM2 antibody comprises a heavy chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO:22 and comprises a heavy chain CDR1-3 comprising the amino acid sequences of SEQ ID NOs:81, 82, and 83, respectively.

In some embodiments, an anti-TREM2 antibody comprises a light chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO:33 and comprises a light chain CDR1-3 comprising the amino acid sequences of SEQ ID NOs:60, 38, and 84, respectively.

In some embodiments, an anti-TREM2 antibody comprises a heavy chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO:22 and comprises a heavy chain CDR1-3 comprising the amino acid sequences of SEQ ID NOs:81, 82, and 83, respectively, and comprises a light chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO:33 and comprises a light chain CDR1-3 comprising the amino acid sequences of SEQ ID NOs:60, 38, and 84, respectively.

In some embodiments, an anti-TREM2 antibody is an antibody that competes for binding with an antibody as described herein (e.g., an antibody comprising a heavy chain CDR1-3 and a light chain CDR1-3 comprising the amino acid sequences of SEQ ID NOs:81, 82, 83, 60, 38, and 84, respectively, or an antibody comprising a heavy chain variable region comprising the amino acid sequence of SEQ ID NO:22 and further comprising a light chain variable region comprising the amino acid sequence of SEQ ID NO:33).

57D7.A1

In some embodiments, an anti-TREM2 antibody comprises a heavy chain CDR1 sequence comprising the amino acid sequence of SEQ ID NO:85, a heavy chain CDR2 sequence comprising the amino acid sequence of SEQ ID NO:86, and a heavy chain CDR3 sequence comprising the amino acid sequence of SEQ ID NO:87. In some embodiments, an anti-TREM2 antibody comprises a light chain CDR1 sequence comprising the amino acid sequence of SEQ ID NO:88, a light chain CDR2 sequence comprising the amino acid sequence of SEQ ID NO:38, and a light chain CDR3 sequence comprising the amino acid sequence of SEQ ID NO:89. In some embodiments, an anti-TREM2 antibody comprises a heavy chain CDR1-3 and a light chain CDR1-3 comprising the amino acid sequences of SEQ ID NOs:85, 86, 87, 88, 38, and 89, respectively.

In some embodiments, an anti-TREM2 antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:23. In some embodiments, an anti-TREM2 antibody comprises a heavy chain variable region comprising the amino acid sequence of SEQ ID NO:23.

In some embodiments, an anti-TREM2 antibody comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:34. In some embodiments, an anti-TREM2 antibody comprises a light chain variable region comprising the amino acid sequence of SEQ ID NO:34.

In some embodiments, an anti-TREM2 antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:23 and further comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:34. In some embodiments, an anti-TREM2 antibody comprises a heavy chain variable region comprising the amino acid sequence of SEQ ID NO:23 and further comprises a light chain variable region comprising the amino acid sequence of SEQ ID NO:34.

In some embodiments, an anti-TREM2 antibody comprises a heavy chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO:23 and comprises a heavy chain CDR1-3 comprising the amino acid sequences of SEQ ID NOs:85, 86, and 87, respectively.

In some embodiments, an anti-TREM2 antibody comprises a light chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO:34 and comprises a light chain CDR1-3 comprising the amino acid sequences of SEQ ID NOs:88, 38, and 89, respectively.

In some embodiments, an anti-TREM2 antibody comprises a heavy chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO:23 and comprises a heavy chain CDR1-3 comprising the amino acid sequences of SEQ ID NOs:85, 86, and 87, respectively, and comprises a light chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO:34 and comprises a light chain CDR1-3 comprising the amino acid sequences of SEQ ID NOs:88, 38, and 89, respectively.

In some embodiments, an anti-TREM2 antibody is an antibody that competes for binding with an antibody as described herein (e.g., an antibody comprising a heavy chain CDR1-3 and a light chain CDR1-3 comprising the amino acid sequences of SEQ ID NOs:85, 86, 87, 88, 38, and 89, respectively, or an antibody comprising a heavy chain variable region comprising the amino acid sequence of SEQ ID NO:23 and further comprising a light chain variable region comprising the amino acid sequence of SEQ ID NO:34). 7B10.A2

In some embodiments, an anti-TREM2 antibody comprises a heavy chain CDR1 sequence comprising the amino acid sequence of SEQ ID NO:307, a heavy chain CDR2 sequence comprising the amino acid sequence of SEQ ID NO:308, and a heavy chain CDR3 sequence comprising the amino acid sequence of SEQ ID NO:309. In some embodiments, an anti-TREM2 antibody comprises a light chain CDR1 sequence comprising the amino acid sequence of SEQ ID NO:311, a light chain CDR2 sequence comprising the amino acid sequence of SEQ ID NO:312, and a light chain CDR3 sequence comprising the amino acid sequence of SEQ ID NO:313. In some embodiments, an anti-TREM2 antibody comprises a heavy chain CDR1-3 and a light chain CDR1-3 comprising the amino acid sequences of SEQ ID NOs:307, 308, 309, 311, 312, and 313, respectively.

In some embodiments, an anti-TREM2 antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:306. In some embodiments, an anti-TREM2 antibody comprises a heavy chain variable region comprising the amino acid sequence of SEQ ID NO:306.

In some embodiments, an anti-TREM2 antibody comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:310. In some embodiments, an anti-TREM2 antibody comprises a light chain variable region comprising the amino acid sequence of SEQ ID NO:310.

In some embodiments, an anti-TREM2 antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:306 and further comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:310. In some embodiments, an anti-TREM2 antibody comprises a heavy chain variable region comprising the amino acid sequence of SEQ ID NO:306 and further comprises a light chain variable region comprising the amino acid sequence of SEQ ID NO:310.

In some embodiments, an anti-TREM2 antibody comprises a heavy chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO:306 and comprises a heavy chain CDR1-3 comprising the amino acid sequences of SEQ ID NOs:307, 308, and 309, respectively.

In some embodiments, an anti-TREM2 antibody comprises a light chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO:310 and comprises a light chain CDR1-3 comprising the amino acid sequences of SEQ ID NOs:311, 312, and 313, respectively.

In some embodiments, an anti-TREM2 antibody comprises a heavy chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO:306 and comprises a heavy chain CDR1-3 comprising the amino acid sequences of SEQ ID NOs:307, 308, and 309, respectively, and comprises a light chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO:310 and comprises a light chain CDR1-3 comprising the amino acid sequences of SEQ ID NOs:311, 312, and 313, respectively.

In some embodiments, an anti-TREM2 antibody is an antibody that competes for binding with an antibody as described herein (e.g., an antibody comprising a heavy chain CDR1-3 and a light chain CDR1-3 comprising the amino acid sequences of SEQ ID NOs:307, 308, 309, 311, 312, and 313, respectively, or an antibody comprising a heavy chain variable region comprising the amino acid sequence of SEQ ID NO:306 and further comprising a light chain variable region comprising the amino acid sequence of SEQ ID NO:310). 51D4

In some embodiments, an anti-TREM2 antibody comprises a heavy chain CDR1 sequence comprising the amino acid sequence of SEQ ID NO:315, a heavy chain CDR2 sequence comprising the amino acid sequence of SEQ ID NO:316, and a heavy chain CDR3 sequence comprising the amino acid sequence of SEQ ID NO:317. In some embodiments, an anti-TREM2 antibody comprises a light chain CDR1 sequence comprising the amino acid sequence of SEQ ID NO:48, a light chain CDR2 sequence comprising the amino acid sequence of SEQ ID NO:319, and a light chain CDR3 sequence comprising the amino acid sequence of SEQ ID NO:50. In some embodiments, an anti-TREM2 antibody comprises a heavy chain CDR1-3 and a light chain CDR1-3 comprising the amino acid sequences of SEQ ID NOs:315, 316, 317, 48, 319, and 50, respectively.

In some embodiments, an anti-TREM2 antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:314. In some embodiments, an anti-TREM2 antibody comprises a heavy chain variable region comprising the amino acid sequence of SEQ ID NO:314.

In some embodiments, an anti-TREM2 antibody comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:318. In some embodiments, an anti-TREM2 antibody comprises a light chain variable region comprising the amino acid sequence of SEQ ID NO:318.

In some embodiments, an anti-TREM2 antibody comprises a heavy chain variable region comprising an amino acid sequence that has at least 90% sequence identity (e.g., at least 91%, 92%, 93%, 94%, 95%, 96%, 97%, 98%, or 99% sequence identity) to SEQ ID NO:314 and further comprises a light chain variable region comprising an amino acid sequence that has at least 90% sequence identity (e.g., at least 91%, 92%, 93%, 94%, 95%, 96%, 97%, 98%, or 99% sequence identity) to SEQ ID NO:318. In some embodiments, an anti-TREM2 antibody comprises a heavy chain variable region comprising the amino acid sequence of SEQ ID NO:314 and further comprises a light chain variable region comprising the amino acid sequence of SEQ ID NO:318.

In some embodiments, an anti-TREM2 antibody comprises a heavy chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO:314 and comprises a heavy chain CDR1-3 comprising the amino acid sequences of SEQ ID NOs:315, 316, and 317, respectively.

In some embodiments, an anti-TREM2 antibody comprises a light chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO:318 and comprises a light chain CDR1-3 comprising the amino acid sequences of SEQ ID NOs:48, 319, and 50, respectively.

In some embodiments, an anti-TREM2 antibody comprises a heavy chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO:314 and comprises a heavy chain CDR1-3 comprising the amino acid sequences of SEQ ID NOs:315, 316, and 317, respectively, and comprises a light chain variable region comprising an amino acid sequence that has at least 75% sequence identity (e.g., at least 80%, 85%, 90%, 91%, 92%, 93%, 94%, 95%, 96%, 97%, 98%, or 99% sequence identity to SEQ ID NO:318 and comprises a light chain CDR1-3 comprising the amino acid sequences of SEQ ID NOs:48, 319, and 50, respectively.

In some embodiments, an anti-TREM2 antibody is an antibody that competes for binding with an antibody as described herein (e.g., an antibody comprising a heavy chain CDR1-3 and a light chain CDR1-3 comprising the amino acid sequences of SEQ ID NOs:315, 316, 317, 48, 319, and 50, respectively, or an antibody comprising a heavy chain variable region comprising the amino acid sequence of SEQ ID NO:314 and further comprising a light chain variable region comprising the amino acid sequence of SEQ ID NO:318).

### Preparation of Antibodies

In some embodiments, antibodies are prepared by immunizing an animal or animals (e.g., mice, rabbits, or rats) with an antigen or a mixture of antigens for the induction of an antibody response. In some embodiments, the antigen or mixture of antigens is administered in conjugation with an adjuvant (e.g., Freund's adjuvant). After an initial immunization, one or more subsequent booster injections of the antigen or antigens may be administered to improve antibody production. Following immunization, antigen-specific B cells are harvested, e.g., from the spleen and/or lymphoid tissue. For generating monoclonal antibodies, the B cells are fused with myeloma cells, which are subsequently screened for antigen specificity. Methods of preparing antibodies are also described in the Examples section below.

The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Alternatively, phage or yeast display technology can be used to identify antibodies and Fab fragments that specifically bind to selected antigens. Antibodies can also be made bispecific, i.e., able to recognize two different antigens. Antibodies can also be heteroconjugates, e.g., two covalently joined antibodies, or immunotoxins.

Antibodies can be produced using any number of expression systems, including prokaryotic and eukaryotic expression systems. In some embodiments, the expression system is a mammalian cell expression, such as a hybridoma, or a CHO cell expression system. Many such systems are widely available from commercial suppliers. In embodiments in which an antibody comprises both a VH and VL region, the VH and VL regions may be expressed using a single vector, e.g., in a di-cistronic expression unit, or under the control of different promoters. In other embodiments, the VH and VL region may be expressed using separate vectors. A VH or VL region as described herein may optionally comprise a methionine at the N-terminus.

In some embodiments, the antibody is a chimeric antibody. Methods for making chimeric antibodies are known in the art. For example, chimeric antibodies can be made in which the antigen binding region (heavy chain variable region and light chain variable region) from one species, such as a mouse, is fused to the effector region (constant domain) of another species, such as a human. As another example, “class switched” chimeric antibodies can be made in which the effector region of an antibody is substituted with an effector region of a different immunoglobulin class or subclass.

In some embodiments, the antibody is a humanized antibody. Generally, a non-human antibody is humanized in order to reduce its immunogenicity. Humanized antibodies typically comprise one or more variable regions (e.g., CDRs) or portions thereof that are non-human (e.g., derived from a mouse variable region sequence), and possibly some framework regions or portions thereof that are non-human, and further comprise one or more constant regions that are derived from human antibody sequences. Methods for humanizing non-human antibodies are known in the art. Transgenic mice, or other organisms such as other mammals, can be used to express humanized or human antibodies. Other methods of humanizing antibodies include, for example, variable domain resurfacing, CDR grafting, grafting specificity-determining residues (SDR), guided selection, and framework shuffling.

As an alternative to humanization, fully human antibodies can be generated. As a non-limiting example, transgenic animals (e.g., mice) can be produced that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. As another example, human antibodies can be produced by hybridoma-based methods, such as by using primary human B cells for generating cell lines producing human monoclonal antibodies.

Human antibodies can also be produced using phage display or yeast display technology. In phage display, repertoires of variable heavy chain and variable light chain genes are amplified and expressed in phage display vectors. In some embodiments, the antibody library is a natural repertoire amplified from a human source. In some embodiments, the antibody library is a synthetic library made by cloning heavy chain and light chain sequences and recombining to generate a large pool of antibodies with different antigenic specificity. Phage typically display antibody fragments (e.g., Fab fragments or scFv fragments), which are then screened for binding to an antigen of interest.

In some embodiments, antibody fragments (such as a Fab, a Fab′, a F(ab′)₂, a scFv, a V_{H}, or a V_{HH}) are generated. Various techniques have been developed for the production of antibody fragments. Traditionally, these fragments were derived via proteolytic digestion of intact antibodies. However, these fragments can now be produced directly using recombinant host cells. For example, antibody fragments can be isolated from antibody phage libraries. Alternatively, Fab′-SH fragments can be directly recovered from E. coli cells and chemically coupled to form F(ab′)₂ fragments. According to another approach, F(ab′)₂ fragments can be isolated directly from recombinant host cell culture. Other techniques for the production of antibody fragments will be apparent to those skilled in the art.

In some embodiments, an antibody or an antibody fragment is conjugated to another molecule, e.g., polyethylene glycol (PEGylation) or serum albumin, to provide an extended half-life in vivo.

### Multispecific Antibodies

In some embodiments, multispecific antibodies comprising an anti-TREM2 antibody (or antigen-binding portion thereof) as described herein are provided, e.g., a bispecific antibody. Multispecific antibodies are antibodies that have binding specificities for at least two different sites. In some embodiments, a multispecific antibody (e.g., a bispecific antibody) has a binding specificity for TREM2 and has a binding specificity for at least one other antigen. In some embodiments, a multispecific antibody (e.g., a bispecific antibody) binds to two different TREM2 epitopes. In some embodiments, a multispecific antibody (e.g., a bispecific antibody) is capable of inducing TREM2 clustering at the cell surface. An illustrative method for measuring receptor clustering using confocal FRET microscopy is described in Wallrabe et al., Biophys. 1, 2003, 85:559-571.

Methods for making multispecific antibodies include, but are not limited to, recombinant co-expression of two pairs of heavy chain and light chain in a host cell, “knobs-into-holes” engineering, intramolecular trimerization, and fusion of an antibody fragment to the N-terminus or C-terminus of another antibody, e.g., tandem variable domains.

### Nucleic Acids, Vectors, and Host Cells

In some embodiments, the anti-TREM2 antibodies as described herein are prepared using recombinant methods. Accordingly, in some aspects, the disclosure provides isolated nucleic acids comprising a nucleic acid sequence encoding any of the anti-TREM2 antibodies as described herein (e.g., any one or more of the CDRs, heavy chain variable regions, and light chain variable regions described herein); vectors comprising such nucleic acids; and host cells into which the nucleic acids are introduced that are used to replicate the antibody-encoding nucleic acids and/or to express the antibodies.

In some embodiments, a polynucleotide (e.g., an isolated polynucleotide) comprises a nucleotide sequence encoding an antibody or antigen-binding portion thereof as described herein (e.g., as described in the Section above entitled “Anti-TREM2 Antibody Sequences”). In some embodiments, the polynucleotide comprises a nucleotide sequence encoding one or more amino acid sequences (e.g., CDR, heavy chain, light chain, and/or framework regions) that is identical to the sequence (e.g., CDR, heavy chain, light chain, and/or framework region sequence) of an antibody clone selected from the group consisting of 2G4.B1, 3D3.A1, 7B10.A2, 8A11.B1, 13B11.A1, 14D5.F1, 14H11.A1, 19F10.F3, 21D4.D1, 21D6.G2, 21D11, 22B8.B1, 22G9.D1, 24B4.A1, 26D2.D1, 26D5.A1, 26D11.B1, 26E2.A3, 30A8.A1, 30F2.A2, 38E9.E5, 39H10.A1, 40H3.A4, 42E8.H1, 43E9.H1, 44E2.H1, 44E3.B1, 49H11.B1, 51D4, 52H9.D1, 53H11.D3, 54C2.A1, 55B9.A1, 57D7.A1, 59C6.F1, 60A4.B1, RS9.E2, RS9.F6, RS9.F10, RS11.1F5, RS11.1G6, RS11.1A10, RS11.1D11, RS11.4A5, RS11.4H6, RS11.4D7, RS11.4D9, RS11.4F11, RS12.1C6, RS12.1C10, RS12.2D1, RS12.2D4, RS12.2E1, RS12.2F2, RS12.2G1, RS12.2H1, and RS12.3C10. In some embodiments, a polynucleotide as described herein is operably linked to a heterologous nucleic acid, e.g., a heterologous promoter.

Suitable vectors containing polynucleotides encoding antibodies of the present disclosure, or fragments thereof, include cloning vectors and expression vectors. While the cloning vector selected may vary according to the host cell intended to be used, useful cloning vectors generally have the ability to self-replicate, may possess a single target for a particular restriction endonuclease, and/or may carry genes for a marker that can be used in selecting clones containing the vector. Examples include plasmids and bacterial viruses, e.g., pUC18, pUC19, Bluescript (e.g., pBS SK+) and its derivatives, mp18, mp19, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Expression vectors generally are replicable polynucleotide constructs that contain a nucleic acid of the present disclosure. The expression vector may replicate in the host cells either as episomes or as an integral part of the chromosomal DNA. Suitable expression vectors include but are not limited to plasmids, viral vectors, including adenoviruses, adeno-associated viruses, retroviruses, and any other vector.

Suitable host cells for cloning or expressing a polynucleotide or vector as described herein include prokaryotic or eukaryotic cells. In some embodiments, the host cell is prokaryotic. In some embodiments, the host cell is eukaryotic, e.g., Chinese Hamster Ovary (CHO) cells or lymphoid cells. In some embodiments, the host cell is a human cell, e.g., a Human Embryonic Kidney (HEK) cell.

In another aspect, methods of making an anti-TREM2 antibody as described herein are provided. In some embodiments, the method includes culturing a host cell as described herein (e.g., a host cell expressing a polynucleotide or vector as described herein) under conditions suitable for expression of the antibody. In some embodiments, the antibody is subsequently recovered from the host cell (or host cell culture medium).

### IV. Fc Polypeptide Modifications for Blood-Brain Barrier (Bbb) Receptor Binding

In some aspects, provided herein are anti-TREM2 antibodies that are capable of being transported across the blood-brain barrier (BBB). Such a protein comprises a modified Fc polypeptide that binds to a BBB receptor. BBB receptors are expressed on BBB endothelia, as well as other cell and tissue types. In some embodiments, the BBB receptor is a transferrin receptor (TfR).

Amino acid residues designated in various Fc modifications, including those introduced in a modified Fc polypeptide that binds to a BBB receptor, e.g., TfR, are numbered herein using EU index numbering. Any Fc polypeptide, e.g., an IgG1, IgG2, IgG3, or IgG4 Fc polypeptide, may have modifications, e.g., amino acid substitutions, in one or more positions as described herein.

In some embodiments, an anti-TREM2 antibody comprises a first and optionally a second Fc polypeptide, each of which can be independently modified. In some embodiments, modifications (e.g., that promote TfR binding) that are made to the first and/or second Fc polypeptides result in an increase in brain uptake of the antibody (or antigen-binding portion thereof) of at least about 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, or more, compared to the uptake without the modifications having been made.

A modified (e.g., enhancing heterodimerization and/or BBB receptor-binding) Fc polypeptide can have at least 70% identity, at least 75% identity, at least 80% identity, at least 85% identity, at least 90% identity, or at least 95% identity to a native Fc region sequence or a fragment thereof, e.g., a fragment of at least 50 amino acids or at least 100 amino acids, or greater in length. In some embodiments, the native Fc amino acid sequence is the Fc region sequence of SEQ ID NO:98. In some embodiments, the modified Fc polypeptide has at least 70% identity, at least 75% identity, at least 80% identity, at least 85% identity, at least 90% identity, or at least 95% identity to amino acids 1-110 of SEQ ID NO:98, or to amino acids 111-217 of SEQ ID NO:98, or a fragment thereof, e.g., a fragment of at least 50 amino acids or at least 100 amino acids, or greater in length.

In some embodiments, a modified (e.g., enhancing heterodimerization and/or BBB receptor-binding) Fc polypeptide comprises at least 50 amino acids, or at least 60, 65, 70, 75, 80, 85, 90, or 95 or more, or at least 100 amino acids, or more, that correspond to a native Fc region amino acid sequence. In some embodiments, the modified Fc polypeptide comprises at least 25 contiguous amino acids, or at least 30, 35, 40, or 45 contiguous amino acids, or 50 contiguous amino acids, or at least 60, 65, 70, 75, 80 85, 90, or 95 or more contiguous amino acids, or 100 or more contiguous amino acids, that correspond to a native Fc region amino acid sequence, such as SEQ ID NO:98.

In some embodiments, the domain that is modified for BBB receptor-binding activity is a human Ig CH3 domain, such as an IgG1 CH3 domain. The CH3 domain can be of any IgG subtype, i.e., from IgG1, IgG2, IgG3, or IgG4. In the context of IgG1 antibodies, a CH3 domain refers to the segment of amino acids from about position 341 to about position 447 as numbered according to the EU numbering scheme.

In some embodiments, the domain that is modified for BBB receptor-binding activity is a human Ig CH2 domain, such as an IgG CH2 domain. The CH2 domain can be of any IgG subtype, i.e., from IgG1, IgG2, IgG3, or IgG4. In the context of IgG1 antibodies, a CH2 domain refers to the segment of amino acids from about position 231 to about position 340 as numbered according to the EU numbering scheme.

In some embodiments, a modified (e.g., BBB receptor-binding) Fc polypeptide comprises at least one, two, or three substitutions; and in some embodiments, at least four five, six, seven, eight, nine, or ten substitutions at amino acid positions comprising 266, 267, 268, 269, 270, 271, 295, 297, 298, and 299, according to the EU numbering scheme.

In some embodiments, a modified (e.g., BBB receptor-binding) Fc polypeptide comprises at least one, two, or three substitutions; and in some embodiments, at least four, five, six, seven, eight, or nine substitutions at amino acid positions comprising 274, 276, 283, 285, 286, 287, 288, 289, and 290, according to the EU numbering scheme.

In some embodiments, a modified (e.g., BBB receptor-binding) Fc polypeptide comprises at least one, two, or three substitutions; and in some embodiments, at least four, five, six, seven, eight, nine, or ten substitutions at amino acid positions comprising 268, 269, 270, 271, 272, 292, 293, 294, 296, and 300, according to the EU numbering scheme.

In some embodiments, a modified (e.g., BBB receptor-binding) Fc polypeptide comprises at least one, two, or three substitutions; and in some embodiments, at least four, five, six, seven, eight, or nine substitutions at amino acid positions comprising 272, 274, 276, 322, 324, 326, 329, 330, and 331, according to the EU numbering scheme.

In some embodiments, a modified (e.g., BBB receptor-binding) Fc polypeptide comprises at least one, two, or three substitutions; and in some embodiments, at least four, five, six, or seven substitutions at amino acid positions comprising 345, 346, 347, 349, 437, 438, 439, and 440, according to the EU numbering scheme.

In some embodiments, a modified (e.g., BBB receptor-binding) Fc polypeptide comprises at least one, two, or three substitutions; and in some embodiments, at least four, five, six, seven, eight, or nine substitutions at amino acid positions 384, 386, 387, 388, 389, 390, 413, 416, and 421, according to the EU numbering scheme.

In some embodiments, an anti-TREM2 antibody comprises two Fc polypeptides, wherein one Fc polypeptide is not modified to bind to a BBB receptor (e.g., TfR) and the other Fc polypeptide is modified to specifically bind to a BBB receptor (e.g., TfR).

### FcRn Binding Sites

In certain aspects, modified (e.g., BBB receptor-binding) Fc polypeptides, or Fc polypeptides that do not specifically bind to a BBB receptor, can also comprise an FcRn binding site. In some embodiments, the FcRn binding site is within the Fc polypeptide or a fragment thereof.

In some embodiments, the FcRn binding site comprises a native FcRn binding site. In some embodiments, the FcRn binding site does not comprise amino acid changes relative to the amino acid sequence of a native FcRn binding site. In some embodiments, the native FcRn binding site is an IgG binding site, e.g., a human IgG binding site. In some embodiments, the FcRn binding site comprises a modification that alters FcRn binding.

In some embodiments, one or more Fc polypeptides (e.g., a first Fc polypeptide, a second Fc polypeptide, or a first and second Fc polypeptide) contain modifications that affect (e.g., increase) FcRn binding. In some embodiments, an FcRn binding site has one or more amino acid residues that are mutated, e.g., substituted, wherein the mutation(s) increase serum half-life or do not substantially reduce serum half-life (i.e., reduce serum half-life by no more than 25% compared to a counterpart modified Fc polypeptide having the wild-type residues at the mutated positions when assayed under the same conditions). In some embodiments, an FcRn binding site has one or more amino acid residues that are substituted at positions 251-256, 428, and 433-436, according to the EU numbering scheme.

In some embodiments, one or more residues at or near an FcRn binding site are mutated, relative to a native human IgG sequence, to extend serum half-life of the modified polypeptide. In some embodiments, a mutation, e.g., a substitution, is introduced at one or more of positions 244-257, 279-284, 307-317, 383-390, and 428-435, according to the EU numbering scheme. In some embodiments, one or more mutations are introduced at positions 251, 252, 254, 255, 256, 307, 308, 309, 311, 312, 314, 385, 386, 387, 389, 428, 433, 434, or 436, according to the EU numbering scheme. In some embodiments, mutations are introduced into one, two, or three of positions 252, 254, and 256. In some embodiments, the mutations are M252Y, S254T, and T256E. In some embodiments, a modified Fc polypeptide further comprises the mutations M252Y, S254T, and T256E. In some embodiments, a modified Fc polypeptide comprises a mutation at one, two, or all three of positions T307, E380, and N434, according to the EU numbering scheme. In some embodiments, the mutations are T307Q and N434A. In some embodiments, a modified Fc polypeptide comprises mutations T307A, E380A, and N434A. In some embodiments, a modified Fc polypeptide comprises mutations at positions T250 and M428, according to the EU numbering scheme. In some embodiments, the Fc polypeptide comprises mutations T250Q and/or M428L. In some embodiments, a modified Fc polypeptide comprises mutations at positions M428 and N434, according to the EU numbering scheme. In some embodiments, the modified Fc polypeptide comprises mutations M428L and N434S. In some embodiments, the modified Fc polypeptide comprises an N434S or N434A mutation.

### Transferrin Receptor-Binding Fc Polypeptides

In some embodiments, an anti-TREM2 antibody as disclosed herein comprises a modified Fc polypeptide that binds to a transferrin receptor (TfR) and is capable of being transported across the blood-brain barrier (BBB). In some embodiments, a modified Fc polypeptide that specifically binds to TfR comprises substitutions in a CH3 domain. In some embodiments, a modified Fc polypeptide that specifically binds to TfR comprises substitutions in a CH2 domain.

TfR-Binding Fc Polypeptides Comprising Mutations in the CH3 Domain

In some embodiments, a modified Fc polypeptide that specifically binds to TfR comprises substitutions in a CH3 domain. In some embodiments, a modified Fc polypeptide comprises a human Ig CH3 domain, such as an IgG CH3 domain, that is modified for TfR-binding activity. The CH3 domain can be of any IgG subtype, i.e., from IgG1, IgG2, IgG3, or IgG4. In the context of IgG antibodies, a CH3 domain refers to the segment of amino acids from about position 341 to about position 447 as numbered according to the EU numbering scheme.

In some embodiments, a modified Fc polypeptide that specifically binds to TfR binds to the apical domain of TfR and may bind to TfR without blocking or otherwise inhibiting binding of transferrin to TfR. In some embodiments, binding of transferrin to TfR is not substantially inhibited. In some embodiments, binding of transferrin to TfR is inhibited by less than about 50% (e.g., less than about 45%, 40%, 35%, 30%, 25%, 20%, 15%, 10%, or 5%). In some embodiments, binding of transferrin to TfR is inhibited by less than about 20% (e.g., less than about 19%, 18%, 17%, 16%, 15%, 14%, 13%, 12%, 11%, 10%, 9%, 8%, 7%, 6%, 5%, 4%, 3%, 2%, or 1%).

In some embodiments, a modified Fc polypeptide that specifically binds to TfR comprises at least two, three, four, five, six, seven, eight, or nine substitutions at positions 384, 386, 387, 388, 389, 390, 413, 416, and 421, according to the EU numbering scheme. Illustrative substitutions that may be introduced at these positions are shown in Tables 11 and 12. In some embodiments, the amino acid at position 388 and/or 421 is an aromatic amino acid, e.g., Trp, Phe, or Tyr. In some embodiments, the amino acid at position 388 is Trp. In some embodiments, the aromatic amino acid at position 421 is Trp or Phe.

In some embodiments, at least one position as follows is substituted: Leu, Tyr, Met, or Val at position 384; Leu, Thr, His, or Pro at position 386; Val, Pro, or an acidic amino acid at position 387; an aromatic amino acid, e.g. Trp at position 388; Val, Ser, or Ala at position 389; an acidic amino acid, Ala, Ser, Leu, Thr, or Pro at position 413; Thr or an acidic amino acid at position 416; or Trp, Tyr, His, or Phe at position 421. In some embodiments, the modified Fc polypeptide may comprise a conservative substitution, e.g., an amino acid in the same charge grouping, hydrophobicity grouping, side chain ring structure grouping (e.g., aromatic amino acids), or size grouping, and/or polar or non-polar grouping, of a specified amino acid at one or more of the positions in the set. Thus, for example, Ile may be present at position 384, 386, and/or position 413. In some embodiments, the acidic amino acid at position one, two, or each of positions 387, 413, and 416 is Glu. In other embodiments, the acidic amino acid at one, two or each of positions 387, 413, and 416 is Asp. In some embodiments, two, three, four, five, six, seven, or all eight of positions 384, 386, 387, 388, 389, 413, 416, and 421 have an amino acid substitution as specified in this paragraph.

In some embodiments, an Fc polypeptide that is modified as described in the preceding two paragraphs comprises a native Asn at position 390. In some embodiments, the modified Fc polypeptide comprises Gly, His, Gln, Leu, Lys, Val, Phe, Ser, Ala, or Asp at position 390. In some embodiments, the modified Fc polypeptide further comprises one, two, three, or four substitutions at positions comprising 380, 391, 392, and 415, according to the EU numbering scheme. In some embodiments, Trp, Tyr, Leu, or Gln may be present at position 380. In some embodiments, Ser, Thr, Gln, or Phe may be present at position 391. In some embodiments, Gln, Phe, or His may be present at position 392. In some embodiments, Glu may be present at position 415.

In certain embodiments, the modified Fc polypeptide comprises two, three, four, five, six, seven, eight, nine, ten, or eleven positions selected from the following: Trp, Leu, or Glu at position 380; Tyr or Phe at position 384; Thr at position 386; Glu at position 387; Trp at position 388; Ser, Ala, Val, or Asn at position 389; Ser or Asn at position 390; Thr or Ser at position 413; Glu or Ser at position 415; Glu at position 416; and/or Phe at position 421. In some embodiments, the modified Fc polypeptide comprises all eleven positions as follows: Trp, Leu, or Glu at position 380; Tyr or Phe at position 384; Thr at position 386; Glu at position 387; Trp at position 388; Ser, Ala, Val, or Asn at position 389; Ser or Asn at position 390; Thr or Ser at position 413; Glu or Ser at position 415; Glu at position 416; and/or Phe at position 421.

In certain embodiments, the modified Fc polypeptide comprises Leu or Met at position 384; Leu, His, or Pro at position 386; Val at position 387; Trp at position 388; Val or Ala at position 389; Pro at position 413; Thr at position 416; and/or Trp at position 421. In some embodiments, the modified Fc polypeptide further comprises Ser, Thr, Gln, or Phe at position 391. In some embodiments, the modified Fc polypeptide further comprises Trp, Tyr, Leu, or Gln at position 380 and/or Gln, Phe, or His at position 392. In some embodiments, Trp is present at position 380 and/or Gln is present at position 392. In some embodiments, the modified Fc polypeptide does not have a Trp at position 380.

In other embodiments, the modified Fc polypeptide comprises Tyr at position 384; Thr at position 386; Glu or Val and position 387; Trp at position 388; Ser at position 389; Ser or Thr at position 413; Glu at position 416; and/or Phe at position 421. In some embodiments, the modified Fc polypeptide comprises a native Asn at position 390. In certain embodiments, the modified Fc polypeptide further comprises Trp, Tyr, Leu, or Gln at position 380; and/or Glu at position 415. In some embodiments, the modified Fc polypeptide further comprises Trp at position 380 and/or Glu at position 415.

In additional embodiments, the modified Fc polypeptide further comprises one, two, or three substitutions at positions comprising 414, 424, and 426, according to the EU numbering scheme. In some embodiments, position 414 is Lys, Arg, Gly, or Pro; position 424 is Ser, Thr, Glu, or Lys; and/or position 426 is Ser, Trp, or Gly.

In some embodiments, the modified Fc polypeptide comprises one or more of the following substitutions: Trp at position 380; Thr at position 386; Trp at position 388; Val at position 389; Thr or Ser at position 413; Glu at position 415; and/or Phe at position 421, according to the EU numbering scheme.

In some embodiments, the modified Fc polypeptide has at least 70% identity, at least 75% identity, at least 80% identity, at least 85% identity, at least 90% identity, or at least 95% identity to amino acids 111-217 of any one of SEQ ID NOs:100-185, 219-298, or 337-460 (e.g., SEQ ID NOs:100-136 or 337-350). In some embodiments, the modified Fc polypeptide has at least 70% identity, at least 75% identity, at least 80% identity, at least 85% identity, at least 90% identity, or at least 95% identity to any one of SEQ ID NOs:100-185, 219-298, or 337-460 (e.g., SEQ ID NOs:100-136 or 337-350). In some embodiments, the modified Fc polypeptide comprises the amino acids at EU index positions 384-390 and/or 413-421 of any one of SEQ ID NOs:100-185, 219-298, or 337-460 (e.g., SEQ ID NOs:100-136 or 337-350). In some embodiments, the modified Fc polypeptide comprises the amino acids at EU index positions 380-390 and/or 413-421 of any one of SEQ ID NOs:100-185, 219-298, or 337-460 (e.g., SEQ ID NOs:100-136 or 337-350). In some embodiments, the modified Fc polypeptide comprises the amino acids at EU index positions 380-392 and/or 413-426 of any one of SEQ ID NOs:100-185, 219-298, or 337-460 (e.g., SEQ ID NOs:100-136 or 337-350).

In some embodiments, the modified Fc polypeptide has at least 75% identity, at least 80% identity, at least 85% identity, at least 90% identity, or at least 95% identity to any one of SEQ ID NOs:100-185, 219-298, or 337-460 (e.g., SEQ ID NOs:100-136 or 337-350), and further comprises at least five, six, seven, eight, nine, ten, eleven, twelve, thirteen, fourteen, fifteen, or sixteen of the positions, numbered according to the EU index, as follows: Trp, Tyr, Leu, Gln, or Glu at position 380; Leu, Tyr, Met, or Val at position 384; Leu, Thr, His, or Pro at position 386; Val, Pro, or an acidic amino acid at position 387; an aromatic amino acid, e.g. Trp, at position 388; Val, Ser, or Ala at position 389; Ser or Asn at position 390; Ser, Thr, Gln, or Phe at position 391; Gln, Phe, or His at position 392; an acidic amino acid, Ala, Ser, Leu, Thr, or Pro at position 413; Lys, Arg, Gly or Pro at position 414; Glu or Ser at position 415; Thr or an acidic amino acid at position 416; Trp, Tyr, His or Phe at position 421; Ser, Thr, Glu or Lys at position 424; and Ser, Trp, or Gly at position 426.

In some embodiments, the modified Fc polypeptide comprises the amino acid sequence of any one of SEQ ID NOs:100-136 or 337-350. In other embodiments, the modified Fc polypeptide comprises the amino acid sequence of any one of SEQ ID NOs: 100-136 or 337-350, but in which one, two, or three amino acids are substituted.

In some embodiments, the modified Fc polypeptide comprises additional mutations such as the mutations described in Section IV below, including, but not limited to, a knob mutation (e.g., T366W as numbered with reference to EU numbering), hole mutations (e.g., T366S, L368A, and Y407V as numbered with reference to EU numbering), mutations that modulate effector function (e.g., L234A, L235A, and/or P329G (e.g., L234A and L235A) as numbered with reference to EU numbering), and/or mutations that increase serum stability (e.g., M252Y, S254T, and T256E, or N434S with or without M428L, as numbered with reference to EU numbering). By way of illustration, SEQ ID NOS:227-298 and 351-460 provide non-limiting examples of modified Fc polypeptides with mutations in the CH3 domain (e.g., clones CH3C.35.20.1, CH3C.35.23.2, CH3C.35.23.3, CH3C.35.23.4, CH3C.35.21.17.2, CH3C.35.23, CH3C.35.21, CH3C.35.20.1.1, CH3C.35.23.2.1, and CH3C.35.23.1.1) comprising one or more of these additional mutations.

In some embodiments, the modified Fc polypeptide comprises a knob mutation (e.g., T366W as numbered with reference to EU numbering) and has at least 85% identity, at least 90% identity, or at least 95% identity to the sequence of any one of SEQ ID NOs:227, 239, 251, 263, 275, 287, 355, 367, and 379. In some embodiments, the modified Fc polypeptide comprises the sequence of any one of SEQ ID NOs:227, 239, 251, 263, 275, 287, 355, 367, and 379.

In some embodiments, the modified Fc polypeptide comprises a knob mutation (e.g., T366W as numbered with reference to EU numbering) and mutations that modulate effector function (e.g., L234A, L235A, and/or P329G (e.g., L234A and L235A) as numbered with reference to EU numbering), and has at least 85% identity, at least 90% identity, or at least 95% identity to the sequence of any one of SEQ ID NOS:228, 229, 240, 241, 252, 253, 264, 265, 276, 277, 288, 289, 351, 356, 357, 368, 369, 380, and 381. In some embodiments, the modified Fc polypeptide comprises the sequence of any one of SEQ ID NOS:228, 229, 240, 241, 252, 253, 264, 265, 276, 277, 288, 289, 351, 356, 357, 368, 369, 380, and 381.

In some embodiments, the modified Fc polypeptide comprises a knob mutation (e.g., T366W as numbered with reference to EU numbering) and mutations that increase serum stability (e.g., M252Y, S254T, and T256E, or N434S with or without M428L, as numbered with reference to EU numbering), and has at least 85% identity, at least 90% identity, or at least 95% identity to the sequence of any one of SEQ ID NOS:230, 242, 254, 266, 278, 290, 358, 370, 382, 392, 399, 406, 413, 420, 427, 434, 441, 448, and 455. In some embodiments, the modified Fc polypeptide comprises the sequence of any one of SEQ ID NOS:230, 242, 254, 266, 278, 290, 358, 370, 382, 392, 399, 406, 413, 420, 427, 434, 441, 448, and 455.

In some embodiments, the modified Fc polypeptide comprises a knob mutation (e.g., T366W as numbered with reference to EU numbering), mutations that modulate effector function (e.g., L234A, L235A, and/or P329G (e.g., L234A and L235A) as numbered with reference to EU numbering), and mutations that increase serum stability (e.g., M252Y, S254T, and T256E, or N434S with or without M428L, as numbered with reference to EU numbering), and has at least 85% identity, at least 90% identity, or at least 95% identity to the sequence of any one of SEQ ID NOS:231, 232, 243, 244, 255, 256, 267, 268, 279, 280, 291, 292, 352, 359, 360, 371, 372, 383, 384, 393, 394, 400, 401, 407, 408, 414, 415, 421, 422, 428, 429, 435, 436, 442, 443, 449, 450, 456, and 457. In some embodiments, the modified Fc polypeptide comprises the sequence of any one of SEQ ID NOS:231, 232, 243, 244, 255, 256, 267, 268, 279, 280, 291, 292, 352, 359, 360, 371, 372, 383, 384, 393, 394, 400, 401, 407, 408, 414, 415, 421, 422, 428, 429, 435, 436, 442, 443, 449, 450, 456, and 457.

In some embodiments, the modified Fc polypeptide comprises hole mutations (e.g., T366S, L368A, and Y407V as numbered with reference to EU numbering) and has at least 85% identity, at least 90% identity, or at least 95% identity to the sequence of any one of SEQ ID NOS:233, 245, 257, 269, 281, 293, 361, 373, and 385. In some embodiments, the modified Fc polypeptide comprises the sequence of any one of SEQ ID NOS:233, 245, 257, 269, 281, 293, 361, 373, and 385.

In some embodiments, the modified Fc polypeptide comprises hole mutations (e.g., T366S, L368A, and Y407V as numbered with reference to EU numbering) and mutations that modulate effector function (e.g., L234A, L235A, and/or P329G (e.g., L234A and L235A) as numbered with reference to EU numbering), and has at least 85% identity, at least 90% identity, or at least 95% identity to the sequence of any one of SEQ ID NOS:234, 235, 246, 247, 258, 259, 270, 271, 282, 283, 294, 295, 353, 362, 363, 374, 375, 386, and 387. In some embodiments, the modified Fc polypeptide comprises the sequence of any one of SEQ ID NOS:234, 235, 246, 247, 258, 259, 270, 271, 282, 283, 294, 295, 353, 362, 363, 374, 375, 386, and 387.

In some embodiments, the modified Fc polypeptide comprises hole mutations (e.g., T366S, L368A, and Y407V as numbered with reference to EU numbering) and mutations that increase serum stability (e.g., M252Y, S254T, and T256E, or N434S with or without M428L, as numbered with reference to EU numbering), and has at least 85% identity, at least 90% identity, or at least 95% identity to the sequence of any one of SEQ ID NOS:236, 248, 260, 272, 284, 296, 364, 376, 388, 395, 402, 409, 416, 423, 430, 437, 444, 451, and 458. In some embodiments, the modified Fc polypeptide comprises the sequence of any one of SEQ ID NOS:236, 248, 260, 272, 284, 296, 364, 376, 388, 395, 402, 409, 416, 423, 430, 437, 444, 451, and 458.

In some embodiments, the modified Fc polypeptide comprises hole mutations (e.g., T366S, L368A, and Y407V as numbered with reference to EU numbering), mutations that modulate effector function (e.g., L234A, L235A, and/or P329G (e.g., L234A and L235A) as numbered with reference to EU numbering), and mutations that increase serum stability (e.g., M252Y, S254T, and T256E, or N434S with or without M428L, as numbered with reference to EU numbering), and has at least 85% identity, at least 90% identity, or at least 95% identity to the sequence of any one of SEQ ID NOS:237, 238, 249, 250, 261, 262, 273, 274, 285, 286, 297, 298, 354, 365, 366, 377, 378, 389, 390, 396, 397, 403, 404, 410, 411, 417, 418, 424, 425, 431, 432, 438, 439, 445, 446, 452, 453, 459, and 460. In some embodiments, the modified Fc polypeptide comprises the sequence of any one of SEQ ID NOS:237, 238, 249, 250, 261, 262, 273, 274, 285, 286, 297, 298, 354, 365, 366, 377, 378, 389, 390, 396, 397, 403, 404, 410, 411, 417, 418, 424, 425, 431, 432, 438, 439, 445, 446, 452, 453, 459, and 460.

In some embodiments, a modified Fc polypeptide that specifically binds to TfR comprises at least two, three, four, five, six, seven, or eight substitutions at positions 345, 346, 347, 349, 437, 438, 439, and 440, according to the EU numbering scheme. Illustrative modified Fc polypeptides are provided in SEQ ID NOs:186-190. In some embodiments, the modified Fc polypeptide comprises Gly at position 437; Phe at position 438; and/or Asp at position 440. In some embodiments, Glu is present at position 440. In certain embodiments, the modified Fc polypeptide comprises at least one substitution at a position as follows: Phe or Ile at position 345; Asp, Glu, Gly, Ala, or Lys at position 346; Tyr, Met, Leu, Ile, or Asp at position 347; Thr or Ala at position 349; Gly at position 437; Phe at position 438; His Tyr, Ser, or Phe at position 439; or Asp at position 440. In some embodiments, two, three, four, five, six, seven, or all eight of positions 345, 346, 347, 349, 437, 438, 439, and 440 and have a substitution as specified in this paragraph. In some embodiments, the modified Fc polypeptide may comprise a conservative substitution, e.g., an amino acid in the same charge grouping, hydrophobicity grouping, side chain ring structure grouping (e.g., aromatic amino acids), or size grouping, and/or polar or non-polar grouping, of a specified amino acid at one or more of the positions in the set.

In some embodiments, the modified Fc polypeptide has at least 70% identity, at least 75% identity, at least 80% identity, at least 85% identity, at least 90% identity, or at least 95% identity to amino acids 111-217 of any one of SEQ ID NOs:186-190. In some embodiments, the modified Fc polypeptide has at least 70% identity, at least 75% identity, at least 80% identity, at least 85% identity, at least 90% identity, or at least 95% identity to SEQ ID NOs:186-190. In some embodiments, the modified Fc polypeptide comprises the amino acid sequence of any one of SEQ ID NOs:186-190. In other embodiments, the modified Fc polypeptide comprises the amino acid sequence of any one of SEQ ID NOs:186-190, but in which one, two, or three amino acids are substituted.

TfR-binding Fc polypeptides comprising mutations in the CH2 domain

In some embodiments, a modified Fc polypeptide that specifically binds to TfR comprises substitutions in a CH2 domain. In some embodiments, a modified Fc polypeptide comprises a human Ig CH2 domain, such as an IgG CH2 domain, that is modified for TfR-binding activity. The CH2 domain can be of any IgG subtype, i.e., from IgG1, IgG2, IgG3, or IgG4. In the context of IgG antibodies, a CH2 domain refers to the segment of amino acids from about position 231 to about position 340 as numbered according to the EU numbering scheme.

In some embodiments, a modified Fc polypeptide that specifically binds to TfR binds to the apical domain of TfR and may bind to TfR without blocking or otherwise inhibiting binding of transferrin to TfR. In some embodiments, binding of transferrin to TfR is not substantially inhibited. In some embodiments, binding of transferrin to TfR is inhibited by less than about 50% (e.g., less than about 45%, 40%, 35%, 30%, 25%, 20%, 15%, 10%, or 5%). In some embodiments, binding of transferrin to TfR is inhibited by less than about 20% (e.g., less than about 19%, 18%, 17%, 16%, 15%, 14%, 13%, 12%, 11%, 10%, 9%, 8%, 7%, 6%, 5%, 4%, 3%, 2%, or 1%).

In some embodiments, a modified Fc polypeptide that specifically binds to TfR comprises at least two, three, four, five, six, seven, eight, or nine substitutions at positions 274, 276, 283, 285, 286, 287, 288, and 290, according to the EU numbering scheme. Illustrative modified Fc polypeptides are provided in SEQ ID NOs:191-195. In some embodiments, the modified Fc polypeptide comprises Glu at position 287 and/or Trp at position 288. In some embodiments, the modified Fc polypeptide comprises at least one substitution at a position as follows: Glu, Gly, Gln, Ser, Ala, Asn, Tyr, or Trp at position 274; Ile, Val, Asp, Glu, Thr, Ala, or Tyr at position 276; Asp, Pro, Met, Leu, Ala, Asn, or Phe at position 283; Arg, Ser, Ala, or Gly at position 285; Tyr, Trp, Arg, or Val at position 286; Glu at position 287; Trp or Tyr at position 288; Gln, Tyr, His, Ile, Phe, Val, or Asp at position 289; or Leu, Trp, Arg, Asn, Tyr, or Val at position 290. In some embodiments, two, three, four, five, six, seven, eight, or all nine of positions 274, 276, 283, 285, 286, 287, 288, and 290 have a substitution as specified in this paragraph. In some embodiments, the modified Fc polypeptide may comprise a conservative substitution, e.g., an amino acid in the same charge grouping, hydrophobicity grouping, side chain ring structure grouping (e.g., aromatic amino acids), or size grouping, and/or polar or non-polar grouping, of a specified amino acid at one or more of the positions in the set.

In some embodiments, the modified Fc polypeptide comprises Glu, Gly, Gin, Ser, Ala, Asn, or Tyr at position 274; Ile, Val, Asp, Glu, Thr, Ala, or Tyr at position 276 Asp, Pro, Met, Leu, Ala, or Asn at position 283; Arg, Ser, or Ala at position 285; Tyr, Trp, Arg, or Val at position 286; Glu at position 287; Trp at position 288; Gin, Tyr, His, Ile, Phe, or Val at position 289; and/or Leu, Trp, Arg, Asn, or Tyr at position 290. In some embodiments, the modified Fc polypeptide comprises Arg at position 285; Tyr or Trp at position 286; Glu at position 287; Trp at position 288; and/or Arg or Trp at position 290.

In some embodiments, the modified Fc polypeptide has at least 70% identity, at least 75% identity, at least 80% identity, at least 85% identity, at least 90% identity, or at least 95% identity to amino acids 1-110 of any one of SEQ ID NOs:191-195. In some embodiments, the modified Fc polypeptide has at least 70% identity, at least 75% identity, at least 80% identity, at least 85% identity, at least 90% identity, or at least 95% identity to SEQ ID NOs:191-195. In some embodiments, the modified Fc polypeptide comprises the amino acid sequence of any one of SEQ ID NOs:191-195. In other embodiments, the modified Fc polypeptide comprises the amino acid sequence of any one of SEQ ID NOs:191-195, but in which one, two, or three amino acids are substituted.

In some embodiments, a modified Fc polypeptide that specifically binds to TfR comprises at least two, three, four, five, six, seven, eight, nine, or ten substitutions at positions 266, 267, 268, 269, 270, 271, 295, 297, 298, and 299, according to the EU numbering scheme. Illustrative modified Fc polypeptides are provided in SEQ ID NOs:196-200. In some embodiments, the modified Fc polypeptide comprises Pro at position 270, Glu at position 295, and/or Tyr at position 297. In some embodiments, the modified Fc polypeptide comprises at least one substitution at a position as follows: Pro, Phe, Ala, Met, or Asp at position 266; Gin, Pro, Arg, Lys, Ala, Ile, Leu, Glu, Asp, or Tyr at position 267; Thr, Ser, Gly, Met, Val, Phe, Trp, or Leu at position 268; Pro, Val, Ala, Thr, or Asp at position 269; Pro, Val, or Phe at position 270; Trp, Gin, Thr, or Glu at position 271; Glu, Val, Thr, Leu, or Trp at position 295; Tyr, His, Val, or Asp at position 297; Thr, His, Gin, Arg, Asn, or Val at position 298; or Tyr, Asn, Asp, Ser, or Pro at position 299. In some embodiments, two, three, four, five, six, seven, eight, nine, or all ten of positions 266, 267, 268, 269, 270, 271, 295, 297, 298, and 299 have a substitution as specified in this paragraph. In some embodiments, a modified Fc polypeptide may comprise a conservative substitution, e.g., an amino acid in the same charge grouping, hydrophobicity grouping, side chain ring structure grouping (e.g., aromatic amino acids), or size grouping, and/or polar or non-polar grouping, of a specified amino acid at one or more of the positions in the set.

In some embodiments, the modified Fc polypeptide comprises Pro, Phe, or Ala at position 266; Gln, Pro, Arg, Lys, Ala, or Ile at position 267; Thr, Ser, Gly, Met, Val, Phe, or Trp at position 268; Pro, Val, or Ala at position 269; Pro at position 270; Trp or Gln at position 271; Glu at position 295; Tyr at position 297; Thr, His, or Gln at position 298; and/or Tyr, Asn, Asp, or Ser at position 299.

In some embodiments, the modified Fc polypeptide comprises Met at position 266; Leu or Glu at position 267; Trp at position 268; Pro at position 269; Val at position 270; Thr at position 271; Val or Thr at position 295; His at position 197; His, Arg, or Asn at position 198; and/or Pro at position 299.

In some embodiments, the modified Fc polypeptide comprises Asp at position 266; Asp at position 267; Leu at position 268; Thr at position 269; Phe at position 270; Gln at position 271; Val or Leu at position 295; Val at position 297; Thr at position 298; and/or Pro at position 299.

In some embodiments, the modified Fc polypeptide has at least 70% identity, at least 75% identity, at least 80% identity, at least 85% identity, at least 90% identity, or at least 95% identity to amino acids 1-110 of any one of SEQ ID NOs:196-200. In some embodiments, the modified Fc polypeptide has at least 70% identity, at least 75% identity, at least 80% identity, at least 85% identity, at least 90% identity, or at least 95% identity to SEQ ID NOs:196-200. In some embodiments, the modified Fc polypeptide comprises the amino acid sequence of any one of SEQ ID NOs: 196-200. In other embodiments, the modified Fc polypeptide comprises the amino acid sequence of any one of SEQ ID NOs:196-200, but in which one, two, or three amino acids are substituted.

In some embodiments, a modified Fc polypeptide that specifically binds to TfR comprises at least two, three, four, five, six, seven, eight, nine, or ten substitutions at positions 268, 269, 270, 271, 272, 292, 293, 294, and 300, according to the EU numbering scheme. Illustrative modified Fc polypeptides are provided in SEQ ID NOs:201-205. In some embodiments, the modified Fc polypeptide comprises at least one substitution at a position as follows: Val or Asp at position 268; Pro, Met, or Asp at position 269; Pro or Trp at position 270; Arg, Trp, Glu, or Thr at position 271; Met, Tyr, or Trp at position 272; Leu or Trp at position 292; Thr, Val, Ile, or Lys at position 293; Ser, Lys, Ala, or Leu at position 294; His, Leu, or Pro at position 296; or Val or Trp at position 300. In some embodiments, two, three, four, five, six, seven, eight, nine, or all ten of positions 268, 269, 270, 271, 272, 292, 293, 294, and 300 have a substitution as specified in this paragraph. In some embodiments, the modified Fc polypeptide may comprise a conservative substitution, e.g., an amino acid in the same charge grouping, hydrophobicity grouping, side chain ring structure grouping (e.g., aromatic amino acids), or size grouping, and/or polar or non-polar grouping, of a specified amino acid at one or more of the positions in the set.

In some embodiments, the modified Fc polypeptide comprises Val at position 268; Pro at position 269; Pro at position 270; Arg or Trp at position 271; Met at position 272; Leu at position 292; Thr at position 293; Ser at position 294; His at position 296; and/or Val at position 300.

In some embodiments, the modified Fc polypeptide comprises Asp at position 268; Met or Asp at position 269; Trp at position 270; Glu or Thr at position 271; Tyr or Trp at position 272; Trp at position 292; Val, Ile, or Lys at position 293; Lys, Ala, or Leu at position 294; Leu or Pro at position 296; and/or Trp at position 300.

In some embodiments, the modified Fc polypeptide has at least 70% identity, at least 75% identity, at least 80% identity, at least 85% identity, at least 90% identity, or at least 95% identity to amino acids 1-110 of any one of SEQ ID NOs:201-205. In some embodiments, the modified Fc polypeptide has at least 70% identity, at least 75% identity, at least 80% identity, at least 85% identity, at least 90% identity, or at least 95% identity to SEQ ID NOs:201-205. In some embodiments, the modified Fc polypeptide comprises the amino acid sequence of any one of SEQ ID NOs:201-205. In other embodiments, the modified Fc polypeptide comprises the amino acid sequence of any one of SEQ ID NOs:201-205, but in which one, two, or three amino acids are substituted.

In some embodiments, a modified Fc polypeptide that specifically binds to TfR has at least two, three, four, five, six, seven, eight, nine, or ten substitutions at positions 272, 274, 276, 322, 324, 326, 329, 330, and 331, according to the EU numbering scheme. An illustrative modified polypeptide comprises Trp at position 330. In some embodiments, the modified Fc polypeptide comprises at least one substitution at a position as follows: Trp, Val, Ile, or Ala at position 272; Trp or Gly at position 274; Tyr, Arg, or Glu at position 276; Ser, Arg, or Gln at position 322; Val, Ser, or Phe at position 324; Ile, Ser, or Trp at position 326; Trp, Thr, Ser, Arg, or Asp at position 329; Trp at position 330; or Ser, Lys, Arg, or Val at position 331. In some embodiments, two, three, four, five, six, seven, eight, or all nine of positions 272, 274, 276, 322, 324, 326, 329, 330, and 331 have a substitution as specified in this paragraph. In some embodiments, the modified Fc polypeptide may comprise a conservative substitution, e.g., an amino acid in the same charge grouping, hydrophobicity grouping, side chain ring structure grouping (e.g., aromatic amino acids), or size grouping, and/or polar or non-polar grouping, of a specified amino acid at one or more of the positions in the set.

In some embodiments, the modified Fc polypeptide comprises two, three, four, five, six, seven, eight, or nine positions selected from the following: position 272 is Trp, Val, Ile, or Ala; position 274 is Trp or Gly; position 276 is Tyr, Arg, or Glu; position 322 is Ser, Arg, or Gln; position 324 is Val, Ser, or Phe; position 326 is Ile, Ser, or Trp; position 329 is Trp, Thr, Ser, Arg, or Asp; position 330 is Trp; and position 331 is Ser, Lys, Arg, or Val. In some embodiments, the modified Fc polypeptide comprises Val or Ile at position 272; Gly at position 274; Arg at position 276; Arg at position 322; Ser at position 324; Ser at position 326; Thr, Ser, or Arg at position 329; Trp at position 330; and/or Lys or Arg at position 331.

In some embodiments, the modified Fc polypeptide has at least 70% identity, at least 75% identity, at least 80% identity, at least 85% identity, at least 90% identity, or at least 95% identity to amino acids 1-110 of any one of SEQ ID NOs:206-210. In some embodiments, the modified Fc polypeptide has at least 70% identity, at least 75% identity, at least 80% identity, at least 85% identity, at least 90% identity, or at least 95% identity to SEQ ID NOs:206-210. In some embodiments, the modified Fc polypeptide comprises the amino acid sequence of any one of SEQ ID NOs:206-210. In other embodiments, the modified Fc polypeptide comprises the amino acid sequence of any one of SEQ ID NOs:206-210, but in which one, two, or three amino acids are substituted.

### Additional Fc Polypeptide Mutations

In some aspects, an anti-TREM2 antibody as disclosed herein comprises first and optionally second Fc polypeptides that may each comprise independently selected modifications or may be a wild-type Fc polypeptide, e.g., a human IgG1 Fc polypeptide. In some embodiments, one or both Fc polypeptides contains one or more modifications that confer binding to a blood-brain barrier (BBB) receptor, e.g., transferrin receptor (TfR). Non-limiting examples of other mutations that can be introduced into one or both Fc polypeptides include, e.g., mutations to increase serum stability, to modulate effector function, to influence glycosylation, to reduce immunogenicity in humans, and/or to provide for knob and hole heterodimerization of the Fc polypeptides.

In some embodiments, the Fc polypeptides include knob and hole mutations to promote heterodimer formation and hinder homodimer formation. Generally, the modifications introduce a protuberance (“knob”) at the interface of a first polypeptide and a corresponding cavity (“hole”) in the interface of a second polypeptide, such that the protuberance can be positioned in the cavity so as to promote heterodimer formation and thus hinder homodimer formation. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine). In some embodiments, such additional mutations are at a position in the Fc polypeptide that does not have a negative effect on binding of the polypeptide to a BBB receptor, e.g., TfR.

In one illustrative embodiment of a knob and hole approach for dimerization, position 366 (numbered according to the EU numbering scheme) of one of the Fc polypeptides comprises a tryptophan in place of a native threonine. The other Fc polypeptide in the dimer has a valine at position 407 (numbered according to the EU numbering scheme) in place of the native tyrosine. The other Fc polypeptide may further comprise a substitution in which the native threonine at position 366 (numbered according to the EU numbering scheme) is substituted with a serine and a native leucine at position 368 (numbered according to the EU numbering scheme) is substituted with an alanine. Thus, one of the Fc polypeptides of has the T366W knob mutation and the other Fc polypeptide has the Y407V mutation, which is typically accompanied by the T366S and L368A hole mutations.

In some embodiments, modifications to enhance serum half-life may be introduced. For example, in some embodiments, one or both Fc polypeptides may comprise a tyrosine at position 252, a threonine at position 254, and a glutamic acid at position 256, as numbered according to the EU numbering scheme. Thus, one or both Fc polypeptides may have M252Y, S254T, and T256E substitutions. Alternatively, one or both Fc polypeptides may have M428L and N434S substitutions, according to EU numbering. Alternatively, one or both Fc polypeptides may have an N434S or N434A substitution.

In some embodiments, one or both Fc polypeptides may comprise modifications that reduce effector function, i.e., having a reduced ability to induce certain biological functions upon binding to an Fc receptor expressed on an effector cell that mediates the effector function. Examples of antibody effector functions include, but are not limited to, Clq binding and complement dependent cytotoxicity (CDC), Fc receptor binding, antibody-dependent cell-mediated cytotoxicity (ADCC), antibody-dependent cell-mediated phagocytosis (ADCP), down-regulation of cell surface receptors (e.g., B cell receptor), and B-cell activation. Effector functions may vary with the antibody class. For example, native human IgG1 and IgG3 antibodies can elicit ADCC and CDC activities upon binding to an appropriate Fc receptor present on an immune system cell; and native human IgG1, IgG2, IgG3, and IgG4 can elicit ADCP functions upon binding to the appropriate Fc receptor present on an immune cell.

In some embodiments, one or both Fc polypeptides may also be engineered to contain other modifications for heterodimerization, e.g., electrostatic engineering of contact residues within a CH3-CH3 interface that are naturally charged or hydrophobic patch modifications.

In some embodiments, one or both Fc polypeptides may include additional modifications that modulate effector function.

In some embodiments, one or both Fc polypeptides may comprise modifications that reduce or eliminate effector function. Illustrative Fc polypeptide mutations that reduce effector function include, but are not limited to, substitutions in a CH2 domain, e.g., at positions 234 and 235, according to the EU numbering scheme. For example, in some embodiments, one or both Fc polypeptides can comprise alanine residues at positions 234 and 235. Thus, one or both Fc polypeptides may have L234A and L235A (“LALA”) substitutions. In some embodiments, an Fc polypeptide that comprises one or more modifications that promote binding to TfR further comprises LALA substitutions. In some embodiments, an Fc polypeptide that does not comprise one or more modifications that promote binding to TfR comprises LALA substitutions. In some embodiments, both Fc polypeptides comprise LALA substitutions.

Additional Fc polypeptide mutations that modulate an effector function include, but are not limited to, one or more substitutions at positions 238, 265, 269, 270, 297, 327 and 329, according to the EU numbering scheme. Illustrative substitutions include the following: position 329 may have a mutation in which proline is substituted with a glycine or arginine or an amino acid residue large enough to destroy the Fc/Fcγ receptor interface that is formed between proline 329 of the Fc and tryptophan residues Trp 87 and Trp 110 of FcγRIII.

Additional illustrative substitutions include S228P, E233P, L235E, N297A, N297D, and P331S, according to the EU numbering scheme. Multiple substitutions may also be present, e.g., L234A and L235A of a human IgG1 Fc region; L234A, L235A, and P329G of a human IgG1 region; S228P and L235E of a human IgG4 Fc region; L234A and G237A of a human IgG1 Fc region; L234A, L235A, and G237A of a human IgG1 Fc region; V234A and G237A of a human IgG2 Fc region; L235A, G237A, and E318A of a human IgG4 Fc region; and S228P and L236E of a human IgG4 Fc region, according to the EU numbering scheme. In some embodiments, one or both Fc polypeptides may have one or more amino acid substitutions that modulate ADCC, e.g., substitutions at positions 298, 333, and/or 334, according to the EU numbering scheme.

### Illustrative Fc Polypeptides Comprising Additional Mutations

By way of non-limiting example, one or both Fc polypeptides present in an anti-TREM2 antibody of the disclosure may comprise additional mutations including a knob mutation (e.g., T366W as numbered according to the EU numbering scheme), hole mutations (e.g., T366S, L368A, and Y407V as numbered according to the EU numbering scheme), mutations that modulate effector function (e.g., L234A, L235A, and/or P329G (e.g., L234A and L235A) as numbered according to the EU numbering scheme), and/or mutations that increase serum stability (e.g., (i) M252Y, S254T, and T256E as numbered according to the EU numbering scheme, or (ii) N434S with or without M428L as numbered with reference to EU numbering).

In some embodiments, an Fc polypeptide may have a knob mutation (e.g., T366W as numbered according to the EU numbering scheme) and at least 85% identity, at least 90% identity, or at least 95% identity to the sequence of any one of SEQ ID NOs:98, 100-210, and 337-350. In some embodiments, an Fc polypeptide may have a knob mutation and the sequence of any one of SEQ ID NOs:98, 100-210, and 337-350. In some embodiments, an Fc polypeptide having the sequence of any one of SEQ ID NOs:98, 100-210, and 337-350 may be modified to have a knob mutation.

In some embodiments, an Fc polypeptide may have a knob mutation (e.g., T366W as numbered according to the EU numbering scheme), mutations that modulate effector function (e.g., L234A, L235A, and/or P329G (e.g., L234A and L235A) as numbered according to the EU numbering scheme), and at least 85% identity, at least 90% identity, or at least 95% identity to the sequence of any one of SEQ ID NOs:98, 100-210, and 337-350. In some embodiments, an Fc polypeptide may have a knob mutation, mutations that modulate effector function, and the sequence of any one of SEQ ID NOs:98, 100-210, and 337-350. In some embodiments, an Fc polypeptide having the sequence of any one of SEQ ID NOs:98, 100-210, and 337-350 may be modified to have a knob mutation and mutations that modulate effector function.

In some embodiments, an Fc polypeptide may have a knob mutation (e.g., T366W as numbered according to the EU numbering scheme), mutations that increase serum stability (e.g., (i) M252Y, S254T, and T256E as numbered according to the EU numbering scheme, or (ii) N434S with or without M428L as numbered with reference to EU numbering), and at least 85% identity, at least 90% identity, or at least 95% identity to the sequence of any one of SEQ ID NOs:98, 100-210, and 337-350. In some embodiments, an Fc polypeptide may have a knob mutation, mutations that increase serum stability, and the sequence of any one of SEQ ID NOs:98, 100-210, and 337-350. In some embodiments, an Fc polypeptide having the sequence of any one of SEQ ID NOs:98, 100-210, and 337-350 may be modified to have a knob mutation and mutations that increase serum stability.

In some embodiments, an Fc polypeptide may have a knob mutation (e.g., T366W as numbered according to the EU numbering scheme), mutations that modulate effector function (e.g., L234A, L235A, and/or P329G (e.g., L234A and L235A) as numbered according to the EU numbering scheme), mutations that increase serum stability (e.g., (i) M252Y, S254T, and T256E as numbered according to the EU numbering scheme, or (ii) N434S with or without M428L as numbered with reference to EU numbering), and at least 85% identity, at least 90% identity, or at least 95% identity to the sequence of any one of SEQ ID NOs:98, 100-210, and 337-350. In some embodiments, an Fc polypeptide may have a knob mutation, mutations that modulate effector function, mutations that increase serum stability, and the sequence of any one of SEQ ID NOs:98, 100-210, and 337-350. In some embodiments, an Fc polypeptide having the sequence of any one of SEQ ID NOs:98, 100-210, and 337-350 may be modified to have a knob mutation, mutations that modulate effector function, and mutations that increase serum stability.

In some embodiments, an Fc polypeptide may have hole mutations (e.g., T366S, L368A, and Y407V as numbered according to the EU numbering scheme) and at least 85% identity, at least 90% identity, or at least 95% identity to the sequence of any one of SEQ ID NOs:98, 100-210, and 337-350. In some embodiments, an Fc polypeptide may have hole mutations and the sequence of any one of SEQ ID NOs:98, 100-210, and 337-350. In some embodiments, an Fc polypeptide having the sequence of any one of SEQ ID NOs:98, 100-210, and 337-350 may be modified to have a hole mutation.

In some embodiments, an Fc polypeptide may have hole mutations (e.g., T366S, L368A, and Y407V as numbered according to the EU numbering scheme), mutations that modulate effector function (e.g., L234A, L235A, and/or P329G (e.g., L234A and L235A) as numbered according to the EU numbering scheme), and at least 85% identity, at least 90% identity, or at least 95% identity to the sequence of any one of SEQ ID NOs:98, 100-210, and 337-350. In some embodiments, an Fc polypeptide may have hole mutations, mutations that modulate effector function, and the sequence of any one of SEQ ID NOs:98, 100-210, and 337-350. In some embodiments, an Fc polypeptide having the sequence of any one of SEQ ID NOs:98, 100-210, and 337-350 may be modified to have hole mutations and mutations that modulate effector function.

In some embodiments, an Fc polypeptide may have hole mutations (e.g., T366S, L368A, and Y407V as numbered according to the EU numbering scheme), mutations that increase serum stability (e.g., (i) M252Y, S254T, and T256E as numbered according to the EU numbering scheme, or (ii) N434S with or without M428L as numbered with reference to EU numbering), and at least 85% identity, at least 90% identity, or at least 95% identity to the sequence of any one of SEQ ID NOs:98, 100-210, and 337-350. In some embodiments, an Fc polypeptide may have hole mutations, mutations that increase serum stability, and the sequence of any one of SEQ ID NOs:98, 100-210, and 337-350. In some embodiments, an Fc polypeptide having the sequence of any one of SEQ ID NOs:98, 100-210, and 337-350 may be modified to have hole mutations and mutations that increase serum stability.

In some embodiments, an Fc polypeptide may have hole mutations (e.g., T366S, L368A, and Y407V as numbered according to the EU numbering scheme), mutations that modulate effector function (e.g., L234A, L235A, and/or P329G (e.g., L234A and L235A) as numbered according to the EU numbering scheme), mutations that increase serum stability (e.g., (i) M252Y, S254T, and T256E as numbered according to the EU numbering scheme, or (ii) N434S with or without M428L as numbered with reference to EU numbering), and at least 85% identity, at least 90% identity, or at least 95% identity to the sequence of any one of SEQ ID NOs:98, 100-210, and 337-350. In some embodiments, an Fc polypeptide may have hole mutations, mutations that modulate effector function, mutations that increase serum stability, and the sequence of any one of SEQ ID NOs:98, 100-210, and 337-350. In some embodiments, an Fc polypeptide having the sequence of any one of SEQ ID NOs:98, 100-210, and 337-350 may be modified to have hole mutations, mutations that modulate effector function, and mutations that increase serum stability.

### V. Therapeutic and Prognostic Methods Using Anti-Trem2 Antibodies

In another aspect, methods for the use of anti-TREM2 antibodies as described herein are provided. In some embodiments, an anti-TREM2 antibody as described in Section III above is used in the practice of the methods described herein.

Treatment with Anti-TREM2 Antibodies

In some embodiments, methods of modulating one or more TREM2 activities in a subject having a neurodegenerative disease are provided. In some embodiments, the methods comprise modulating recruitment or phosphorylation of a kinase that interacts with a TREM2/DAP12 signaling complex (e.g., Syk kinase); modulating phagocytosis (e.g., phagocytosis of cell debris, amyloid beta particles, etc.); modulating cell migration (e.g., migration of myeloid cells, macrophages, microglia, and disease associated microglia); and/or modulating cell differentiation (e.g., for myeloid cells, macrophages, microglia, and disease associated microglia). In some embodiments, methods of enhancing one or more TREM2 activities in a subject having a neurodegenerative disease are provided. In some embodiments, methods of inhibiting one or more TREM2 activities in a subject having a neurodegenerative disease are provided. In some embodiments, the method of modulating one or more TREM2 activities in a subject comprises administering to the subject an isolated antibody or an antigen-binding portion thereof that specifically binds to a human TREM2 protein, e.g., an anti-TREM2 antibody as describe herein, or a pharmaceutical composition comprising an anti-TREM2 antibody as described herein.

In some embodiments, methods of modulating levels of sTREM2 in a subject having a neurodegenerative disease are provided. In some embodiments, methods of decreasing levels of sTREM2 in a subject having a neurodegenerative disease are provided. In some embodiments, methods of increasing levels of sTREM2 in a subject having a neurodegenerative disease are provided. In some embodiments, the method of modulating levels of sTREM2 in a subject comprises administering to the subject an isolated antibody or an antigen-binding portion thereof that specifically binds to a human TREM2 protein, e.g., an anti-TREM2 antibody as described herein, or a pharmaceutical composition comprising an anti-TREM2 antibody as described herein.

In some embodiments, methods of treating a neurodegenerative disease are provided. In some embodiments, the neurodegenerative disease is selected from the group consisting of Alzheimer's disease, primary age-related tauopathy, progressive supranuclear palsy (PSP), frontotemporal dementia, frontotemporal dementia with parkinsonism linked to chromosome 17, argyrophilic grain dementia, amyotrophic lateral sclerosis, amyotrophic lateral sclerosis/parkinsonism-dementia complex of Guam (ALS-PDC), corticobasal degeneration, chronic traumatic encephalopathy, Creutzfeldt-Jakob disease, dementia pugilistica, diffuse neurofibrillary tangles with calcification, Down's syndrome, familial British dementia, familial Danish dementia, Gerstmann-Straussler-Scheinker disease, globular glial tauopathy, Guadeloupean parkinsonism with dementia, Guadelopean PSP, Hallevorden-Spatz disease, hereditary diffuse leukoencephalopathy with spheroids (HDLS), Huntington's disease, inclusion-body myositis, multiple system atrophy, myotonic dystrophy, Nasu-Hakola disease, neurofibrillary tangle-predominant dementia, Niemann-Pick disease type C, pallido-ponto-nigral degeneration, Parkinson's disease, Pick's disease, postencephalitic parkinsonism, prion protein cerebral amyloid angiopathy, progressive subcortical gliosis, subacute sclerosing panencephalitis, and tangle only dementia. In some embodiments, the neurodegenerative disease is Alzheimer's disease. In some embodiments, the neurodegenerative disease is Nasu-Hakola disease. In some embodiments, the neurodegenerative disease is frontotemporal dementia. In some embodiments, the neurodegenerative disease is Parkinson's disease. In some embodiments, the method comprises administering to the subject an isolated antibody or an antigen-binding portion thereof that specifically binds to a human TREM2 protein, e.g., an anti-TREM2 antibody as described herein, or a pharmaceutical composition comprising an anti-TREM2 antibody as described herein.

In some embodiments, an anti-TREM2 antibody (or antigen-binding portion or pharmaceutical composition thereof) as described herein is used in treating a neurodegenerative disease that is characterized by a mutation in TREM2. In some embodiments, the neurodegenerative disease that is characterized by a mutation in TREM2 is Alzheimer's disease, e.g., Alzheimer's disease that is characterized by a R47H mutation in TREM2.

In some embodiments, the subject to be treated is a human, e.g., a human adult or a human child.

In some embodiments, methods of reducing plaque accumulation in a subject having a neurodegenerative disease are provided. In some embodiments, the method comprises administering to the subject an antibody or pharmaceutical composition as described herein. In some embodiments, the subject has Alzheimer's disease. In some embodiments, the subject is an animal model of a neurodegenerative disease (e.g., a 5XFAD or APP/PS1 mouse model). In some embodiments, plaque accumulation is measured by amyloid plaque imaging and/or Tau imaging, e.g., using positron emission tomography (PET) scanning. In some embodiments, administration of an anti-TREM2 antibody reduces plaque accumulation by at least 20%, at least 30%, at least 40%, at least 50%, at least 60%, at least 70%, at least 80%, or at least 90% as compared to a baseline value (e.g., the level of plaque accumulation in the subject prior to administration of the anti-TREM2 antibody).

In some embodiments, an anti-TREM2 antibody is administered to a subject at a therapeutically effective amount or dose. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied according to several factors, including the chosen route of administration, the formulation of the composition, patient response, the severity of the condition, the subject's weight, and the judgment of the prescribing physician. The dosage can be increased or decreased over time, as required by an individual patient. In certain instances, a patient initially is given a low dose, which is then increased to an efficacious dosage tolerable to the patient. Determination of an effective amount is well within the capability of those skilled in the art.

The route of administration of an anti-TREM2 antibody as described herein can be oral, intraperitoneal, transdermal, subcutaneous, intravenous, intramuscular, intrathecal, inhalational, topical, intralesional, rectal, intrabronchial, nasal, transmucosal, intestinal, ocular or otic delivery, or any other methods known in the art. In some embodiments, the antibody is administered orally, intravenously, or intraperitoneally.

In some embodiments, the anti-TREM2 antibody (and optionally another therapeutic agent) is administered to the subject over an extended period of time, e.g., for at least 30, 40, 50, 60, 70, 80, 90, 100, 150, 200, 250, 300, 350 days or longer.

Identifying Subjects as Candidates for Treatment with Anti-TREM2 Antibodies

In another aspect, methods of identifying a subject having a neurodegenerative disease as a candidate for treatment with an anti-TREM2 antibody are provided.

In some embodiments, the method comprises:
measuring the level of sTREM2 in a sample from the subject;
comparing the level of sTREM2 in the sample from the subject to a control value, wherein a level of sTREM2 in the sample from the subject that is elevated relative to the control value identifies the subject as a candidate for treatment; and
for a subject identified as a candidate for treatment, administering to the subject an isolated antibody or an antigen-binding portion thereof that specifically binds to a human TREM2 protein (e.g., an antibody as described herein).

In some embodiments, the isolated antibody or antigen-binding portion thereof is an antibody that decreases levels of sTREM2. In some embodiments, the antibody further has one or more TREM2-associated activities as described herein, recognizes an epitope of human TREM2 that is the same or substantially the same as an epitope recognized by an antibody clone as described herein, and/or comprises one or more CDR, heavy chain, and/or light chain sequences of an antibody clone as described herein.

In some embodiments, the method comprises:
measuring the level of sTREM2 in a sample from the subject;
comparing the level of sTREM2 in the sample from the subject to a control value, wherein a level of sTREM2 in the sample from the subject that is reduced relative to the control value identifies the subject as a candidate for treatment; and
for a subject identified as a candidate for treatment, administering to the subject an isolated antibody or an antigen-binding portion thereof that specifically binds to a human TREM2 protein (e.g., an antibody as described herein).

In some embodiments, the isolated antibody or antigen-binding portion thereof is an antibody that increases levels of sTREM2. In some embodiments, the antibody further has one or more TREM2-associated activities as described herein, recognizes an epitope of human TREM2 that is the same or substantially the same as an epitope recognized by an antibody clone as described herein, and/or comprises one or more CDR, heavy chain, and/or light chain sequences of an antibody clone as described herein.

In another aspect, methods of treating a subject having a neurodegenerative disease that has been identified as a candidate for treatment with an anti-TREM2 antibody are provided. In some embodiments, the method comprises administering to the subject an isolated antibody or an antigen-binding portion thereof that specifically binds to a human TREM2 protein (e.g., an antibody as described herein), wherein the subject has been identified as having an increased level of sTREM2, relative to a control value. In some embodiments, the isolated antibody or antigen-binding portion thereof is an antibody that decreases levels of sTREM2.

In some embodiments, the method comprises administering to the subject an isolated antibody or an antigen-binding portion thereof that specifically binds to a human TREM2 protein (e.g., an antibody as described herein), wherein the subject has been identified as having a reduced level of sTREM2, relative to a control value. In some embodiments, the isolated antibody or antigen-binding portion thereof is an antibody that increases levels of sTREM2.

In some embodiments, the level of sTREM2 is compared to a control value that is determined for a healthy control or population of healthy controls (i.e., not afflicted with a neurodegenerative disease). In some embodiments, a subject is identified as a candidate for treatment if the level of sTREM2 in a sample from the subject differs by at least 20%, at least 30%, at least 40%, at least 50%, at least 60%, at least 70%, at least 80%, at least 90% or more as compared to the control value. In some embodiments, a subject is identified as a candidate for treatment if the level of sTREM2 in a sample from the subject differs by at least 2-fold, 3-fold, 4-fold, 5-fold, 6-fold, 7-fold, 8-fold, 9-fold, 10-fold or more as compared to the control value. In some embodiments, the healthy control value is determined by assessing the level of sTREM2 in a subject or population of subjects (e.g., 10, 20, 50, 100, 200, 500, 1000 subjects or more) that all are known not to have a neurodegenerative disease.

In some embodiments, the level of sTREM2 is compared to a control value that is determined for a disease control or population of disease controls (e.g., a person or population afflicted with Alzheimer's, or a person or population afflicted with a neurodegenerative disease that is characterized by a mutation in TREM2, such as Alzheimer's disease that is characterized by a R47H mutation in TREM2). In some embodiments, a subject is identified as a candidate for treatment if the level of sTREM2 in a sample from the subject is comparable to (e.g., is within 20%, 10%, 5%, 4%, 3%, 2%, or 1%) of the level of sTREM2 in the disease control or population of disease controls. In some embodiments, the disease control value is determined by assessing the level of sTREM2 in a subject or population of subjects (e.g., 10, 20, 50, 100, 200, 500, 1000 subjects or more) that all are known to have the neurodegenerative disease, e.g., Alzheimer's disease.

In some embodiments, the population of subjects is matched to a test subject according to one or more patient characteristics such as age, sex, ethnicity, or other criteria. In some embodiments, the control value is established using the same type of sample from the population of subjects (e.g., a sample comprising cerebrospinal fluid) as is used for assessing the level of sTREM2 in the test subject.

In some embodiments, sTREM2 levels are measured using a sample that comprises a fluid, e.g., blood, plasma, serum, urine, or cerebrospinal fluid. In some embodiments, the sample comprises cerebrospinal fluid.

STREM2 levels can be measured according to methods described herein, e.g., as described in Section III above. In some embodiments, sTREM2 levels are measured using an ELISA assay.

Monitoring Efficacy of Treatment with Anti-TREM2 Antibodies

In another aspect, methods of monitoring the efficacy of treatment with an anti-TREM2 antibody for a subject having a neurodegenerative disease are provided. In some embodiments, the subject being treated has been diagnosed as having a neurodegenerative disease as described herein. In some embodiments, the subject has been diagnosed as having Alzheimer's disease. In some embodiments, the subject has been diagnosed as having a neurodegenerative disease that is characterized by a mutation in TREM2, such as Alzheimer's disease that is characterized by a R47H mutation in TREM2.

In some embodiments, the method comprises:
measuring the level of sTREM2 in a first sample from the subject taken prior to an administration of an anti-TREM2 antibody (e.g., the first administration to the subject);
treating the subject with an isolated antibody or an antigen-binding portion thereof that specifically binds to a human TREM2 protein (e.g., an antibody as described herein); and
measuring the level of sTREM2 in a second sample from the subject taken subsequent to the administration of the anti-TREM2 antibody;
wherein a decrease in sTREM2 level in the second sample from the subject, as compared to the first sample from the subject, indicates that the subject is responding to treatment with the anti-TREM2 antibody.

In some embodiments, a decrease of at least at least 20%, at least 30%, at least 40%, at least 50%, at least 60%, at least 70%, at least 80%, or at least 90% in the level of sTREM2 in the second sample from the subject, as compared to the first sample from the subject, indicates that the subject is responding to treatment with the anti-TREM2 antibody.

In some embodiments, the method comprises:
measuring the level of sTREM2 in a first sample from the subject taken prior to an administration of an anti-TREM2 antibody (e.g., the first administration to the subject);
treating the subject with an isolated antibody or an antigen-binding portion thereof that specifically binds to a human TREM2 protein (e.g., an antibody as described herein); and
measuring the level of sTREM2 in a second sample from the subject taken subsequent to the administration of the anti-TREM2 antibody;
wherein an increase in sTREM2 level in the second sample from the subject, as compared to the first sample from the subject, indicates that the subject is responding to treatment with the anti-TREM2 antibody.

In some embodiments, an increase of at least at least 20%, at least 30%, at least 40%, at least 50%, at least 60%, at least 70%, at least 80%, or at least 90% in the level of sTREM2 in the second sample from the subject, as compared to the first sample from the subject, indicates that the subject is responding to treatment with the anti-TREM2 antibody.

In some embodiments, the measuring steps comprise using an assay for sTREM2 levels as described herein, e.g., as described in Section III above. In some embodiments, the levels of sTREM2 in the samples are measured using an immunoassay, e.g., an ELISA assay.

In some embodiments, the sample is a sample as described herein (e.g., as described in Section III above). In some embodiments, the sample comprises a fluid, e.g., blood, plasma, serum, urine, or cerebrospinal fluid. In some embodiments, the sample comprises cerebrospinal fluid. In some embodiments, the first sample and the second sample are the same type of sample (e.g., each of the first sample and the second sample is a cerebrospinal fluid sample).

In some embodiments, the subject has been treated with an anti-TREM2 antibody (e.g., an antibody or antigen-binding portion thereof that has one or more TREM2-associated activities as described herein, recognizes an epitope of human TREM2 that is the same or substantially the same as an epitope recognized by an antibody clone as described herein, and/or comprises one or more CDR, heavy chain, and/or light chain sequences of an antibody clone as described herein) for at least 1 week, at least 2 weeks, at least 3 weeks, at least 4 weeks, at least 5 weeks, at least 6 weeks, at least 7 weeks, at least 8 weeks, at least 9 weeks, at least 10 weeks, or longer. In some embodiments, the subject has been treated with an anti-TREM2 antibody for at least 1 month, at least 2 months, at least 3 months, at least 4 months, at least 5 months, at least 6 months, or longer.

In some embodiments, depending on the level of change in sTREM2 levels that is detected in the second sample as compared to the first sample, the method can further comprise adjusting the dosage of the anti-TREM2 antibody that is administered to the subject (e.g., increasing or decreasing the dosage and/or frequency of administration of the anti-TREM2 antibody). In some embodiments, the method can further comprise adjusting the anti-TREM2 antibody that is administered (e.g., administering a different anti-TREM2 antibody). In some embodiments, the method can further comprise discontinuing treatment with the anti-TREM2 antibody.

### VI. Pharmaceutical Compositions and Kits

In still another aspect, pharmaceutical compositions and kits comprising an antibody that specifically binds to a human TREM2 protein are provided. In some embodiments, the pharmaceutical compositions and kits are for use in treating a neurodegenerative disease, e.g., a neurodegenerative disease that is characterized by a mutation in TREM2. In some embodiments, the pharmaceutical compositions and kits are for use in modulating (e.g., enhancing or inhibiting) one or more TREM2 activities, e.g., Syk phosphorylation. In some embodiments, the pharmaceutical compositions and kits are for use in modulating (e.g., decreasing or increasing) sTREM2 levels. In some embodiments, pharmaceutical compositions and kits are for use in identifying whether a subject having a neurodegenerative disease is a suitable candidate for treatment with an anti-TREM2 antibody. In some embodiments, the pharmaceutical compositions and kits are for use in monitoring the efficacy of treatment with an anti-TREM2 antibody in a subject having a neurodegenerative disease.

### Pharmaceutical Compositions

In some embodiments, pharmaceutical compositions comprising an anti-TREM2 antibody are provided. In some embodiments, the anti-TREM2 antibody is an antibody (or antigen-binding portion) as described in Section III above.

In some embodiments, a pharmaceutical composition comprises an anti-TREM2 antibody as described herein and further comprises one or more pharmaceutically acceptable carriers and/or excipients. A pharmaceutically acceptable carrier includes any solvents, dispersion media, or coatings that are physiologically compatible and that does not interfere with or otherwise inhibit the activity of the active agent. Various pharmaceutically acceptable excipients are well-known in the art.

In some embodiments, the carrier is suitable for intravenous, intramuscular, oral, intraperitoneal, intrathecal, transdermal, topical, or subcutaneous administration. Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, compositions that reduce the clearance or hydrolysis of the active agents, or excipients or other stabilizers and/or buffers. Other pharmaceutically acceptable carriers and their formulations are well-known in the art.

The pharmaceutical compositions described herein can be manufactured in a manner that is known to those of skill in the art, e.g., by means of conventional mixing, dissolving, granulating, dragee-making, emulsifying, encapsulating, entrapping or lyophilizing processes. The following methods and excipients are merely exemplary and are in no way limiting.

For oral administration, an anti-TREM2 antibody can be formulated by combining it with pharmaceutically acceptable carriers that are well known in the art. Such carriers enable the compounds to be formulated as tablets, pills, dragees, capsules, emulsions, lipophilic and hydrophilic suspensions, liquids, gels, syrups, slurries, suspensions and the like, for oral ingestion by a patient to be treated. Pharmaceutical preparations for oral use can be obtained by mixing the compounds with a solid excipient, optionally grinding a resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents can be added, such as a cross-linked polyvinyl pyrrolidone, agar, or alginic acid or a salt thereof such as sodium alginate.

An anti-TREM2 antibody can be formulated for parenteral administration by injection, e.g., by bolus injection or continuous infusion. For injection, the compound or compounds can be formulated into preparations by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. In some embodiments, compounds can be formulated in aqueous solutions, e.g., in physiologically compatible buffers such as Hanks's solution, Ringer's solution, or physiological saline buffer. Formulations for injection can be presented in unit dosage form, e.g., in ampules or in multi-dose containers, with an added preservative. The compositions can take such forms as suspensions, solutions or emulsions in oily or aqueous vehicles, and can contain formulatory agents such as suspending, stabilizing and/or dispersing agents.

In some embodiments, an anti-TREM2 antibody is prepared for delivery in a sustained-release, controlled release, extended-release, timed-release or delayed-release formulation, for example, in semi-permeable matrices of solid hydrophobic polymers containing the active agent. Various types of sustained-release materials have been established and are well known by those skilled in the art. Current extended-release formulations include film-coated tablets, multiparticulate or pellet systems, matrix technologies using hydrophilic or lipophilic materials and wax-based tablets with pore-forming excipients. Sustained-release delivery systems can, depending on their design, release the compounds over the course of hours or days, for instance, over 4, 6, 8, 10, 12, 16, 20, 24 hours or more. Usually, sustained release formulations can be prepared using naturally-occurring or synthetic polymers, for instance, polymeric vinyl pyrrolidones, such as polyvinyl pyrrolidone (PVP); carboxyvinyl hydrophilic polymers; hydrophobic and/or hydrophilic hydrocolloids, such as methylcellulose, ethylcellulose, hydroxypropylcellulose, and hydroxypropylmethylcellulose; and carboxypolymethylene.

Typically, a pharmaceutical composition for use in in vivo administration is sterile. Sterilization can be accomplished according to methods known in the art, e.g., heat sterilization, steam sterilization, sterile filtration, or irradiation.

Dosages and desired drug concentration of pharmaceutical compositions of the disclosure may vary depending on the particular use envisioned. The determination of the appropriate dosage or route of administration is well within the skill of one in the art. Suitable dosages are also described in Section V above.

### Kits

In some embodiments, kits comprising an anti-TREM2 antibody are provided. In some embodiments, the anti-TREM2 antibody is an antibody (or antigen-binding portion) as described in Section III above.

In some embodiments, the kit further comprises one or more additional therapeutic agents. For example, in some embodiments, the kit comprises an anti-TREM2 antibody as described herein and further comprises one or more additional therapeutic agents for use in the treatment of a neurodegenerative disease, e.g., Alzheimer's disease. In some embodiments, the therapeutic agent is an agent for use in treating a cognitive or behavioral symptom of a neurodegenerative disease (e.g., an antidepressant, a dopamine agonist, or an anti-psychotic). In some embodiments, the therapeutic agent is a neuroprotective agent (e.g., carbidopa/levodopa, an anticholinergic agent, a dopaminergic agent, a monoamine oxidase B (MAO-B) inhibitor, a catechol-O-methyl transferase (COMT) inhibitor, a glutamatergic agent, a histone deacetylase (HDAC) inhibitor, a cannabinoid, a caspase inhibitor, melatonin, an anti-inflammatory agent, a hormone (e.g., estrogen or progesterone), or a vitamin).

In some embodiments, the kit comprises an anti-TREM antibody as described herein and further comprises one or more reagents for measuring sTREM2 levels. In some embodiments, the kit comprises an anti-TREM antibody as described herein and further comprises one or more reagents for measuring TREM2 activity (e.g., for measuring Syk phosphorylation).

In some embodiments, the kit further comprises instructional materials containing directions (i.e., protocols) for the practice of the methods described herein (e.g., instructions for using the kit for a therapeutic or prognostic method as described in Section V above). While the instructional materials typically comprise written or printed materials they are not limited to such. Any medium capable of storing such instructions and communicating them to an end user is contemplated by this disclosure. Such media include, but are not limited to electronic storage media (e.g., magnetic discs, tapes, cartridges, chips), optical media (e.g., CD-ROM), and the like. Such media may include addresses to internet sites that provide such instructional materials.

### VII. Examples

The present invention will be described in greater detail by way of specific examples. The following examples are offered for illustrative purposes only, and are not intended to limit the invention in any manner.

### Example 1. Methods of Generating and Characterizing Anti-TREM2 Antibodies

### Recombinant Expression and Purification of Mouse Fc Fused Human TREM2 ECD

The ecto domain (residues 19-172) of human TREM2 (UniProtKB ID—Q9NZC2) was subcloned into pRK vector with the secretion signal from mouse IgG kappa chain V-III, amino acids 1-20 (UniProtKB ID—P01661) at the N-terminal region, and a mouse Fc tag at the C-terminal region with a GGGGS between TREM2 ECD and Fc.

Purified plasmid was transfected into Expi293F™ cells (Thermo Fisher) using the Expi293F™ Expression System Kit according to the manufacturer's instructions. To inhibit maturation of N-linked glycans and reduce glycosylation heterogeneity, kifunensine (Sigma), an inhibitor of high mannosidase I was added to the culture at 1 μg/mL concentration immediately after transfection. Transfected cells were incubated in an orbital shaker (Infors HT Multitron) at 125 rpm and 37° C. in a humidified atmosphere of 6% CO₂. ExpiFectamine™ 293 Transfection Enhancer 1 and 2 were added to the cells 16 hours post transfection and the media supernatant was harvested 96 hours post transfection. The clarified supernatant was supplemented with EDTA-free protease inhibitor (Roche) and was stored at −80° C.

For rhTREM2-mFc isolation, clarified media supernatant was loaded on HiTrap MabSelect SuRe Protein A affinity column (GE Healthcare Life Sciences) and washed with 200 mM arginine and 137 mM succinate buffer pH 5.0. The fusion protein was eluted in 100 mM QB citrate buffer pH 3.0 and 50 mM NaCl. Immediately after elution, 1M Tris-HCl buffer pH 8.0 was added to the protein solution to neutralize the pH. Protein aggregates were separated by size exclusion chromatography (SEC) on Superdex 200 increase 10/300 GL column (GE Healthcare Life Sciences). The SEC mobile phase buffer was kept at 20 mM Tris-HCl pH 8.0, 100 mM NaCl and 50 mM arginine, which was also the protein storage buffer. All chromatography steps were performed on AKTA pure or AKTA Avant systems (GE Healthcare Life Sciences).

### Recombinant Expression and Purification of His-Tagged TREM2 ECD

The ecto domain (residues 19-172) of TREM2 (UniProtKB—Q9NZC2) was sub cloned in the pRK vector with the secretion signal from mouse Ig kappa chain V-III, amino acids 1-20 (UniProtKB ID—P01661) at the N-terminal region, and a 6X-His tag at the C-terminal region. The insert was verified by sequencing and maxi prep plasmid purification was performed.

Purified plasmid was transfected into Expi293F™ cells (Thermo Fisher) using the Expi293F™ Expression System Kit according to the manufacturer's instructions. Transfected cells were incubated in an orbital shaker (Infors HT Multitron) at 125 rpm and 37° C. in a humidified atmosphere of 6% CO₂. ExpiFectamine™ 293 Transfection Enhancer 1 and 2 were added to the cells 16 hours post transfection and the media supernatant was harvested 96 hours post transfection.

Harvested media was supplemented with 1M imidazole pH 8.0 to a final concentration of 10 mM and filtered using the Nalgene™ Rapid-Flow™ disposable filter units (Thermo Fisher) with a pore size of 0.4 microns. HisPur™ Ni-NTA Resin (Thermo Fisher) was washed with MQ water and equilibrated with load buffer (20 mM Tris pH 8.0, 150 mM NaCl, and 10 mM imidazole). Affinity purification was performed using the gravity flow method. The harvested media was loaded onto the resin and nonspecifically bound proteins were washed with load buffer supplemented with 50 and 100 mM imidazole. The bound His-tagged TREM2 eco domain was eluted with 20 mM Tris pH 8.0, 150 mM NaCl, and 200 mM imidazole. Eluted protein was concentrated using Amicon 10 kDa concentrators and the concentrated protein was further purified by gel filtration chromatography using the AKTA Avant system (GE Healthcare Life Sciences). The protein was loaded onto a HiLoad Superdex 200 16/600 (GE Healthcare Life Sciences) column equilibrated with 1×PBS and eluted and fractionated using 1×PBS as the running buffer. Eluted fractions were analyzed by electrophoresis on polyacrylamide (PAGE) gels under denaturing and native conditions. Eluted fractions were further characterized by analytical size exclusion chromatography and the intact protein mass determination. Results from the PAGE and analytical characterization were used to pool the heavily glycosylated protein fractions and these were aliquoted and stored at −80° C.

### Immunization of Mice

Wild Type Balb/c and KO C57B16 mice were immunized with TREM2Fc protein and alternating injections of BWZ cells expressing TREM2 and DAP12 (“Trem2Dap12”). Immunizations were performed via footpad bi-weekly with 5-10 μg of antigen in Sigma adjuvant for 4-6 weeks. The serum titer was screened by a cell based ELISA. Animals with titers >10⁴ were selected for a final boost. Mice were given a final boost without adjuvant via footpad and sacrificed 3 days after the boost. Popliteal and inguinal lymph nodes were harvested, made into single cell suspensions by passing through cell strainers, and then the lymphocytes were used for hybridoma generation as described below.

### Generation of Hybridoma Library

B cells harvested from lymph nodes were processed and counted. They were mixed with P3X63Ag8 cells 1:1 and fused using a BTX Hybrimune Electrofusion apparatus. The fused hybridomas were plated in 60-96 well plates with 100 μL/well of HAT (hypoxanthine-aminopterin-thymidine) selection media. The plates were fed with HT (hypoxanthine thymidine) after a week. After two weeks, 50 μL/well of supernatant was collected and screened for antigen specific binding by cell ELISA as described below.

### Generation of Human TREM2/DAP12 Stable Expression HEK Cell Line

HEK293 cells were transfected with a vector expressing wild type human TREM2 and DAP12, variant TREM2 R47H and Dap12, and DAP12 alone, respectively. Stable expressing clones were selected and the cell surface TREM2 expression was evaluated by flow cytometer. APC-conjugated rat-anti-human/mouse-TREM2 monoclonal antibody (R&D MAB17291) was used for surface TREM2 expression analysis. Clone #6 showed the highest wild type TREM2 expression level and was selected and named as HEK293-H6. The clone with highest surface expression of variant TREM2 R47H was named HEK293-R4. The clones stably expressing DAP12 were analyzed by Western blot, and the selected clone was named HEK293-DAP12#1.

### Screening Antibodies for Binding by Protein ELISA

Hybridoma supernatants were screened in a Protein ELISA in a 384 well format. Four different proteins were coated in the 4 quadrants: TREM2-His, ADAM10 peptide 115-143 amino acids, 134-154 amino acids and 149-170 amino acids were coated in a 384 well plate in PBS at 1 jag/ml.

25 μL/well of hybridoma supernatants were added to the plate. Plates were incubated at room temperature for 1 hour, washed with PBST buffer 3×. A secondary detection antibody goat anti-mouse HRP (Southern Biotech) at 1:7000 dilution in media, 25 μL/well was added to the plate and incubated at room temperature for 1 hour. After an hour, plates were washed three times with PBST buffer. Plates were developed with 25 μL/well of TMB substrate (ThermoFisher) and quenched with 25 μL/well of 1 N sulfuric acid. The signal was quantified on a BioTek® plate reader at A450. Wells with an OD three times the background for TREM2 and/or on peptides were considered positive and carried forward for secondary screening.

Antibody binding data for anti-TREM2 antibodies is shown in Table 1 below.

TABLE 1
Binding Data for TREM2 Antibodies

| Antibody Clone ID | Isotype | Human TREM2 Kd |
|---|---|---|
| RS9.F10 | Ig2a, k | 8.52E−10 |
| RS9.F6 | Ig2a, k | 7.33E−10 |
| 13B11.A1 | Ig2a, k | 9.67E−07 |
| 21D4.D1 | Ig2a, k | 1.82E−09 |
| 22B8.B1 | IgG3, k | 3.82E−08 |
| 3D3.A1 | Ig2a, k | 7.43E−09 |
| 42E8.H1 | Ig2a, k | 1.70E−08 |
| 43E9.H1 | Ig2b, k | 2.99E−07 |
| 30A8.A1 | Ig2a, k | 6.76E−08 |
| 21D6.G2 | Ig2b, k | 4.00E−07 |
| 57D7.A1 | Ig2a, k | 2.41E−09 |
| 59C6.F1 | N.D. | 2.38E−05 |
| 53H11.D3 | IgG1, k | 2.56E−08 |
| 60A4.B1 | IgG1, k | 6.75E−08 |
| 24B4.A1 | Ig2a, k | 1.24E−08 |
| 39H10.A1 | Ig2b, k | 1.84E−07 |
| 55B9.A1 | Ig2a, k | 1.12E−07 |
| 26E2.A3 | Ig2a, k | 1.28E−08 |
| 54C2.A1 | Ig2b, k | 2.19E−09 |
| 44E2.H1 | Ig2b, k | 7.68E−08 |
| 22G9.D1 | IgG1, k | 2.26E−08 |
| 14H11.A1 | IgG1, k | 5.75E−08 |
| 49H11.B1 | IgG3, k | 6.34E−08 |
| 40H3.A4 | N.D. | 8.98E−10 |
| 14D5.F1 | IgG3, k | 1.18E−08 |
| 38E9.E5 | IgG1, k | 2.01E−07 |
| RS9.E2 | N.D. | 1.97E−06 |
| 26D11.B1 | N.D. | 9.17E−09 |
| 44E3.B1 | IgG1, k | 2.28E−08 |
| 2G4.B1 | IgG2a, k | 1.04E−07 |
| 30F2.A2 | IgG2a, k | 2.94E−09 |
| 51D4 | IgG2a, k | 2.67E−09 |
| 52H9.D1 | N.D. | N.D. |
| 26D2.D1 | IgG2a, k | 1.50E−08 |
| 21D11 | IgG2a, k | 5.25E−10 |
| 26D5.A1 | IgG2b, k | 1.30E−08 |
| 8A11.B1 | IgG2a, k | N.B. |
| 7B10.A2 | N.D. | N.D. |
| 19F10.F3 | IgG2a, k | N.B. |

N.D. = not determined
N.B. = no binding detected

### Biacore

Anti-murine Fc antibody (obtained from GE Healthcare) was immobilized on the surface of a CM5 chip (obtained from GE Healthcare) through amine-coupling to reach about 6,000 to 8,000 response units (RU). The surface was activated by injection of a mixture of 1-ethyl-3-(3-dimethylaminopropyl)-carbodiimide (EDC) and N-hydroxysuccinimide (NHS), both obtained from GE Healthcare, for 7 minutes. Anti-murine Fc antibody was diluted in sodium acetate (pH 5.0) at 25 μg/mL and injected for 10 minutes at a flow rate of 5 μL/minute, followed by injection of ethanolamine (obtained from GE Healthcare) for 7 minutes.

Purified anti-TREM2 hybridoma (20 μg/mL) was captured to reach 1000-1500 RU. A range of serially-diluted TREM2-His protein (e.g., 3.4 nM to 300 nM) was injected at a flow rate of 30 μL/minute, using either single-cycle kinetics methods. Sensorgrams were fitted using a 1:1 Langmuir model to estimate kₒₙ and k_{off}.

### Screening Antibodies for Binding to Trem2Dap12 by Cell ELISA

Primary screening was performed by plating Trem2Dap12 expressing HEK 293 in 96 well cell culture Nunc plates in media. 50,000 cells/well were plated in assay medium and incubated at 37° C. On day two, the media from the plates was removed and 50 μL/well of hybridoma supernatants was added to the plates with incubation at 4° C. for 1 hour. After incubation, the plates were washed six times with HBSS buffer using the Biotek plate washer. A secondary detection antibody goat anti-mouse HRP (Southern Biotech) at 1:2000 dilution in media, 50 μL/well was added to the plate and incubated at 4° C. for 1 hour. After an hour, plates were washed six times with HBSS buffer. Plates were developed with 50 μL/well of TMB substrate (ThermoFisher) and quenched with 50 μL/well of 1 N sulfuric acid. The signal was quantified on a BioTek® plate reader at A450. Wells with an OD three times the background for TREM2 were considered positive and carried forward for secondary screening.

Positives from primary screening were carried forward into the secondary screening where they were screened in functional assays like sTREM2, pSyk and FACS binding.

### FACS Binding on Mixed Cell Lines

HEK 293 overexpressing human TREM2 (H6) and HEK 293 overexpressing GFP (B5) were harvested by 0.05% trypsin and incubated at 37° C. for 2 hours for surface TREM2 recovery. 293F overexpression mouse TREM2 were harvested and labelled with NucBlue live cell stain ReadyProbes reagent for 10 minutes (2 drops of reagent added per mL of media). After labeling, cells were washed twice with 1×PBS. NucBlue labeled 293F cells were mixed with H6 and B5 in FACS buffer (PBS-+-0.5% BSA) with human Trustain FcX solution (Biolegend, cat #422302) at a density of 10⁶/mL per cell line. Mixed cell lines were seeded at 300,000 cells per well in a 96-well round-bottom and incubated 20 minutes at room temperature. After incubation, cells were centrifuged and test anti-TREM2 antibodies were added, then incubated for 45 minutes on ice. After incubation, cells were centrifuged and washed with FACS buffer three times. Cells were then incubated with secondary antibody (Alexa Fluor 647 AffiniPure F(ab′)₂ fragment goat anti-mouse IgG, Fcγ fragment specific (1:200, Jackson ImmunoResearch, cat #115-606-071) for 30 minutes on ice. After incubation, cells were washed with FACS buffer three times and resuspended in 90 μL of FACS buffer, then analyzed by flow cytometry (BD FACSCanto II, San Jose, Calif.). 20,000 events were obtained for each sample.

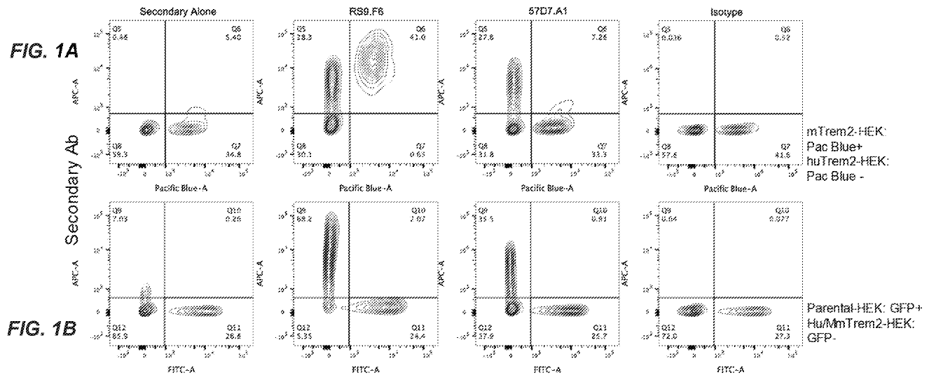

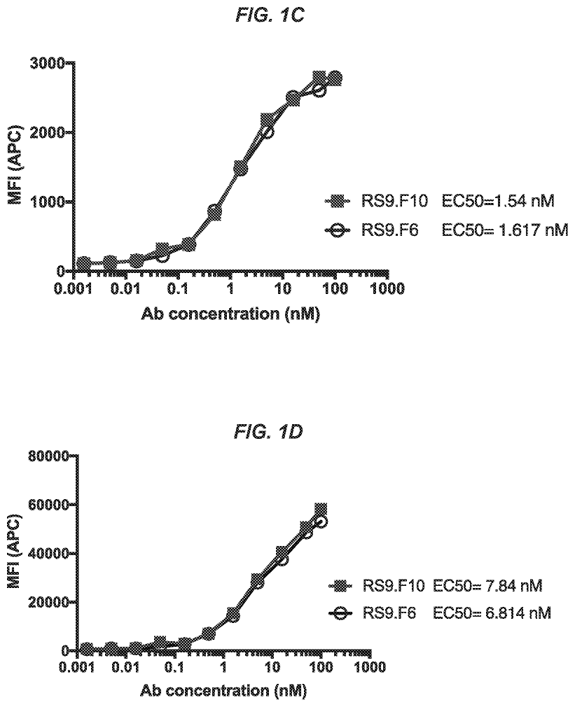

Antibody surface binding data for anti-TREM2 antibodies binding to human or mouse TREM2-expressing cells is shown in FIGS. 1A-D and in Table 2 below. The data in Table 2 is presented as fold over binding (FOB).

TABLE 2
Antibody surface binding to human
or mouse TREM2-expressing HEK cells

| | Human TREM2 | Mouse TREM2 |
|---|---|---|
| Antibody | expressing | expressing |
| Clone ID | cells - FOB | cells - FOB |
| RS9.F10 | 12.9 | 75.8 |
| RS9.F6 | 13.2 | 73.0 |
| 13B11.A1 | 4.8 | 2.6 |
| 21D4.D1 | 12.3 | 4.0 |
| 22B8.B1 | 12.3 | 3.0 |
| 3D3.A1 | 11.7 | 8.4 |
| 42E8.H1 | 10.6 | 3.5 |
| 43E9.H1 | 10.6 | 7.0 |
| 30A8.A1 | 5.8 | 2.1 |
| 21D6.G2 | 5.4 | 7.1 |
| 57D7.A1 | 11.5 | 1.7 |
| 59C6.F1 | 11.5 | 1.2 |
| 53H11.D3 | 11.6 | 1.5 |
| 60A4.B1 | 10.8 | 1.5 |
| 24B4.A1 | 8.6 | 1.5 |
| 39H10.A1 | 7.0 | 1.2 |
| 55B9.A1 | 4.0 | 1.1 |
| 26E2.A3 | 10.3 | 1.5 |
| 54C2.A1 | 12.8 | 1.7 |
| 44E2.H1 | 7.8 | 1.7 |
| 22G9.D1 | 11.9 | 1.6 |
| 14H11.A1 | 3.0 | 1.2 |
| 49H11.B1 | 7.0 | 1.2 |
| 40H3.A4 | 1.0 | 0.8 |
| 14D5.F1 | 0.9 | 0.8 |
| 38E9.E5 | 2.9 | 1.0 |
| RS9.E2 | 1.0 | 0.8 |
| 26D11.B1 | 0.9 | 1.1 |
| 44E3.B1 | 4.0 | 1.5 |

| | Human TREM2 | Mouse TREM2 |
|---|---|---|
| Antibody | expressing | expressing |
| Clone ID | cells - FOB | cells - FOB |
| 2G4.B1 | 1.6 | 1.1 |
| 30F2.A2 | 8.0 | 1.3 |
| 51D4.A1 | 7.6 | 1.3 |
| 52H9.D1 | N.D | N.D |
| 44E2.H1.F2 | 6.1 | 1.9 |
| 26D2.D1 | 4.9 | 2.8 |
| 21D11.B1 | 8.4 | 1.5 |
| 26D5.A1 | 7.3 | 1.3 |
| 8A11.B1 | 0.9 | 0.9 |
| 7B10.A2 | N.D | N.D |
| 19F10.F3 | 1.1 | 1.1 |

N.D. = not determined

### FACS Binding on Primary Macrophages

Human monocytes were isolated following the RosetteSep human monocyte enrichment cocktail protocol (Stemcell Technologies, Cat #15068). Isolated monocytes were washed in wash buffer (PBS+2% FBS) and resuspended in 10 mL ACK lysis solution to lyse red blood cells. 20 mL wash buffer was added to stop the ACK lysis, then centrifuged and washed one more time with culture media (RPMI1640+10% FBS+P/S). Human monocytes were differentiated into macrophage in culture media (RPMI 1640+10% FBS+P/S) in the presence of 50 ng/mL human M-CSF at 250 mL flask. Fresh human M-CSF was spiked on day 3 and human macrophages were harvested on day 5. Human macrophages were resuspended in FACS buffer with human Trustain FcX solution (Biolegend, cat #422302) and 1% human serum at a density of 10⁶/mL and seeded at 100,000 cells per well in a 96-well round-bottom, then incubated 20 minutes at room temperature. Cells were centrifuged and the supernatant discarded, then test anti-TREM2 antibody was added (100 nM) into 96 well plates and incubated for 45 minutes on ice. After incubation, cells were centrifuged and washed with FACS buffer three times. Cells were then incubated with secondary antibody (APC goat anti-mouse Ig, multiple adsorption, BD Pharmingen, #550826, 1:500) for 30 minutes on ice. After incubation, cells were washed with FACS buffer three times and resuspended in 90 μL FACS buffer, then analyzed by flow cytometry (BD FACSCanto I1, San Jose, Calif.). 20,000 events were obtained for each sample.

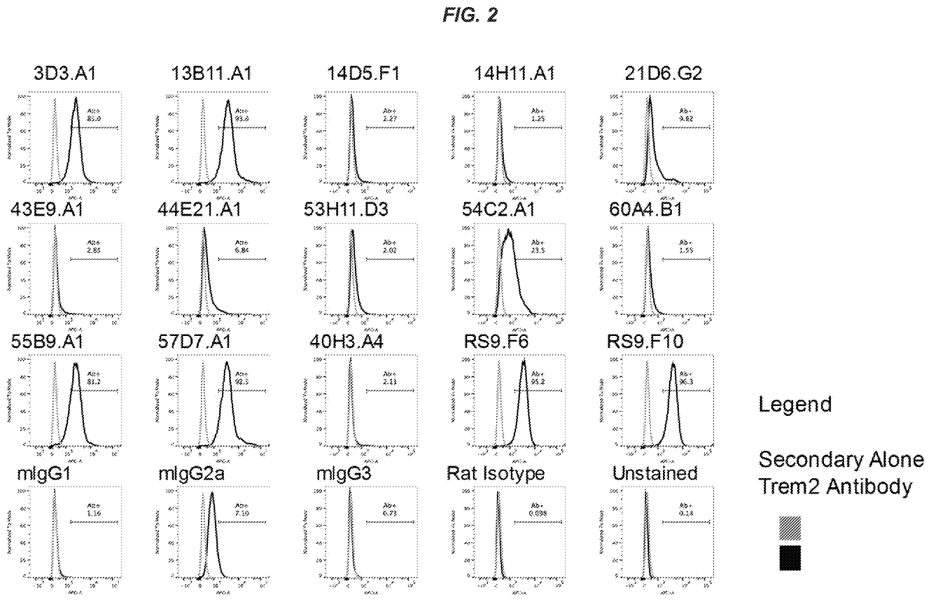

Anti-TREM2 antibody binding to primary human macrophages is shown in FIG. 2.

### TREM2 Protein/Peptide ELISA

TREM2-His tagged protein or streptavidin were adsorbed to 384-well high-binding plates. Wells were blocked by the addition of 3% BSA/Tris-buffered saline containing 0.05% Tween-20 (TBST). Upon washing with TBST, biotinylated peptides corresponding to TREM2 amino acids 115-143, 134-154, or 149-170 were added to wells containing streptavidin. Plates were washed prior to addition of hybridoma supernatants or control antibodies. Primary detection antibodies were incubated for 1 hour at room temperature, followed by five TBST washes, prior to addition of an anti-mouse IgG-HRP conjugated secondary antibody. Following five TBST washes, detection reagent (One-step TMB Ultra, Thermo) was added and incubated for 7 minutes prior to addition of the stop reagent (2N sulfuric acid). Absorbance at 450 nm was measured using a Biotek plate reader. All steps were carried out using a Hamilton Nimbus liquid handler and a Biotek 405 plate washer.

TREM2 pSYK AlphaLisa

Activation of TREM2-dependent pSyk signaling was measured using a commercial AlphaLisa assay from Perkin-Elmer. This assay used a cell line termed H6, an engineered HEK 293 cell line that overexpresses TREM2 and DAP12 (an adaptor protein in TREM2 signaling). The cells were grown for 2-3 days in T-150 flasks prior to the assay in DMEM containing 1× glutamax, 10% FBS, 1× Pen/Strep solution, and 200 μg/mL zeomycin. Prior to the assay the cells were recovered by trypsinization, centrifugation, and resuspension in fresh antibiotic free media. They then were stored in a 50 mL conical tube for 2-6 hours in a 37° C. tissue culture incubator, after which they were centrifuged and resuspended in HBSS for use in the pSyk assay.

The samples containing anti-TREM2 antibodies for screening/testing in the pSyk assay were coated onto magnetic Protein G coated Dynabeads (Thermo Scientific) in 96 well plates, using a 1 hour incubation at room temperature with vigorous shaking. After coating the suspension of H6 cells was added to the bead-coated antibody (100 μL/well from a suspension of 3,000,000 cells/mL, for 300,000 cells per well). The 96-well plates containing antibody-coated beads and H6 cells were briefly incubated in a 37° C. tissue culture incubator for 5 minutes. Afterwards plates were removed and centrifuged. Supernatant was removed by pipetting and lysates prepared by addition of 25 μL/well of lysis buffer (from Cell Signaling Technology, supplemented with 1 mM PMSF), with mixing by pipetting. Before assaying the lysates were incubated for 30 minutes on ice.

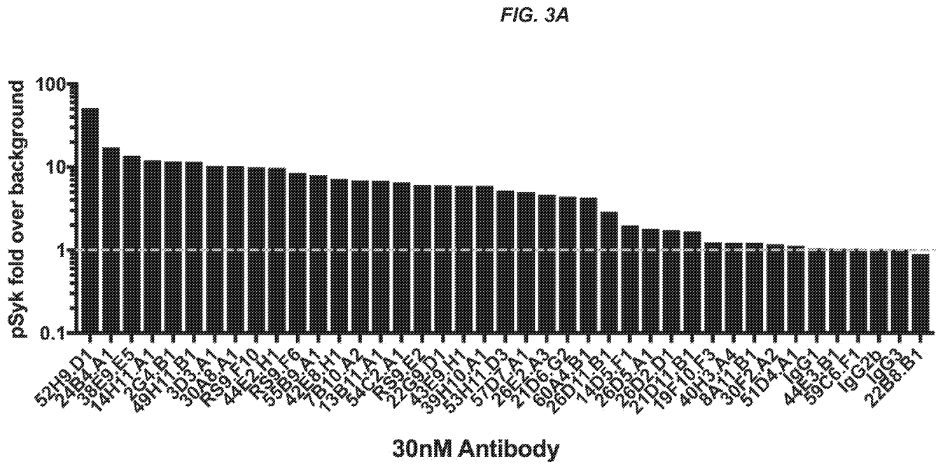

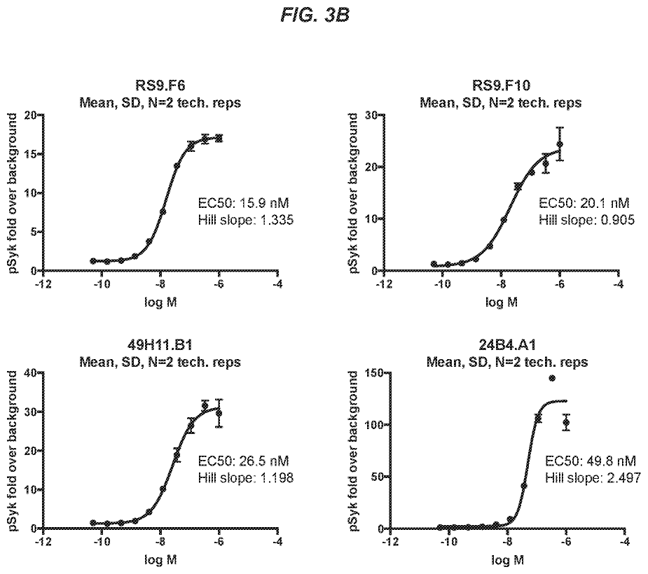

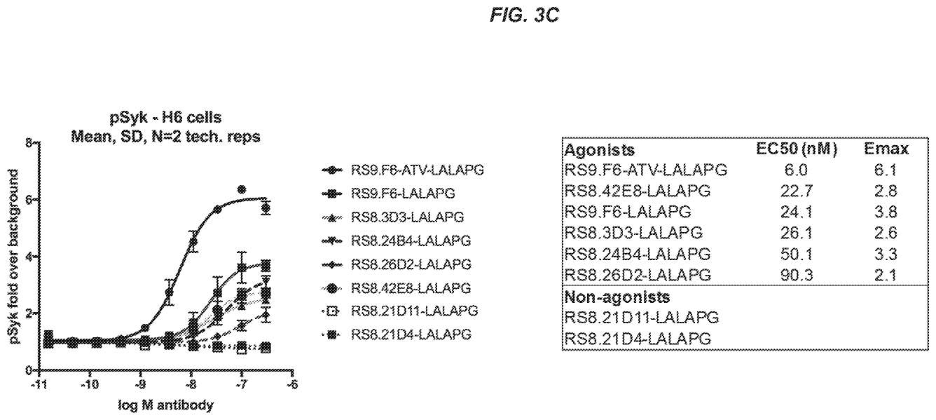

After preparation and incubation of lysates, the lysates were assayed for pSyk using the standard protocol for the Perkin Elmer pSyk Alpha Lisa kit. In brief, 10 μL of lysate/well was transferred to a white opaque 384 well Optiplate (Perkin Elmer). Next 5 μL of Acceptor Mix (containing the working solution of acceptor beads) was added per well followed by sealing of plates with foil seals and incubation 1 hour at room temperature. After this 5 μL of Donor Mix (containing the working solution of donor beads) was added to each well under reduced light conditions. Plates were again sealed and incubated 1 hour at room temperature. Finally plates were read using Alpha Lisa settings on a Perkin Elmer EnVision plate reader. pSyk induction by anti-TREM2 antibodies is shown in FIG. 3A and in Table 3 below, and is presented as fold over background (FOB). FIG. 3B and Table 4 show EC50 values for selected antibody clones on human TREM2-expressing HEK cells.

TABLE 3
pSyk induction by TREM2 antibodies

| Antibody Clone ID | pSyk FOB (30 nM Ab) |
|---|---|
| 52H9.D1 | 51.67 |
| 24B4.A1 | 17.29 |
| 38E9.E5 | 13.67 |
| 14H11.A1 | 12.02 |
| 2G4.B1 | 11.70 |
| 49H11.B1 | 11.56 |
| 3D3.A1 | 10.35 |
| 30A8.A1 | 10.24 |
| RS9.F10 | 9.89 |
| 44E2.H1 | 9.82 |
| RS9.F6 | 8.49 |
| 55B9.A1 | 7.97 |
| 42E8.H1 | 7.17 |
| 7B10.A2 | 6.89 |
| 13B11.A1 | 6.87 |
| 54C2.A1 | 6.56 |
| RS9.E2 | 6.11 |
| 22G9.D1 | 6.03 |
| 43E9.H1 | 5.95 |
| 39H10.A1 | 5.94 |
| 53H11.D3 | 5.20 |
| 57D7.A1 | 5.02 |
| 26E2.A3 | 4.67 |
| 21D6.G2 | 4.40 |
| 60A4.B1 | 4.28 |
| 26D11.B1 | 2.89 |
| 14D5.F1 | 1.99 |
| 26D5.A1 | 1.81 |
| 26D2.D1 | 1.73 |
| 21D11.B1 | 1.68 |
| 19F10.F3 | 1.24 |
| 40H3.A4 | 1.23 |
| 8A11.B1 | 1.22 |
| 30F2.A2 | 1.17 |
| 51D4.A1 | 1.12 |
| IgG1 | 1.05 |
| 44E3.B1.A2 | 1.04 |
| 59C6.F1 | 1.04 |
| IgG2b | 1.03 |
| IgG3 | 1.02 |
| 22B8.B1 | 0.89 |

TABLE 4
TREM2 Antibody EC50s on human TREM2-expressing HEK cells

| Antibody | Cell | | STREM2 |
|---|---|---|---|
| Clone ID | binding (nM) | pSyk (nM) | decrease (nM) |
| RS9.F6 | 1.62 | 15.87 | 0.3 |
| RS9.F10 | 1.54 | 20.50 | N.D. |
| 49H11.B1 | N.D. | 26.48 | N.D. |
| 24B4.A1 | 7.5 | 49.78 | N.D. |
| 54C2 | 4.09 | 455.6 | N.D. |

Additional data is shown in FIG. 3C, which confirms that non-agonists 21D11 and 21D4 do not induce p-Syk. These data were generated as follows. Two days in advance of the experiment, HEK293 cells stably overexpressing TREM2 and DAP12 were plated at 40,000 cells/well on a 96 well poly-D-lysine-coated plate. In PAM experiments utilizing lipid vesicles, the lipid vesicles (see protocol below) were then mixed with the antibodies (at a constant concentration) on a 96-well plate using a Hamilton Nimbus liquid handler at a liposome concentration of 0, liposome EC20, EC50, or EC80 (see below) in PBS. In agonist experiments alone (no liposomes), antibodies were diluted without liposomes in PBS. The cells were washed 3× with HBSS with a Biotek 405/406 plate washer, then 50 μL of the liposome/antibody mixture was added per well using a Hamilton Nimbus liquid handler. The cell plate containing the liposome/antibodies was then transferred to a 37° C. incubator for 5 minutes. The liposome/antibody solution was removed by flicking the plate, and 40 μL lysis buffer (Cell Signaling Technologies, CST) was added using the liquid handler. The lysate was incubated at 4° C. for 30 minutes, then either frozen at −80° C. or immediately carried forward to the alpha-LISA assay as described above.

### Soluble TREM2 (TREM2 Shedding) Assay

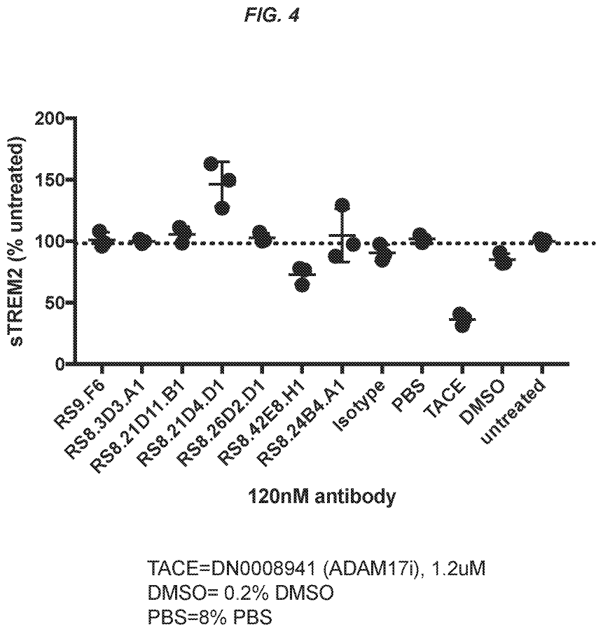

HEK293 cells stably expressing human TREM2 and DAP12 were plated onto 96-well plates pre-coated with poly-D-lysine 4-16 hours prior to antibody treatment. Antibody supernatants or control antibodies were diluted into fresh complete media and added onto cells in triplicate. Cells were incubated with antibody containing media for 18 hours prior to removal of the cell supernatant for assay by TREM2 ELISA. Samples and standards were diluted 1:10 into blocking buffer (3% BSA/TBST) in the assay plate. Briefly, MSD small spot streptavidin plates were coated with biotinylated anti-hTREM2 polyclonal antibody (R&D Systems) overnight at 4° C. or 1 hour at room temperature. Plates were then blocked overnight at 4° C., or 1 hour at room temperature, with blocking buffer, 3% BSA/TBST. Plates were washed three times with TBST in a Biotek plate washer (used for all washes) prior to addition of diluent (blocking buffer) and denatured supernatants from cell culture. A TREM2-His protein diluted in 3% BSA/TBST was used as a standard for absolute quantification. Following 1 hour incubation at room temperature, plates were washed with TBST. The primary detection antibody, sulfo-tagged goat anti-human TREM2 (R&D Systems), diluted in 3% BSA/TBST was added and incubated for 1 hour at room temperature. After washing with TBST, MSD plates were developed using 2×MSD read buffer T, followed by detection using an MSD Sector plate reader. MSD values were converted to absolute quantities of sTREM2 by fitting a standard curve using Prism 7.0 software (Graphpad). Modulation of TREM2 shedding was represented as a ratio to cells cultured with no specific TREM2 antibody in the media. Results from the TREM2 shedding assay are shown in FIG. 4. As shown in FIG. 4, 42E8 defines a class of antibody that blocks TREM2 shedding, while 21D4 defines a class of antibody that enhances TREM2 shedding.

Positive Allosteric Modulator (PAM) and Single Point pSyk Screen

Two days in advance of the experiment, HEK293 cells stably overexpressing TREM2 and DAP12 were plated at 40,000 cells/well on a 96 well poly-D-lysine-coated plate. The lipid vesicles (see protocol below) were then mixed with the antibodies on a 96-well plate using a Hamilton Nimbus liquid handler at a concentration of 0, EC20, EC50, or EC80 (see below). The cells were washed 3× with HBSS with a Biotek 405/406 plate washer, then 50 μL of the liposome/antibody mixture was added per well using a Hamilton Nimbus liquid handler. The cell plate containing the liposome/antibodies was then transferred to a 37° C. incubator for 5 minutes. The liposome/antibody solution was removed by flicking the plate, and 40 μL lysis buffer (Cell Signaling Technologies, CST) was added using the liquid handler. The lysate was incubated at 4° C. for 30 minutes, then either frozen at −80° C. or immediately carried forward to the alpha-LISA assay as described above.

Liposome Formation and Titration to Determine EC20, EC50, and EC80 of Liposome-Mediated pSyk Activity on TREM2/DAP12-Overexpressing Cells

Liposomes were prepared on the same day as the experiment as follows: 7 mg DOPC (1,2-dioleoyl-sn-glycero-3-phosphocholine) and 3 mg POPS (1-palmitoyl-2-oleoyl-sn-glycero-3-phospho-L-serine) were combined in chloroform in a glass vial and dried under a stream of N2 gas for 1-2 hours, or until completely dry. The lipid mixture was resuspended in 1 mL HBSS (10 mg/mL final lipid concentration) and vortexed for 2-3 minutes. Subsequently, the lipid suspension was extruded 29 times using an Avanti mini-extruder constructed with one 100 nm pore size membrane to form small unilamellar vesicles at 10 mg/mL.

Liposome EC20, EC50, and EC80 were determined separately by titrating liposomes starting at 6 mg/mL (calculated using the mole-percent weighted average of the molecular weights, which is equal to 793 g/mol), down to 0.00157 mg/mL in a ten point dilution curve. The liposomes were diluted in HBSS, and 50 μL of liposome solution was added per well to TREM2/DAP12-overexpressing cells using the Hamilton Nimbus liquid handler as in the above protocol. The cells were incubated for 5 minutes, then lysed in 40 μL CST lysis buffer, incubated 30 minutes at 4° C., then either frozen at −80° C. or carried forward to the alpha-LISA assay. The pSyk response was measured as described above from 10 μL lysate, and the curve fit using a non-linear regression (4 parameter dose response) fit in Prism. The determined EC20 was 0.046 mg/mL, EC50 was 0.212 mg/mL, and EC80 was 0.967 mg/mL.

### Human Macrophage Chemotaxis Assay

Human monocytes are isolated following the RosetteSep human monocyte enrichment cocktail protocol (Stemcell Technologies, Cat #15068). Isolated monocytes are washed in wash buffer (PBS+2% FBS) and resuspended in 10 mL ACK lysis solution to lyse red blood cells. 20 mL wash buffer is added to stop the ACK lysis, then the sample is centrifuged and washed one more time with culture media (RPMI1640+10% FBS+P/S). Cells are resuspended in culture media at density of 1 million/mL and differentiated to macrophages in the presence of human MCSF (50 ng/mL, Gibco, Cat # PHC9501) in 125 mL flask (25 million/flask). Fresh human M-CSF are spiked at day 3. Human macrophages are harvested at day 5 and washed with migration assay buffer (RPMI, no phenol red +0.1% BSA). After washing, cells are resuspended in assay buffer at a density of 1,000,000 cells/mL and labeled with 1.33 μM calcein AM fluorescent dye (Coming, cat. #354217) for 45 minutes in a cell culture incubator. Following incubation, cells are washed once with assay buffer and resuspended in assay buffer at a density of 1,000,000/mL. Human macrophage chemotaxis assay is performed using a Corning FluoroBlok 24-Multiwell insert system (Corning, cat #351158). Labeled cell suspension is added onto inserts (100 μl/well) and set aside. In a separate 24-well plate, 650 μL of chemoattractant C5a (1 ng/mL) is added. The multiwell insert containing cells is gently lowered into the plate containing chemoattractants and immediately placed into a bottom fluorescence plate reader at 485/530 nm (Ex/Em) wavelength (BioTeck, SYNERGY microplate reader). Fluorescence emitted from cells that have migrated to the bottom surface of the insert is measured at various time points.

Phagocytosis Assay

Phagocytosis assays using pHrodo™Red-labeled myelin preparation can be used to examine the phagocytic activity of human and mouse primary microglia, macrophages or macrophage/microglia cell lines (such as THP-1 and ECO20 respectively). Briefly, phagocytic cells are plated on 96-well plates. The cell is pre-treated with antibody (or left untreated for a negative control, or treated with control reagents), the proper amount of labeled substrate is added to the cells, and the cells are incubated at 37° C. for 2 to 6 hours. The phagocytosis activity is measured by Opera Phenix High-Content Screening System (PerkinElmer).

### Cell Survival Assay

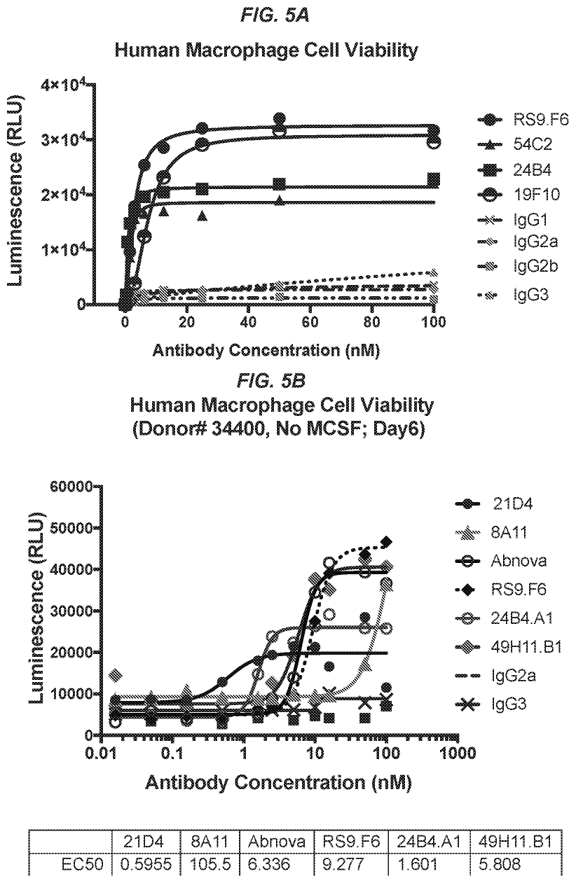

Previous studies have indicated that TREM2 plays a critical role in macrophage differentiation and survival (Wu et al., J. Exp. Med., 2009, 212:681-697). Recent studies using microglia cells from wild-type and TREM2 knockout mice showed that the microglia from the knockout mice had cell survival deficits (Zheng et al., J Neurosci, 2017, 37:1772-1784). TREM2 can support microglia energy homeostasis (Ulland et al., Cell, 2017, 170:649-663). The studies using TREM2 knockout mice suggest TREM2 is necessary for myeloid cell survival. To understand if enhancing TREM2 activity with TREM2 agonistic antibodies is sufficient for macrophage/microglia survival under limited M-CSF conditions, human macrophage differentiation and survival studies were performed using antibodies as described herein. Human monocytes isolated from peripheral blood were incubated with 5 ng/mL M-CSF in the presence of titrated concentrations of plate coated TREM2 antibodies or isotype controls. On day 6 cell viability was determined by CellTiter Glo viability assay. As shown in FIGS. 5A-B, anti-TREM2 antibodies, such as the anti-TREM2 agonistic antibodies RS9.F6, 54C2.A1, 24B4.A1, and 19F10.F3, increased the survival of human macrophages cultured in restricted or no M-CSF.

Western and AlphaScreen assays can be used to look at the activation of the signaling components in cell survival pathways, pS9-GSK3beta, pT374-AKT and p-Erk. For these assays, monocytes are isolated from human blood cells and plated at 100,000 cells per well in a 96-well cell culture plate with RPMI-1640 medium containing 10% hyclone FBS, antibiotics and 50 ng/mL M-CSF, and the macrophages are differentiated from human monocytes. On day 6 of differentiation, cells are treated under M-CSF withdrawal for 4-6 hours. Anti-TREM2 antibodies and the proper IgG isotype controls, are added to the cell at proper concentration, respectively, and incubated in at 37° C. for 5 minutes. Media is removed rapidly, plates are placed on ice and washed with 200 μL of ice cold TBS, and the solution is completely removed. Cells are lysed with 40-50 μL of Cell Lysate Buffer, shaking at 4° C. for 1 hour. Lysates can be stored at −80° C. AlphaScreen kits from PerkinElmer and pS9-GSK3beta, pT374-AKT and p-Erk can be used to measure phosphorylation according the manufacturer's instructions.

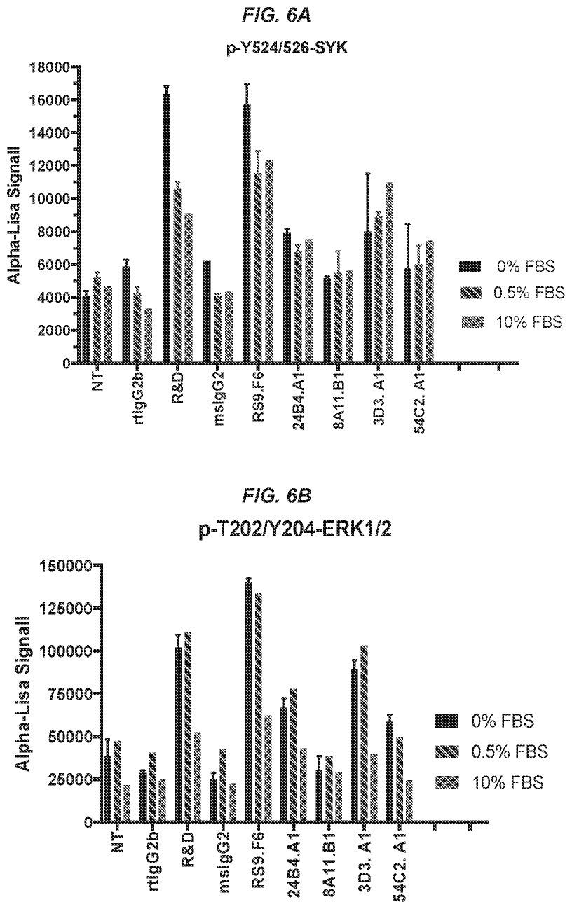

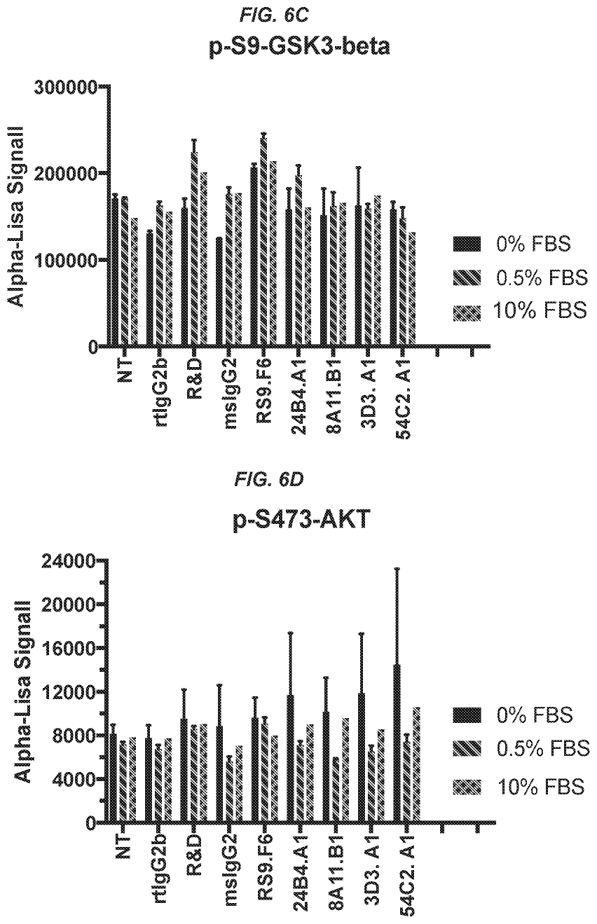

Anti-TREM2 antibodies were characterized for activation of several signaling pathways, SYK, ERK1/2, AKT and GSK3-beta. Human macrophages differentiated from peripheral monocytes were plated in 96-well plates at a density of 100,000 cell/well. The assay was tested in three conditions (0%, 0.5%, and 10% FBS in the media) for 3 hours. Cells were stimulated with the antibody RS9.F6, 24B4.A1, 8A11.B1, 3D3.A1, 54C2.A1, isotype control IgG2ak, rat anti-TREM2 from R&D, isotype control rat IgG2b, or no treatment (NT) for 15 minutes. The cell lysates were measured for p-Y525/526-SYK, p-T202/Y204-ERK1/2, p-S9-GSK3-beta, and p-S473-AKT using Alpha-LISA kits (Perkin Elmer). As shown in FIGS. 6A, 6B, and 6D, RS9.F6, 24B4.A1, 3D3.A1, 54C2.A1 and rat-anti-TREM2 antibodies induced robust p-SYK and pERK1/2 signal. The antibodies stimulated p-GSK signal moderately (FIG. 6C).

A summary of the cell binding characteristics, activation of pSyk, change in sTREM2 levels, and interaction with lipid ligand to activate pSyk for anti-TREM2 antibodies as described herein is shown in Table 5 below.

TABLE 5
Summary of TREM2 Antibodies

| | Cell Binding | | | | | | | | |
|---|---|---|---|---|---|---|---|---|---|
| | Human | Primary | | Mouse | | | | With Liposomes | |
| | TREM2 | Human | Primary | TREM2 | Primary | | | Additive | Inhibitory |
| Clone ID | HEK | Macrophage | Cyno | HEK | Mouse | pSyk | STREM2 | pSyk | pSyk |
| RS9.F10 | X | X | X | X | X | ++ | NC | X | |
| RS9.F6 | X | X | X | X | X | ++ | NC | X | |
| RS8.13B11.A1 | X | X | N.D. | | N.D. | ++ | | | X |
| RS8.21D4.D1 | X | X | N.D. | | N.D. | | ++ | | XX |
| RS8.22B8.B1 | X | X | N.D. | | N.D. | | | X | |
| RS8.3D3.A1 | X | X | N.D. | X | N.D. | ++ | NC | X | |
| RS8.42E8.H1 | X | X | N.D. | | N.D. | + | −− | X | |
| RS8.43E9.H1 | X | X | N.D. | X | N.D. | ++ | | X | |
| RS8.30A8.A1 | X | X | N.D. | | N.D. | ++ | | | X |
| RS8.21D6.G2 | X | X | N.D. | X | N.D. | + | | X | |
| RS8.57D7.A1 | X | X | N.D. | | N.D. | + | | | X |
| RS8.59C6.F1 | X | X | N.D. | | N.D. | | | X | |
| RS8.53H11.D3 | X | X | N.D. | | N.D. | ++ | | X | |
| RS8.60A4.B1 | X | X | N.D. | | N.D. | ++ | | X | |
| RS8.24B4.A1 | X | X | N.D. | | N.D. | ++ | NC | | X |
| RS8.39H10.A1 | X | X | N.D. | | N.D. | ++ | | | X |
| RS8.55B9.A1 | X | X | N.D. | | N.D. | ++ | | | X |
| RS8.26E2.A3 | X | X | N.D. | | N.D. | ++ | | X | |
| RS8.54C2.A1 | X | X | N.D. | | N.D. | ++ | NC | X | |
| RS8.44E2.H1 | X | X | N.D. | | N.D. | ++ | | X | |
| RS8.22G9.D1 | X | X | N.D. | | N.D. | + | | X | |
| RS8.14H11.A1 | X | X | N.D. | | N.D. | ++ | | | X |
| RS8.49H11.B1 | X | X | N.D. | | N.D. | ++ | | X | |
| RS8.40H3.A4 | X | | N.D. | | N.D. | | | | X |
| RS8.14D5.F1 | X | | N.D. | | N.D. | | | X | |
| RS8.26D11.B1 | | | N.D. | | N.D. | | | X | |
| RS8.52H9.D1 | N.D. | N.D. | N.D. | N.D. | N.D. | +++ | | X | |
| RS8.8A11.B1 | | X | N.D. | | N.D. | | NC | X | |
| RS8.7B10.A2 | N.D. | N.D. | N.D. | N.D. | N.D. | ++ | | X | |
| RS8.19F10.F3 | | X | N.D. | | N.D. | | | X | |
| RS8.30F2.A2 | X | | N.D. | | N.D. | | | | X |
| RS8.51D4.A1 | X | | N.D. | | N.D. | | | | X |
| RS8.26D2.D1 | X | | N.D. | | N.D. | + | NC | | X |
| RS8.21D11.B1 | X | | N.D. | | N.D. | | NC | | X |
| RS8.44E3.B1 | X | | N.D. | | N.D. | | NC | | X |
| RS8.26D5.A1 | X | | N.D. | | N.D. | | | | X |
| RS8.38E9.E5 | X | | N.D. | | N.D. | ++ | | | X |
| RS9.E2 | | | N.D. | | N.D. | + | | | X |
| RS8.2G4.B1 | | | N.D. | | N.D. | ++ | | | X |

N.D. = Not determined
* In cell binding columns, blank indicates no cell binding
** In pSyk column, blank indicates no effect with a cutoff of 5X above background
*** In sTREM2 column, the following legend is used: NC = no change; −− = reduced sTREM2; ++ = increased sTREM2

### Example 2. Hybridoma Sequencing

Total RNA was extracted from approximately 5×10⁶ hybridoma cells producing each antibody using Qiagen RNeasy Mini Kit (Qiagen). The first-stranded cDNA was synthesized using the SMART RACE cDNA Amplification Kit (BD Biosciences Clontech) following the supplier's protocol. The variable region cDNAs for the heavy and light chains were amplified by polymerase chain reaction (PCR) using 3′ primers that anneal respectively to the mouse gamma and kappa chain C regions (sequences listed below), and a 5′ universal primer provided in the SMART RACE cDNA Amplification Kit.

For VH PCR, the 3′ primers were as follows:

| (SEQ ID NO: 301) | |
|---|---|
| muIgG1: | GGACAGGGATCCAGAGTTCC |
| (SEQ ID NO: 302) | |
| muIgG2: | AGCTGGGAAGGTGTGCACAC |
| (SEQ ID NO: 303) | |
| muIgG3: | CAGGGGCCAGTGGATAGAC |

For VL PCR, the 3′ primers were as follows:

| (SEQ ID NO: 304) | |
|---|---|
| muCkappa.1: | GACATTGATGTCTTTGGGGT |
| (SEQ ID NO: 305) | |
| muCkappa.2: | TTCACTGCCATCAATCTTCC |

The PCR products were separated by agarose gel electrophoresis. The DNA fragments corresponding to VH and VL genes were purified by QIA quick Gel Extraction Kit (Qiagen), and subcloned into the pCRII-TOPO vector using TOPO TA cloning kit (Invitrogen). Each successful clone was sequenced by Sanger sequencing and at least 8 clones were sequenced for each sample.

Heavy chain sequences, light chain sequences, and CDR sequences for sequenced antibodies are shown in Table 15 below.

### Example 3. Epitope Binning and Epitope Mapping for Anti-TREM2 Antibodies

### Epitope Binning

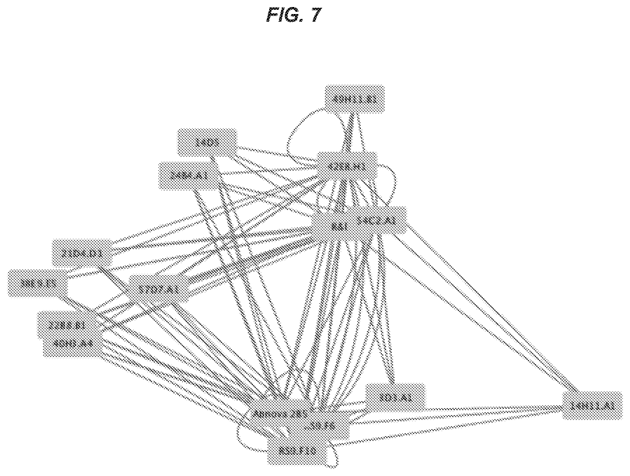

Limiting amounts (0.25 ng/mL) of TREM2-Fc proteins were coated onto high-binding polystyrene 96 well plates (e.g., Coming 3690). Plates were subsequently blocked by filling with Tris-buffered saline (10 mM, Tris, pH 7.5, 150 mM NaCl) supplemented with 0.05% Tween 20 and 3% bovine serum albumin (3% BSA-TBST); all subsequent antibody dilutions were in 3% BSA-TBST. Wells were washed five times with TBST. Wells were incubated simultaneously with test antibodies (10 μg/ml) and biotinylated probe antibody (0.25-1 ng/mL) for 1 hour at room temperature. After washing (5×TBST), wells were incubated with streptavidin-horseradish peroxidease conjugate for 1 hour at room temperature. After washing (5×TBST) and removal of residual buffer, plates were developed for ˜1-2 minutes by the addition of TMB substrate before stopping by the addition of 2N sulfuric acid. Absorbance at 450 nm was determined using a Perkin Elmer Envision plate reader. Binding competition was determined as a percentage reduction in probe antibody binding in the presence of competitor antibody compared to the absence of competitor antibody. Epitope bins were determined by computing relative competition binding across several different antibody probes. In FIG. 7, antibodies are shown clustered by similar epitope. Clustering was computed using Cytoscape version 3.5.1.

### FACS Binning on Human TREM2-HEK Cell Lines

HEK293 overexpressing human TREM2 (H6) were harvested by 0.05% trypsin and incubated at 37° C. incubator for 2 hours for surface TREM2 recovery. After incubation, cells were washed with 1×PBS and FACS buffer (PBS+0.5% BSA) with human Trustain FcX solution (Biolegend, Cat #422302) was added at a density of 10⁶/mL. Cells were seeded at 100,000 cells per well in a 96-well round-bottom and incubated 20 minutes at room temperature. After incubation, anti-TREM2 antibodies (100 nM) were added to the cells and the cells were incubated for 45 minutes on ice. After incubation, rat anti-h/mTREM2-APC antibody (1:100, R&D, Cat # FAB17291A) was added to the cells, and the cells were incubated for 30 minutes on ice. After incubation, cells were washed with FACS buffer three times and resuspended in 10 μL FACS buffer, then analyzed by flow cytometry (BD FACSCanto II, San Jose, Calif.). 20,000 events were obtained for each sample. The ability to compete with R&D antibody was determined by MFI analysis compared to isotype or buffer control wells. The results of the FACS binning are shown in Table 6 below.

### Octet Binning of Anti-TREM2 Antibodies

Competition with R&D antibody was done by Octet Red using biotinylated human Trem2-His protein generated in house. Biotinylated human TREM2-His protein was captured on a streptavidin tip, and the tip was then incubated in either 100 nM of R&D Ab or buffer for 3 minutes, followed by incubation for 3 minutes in 100 nM of testing hybridoma. The difference of hybridoma binding curve with or without pre-binding to R&D antibody was analyzed to determine the epitope. The results of the Octet binning are shown in Table 6 below.

TABLE 6
Epitope binning by FACS and Octet

| Clone ID | Bin by FACS | Bin by Octet |
|---|---|---|
| RS9.F6 | 2 | 2 |
| 3D3.A1 | 3 | 3 |
| 24B4.A1 | 1 | 1 |
| 54C2.A1 | 1 | 1 |
| 42E8.H1 | 1 | 1 |
| 21D4.D1 | 1 | 1-2 |
| 30F2 | 1-2 | 1-2 |
| 51D4 | 1-2 | 1-2 |
| 21D11 | 1-2 | 1-2 |
| 26D2 | 3 | 1-2 |
| 14H11.A1 | 1-2 | 1 |
| 26D5 | 1-2 | 1-2 |
| 2G4.B1 | 1-2 | N.D. |
| RS9.E2 | 1-2 | 1 |
| 49H11.B1 | 1 | 1 |
| 57D7.A1 | 1-2 | 1 |

N.D. = not determined

### Epitope Mapping Using Peptide Microarrays

The human TREM2 (SEQ ID NO:96; UniprotKB accession number Q9NZC2) extracellular domain was divided into 15 amino acid peptides, offset by 5 amino acids (overlapping by 10 amino acids). Peptides were synthesized and covalently attached to silica slides in triplicate with a spot size of 0.5 mm (JPT Technologies, Berlin, Germany). Antibodies were diluted to 30 μg/mL in 3% bovine serum albumin in Tris-buffered saline (10 mM, Tris, pH 7.5, 150 mM NaCl) supplemented with 0.05% Tween 20 (3% BSA-TBST). Diluted antibodies were allowed to bind to peptides printed onto slides for 2 hours at room temperature as described in the Pepstar user manual (JPT). Following extensive washing (5×5 min TBST), slides were incubated with secondary antibodies (donkey anti-mouse IgG, Alexafluor 647 conjugate, 5 μg/mL in 3% BSA-TBST) for 1 hour at room temperature. After extensive washing, (5×5 min TBST, 5×5 min ultrapure water), slides were dried under nitrogen and imaged on the Opera Phenix in the 647 nm channel. Images were aligned to peptide array definition file (galviewer, JPT) using ImageJ with control mouse IgG serving as landmarks. The results of the epitope mapping are shown in Table 7 below.

TABLE 7
Epitope mapping of anti-TREM2 antibodies

| Antibody | Tiled TREM2 peptides | | | | | |
|---|---|---|---|---|---|---|
| 42E8.H1 | 24-43 | | | 94-108 | 124-153 | |
| 3D3.A1 | | | | | 134-153 | 159-174 |
| 21D4.D1 | N.D. | | | | | |
| 49H11.B1 | N.D. | | | | | |
| RS9.F10 | | | | | 129-153 | |
| 24B4.A1 | N.D. | | | | | |
| 54C2.A1 | | | 64-78 | 89-103 | 129-143 | |
| 57D7.A1 | | 44-58 | 74-88 | | 134-148 | |
| RS9.F6 | | 44-58 | | 94-108 | 129-153 | |
| RS9.E2 | | 44-58 | | | | |
| 2G4 | N.D. | | | | | |
| 30F2 | | | 64-78 | | | |
| 26D2 | | 44-58 | 64-78 | | | |
| 21D11 | | 44-58 | 64-78 | | | |
| 51D4 | | 44-58 | 64-78 | | | |
| 21D5 | | 44-58 | | 89-108 | | |

N.D. = not determined

### Example 4. Screen for Novel TREM2-pSyk Activating Lipids

Lipids are physiological ligands for TREM2. Understanding the potential endogenous lipid ligands for TREM2, such as the endogenous lipid ligands in specific cell or tissue types or in specific disease states, enables analysis of antibody-lipid biological interactions and prediction of function in vivo. A screen was conducted to identify lipid ligands that induce p-Syk in HEK cells expressing wild-type TREM2 and DAP12, mutant TREM2 and DAP12, or DAP12 alone, as well as macrophage cells that endogenously express TREM2. Selected anti-TREM2 antibodies were also tested to characterize the interaction of the antibodies with lipid ligand to activate p-Syk.

### Liposome Preparation

Lipids were purchased from Avanti Polar Lipids (Alabaster, Ala.) or Echelon Biosciences (Salt Lake City, Utah). Each screened lipid was resuspended in chloroform and added at 30 mole percent composition to 70 mole percent phosphatidylcholine (PC; 1,2-dioleoyl-sn-glycero-3-phosphocholine, Avanti Polar Lipids). Kdo2-Lipid A was added at a 10 mole percent composition with 90 mole percent PC. Lipid mixes were dried under nitrogen gas and resuspended in HBSS at a concentration of 4 mg/mL. Mixes were vortexed or briefly sonicated until resuspended, then bath sonicated for 1-5 minutes. Liposomes were used for 1-2 weeks. 3 independent batches of liposomes were used in experiments.

### HEK293 Overexpressing Cell Line Culture

HEK293 cell lines overexpressing TREM2 and DAP12, mutant TREM2-R47H and DAP12, or DAP12 alone were plated in 96-well PDL-coated plates at a density of 40,000 cells per well and cultured for 36-48 hours until 90-100% confluent.

### Human Macrophage Differentiation Culture

10 mL human blood was obtained from Blood Centers of the Pacific. Monocytes were extracted using RosetteSep Human Monocyte Enrichment Cocktail (STEMCELL Technologies Inc.; Vancouver, BC; #15068) as per manufacturer protocols. Density gradient was performed with Ficoll-Paque Premium (GE Healthcare Life Sciences; Pittsburgh, Pa.; #17-5442-02). Isolated monocytes were resuspended at ˜1-1.3 million cells/mL and cultured for 5-6 days in RPMI, 10% Hyclone Fetal Bovine Serum, and penicillin/streptomycin supplemented with 50 ng/mL human M-CSF (Gibco, # PHC9501) in T175 tissue culture treated flasks. M-CSF was replenished every 2-3 days. Cells were washed once with PBS, 15 mL fresh medium was added, and cells were harvested by scraping. Cells were cultured overnight at a density of 100,000 cells per well on tissue culture treated 96-well plates supplemented with 10-25 ng/mL M-CSF.

### Liposome Stimulation Assay for HEK293 Overexpressing Cell Lines and Human Macrophages

Cell culture medium was removed and cells were washed once with 200ul HBSS. Liposomes were diluted in HBSS in concentrations ranging from 0.03125 mg/mL to 2 mg/mL, then 50 μl was added to wells in 2-3 technical replicates per plate. Each plate contained HBSS negative control wells, 100% PC liposome control, 5 μg/mL agonist anti-TREM2 positive control (Abnova; Taiwan; # MAB2056), and 5 μg/mL mouse IgG3 isotype control (R&D Systems; Minneapolis, Minn.; # MAB007). Stimuli were incubated with cells for 5 minutes at 37° C., 5% CO₂, liposomes were removed, and plates were frozen at −80° C.

### Phosphorylated-Syk Assay for HEK293 Overexpressing Cell Lines and Human Macrophages

Cells were lysed for 30 minutes to 1 hour on ice in 50 ul/well Cell Lysis Buffer (Cell Signaling Technology; Danvers, Mass.; #9803) with 1 mM PMSF. AlphaLISA SureFire Ultra p-Syk Tyr525/526 assay kit (PerkinElmer, ALSU-PSYK-A10OK) was used to measure pSyk levels in lysate as per manufacturer protocol. 10 μl lysate was added to 384-well OptiPlates (PerkinElmer; #6007290) and an EnVision Multilabel Plate Reader (PerkinElmer) was used to measure AlphaLISA fluorescence.

### Liposome Screen Analysis

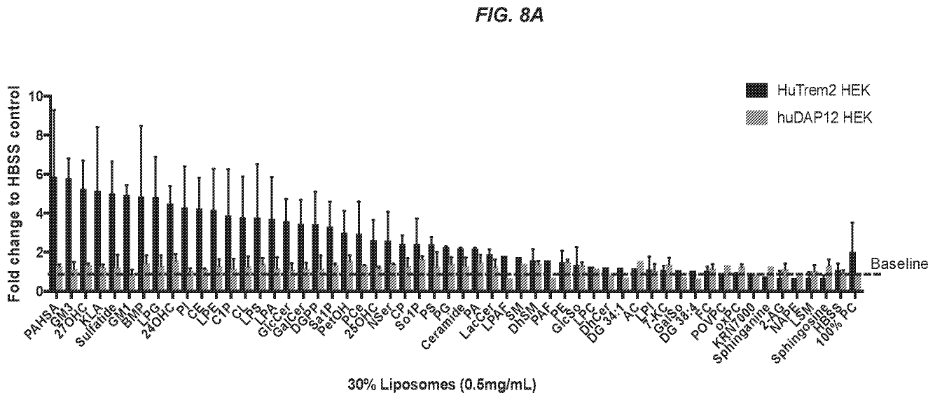

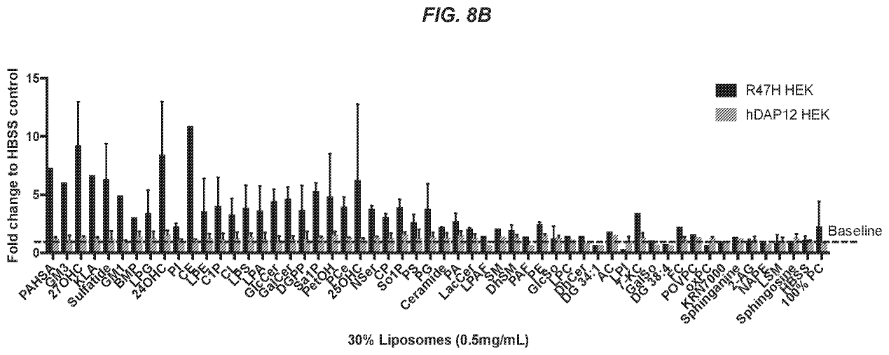

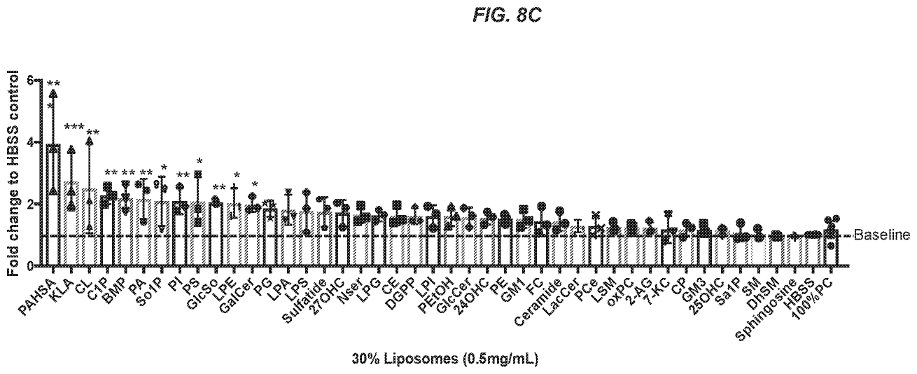

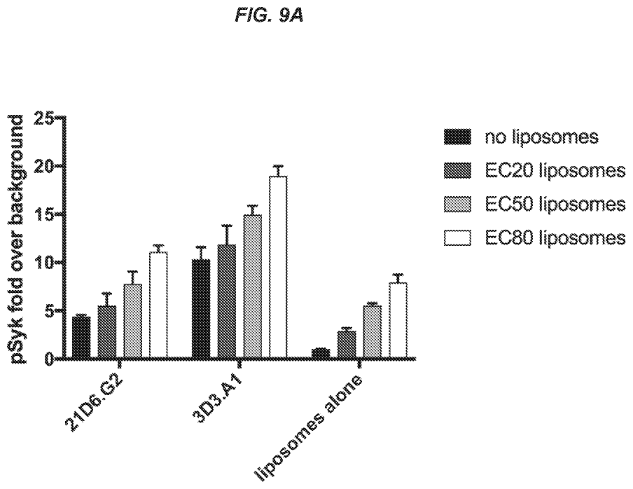

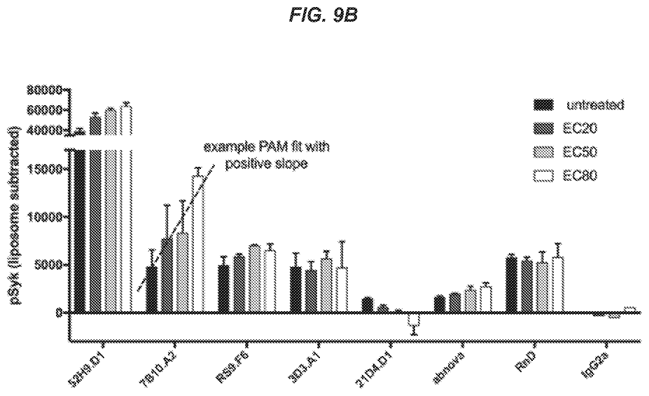

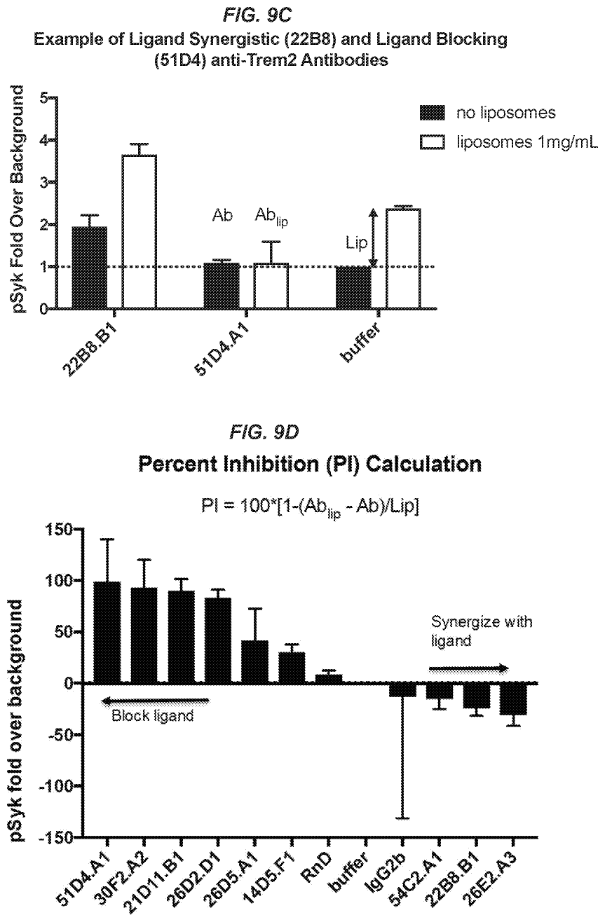

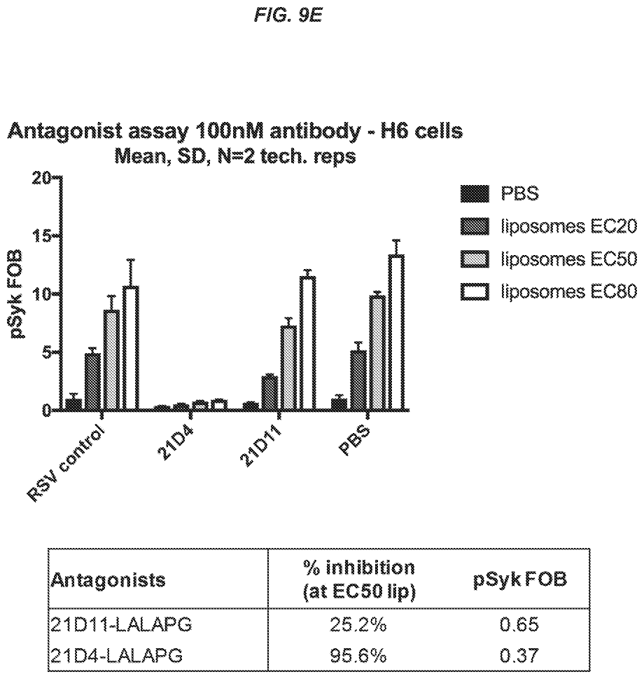

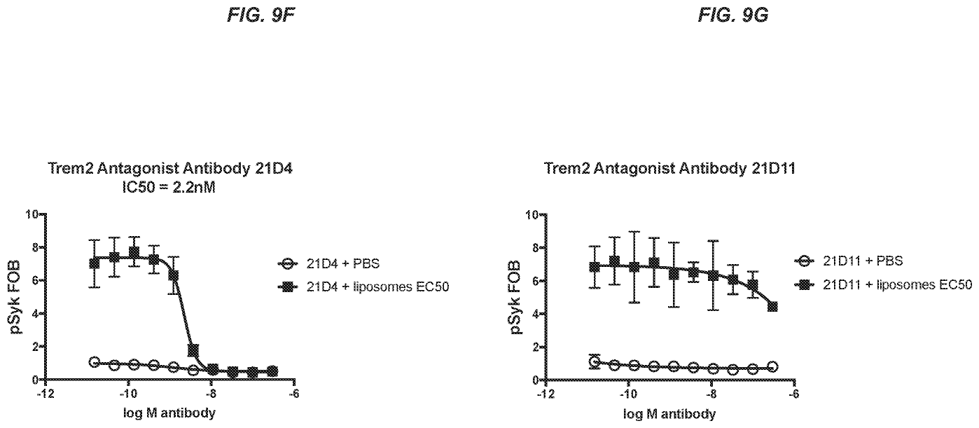

PSyk AlphaLISA fluorescence was converted to a log 2 scale. Fluorescence values from liposomes and controls were each subtracted from average fluorescence values from HBSS stimulated wells on the same plate (log 2(liposome)−log 2(HBSSave)=log 2(signal)). The result was converted back to a standard scale to measure fold change (2^{log 2(signal)}=fold change). Technical replicate fold changes values were averaged to obtain the fold change over background listed (pSyk FOB). Graphs represent values from independent experiments or individual human donors. Results from the screen on the HEK293 overexpressing cell lines are shown in Table 9 below and in FIG. 8A-8B. Results from the screen on primary human macrophages are shown in Table 10 below and in FIG. 8C. HEK293 results showing specificity for TREM2 over DAP12 expressed alone can be used to extrapolate which lipid species are likely acting as TREM2 ligands on human macrophages. PAHSA, KLA, CL, C1P, BMP, PI, PS, LPE, and GalCer likely signal via TREM2 because pSyk levels are elevated above DAP12 control cells, whereas PA, So1P, and GlcSo may not signal through TREM2. FIG. 9A shows the characterization of selected anti-TREM2 antibodies' interaction with lipid ligand to activate p-Syk. As shown in FIG. 9A, 21D6.G2 and 3D3.A1 define a class of TREM2 antibody that act as additive with lipid TREM2 activators. 21D4.D1 defines a class of TREM2 antibody that alone induce pSyk signaling yet block lipid ligand activation of TREM2.

### TREM2 PAM/pSyk Assay on Human Macrophages

Human macrophages were differentiated from donor blood using a human monocyte enrichment cocktail (Stem Cell Technologies) and culturing monocytes with human mCSF for five days in macrophage media (RPMI+10% Hyclone FBS+Glutamax+Pen/Strep+non-essential amino acids+sodium pyruvate). On the fifth day, the human macrophages were scraped from the flasks and were re-plated at 100,000 cells/well on a 96-well poly-D-lysine-coated plate in the macrophage media (without mCSF). The cells were incubated for 24 hours to adhere, then the media was removed.

Antibodies pre-mixed with lipid vesicles comprised of 70% DOPC and 30% POPS (made according to the procedure for making vesicles described above) diluted in HBSS were immediately added to the cells, and allowed to incubate for 5 minutes at 37° C. The liposome/antibody solution was removed, and 35 μL lysis buffer (Cell Signaling Technologies, CST) was added using the liquid handler. The lysate was then incubated at 4° C. for 30 minutes, then either frozen at −80° C. or immediately carried forward to a pSyk AlphaLISA assay as described above. The signal due to liposomes alone was subtracted at each value to determine if the antibodies had a synergistic, neutral, or inhibitor effect on lipid ligand driven pSyk activation. The results of the assay are shown in FIG. 9B and in Table 8 below. 52H9.D1 and 7B10.A2 define a class of TREM2 antibody that act as synergistic with lipid TREM2 activators. RS9.F6 and 3D3.A1 define a class of TREM2 antibody that have at least an additive effect on lipid ligand activation of TREM2. 21D4.D1 defines a class of TREM2 antibody that has an inhibitor effect on lipid ligand activation of TREM2.

TABLE 8
Induction of pSyk in the presence of TREM2 lipid ligand

| Antibody | Slope + Liposome |
|---|---|
| 52H9.D1 | 13,314 |
| 7B10.A2 | 5,370 |
| RS9.F6 | 878 |
| 3D3.A1 | 94 |
| 21D4.D1 | −1.632 |
| abnova | 700 |
| RnD | −269 |
| IgG2a | 366 |

TREM2 pSyk Antagonist Assay

Human macrophages were differentiated from donor blood using a human monocyte enrichment cocktail (Stem Cell Technologies) and culturing monocytes with human mCSF for five days in macrophage media (RPMI+10% Hyclone FBS+Glutamax+Pen/Strep+non-essential amino acids+sodium pyruvate). On the fifth day, the human macrophages were scraped from the flasks and were re-plated at 100,000 cells/well on a 96-well poly-D-lysine-coated plate in the macrophage media (without mCSF). The cells were incubated for 24 hours to adhere, then the media was removed.

Antibodies diluted in RPMI alone were immediately added to the cells, and allowed to incubate for 30 minutes at 37° C. The antibodies were then removed. Lipid vesicles comprised of 70% DOPC and 30% POPS (made according to the procedure for making vesicles described above) in HBSS were added to the cells and incubated for minutes at 37° C. The liposome/antibody solution was removed, and 35 μL lysis buffer (Cell Signaling Technologies, CST) was added using the liquid handler. The lysate was then incubated at 4° C. for 30 minutes, then either frozen at −80° C. or immediately carried forward to a pSyk AlphaLISA assay as described above. The signal due to liposomes alone was subtracted at each value to determine if the antibodies had a synergistic, neutral, or inhibitor effect on lipid ligand driven pSyk activation. The results of the assay are shown in FIGS. 9C-9D. As shown in FIGS. 9C and 9D, 51D4.A1, 30F2.A2, 21D11.B1, and 26D2.D1 all blocked liposome mediated pSyk signaling, and 54C2.A1, 22B8.B1, and 26E2.A3 all enhanced liposome mediated pSyk signaling.

FIG. 9E shows inhibition of liposome mediated pSyk by 21D4 and 21D11. These experiments were performed as follows. Two days in advance of the experiment, HEK293 cells stably overexpressing TREM2 and DAP12 were plated at 40,000 cells/well on a 96 well poly-D-lysine-coated plate. Antibodies diluted in DMEM alone were immediately added to the cells, and allowed to incubate for 30 minutes at 37° C. The antibodies were then removed. Lipid vesicles comprised of 70% DOPC and 30% POPS at their EC20, EC50, or EC80, or PBS alone, were added to the cells and incubated for 5 minutes at 37° C. The liposome/antibody solution was removed, and 35 μL lysis buffer (Cell Signaling Technologies, CST) was added using the liquid handler. The lysate was then incubated at 4° C. for 30 minutes, then either frozen at −80° C. or immediately carried forward to a pSyk AlphaLISA assay as described above. The percent inhibition was calculated as in FIG. 9C.

FIGS. 9F and 9G show inhibition by 21D4 and 21D11, respectively, at increasing antibody concentrations. These experiments were performed similarly to those described in FIG. 9E. These figures show that 21D4 exhibited antagonistic activity and 21D11 exhibited weaker antagonistic activity.

TABLE 9
Novel TREM2-pSyk activating lipids identified
from screen on HEK overexpressing cell lines

| | Human | R47H TREM2 | |
|---|---|---|---|
| | TREM2 | mutant | DAP12 alone |
| Lipid | (pSyk FOB) | (pSyk FOB) | (pSyk FOB) |
| Palmitic-acid-9-hydroxy-stearic- | 5.87 | 7.31 | 1.27 |
| acid (PAHSA) | | | |
| Ganglioside GM3 | 5.82 | 6.05 | 1.14 |
| 27-hydroxycholesterol (27OHC) | 5.26 | 9.25 | 1.36 |
| Kdo2-Lipid A (KLA) | 5.15 | 6.65 | 1.25 |
| Sulfatide | 5.02 | 6.34 | 1.21 |
| Ganglioside GM1 | 4.94 | 4.93 | 0.92 |
| Bis(monoacylglycero)phosphate | 4.85 | 3.04 | 1.42 |
| (BMP) | | | |
| Lysophosphatidylglycerol (LPG) | 4.83 | 3.44 | 1.28 |
| 24(S)hydroxycholesterol (24OHC) | 4.50 | 8.44 | 1.57 |
| Phosphatidylinositol (PI) | 4.31 | 2.27 | 1.04 |
| Cholesteryl ester (CE) | 4.24 | 10.90 | 1.14 |
| LysophosphatidyLethanolamine (LPE) | 4.16 | 3.58 | 1.28 |
| Ceramide-1-phosphate (C1P) | 3.89 | 4.00 | 1.16 |
| Cardiolipin (CL) | 3.79 | 3.31 | 1.26 |
| Lysophosphatidylserine (LPS) | 3.77 | 3.88 | 1.39 |
| Lysophosphatidic acid (LPA) | 3.69 | 3.62 | 1.17 |
| Galactosylceramide (GalCer) | 3.45 | 4.65 | 1.16 |
| Diacylglycerol pyrophosphate | 3.42 | 3.70 | 1.16 |
| (DGPP) | | | |
| Sphinganine-1-phosphate (SalP) | 3.31 | 5.32 | 1.34 |
| Phosphatidylethanol (PEtOH) | 3.00 | 4.83 | 1.57 |
| Ether phosphatidylcholine (PCe) | 2.94 | 3.97 | 1.33 |
| 25(S)hydroxycholesterol (25OHC) | 2.61 | 6.27 | 1.20 |
| N-Acyl-Serine (NSer) | 2.59 | 3.76 | 1.39 |
| Cholesterol phosphate (CP) | 2.43 | 3.09 | 1.27 |
| Sphingosine-1-phosphate (SolP) | 2.42 | 3.94 | 1.64 |
| Phosphatidylserine (PS) | 2.42 | 2.65 | 1.27 |
| Phosphatidylglycerol (PG) or | 2.25 | 3.77 | 1.40 |
| DSPG | | | |
| Ceramide | 2.20 | 2.21 | 1.30 |
| Phosphatidic acid (PA) | 2.17 | 2.72 | 1.48 |
| 100% Phosphatidylcholine (PC) | 2.01 | 2.27 | 0.86 |
| Lactosylceramide (LacCer) | 1.89 | 2.06 | 1.27 |
| lysoalkylacylglycerophosphocholine | 1.81 | 1.46 | 0.67 |
| (LPAF) | | | |
| Sphingomyelin (SM) | 1.74 | 2.09 | 1.40 |
| Dihydrosphingomyelin (DhSM) | 1.59 | 1.93 | 1.40 |
| alkylacylglycerophosphocholine | 1.58 | 1.41 | 0.69 |
| (PAF) | | | |
| Phosphatidylethanolamine (PE) | 1.49 | 2.49 | 1.51 |
| or DSPE | | | |
| Glucosyl sphingosine (GlcSo) | 1.34 | 1.27 | 1.31 |
| Lysophosphatidylcholine (LPC) | 1.26 | 1.47 | 1.14 |
| Dihyrdoceramide (DhCer) | 1.22 | 1.47 | 0.84 |
| Diacylglycerol 34:1 (DG34:1) | 1.20 | 0.68 | 0.68 |
| Acyl Carnitine (AC) | 1.17 | 1.83 | 1.55 |
| Lysophosphatidylinositol (LPI) | 1.12 | 0.32 | 1.03 |
| Hank's Balanced Salt Solution | 1.10 | 1.12 | 0.96 |
| (HBSS) | | | |
| 7-ketocholesterol (7-KC) | 1.10 | 3.42 | 1.36 |
| Galactosylsphingosine (GalSo) | 1.09 | 1.07 | 0.71 |
| Diacylglycerol 38:4 (DG 38:4) | 1.05 | 0.73 | 0.64 |
| Free cholesterol (FC) | 1.04 | 2.24 | 1.14 |
| 1-palmitoyl-2-(5′-oxo-valeroyl)- | 0.86 | 1.57 | 1.34 |
| sn-glycero-3-phosphocholine (POVPC) | | | |
| Oxidized phosphatidylcholine (oxPC) | 0.81 | 0.68 | 1.26 |
| α-galactosylceramide (KRN7000) | 0.79 | 0.97 | 0.85 |
| Sphinganine | 0.76 | 1.39 | 1.26 |
| 2-Arachidonoylglycerol (2-AG) | 0.68 | 0.84 | 1.11 |
| N-Acyl-phosphatidylethanolamine | 0.67 | 0.94 | 0.78 |
| (NAPE) | | | |
| Lysosphingomyelin (LSM) | 0.67 | 0.85 | 1.03 |
| Sphingosine | 0.67 | 0.85 | 1.32 |

TABLE 10
Novel TREM2-pSyk activating lipids identified
from screen on primary human macrophages

| Lipid | pSyk FOB |
|---|---|
| Palmitic-acid-9-hydroxy-stearic- | 3.94 |
| acid (PAHSA) | |
| Kdo2-Lipid A (KLA) | 2.73 |
| Cardiolipin (CL) | 2.50 |
| Ceramide-1-phosphate (C1P) | 2.28 |
| Bis(monoacylglycero)phosphate (BMP) | 2.19 |
| Phosphatidic acid (PA) | 2.17 |
| Sphingosine-1-phosphate (SolP) | 2.10 |
| Phosphatidylinositol (PI) | 2.09 |
| Phosphatidylserine (PS) | 2.09 |
| Glucosyl sphingosine (GlcSo) | 2.05 |
| Lysophosphatidylethanolamine (LPE) | 2.03 |
| Galactosylceramide (GalCer) | 1.98 |
| Phosphatidylglycerol (PG) or DSPG | 1.85 |
| Lysophosphatidic acid (LPA) | 1.82 |
| Lysophosphatidylserine (LPS) | 1.78 |
| Sulfatide | 1.75 |
| 27-hydroxycholesterol (27OHC) | 1.72 |
| N-Acyl-Serine (NSer) | 1.66 |
| Lysophosphatidylglycerol (LPG) | 1.64 |
| Cholesteryl ester (CE) | 1.62 |
| Diacylglycerol pyrophosphate (DGPP) | 1.61 |
| Lysophosphatidylinositol (LPI) | 1.61 |
| Phosphatidylethanol (PEtOH) | 1.61 |
| Glucosylceramide (GlcCer) | 1.58 |
| 24(S)hydroxycholesterol (24OHC) | 1.56 |
| Phosphatidylethanolamine (PE) or DSPE | 1.53 |
| Ganglioside GM1 | 1.52 |
| Free cholesterol (FC) | 1.44 |
| Ceramide | 1.44 |
| Lactosylceramide (LacCer) | 1.29 |
| Ether phosphatidylcholine (PCe) | 1.29 |
| Lysosphingomyelin (LSM) | 1.26 |
| Oxidized PC | 1.25 |
| 2-Arachidonoylglycerol (2-AG) | 1.23 |
| 7-ketocholesterol (7-KC) | 1.18 |
| Cholesterol phosphate (CP) | 1.18 |
| 100% Phosphatidylcholine (PC) | 1.18 |
| Ganglioside GM3 | 1.17 |
| 25-hydroxycholesterol (25OHC) | 1.14 |
| Sphinganine-1-phosphate (Sa1P) | 1.08 |
| Sphingomyelin (SM) | 1.02 |
| Hank's Balanced Salt Solution (HBSS) | 1.01 |
| Dihydrosphingomyelin (DhSM) | 0.96 |
| Sphingosine | 0.96 |

### Example 5. Modified Fc Polypeptides that Bind to TfR

This example describes modifications to Fc polypeptides to confer transferrin receptor (TfR) binding and transport across the blood-brain barrier (BBB). Unless otherwise indicated, the positions of amino acid residues in this example are numbered based on EU index numbering for a human IgG1 wild-type Fc region.

### Generation and Characterization of Fc Polypeptides Comprising Modifications at Positions 384, 386, 387, 388, 389, 390, 413, 416, and 421 (CH3C Clones)

Yeast libraries containing Fc regions having modifications introduced into positions including amino acid positions 384, 386, 387, 388, 389, 390, 413, 416, and 421 were generated as described below. Illustrative clones that bind to TfR are shown in Tables 11 and 12.

After an additional two rounds of sorting, single clones were sequenced and four unique sequences were identified. These sequences had a conserved Trp at position 388, and all had an aromatic residue (i.e., Trp, Tyr, or His) at position 421. There was a great deal of diversity at other positions. The four clones selected from the library were expressed as Fc fusions to Fab fragments in CHO or 293 cells, and purified by Protein A and size-exclusion chromatography, and then screened for binding to human TfR in the presence or absence of holo-Tf by ELISA. The clones all bound to human TfR and the binding was not affected by the addition of excess (5 μM) holo-Tf. Clones were also tested for binding to 293F cells, which endogenously express human TfR. The clones bound to 293F cells, although the overall binding was substantially weaker than the high-affinity positive control.

Next, it was tested whether clones could internalize in TfR-expressing cells using clone CH3C.3 as a test clone. Adherent HEK 293 cells were grown in 96-well plates to about 80% confluence, media was removed, and samples were added at 1 μM concentrations: clone CH3C.3, anti-TfR benchmark positive control antibody (Ab204), anti-BACE1 benchmark negative control antibody (Ab107), and human IgG isotype control (obtained from Jackson Immunoresearch). The cells were incubated at 37° C. and 8% CO₂ concentration for 30 minutes, then washed, permeabilized with 0.1% Triton™ X-100, and stained with anti-human-IgG-Alexa Fluor® 488 secondary antibody. After additional washing, the cells were imaged under a high content fluorescence microscope (i.e., an Opera Phenix™ system), and the number of puncta per cell was quantified. At 1 M, clone CH3C.3 showed a similar propensity for internalization to the positive anti-TfR control, while the negative controls showed no internalization.

### Further Engineering of Clones

Additional libraries were generated to improve the affinity of the initial hits against human TfR using a soft randomization approach, wherein DNA oligos were generated to introduce soft mutagenesis based on each of the original four hits. Additional clones were identified that bound TfR and were selected. The selected clones fell into two general sequence groups. Group 1 clones (i.e., clones CH3C.18, CH3C.21, CH3C.25, and CH3C.34) had a semi-conserved Leu at position 384, a Leu or His at position 386, a conserved and a semi-conserved Val at positions 387 and 389, respectively, and a semi-conserved P-T-W motif at positions 413, 416, and 421, respectively. Group 2 clones had a conserved Tyr at position 384, the motif TXWSX at positions 386-390, and the conserved motif S/T-E-F at positions 413, 416, and 421, respectively. Clones CH3C.18 and CH3C.35 were used in additional studies as representative members of each sequence group.

### Epitope Mapping

To determine whether the engineered Fc regions bound to the apical domain of TfR, TfR apical domain was expressed on the surface of phage. To properly fold and display the apical domain, one of the loops had to be truncated and the sequence needed to be circularly permuted. Clones CH3C.18 and CH3C.35 were coated on ELISA plates and a phage ELISA protocol was followed. Briefly, after washing and blocking with 1% PBSA, dilutions of phage displaying were added and incubated at room temperature for 1 hour. The plates were subsequently washed and anti-M13-HRP was added, and after additional washing the plates were developed with TMB substrate and quenched with 2N H₂SO₄. Both clones CH3C.18 and CH3C.35 bound to the apical domain in this assay.

Paratope mapping

To understand which residues in the Fc domain were most important for TfR binding, a series of mutant clone CH3C.18 and clone CH3C.35 Fc regions was created in which each mutant had a single position in the TfR binding register mutated back to wild-type. The resulting variants were expressed recombinantly as Fab-Fc fusions and tested for binding to human or cyno TfR. For clone CH3C.35, positions 388 and 421 were important for binding; reversion of either of these to wild-type completely ablated binding to human TfR.

### Binding Characterization of Maturation Clones

Binding ELISAs were conducted with purified Fab-Fc fusion variants with human or cyno TfR coated on the plate, as described above. The variants from the clone CH3C.18 maturation library, clone CH3C.3.2-1, clone CH3C.3.2-5, and clone CH3C.3.2-19, bound human and cyno TfR with approximately equivalent EC5o values, whereas the parent clones CH3C.18 and CH3C.35 had greater than 10-fold better binding to human versus cyno TfR.

Next, it was tested whether the modified Fc polypeptides internalized in human and monkey cells. Using the protocol described above, internalization in human HEK 293 cells and rhesus LLC-MK2 cells was tested. The variants that similarly bound human and cyno TfR, clones CH3C.3.2-5 and CH3C.3.2-19, had significantly improved internalization in LLC-MK2 cells as compared with clone CH3C.35.

### Additional Engineering of Clones

Additional engineering to further affinity mature clones CH3C.18 and CH3C.35 involved adding additional mutations to the positions that enhanced binding through direct interactions, second-shell interactions, or structure stabilization. This was achieved via generation and selection from an “NNK walk” or “NNK patch” library. The NNK walk library involved making one-by-one NNK mutations of residues that are near to the paratope. By looking at the structure of Fc bound to FcγRI (PDB ID: 4W40), 44 residues near the original modification positions were identified as candidates for interrogation. Specifically, the following residues were targeted for NNK mutagenesis: K248, R255, Q342, R344, E345, Q347, T359, K360, N361, Q362, S364, K370, E380, E382, S383, G385, Y391, K392, T393, D399, S400, D401, S403, K409, L410, T411, V412, K414, S415, Q418, Q419, G420, V422, F423, S424, S426, Q438, S440, S442, L443, S444, P4458, G446, and K447. The 44 single point NNK libraries were generated using Kunkel mutagenesis, and the products were pooled and introduced to yeast via electroporation, as described above for other yeast libraries.

The combination of these mini-libraries (each of which had one position mutated, resulting in 20 variants) generated a small library that was selected using yeast surface display for any positions that lead to higher affinity binding. Selections were performed as described above, using TfR apical domain proteins. After three rounds of sorting, clones from the enriched yeast library were sequenced, and several “hot-spot” positions were identified where certain point mutations significantly improved the binding to apical domain proteins. For clone CH3C.35, these mutations included E380 (mutated to Trp, Tyr, Leu, or Gln) and S415 (mutated to Glu). The sequences of the clone CH3C.35 single and combination mutants are set forth in SEQ ID NOs:100-104 and 160-166. For clone CH3C.18, these mutations included E380 (mutated to Trp, Tyr, or Leu) and K392 (mutated to Gln, Phe, or His). The sequences of the clone CH3C.18 single mutants are set forth in SEQ ID NOs:154-159.

### Additional Maturation Libraries to Improve Clone CH3C.35 Affinity

An additional library to identify combinations of mutations from the NNK walk library, while adding several additional positions on the periphery of these, was generated as described for previous yeast libraries. In this library, the YxTEWSS (SEQ ID NO:299) and TxxExxxxF (SEQ ID NO:300) motifs were kept constant, and six positions were completely randomized: E380, K392, K414, S415, S424, and S426. Positions E380 and S415 were included because they were “hot spots” in the NNK walk library. Positions K392, S424, and S426 were included because they make up part of the core that may position the binding region, while K414 was selected due to its adjacency to position 415.

This library was sorted, as previously described, with the cyno TfR apical domain only. The enriched pool was sequenced after five rounds, and the sequences of the modified regions of the identified unique clones are set forth in SEQ ID NOs:105 and 169-185.

The next libraries were designed to further explore acceptable diversity in the main binding paratope. Each of the original positions (384, 386, 387, 388, 389, 390, 413, 416, and 421) plus the two hot spots (380 and 415) were individually randomized with NNK codons to generate a series of single-position saturation mutagenesis libraries on yeast. In addition, each position was individually reverted to the wild-type residue, and these individual clones were displayed on yeast. It was noted that positions 380, 389, 390, and 415 were the only positions that retained substantial binding to TfR upon reversion to the wild-type residue (some residual but greatly diminished binding was observed for reversion of 413 to wild-type).

The single-position NNK libraries were sorted for three rounds against the human TfR apical domain to collect the top ˜5% of binders, and then at least 16 clones were sequenced from each library. The results indicate what amino acids at each position can be tolerated without significantly reducing binding to human TfR, in the context of clone CH3C.35. A summary is below:
Position 380: Trp, Leu, or Glu;
Position 384: Tyr or Phe;
Position 386: Thr only;
Position 387: Glu only;
Position 388: Trp only;
Position 389: Ser, Ala, or Val (although the wild type Asn residue seems to retain some binding, it did not appear following library sorting);
Position 390: Ser or Asn;
Position 413: Thr or Ser;
Position 415: Glu or Ser;
Position 416: Glu only; and
Position 421: Phe only.

The above residues, when substituted into clone CH3C.35 as single changes or in combinations, represent paratope diversity that retains binding to TfR apical domain. Clones having mutations at these positions include those shown in Table 12, and the sequences of the CH3 domains of these clones are set forth in SEQ ID NOs:100-136 and 344-350.

Additional Fc Positions that can be Modified to Confer TfR Binding

Additional modified Fc polypeptides that bind to transferrin receptor (TfR) were generated having modifications at alternative sites in the Fc region, e.g., at the following positions:
positions 274, 276, 283, 285, 286, 287, 288, and 290 (CH2A2 clones);
positions 266, 267, 268, 269, 270, 271, 295, 297, 298, and 299 (CH2C clones);
positions 268, 269, 270, 271, 272, 292, 293, 294, and 300 (CH2D clones);
positions 272, 274, 276, 322, 324, 326, 329, 330, and 331 (CH2E3 clones); or
positions 345, 346, 347, 349, 437, 438, 439, and 440 (CH3B clones).

Illustrative CH3B clones that bind to TfR are set forth in SEQ ID NOs:186-190. Illustrative CH2A2 clones that bind to TfR are set forth in SEQ ID NOs: 191-195. Illustrative CH2C clones that bind to TfR are set forth in SEQ ID NOs:196-200. Illustrative CH2D clones that bind to TfR are set forth in SEQ ID NOs:201-205. Illustrative CH2E3 clones that bind to TfR are set forth in SEQ ID NOs:206-210.

### Methods

### Generation of Phage-Display Libraries

A DNA template coding for the wild-type human Fc sequence was synthesized and incorporated into a phagemid vector. The phagemid vector contained an ompA or pelB leader sequence, the Fc insert fused to c-Myc and 6xHis epitope tags, and an amber stop codon followed by M13 coat protein pIII.

Primers containing “NNK” tricodons at the desired positions for modifications were generated, where N is any DNA base (i.e., A, C, G, or T) and K is either G or T. Alternatively, primers for “soft” randomization were used, where a mix of bases corresponding to 70% wild-type base and 10% of each of the other three bases was used for each randomization position. Libraries were generated by performing PCR amplification of fragments of the Fc region corresponding to regions of randomization and then assembled using end primers containing SfiI restriction sites, then digested with SfiI and ligated into the phagemid vectors. Alternatively, the primers were used to conduct Kunkel mutagenesis. The ligated products or Kunkel products were transformed into electrocompetent E. coli cells of strain TG1 (obtained from Lucigen®). The E. coli cells were infected with M13K07 helper phage after recovery and grown overnight, after which library phage were precipitated with 5% PEG/NaCl, resuspended in 15% glycerol in PBS, and frozen until use. Typical library sizes ranged from about 10⁹ to about 10¹¹ transformants. Fc-dimers were displayed on phage via pairing between pIII-fused Fc and soluble Fc not attached to pIII (the latter being generated due to the amber stop codon before pIII).

### Generation of Yeast-Display Libraries

A DNA template coding for the wild-type human Fc sequence was synthesized and incorporated into a yeast display vector. For CH2 and CH3 libraries, the Fc polypeptides were displayed on the Aga2p cell wall protein. Both vectors contained prepro leader peptides with a Kex2 cleavage sequence, and a c-Myc epitope tag fused to the terminus of the Fc.

Yeast display libraries were assembled using methods similar to those described for the phage libraries, except that amplification of fragments was performed with primers containing homologous ends for the vector. Freshly prepared electrocompetent yeast (i.e., strain EBY100) were electroporated with linearized vector and assembled library inserts. Electroporation methods will be known to one of skill in the art. After recovery in selective SD-CAA media, the yeast were grown to confluence and split twice, then induced for protein expression by transferring to SG-CAA media. Typical library sizes ranged from about 10⁷ to about 10⁹ transformants. Fc-dimers were formed by pairing of adjacently displayed Fc monomers.

### General Methods for Phage Selection

Phage methods were adapted from Phage Display: A Laboratory Manual (Barbas, 2001). Additional protocol details can be obtained from this reference.

Plate Sorting Methods

Antigen was coated on MaxiSorp® microtiter plates (typically 1-10 μg/mL) overnight at 4° C. The phage libraries were added into each well and incubated overnight for binding. Microtiter wells were washed extensively with PBS containing 0.05% Tween® 20 (PBST) and bound phage were eluted by incubating the wells with acid (typically 50 mM HCl with 500 mM KCl, or 100 mM glycine, pH 2.7) for 30 minutes. Eluted phage were neutralized with 1 M Tris (pH 8) and amplified using TG1 cells and M13/KO7 helper phage and grown overnight at 37° C. in 2YT media containing 50 μg/mL carbenacillin and 50 ug/mL Kanamycin. The titers of phage eluted from a target-containing well were compared to titers of phage recovered from a non-target-containing well to assess enrichment. Selection stringency was increased by subsequently decreasing the incubation time during binding and increasing washing time and number of washes.

Bead Sorting Methods

Antigen was biotinylated through free amines using NHS-PEG4-Biotin (obtained from Pierce™). For biotinylation reactions, a 3- to 5-fold molar excess of biotin reagent was used in PBS. Reactions were quenched with Tris followed by extensive dialysis in PBS. The biotinylated antigen was immobilized on streptavidin-coated magnetic beads, (i.e., M280-streptavidin beads obtained Thermo Fisher). The phage display libraries were incubated with the antigen-coated beads at room temperature for 1 hour. The unbound phage were then removed and beads were washed with PBST. The bound phage were eluted by incubating with 50 mM HCl containing 500 mM KCl (or 0.1 M glycine, pH 2.7) for 30 minutes, and then neutralized and propagated as described above for plate sorting.

After three to five rounds of panning, single clones were screened by either expressing Fc on phage or solubly in the E. coli periplasm. Such expression methods will be known to one of skill in the art. Individual phage supernatants or periplasmic extracts were exposed to blocked ELISA plates coated with antigen or a negative control and were subsequently detected using HRP-conjugated goat anti-Fc (obtained from Jackson Immunoresearch) for periplasmic extracts or anti-M13 (GE Healthcare) for phage, and then developed with TMB reagent (obtained from Thermo Fisher). Wells with OD₄₅₀ values greater than around 5-fold over background were considered positive clones and sequenced, after which some clones were expressed either as a soluble Fc fragment or fused to Fab fragments.

### General Methods for Yeast Selection

Bead Sorting (Magnetic-Assisted Cell Sorting (MACS)) Methods

MACS and FACS selections were performed similarly to as described in Ackerman et al., Biotechnol. Prog., 2009 25(3):774. Streptavidin magnetic beads (e.g., M-280 streptavidin beads from ThermoFisher) were labeled with biotinylated antigen and incubated with yeast (typically 5-10× library diversity). Unbound yeast were removed, the beads were washed, and bound yeast were grown in selective media and induced for subsequent rounds of selection.

Fluorescence-Activated Cell Sorting (FACS) Methods

Yeast were labeled with anti-c-Myc antibody to monitor expression and biotinylated antigen (concentration varied depending on the sorting round). In some experiments, the antigen was pre-mixed with streptavidin-Alexa Fluor® 647 in order to enhance the avidity of the interaction. In other experiments, the biotinylated antigen was detected after binding and washing with streptavidin-Alexa Fluor® 647. Singlet yeast with binding were sorted using a FACS Aria III cell sorter. The sorted yeast were grown in selective media then induced for subsequent selection rounds.

After an enriched yeast population was achieved, yeast were plated on SD-CAA agar plates and single colonies were grown and induced for expression, then labeled as described above to determine their propensity to bind to the target. Positive single clones were subsequently sequenced for binding antigen, after which some clones were expressed either as a soluble Fc fragment or as fused to Fab fragments.

### General Methods for Screening

Screening by ELISA

Clones were selected from panning outputs and grown in individual wells of 96-well deep-well plates. The clones were either induced for periplasmic expression using autoinduction media (obtained from EMD Millipore) or infected with helper phage for phage-display of the individual Fc variants on phage. ELISA plates were coated with antigen, typically at 0.5 mg/mL overnight, then blocked with 1% BSA before addition of phage or periplasmic extracts. After a 1-hour incubation and washing off unbound protein, HRP-conjugated secondary antibody was added (i.e., anti-Fc or anti-M13 for soluble Fc or phage-displayed Fc, respectively) and incubated for 30 minutes. The plates were washed again, and then developed with TMB reagent and quenched with 2N sulfuric acid. Absorbance at 450 nm was quantified using a plate reader (BioTek®) and binding curves were polotted using Prism software where applicable. In some assays, soluble transferrin or other competitor was added during the binding step, typically at significant molar excess.

Screening by Flow Cytometry

Fc variant polypeptides (expressed either on phage, in periplasmic extracts, or solubly as fusions to Fab fragments) were added to cells in 96-well V-bottom plates (about 100,000 cells per well in PBS+1% BSA (PBSA)), and incubated at 4° C. for 1 hour. The plates were subsequently spun and the media was removed, and then the cells were washed once with PBSA. The cells were resuspended in PBSA containing secondary antibody (typically goat anti-human-IgG-Alexa Fluor® 647 (obtained from Thermo Fisher)). After 30 minutes, the plates were spun and the media was removed, the cells were washed 1-2 times with PBSA, and then the plates were read on a flow cytometer (i.e., a FACSCanto™ II flow cytometer). Median fluorescence values were calculated for each condition using FlowJo software and binding curves were plotted with Prism software.

TABLE 11
CH3 Domain Modification

| Clone name | Group | 384 | 385 | 386 | 387 | 388 | 389 | 390 | 391 | . . . | 413 | 414 | 415 | 416 | 417 | 418 | 419 | 420 | 421 |
|---|---|---|---|---|---|---|---|---|---|---|---|---|---|---|---|---|---|---|---|
| Wild-type | n/a | N | G | Q | P | E | N | N | Y | . . . | D | K | S | R | W | Q | Q | G | N |
| 1 | | L | G | L | V | W | V | G | Y | . . . | A | K | S | T | W | Q | Q | G | W |
| 2 | | Y | G | T | V | W | S | H | Y | . . . | S | K | S | E | W | Q | Q | G | Y |
| 3 | | Y | G | T | E | W | S | Q | Y | . . . | E | K | S | D | W | Q | Q | G | H |
| 4 | | Y | G | T | P | W | A | L | Y | . . . | L | K | S | E | W | Q | Q | G | W |
| 17 | 2 | Y | G | T | V | W | S | K | Y | . . . | S | K | S | E | W | Q | Q | G | F |
| 18 | 1 | L | G | H | V | W | A | V | Y | . . . | P | K | S | T | W | Q | Q | G | W |
| 21 | 1 | L | G | L | V | W | V | G | Y | . . . | P | K | S | T | W | Q | Q | G | W |
| 25 | 1 | M | G | H | V | W | V | G | Y | . . . | D | K | S | T | W | Q | Q | G | W |
| 34 | 1 | L | G | L | V | W | V | F | S | . . . | P | K | S | T | W | Q | Q | G | W |
| 35 | 2 | Y | G | T | E | W | S | S | Y | . . . | T | K | S | E | W | Q | Q | G | F |
| 44 | 2 | Y | G | T | E | W | S | N | Y | . . . | S | K | S | E | W | Q | Q | G | F |
| 51 | 1/2 | L | G | H | V | W | V | G | Y | . . . | S | K | S | E | W | Q | Q | G | W |
| 3.1-3 | 1 | L | G | H | V | W | V | A | T | . . . | P | K | S | T | W | Q | Q | G | W |
| 3.1-9 | 1 | L | G | P | V | W | V | H | T | . . . | P | K | S | T | W | Q | Q | G | W |
| 3.2-5 | 1 | L | G | H | V | W | V | D | Q | . . . | P | K | S | T | W | Q | Q | G | W |
| 3.2-19 | 1 | L | G | H | V | W | V | N | Q | . . . | P | K | S | T | W | Q | Q | G | W |
| 3.2-1 | 1 | L | G | H | V | W | V | N | F | . . . | P | K | S | T | W | Q | Q | G | W |
| 3.4-1 | | W | G | F | V | W | S | T | Y | . . . | P | K | S | N | W | Q | Q | G | F |
| 3.4-19 | | W | G | H | V | W | S | T | Y | . . . | P | K | S | N | W | Q | Q | G | Y |
| 3.2-3 | | L | G | H | V | W | V | E | Q | . . . | P | K | S | T | W | Q | Q | G | W |
| 3.2-14 | | L | G | H | V | W | V | G | V | . . . | P | K | S | T | W | Q | Q | G | W |
| 3.2-24 | | L | G | H | V | W | V | H | T | . . . | P | K | S | T | W | Q | Q | G | W |
| 3.4-26 | | W | G | T | V | W | G | T | Y | . . . | P | K | S | N | W | Q | Q | G | Y |
| 3.2-17 | | L | G | H | V | W | V | G | T | . . . | P | K | S | T | W | Q | Q | G | W |

TABLE 12
Additional CH3 Domain Modifications

| Clone name | 378 | 379 | 380 | 381 | 382 | 383 | 384 | 385 | 386 | 387 | 388 | 389 | 390 | 391 | 392 | 411 | 412 | 413 | 414 | 415 | 416 | 417 | 418 | 419 | 420 | 421 | 422 | 423 |
|---|---|---|---|---|---|---|---|---|---|---|---|---|---|---|---|---|---|---|---|---|---|---|---|---|---|---|---|---|
| Wild-type | A | V | E | W | E | S | N | G | Q | N | E | N | N | Y | K | T | V | D | K | S | R | W | Q | Q | G | N | V | F |
| 35.20.1 | • | • | • | • | • | • | F | • | T | E | W | S | S | • | • | • | • | T | • | E | E | • | • | • | • | F | • | • |
| 35.20.2 | • | • | • | • | • | • | Y | • | T | E | W | A | S | • | • | • | • | T | • | E | E | • | • | • | • | F | • | • |
| 35.20.3 | • | • | • | • | • | • | Y | • | T | E | W | V | S | • | • | • | • | T | • | E | E | • | • | • | • | F | • | • |
| 35.20.4 | • | • | • | • | • | • | Y | • | T | E | W | S | S | • | • | • | • | S | • | E | E | • | • | • | • | F | • | • |
| 35.20.5 | • | • | • | • | • | • | F | • | T | E | W | A | S | • | • | • | • | T | • | E | E | • | • | • | • | F | • | • |
| 35.20.6 | • | • | • | • | • | • | F | • | T | E | W | V | S | • | • | • | • | T | • | E | E | • | • | • | • | F | • | • |
| 35.21.a.1 | • | • | W | • | • | • | F | • | T | E | W | S | S | • | • | • | • | T | • | E | E | • | • | • | • | F | • | • |
| 35.21.a.2 | • | • | W | • | • | • | Y | • | T | E | W | A | S | • | • | • | • | T | • | E | E | • | • | • | • | F | • | • |
| 35.21.a.3 | • | • | W | • | • | • | Y | • | T | E | W | V | S | • | • | • | • | T | • | E | E | • | • | • | • | F | • | • |
| 35.21.a.4 | • | • | W | • | • | • | Y | • | T | E | W | S | S | • | • | • | • | S | • | E | E | • | • | • | • | F | • | • |
| 35.21.a.5 | • | • | W | • | • | • | F | • | T | E | W | A | S | • | • | • | • | T | • | E | E | • | • | • | • | F | • | • |
| 35.21.a.6 | • | • | W | • | • | • | F | • | T | E | W | V | S | • | • | • | • | T | • | E | E | • | • | • | • | F | • | • |
| 35.23.1 | • | • | • | • | • | • | F | • | T | E | W | S | • | • | • | • | • | T | • | E | E | • | • | • | • | F | • | • |
| 35.23.2 | • | • | • | • | • | • | Y | • | T | E | W | A | • | • | • | • | • | T | • | E | E | • | • | • | • | F | • | • |
| 35.23.3 | • | • | • | • | • | • | Y | • | T | E | W | V | • | • | • | • | • | T | • | E | E | • | • | • | • | F | • | • |
| 35.23.4 | • | • | • | • | • | • | Y | • | T | E | W | S | • | • | • | • | • | S | • | E | E | • | • | • | • | F | • | • |
| 35.23.5 | • | • | • | • | • | • | F | • | T | E | W | A | • | • | • | • | • | T | • | E | E | • | • | • | • | F | • | • |
| 35.23.6 | • | • | • | • | • | • | F | • | T | E | W | V | • | • | • | • | • | T | • | E | E | • | • | • | • | F | • | • |
| 35.24.1 | • | • | W | • | • | • | F | • | T | E | W | S | • | • | • | • | • | T | • | E | E | • | • | • | • | F | • | • |
| 35.24.2 | • | • | W | • | • | • | Y | • | T | E | W | A | • | • | • | • | • | T | • | E | E | • | • | • | • | F | • | • |
| 35.24.3 | • | • | W | • | • | • | Y | • | T | E | W | V | • | • | • | • | • | T | • | E | E | • | • | • | • | F | • | • |
| 35.24.4 | • | • | W | • | • | • | Y | • | T | E | W | S | • | • | • | • | • | S | • | E | E | • | • | • | • | F | • | • |
| 35.24.5 | • | • | W | • | • | • | F | • | T | E | W | A | • | • | • | • | • | T | • | E | E | • | • | • | • | F | • | • |
| 35.24.6 | • | • | W | • | • | • | F | • | T | E | W | V | • | • | • | • | • | T | • | E | E | • | • | • | • | F | • | • |
| 35.21.17.1 | • | • | L | • | • | • | F | • | T | E | W | S | S | • | • | • | • | T | • | E | E | • | • | • | • | F | • | • |
| 35.21.17.2 | • | • | L | • | • | • | Y | • | T | E | W | A | S | • | • | • | • | T | • | E | E | • | • | • | • | F | • | • |
| 35.21.17.3 | • | • | L | • | • | • | Y | • | T | E | W | V | S | • | • | • | • | T | • | E | E | • | • | • | • | F | • | • |
| 35.21.17.4 | • | • | L | • | • | • | Y | • | T | E | W | S | S | • | • | • | • | S | • | E | E | • | • | • | • | F | • | • |
| 35.21.17.5 | • | • | L | • | • | • | F | • | T | E | W | A | S | • | • | • | • | T | • | E | E | • | • | • | • | F | • | • |
| 35.21.17.6 | • | • | L | • | • | • | F | • | T | E | W | V | S | • | • | • | • | T | • | E | E | • | • | • | • | F | • | • |
| 35.20 | • | • | • | • | • | • | Y | • | T | E | W | S | S | • | • | • | • | T | • | E | E | • | • | • | • | F | • | • |
| 35.21 | • | • | W | • | • | • | Y | • | T | E | W | S | S | • | • | • | • | T | • | E | E | • | • | • | • | F | • | • |
| 35.22 | • | • | W | • | • | • | Y | • | T | E | W | S | • | • | • | • | • | T | • | E | E | • | • | • | • | F | • | • |
| 35.23 | • | • | • | • | • | • | Y | • | T | E | W | S | • | • | • | • | • | T | • | E | E | • | • | • | • | F | • | • |
| 35.24 | • | • | W | • | • | • | Y | • | T | E | W | S | • | • | • | • | • | T | • | E | E | • | • | • | • | F | • | • |
| 35.21.17 | • | • | L | • | • | • | Y | • | T | E | W | S | S | • | • | • | • | T | • | E | E | • | • | • | • | F | • | • |
| 35.N390 | • | • | • | • | • | • | Y | • | T | E | W | S | • | • | • | • | • | T | • | E | E | • | • | • | • | F | • | • |
| 35.20.1.1 | • | • | • | • | • | • | F | • | T | E | W | S | S | • | • | • | • | S | • | E | E | • | • | • | • | F | • | • |
| 35.23.2.1 | • | • | • | • | • | • | Y | • | T | E | W | A | • | • | • | • | • | S | • | E | E | • | • | • | • | F | • | • |
| 35.23.1.1 | • | • | • | • | • | • | F | • | T | E | W | S | • | • | • | • | • | S | • | E | E | • | • | • | • | F | • | • |
| 35.S413 | • | • | • | • | • | • | Y | • | T | E | W | S | S | • | • | • | • | S | • | E | E | • | • | • | • | F | • | • |
| 35.23.3.1 | • | • | • | • | • | • | Y | • | T | E | W | V | • | • | • | • | • | S | • | E | E | • | • | • | • | F | • | • |
| 35.N390.1 | • | • | • | • | • | • | Y | • | T | E | W | S | • | • | • | • | • | S | • | E | E | • | • | • | • | F | • | • |
| 35.23.6.1 | • | • | • | • | • | • | F | • | T | E | W | V | • | • | • | • | • | S | • | E | E | • | • | • | • | F | • | • |

### Example 6. Generation and Characterization of Anti-TREM2 Antibody Comprising Modified Fc Polypeptide

### Generation of TREM2 Fabs Fused to BBB-Penetrating Fc Polypeptide

### RS9.F6/3C. 35.21.17

A first RS9.F6 heavy chain was constructed by cloning the Fd (VH+CH1 regions) of clone RS9.F6 into an expression vector comprising an Fc engineered to bind to transferrin receptor and also comprising L234A, L235A, and P331G substitutions (according to the EU numbering scheme) to alter effector function and a “knob” mutation (T366W) to prevent homodimerization and promote heterodimerization with an Fc comprising “hole” mutations (T366W/L368A/Y407V). The first RS9.F6 heavy chain was designed to express the sequence of SEQ ID NO:91.

A second RS9.F6 heavy chain was constructed by cloning the Fd (VH+CH1 regions) of clone RS9.F6 into an expression vector comprising an Fc comprising the “hole” mutations (T366W/L368A/Y407V) and also comprising L234A, L235A, and P331G substitutions (according to the EU numbering scheme) to alter effector function, but lacking the transferrin receptor binding mutations. The second RS9.F6 heavy chain was designed to express the sequence of SEQ ID NO:92.

The light chain for RS9.F6 was constructed using an expression vector comprising a polynucleotide encoding the sequence of SEQ ID NO:35.

The vectors comprising polynucleotides encoding the aforementioned sequences of SEQ ID NOs:35, 91, and 92 (for RS9.F6) were co-transfected to ExpiCHO or Expi293 cells in the ratio of 1:1:2 (first heavy chain:second heavy chain:light chain). The expressed protein (referred to as “RS9.F6/3C.35.21.17”) was purified by Protein A chromatography followed by preparative size-exclusion chromatography (SEC) by methods familiar to those with skill in the art.

### 3D3.A1/3C.35.21.17

A first 3D3 heavy chain is constructed by cloning the Fd (VH+CH1 regions) of clone 3D3.A1 into an expression vector comprising an Fc engineered to bind to transferrin receptor and also comprising L234A, L235A, and P331G substitutions (according to the EU numbering scheme) to alter effector function and a “knob” mutation (T366W) to prevent homodimerization and promote heterodimerization with an Fc comprising “hole” mutations (T366W/L368A/Y407V). The first 3D3.A1 heavy chain is designed to express the sequence of SEQ ID NO:94. A second 3D3.A1 heavy chain is constructed by cloning the Fd (V_{H}+CH1 regions) of clone 3D3.A1 into an expression vector comprising an Fc comprising the “hole” mutations (T366W/L368A/Y407V) and also comprising L234A, L235A, and P331G substitutions (according to the EU numbering scheme) to alter effector function, but lacking the transferrin receptor binding mutations. The second 3D3.A1 heavy chain is designed to express the sequence of SEQ ID NO:95.

The light chain for 3D3.A1 is constructed using an expression vector comprising a polynucleotide encoding the sequence of SEQ ID NO:29.

The vectors comprising polynucleotides encoding the aforementioned sequences of SEQ ID NOs:29, 94, and 95 (for 3D3.A1) are co-transfected to ExpiCHO or Expi293 cells in the ratio of 1:1:2 (first heavy chain:second heavy chain:light chain). The expressed proteins (referred to as “3D3/3C.35.21.17”) are purified by Protein A chromatography followed by preparative size-exclusion chromatography (SEC) by methods familiar to those with skill in the art.

### Binding of RS9.F6/3C35.21.17 to TREM2 and Transferrin Receptor (TfR)

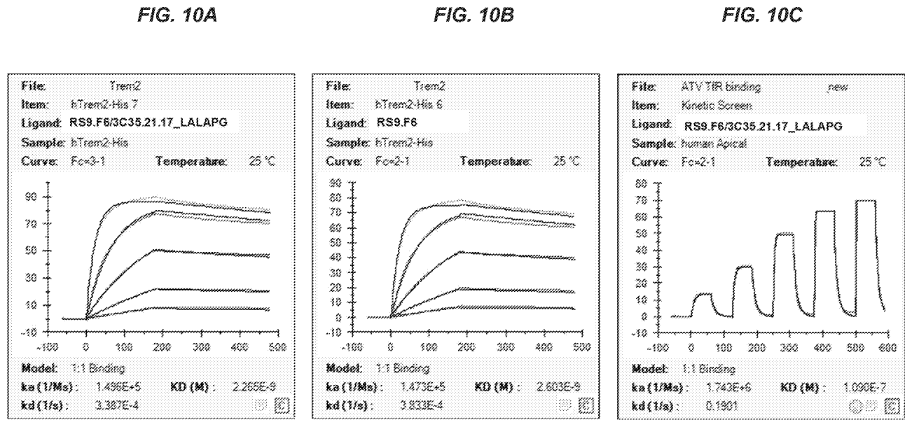

The binding of the TREM2/TfR-binding protein RS9.F6/3C35.21.17 to TREM2 and TfR was measured using SPR on a Biacore T200 instrument. To measure TfR binding, anti-human-Fab was immobilized on a CM5 chip, and the TREM2/TfR-binding protein was captured. Full-length human TfR or human TfR apical domain at serial dilution (e.g., concentrations of 1-1000 nM) was flowed over the chip (180 second association time) and then allowed to dissociate. Fitting was performed using a 1:1 binding model. To measure TREM2 binding, anti-human-Fc antibody was immobilized on a CM5 chip, and the TREM2/TfR-binding protein was captured. A range of concentrations of recombinant TREM2-His protein were flowed over the chip, and allowed to associate and dissociate. The resulting sensograms were fitted using a 1:1 Langmuir model to estimate kₒₙ and k_{off} (FIG. 10).

### Biacore Assessment of RS9.F6/3C35.21.17

The affinities of RS9.F6/3C35.21.17 for human TREM2 and TfR were determined by surface plasmon resonance using a Biacore™ T200. Biacore Series S CM5 sensor chips were immobilized with a mixture of two monoclonal mouse anti-Fab antibodies (Human Fab capture kit from GE Healthcare). Serial 3-fold dilutions of each antigen were injected at a flow rate of 30 μL/min. The binding of the antigens to captured antibody was monitored for 30 to 180 seconds and then their dissociation was monitored for 30-300 seconds in HBS-EP+running buffer (GE, # BR100669). Binding response was corrected by subtracting the RU from a blank flow cell. A 1:1 Languir model of simultaneous fitting of kₒₙ and k_{off} was used for kinetics analysis. The binding kinetics of the RS9.F6/3C35.21.17 are shown in Table 13 below. Biacore binding showed the bispecific TREM2/TfR-binding protein was capable of binding with high affinity to TREM2 and expected affinity to hTfR.

TABLE 13
Summary of binding kinetics of RS9.F6/3C35.21.17 and controls

| | | | | KD |
|---|---|---|---|---|
| Ligand | Analyte | ka (1/Ms) | kd (1/s) | (nM) |
| RS9.F6/ | hTREM2 | 1.5E+05 | 3.8E−04 | 2.6 |
| 3C35.21.17_LALAPG | | | | |
| RS9.F6/ | hTfR | 1.7E+06 | 1.9E−01 | 110 |
| 3C35.21.17_LALAPG | apical | | | |
| | domain | | | |
| RS9.F6 | hTREM2 | 1.5E+05 | 3.4E−04 | 2.3 |
| RS9.F6 | hTfR | N.D. | N.D. | N.D. |
| | apical | | | |
| | domain | | | |
| 3D3 | hTREM2 | 4.8E+05 | 1.9E−02 | 40 |
| 3D3 | hTfR | N.D. | N.D. | N.D. |
| | apical | | | |
| | domain | | | |

N.D. = not determined

### Example 7. Epitope Mapping and X-Ray Crystallography of TREM2 Peptide-F6 Fab Co-Complex

As described below, the epitope of antibody RS9.F6 was determined using hydrogen-deuterium exchange mass spectroscopy. In addition, the crystal structure of RS9.F6 bound to a TREM2 peptide was also elucidated.

### F6 Fab Epitope Mapping by Hydrogen-Deuterium Exchange-Mass Spectroscopy (HDX-MS)

For epitope mapping of the binding of a Fab fragment of anti-TREM2 antibody RS9.F6 (“F6”) to human TREM2, a TREM2 extracellular domain (ECD) amino acid sequence without signal peptide and His tag was used:

(SEQ ID NO: 333)
SGAHNTTVFQGVAGQSLQVSCPYDSMKHWGRRKAWCRQLGEKGPCQRVVS
THNLWLLSFLRRWNGSTAITDDTLGGTLTITLRNLQPHDAGLYQCQSLHG
SEADTLRKVLVEVLADPLDHRDAGDLWFPGESESFEDAHVEHSISRSLLE
GEIPFPPTS

Pepsin/Protease XIII Digestion and LC-MS of TREM2 ECD to Determine Sequence Coverage

5.77 μg of native or 7.4 μg TREM2 in 130 μL of control buffer (30 mM Tris, 200 mM sodium chloride, 3% Glycerol at pH 8.0) was denatured by adding 130 μL of 4 M guanidine hydrochloride, 0.85 M TCEP buffer (final pH was 2.5) and incubating the mixture for 3 min at 20° C. Then, the mixture was subjected to online pepsin/protease XIII digestion using an in-house packed pepsin/protease XIII column (2.1×30 mm). The resultant peptides were analyzed using an UPLC-MS system comprised of a Waters Acquity UPLC coupled to a Q Exactive™ Hybrid Quadrupole-Orbitrap Mass Spectrometer (Thermo). The peptides were separated on a 50×1 mm C8 column with a 16.5 min gradient from 2-31% solvent B (0.2% formic acid in acetonitrile). Solvent A was 0.2% formic acid in water. The injection valve and pepsin/protease XIII column and their related connecting tubings were inside a cooling box maintained at 10° C. for native TREM2, respectively. The second switching valve, C8 column and their related connecting stainless steel tubings were inside another chilled circulating box maintained at −6° C. Peptide identification was done through searching MS/MS data against the TREM2 sequence with Mascot. The mass tolerance for the precursor and product ions were 7 ppm and 0.02 Da, respectively.

HDXMS for Native TREM2 with and without the Presence of Fab

12 μL of TREM2 (5.77 μg) or 12 μL of TREM2 & TREM2 Fab mixture (5.77 μg: 28.85 μg) was incubated with 118 μL deuterium oxide labeling buffer (30 mM Tris, 200 mM sodium chloride, 3% Glycerol at pD 7.6) for 0 s, 10 s, 60 s, 600 s or 3600 s at 20° C. Hydrogen/deuterium exchange was quenched by adding 130 μL of 4 M guanidine hydrochloride, 0.85 M TCEP buffer (final pH was 2.5). Subsequently, the quenched samples were subjected to on column pepsin/protease XIII digestion and LC-MS analysis as described above. The mass spectra were recorded in MS only mode.

Raw MS data was processed using HDX WorkBench, software for the analysis of H/D exchange MS data (J. Am. Soc. Mass Spectrom. 2012, 23 (9), 1512-1521). The deuterium levels were calculated using the average mass difference between the deuterated peptide and its native form (t₀).

Results

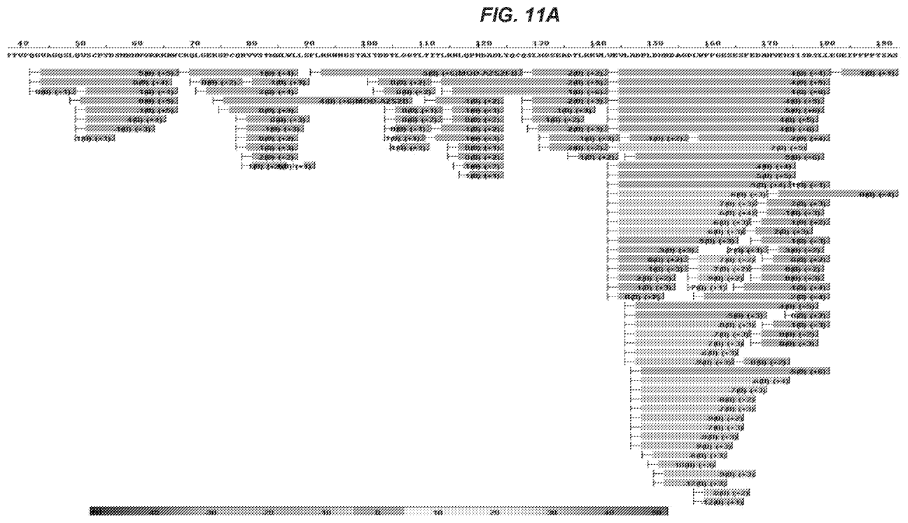

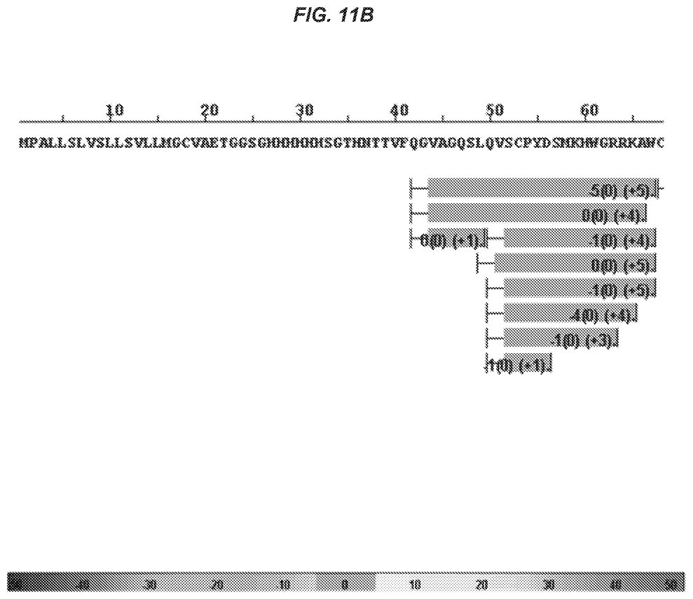

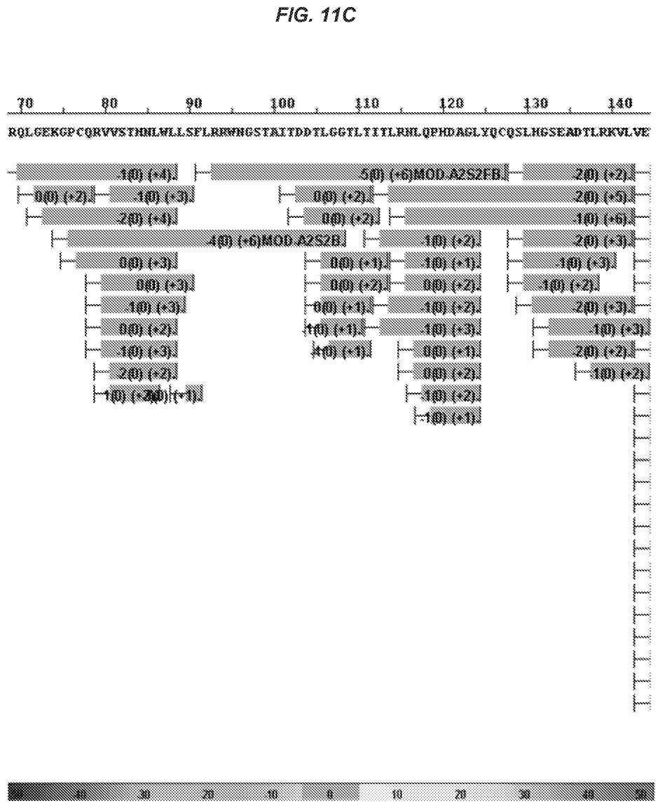

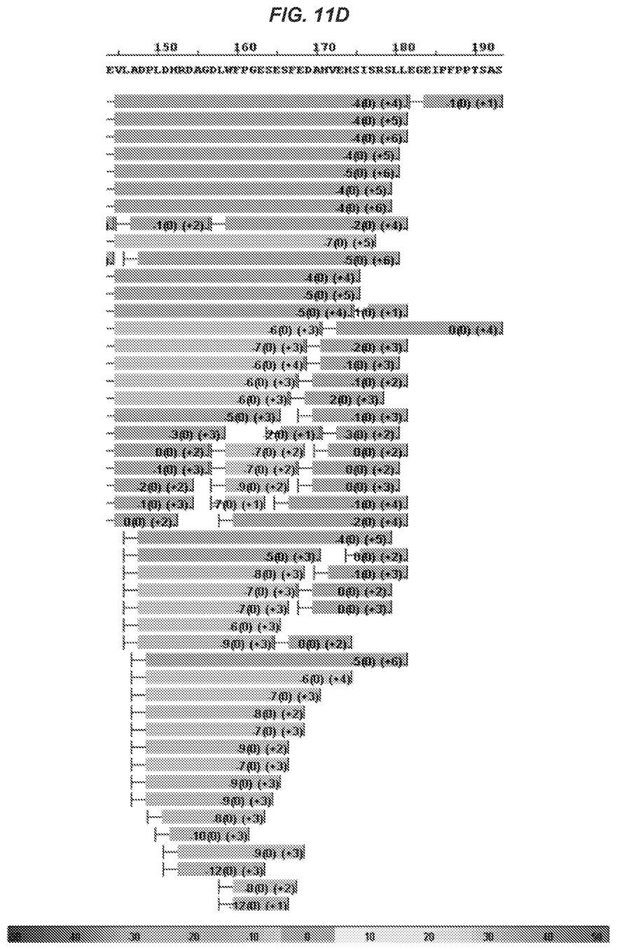

78.8% sequence coverage was achieved for native TREM2. TREM2 was incubated in deuterium oxide, either alone or in complex with Fab. The deuterium exchange was carried at 20° C. for 0 s, 10 s, 60 s, 600 s, or 3600 s. The exchange reaction was quenched by low pH and the proteins were digested with pepsin/protease XIII. The deuterium levels at the identified peptides were monitored from the mass shift on LC-MS. The deuterium buildup curves over exchange time for all the peptides were plotted. A differential heat map comparing hydrogen/deuterium exchange of TREM2 alone to that of TREM2 & Fab mixture is shown in FIGS. 11A-11D. As shown in FIGS. 11A and 11D, TREM2 showed a reduction in deuterium uptake at sequence AA157-166 (DLWFPGESES (SEQ ID NO:334), corresponding to residues 140-149 of the human TREM2 of SEQ ID NO:96) upon binding to Fab, thereby suggesting that the epitope targeted by the Fab for binding to native TREM2 is within this peptide region.

### TREM2-F6 Fab Co-Crystallization Method

Human TREM2 Synthetic Peptide 9-Mer Amino Acid Sequence

The synthetic 9-mer peptide DLWFPGESE (SEQ ID NO:335), corresponding to amino acid residues 140-148 of human TREM2 (according to UniProtKB entry TREM2_HUMAN) was used for co-crystallization.

F6 Fab Expression

The Fab fragment of the F6 anti-TREM2 antibody was expressed in Expi-293 cells at an initial cell density of 2.5×10⁶ cells/ml. Cells were harvested 96 hours post-transfection.

F6 Purification

The Fab was purified with Protein L resin. Immobilized Fab was washed with 20 mM Tris pH 8.5 and eluted with 0.1 M glycine pH 2.5. Protein eluate was immediately neutralized with 1M Tris pH 8.0. Protein eluate from Protein L resin was further purified by Superdex 200 size exclusion chromatography with 30 mM Tris pH 8.0, 200 mM NaCl, 3% glycerol in mobile phase.

Crystallization

Purified Fab solution was concentrated to 25 mg/mL in 20 mM M Tris pH 8.0, 0.2 M NaCl, 3% glycerol. The TREM2 9-mer peptide was reconstituted into 20 mM Tris pH 8.5 at 50 mg/ml. The Fab:peptide complex was obtained by mixing the components at 1:10 molar ratio with the excess of peptide at 23 mg/ml final concentration.

Crystallization experiments were performed at room temperature in a sitting drop format in Intelli 96-3 low-profile plates following the nano crystallization protocol. The setups included 20 commercially available and in-house screens with 96 conditions each. Crystals suitable for X-ray analysis grew from 25% PEG 8000 in 0.1 M HEPES buffer, pH 7.5. The crystals were cryoprotected by soaking in the mother liquor supplemented with 20% glycerol and flash frozen in liquid nitrogen.

X-Ray Data Collection

X-ray diffraction data were collected at the IMCA-CAT beamline 17-ID at the Advanced Photon Source (APS) at the Argonne National Laboratory (ANL) using a Dectris Pilatus 6M detector. The wavelength was 1.000 Å, exposure time was 0.25 s per 0.25° image. The diffraction images were processed with XDS to a resolution of 2.4 Å in space group P21 with unit cell dimensions: a=48.66 Å, b=65.37 Å, c=69.36 Å, and P=107.29°. The asymmetric unit contains one Fab:peptide complex.

The structure of the Fab:peptide complex was solved by molecular replacement with Phaser using a search model derived from PDB entry 5i16. The structure was manually rebuilt with Coot and refined using Refmac5. The Fab model contains all residues except Gln1 and Ser131-Ser135 of the heavy chain and a few residues at the C-terminus of each chain, all of which are disordered. Residues 140-146 of the TREM2 peptide are clearly visible in the electron density, while residues 147-148 are disordered. X-ray data and refinement statistics are given in Table 14 below.

TABLE 14
X-Ray Data and Refinement Statistics
X-Ray data statistics

| | |
|---|---|
| Space group | P21 |

Unit cell parameters

| | |
|---|---|
| a, b, c (Å) | 48.66, 65.37, 69.36 |
| α, β, γ (°) | 90, 107.29, 90 |

Refinement statistics

| | |
|---|---|
| Resolution range (Å) | 20.0-2.40 |
| Final R_{work} | 0.187 |
| Final R_{free} | 0.264 |
| Mean B value (Å²) | 56.5 |
| R.m.s. deviations | |
| Bonds (Å) | 0.006 |
| Angles (°) | 1.13 |
| Chiral volumes (Å³) | 0.070 |
| Ramachandran plot | |
| Preferred regions (%) | 97.4 |
| Allowed regions (%) | 2.6 |
| Outliers (%) | 0.0 |

Structure Analysis

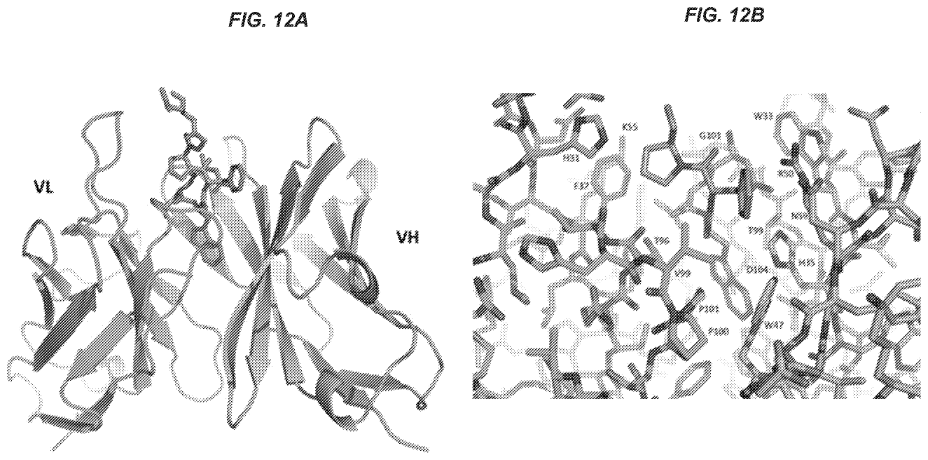

FIG. 12A shows that the F6 Fab binds the TREM2 peptide in the central cavity between the variable domains. The peptide is in the folded loop-like conformation, with the N-terminal Trp142 side chain buried deep in the binding cleft making contacts with CDR framework residues. As shown in FIG. 12B, the residues DLWFP (SEQ ID NO:336; residues 140-144 of the hTREM2 protein) make direct contacts with the Fab. The last two residues of the peptide, SE (residues 147-148 of the hTREM2 protein), are unstructured and do not have electron density in the structure. This type of immersed mode of antigen binding is not known for many protein-protein interactions.

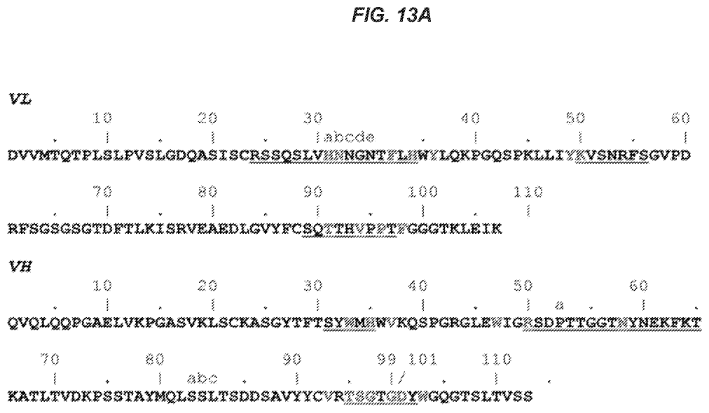

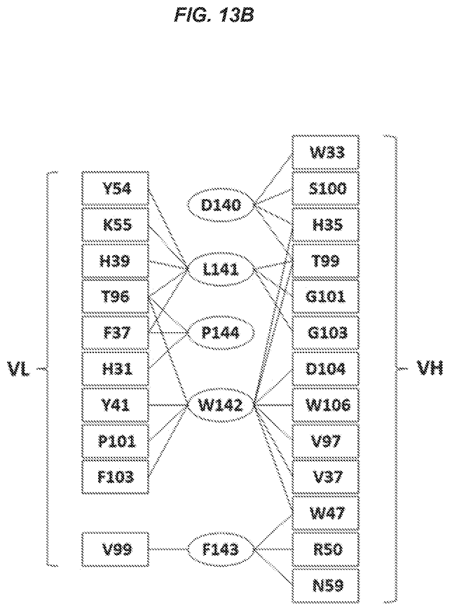

All six CDRs are involved in antigen binding. In total, 24 Fab residues are in direct contact (<4.0 Å) with the peptide, 7 of which are in the framework regions (FIGS. 13A-13B). These results show that an intricate network of contacts support F6 high-affinity binding, and that the contacts with Trp142 may be the primary driver to maintain high affinity.

TABLE 15
Informal Sequence Listing

| SEQ ID NO | Sequence | Description |
|---|---|---|
| 1 | GGACAGGGATCCAGAGTTCC | muIgG1 3′ primer |
| 2 | AGCTGGGAAGGTGTGCACAC | muIgG2 3′ primer |
| 3 | CAGGGGCCAGTGGATAGAC | muIgG3 3′ primer |
| 4 | GACATTGATGTCTTTGGGGT | muCkappa.1 3′ primer |
| 5 | TTCACTGCCATCAATCTTCC | muCkappa.2 3′ primer |
| 6 | QVQLQQPGAELVKPGASVKLSCKASGYTFTSYWMHWVKQSPGRGLEWIGRSDP | RS9.F6 VH amino acid |
| | TTGGTNYNEKFKTKATLTVDKPSSTAYMQLSSLTSDDSAVYYCVRTSGTGDYW | sequence |
| | GQGTSLTVSSAKTTAPSVYPLAPVCGGTTGSSVT | |
| 7 | DVVMTQTPLSLPVSLGDQASISCRSSQSLVHNNGNTFLHWYLQKPGQSPKLLI | RS9.F6 VL amino acid |
| | YKVSNRFSGVPDRFSGSGSGTDFTLKISRVEAEDLGVYFCSQTTHVPPTFGGG | sequence |
| | TKLEIKRADAAPTVSIFPPSSEQLTSGGASVVCF | |
| 8 | GYTFTSY | RS9.F6 CDR-H1 amino acid |
| | | sequence |
| 9 | IGRSDPTTGGTNYNE | RS9.F6 CDR-H2 amino acid |
| | | sequence |
| 10 | VRTSGTGDY | RS9.F6 and RS.F10 CDR-H3 |
| | | amino acid sequence; |
| 11 | RSSQSLVHNNGNTFLH | RS9.F6 and RS.F10 CDR-L1 |
| | | amino acid sequence |
| 12 | VSNRFS | RS9.F6 CDR-L2 amino acid |
| | | sequence |
| 13 | SQTTHVPPT | RS9.F6 and RS.F10 CDR-L3 |
| | | amino acid sequence |
| 14 | QVQLQQSGAELARPGASVKLSCKASGYTFTSYWIQWVKQRPGQGLEWIGTIYP | 21D11 VH amino acid |
| | GDGDARYTQKFKGKATLTADKSSSTTYMQLNSLASEDSAVYYCARNGITTAGY | sequence |
| | YAMDYWGQGTSVTVSS | |
| 15 | QVQLQQSGADLLRPGVSVKISCKGSGYTFTDHAMHWVKQSHAELEWIGVISTY | 21D4.D1 VH amino acid |
| | SGDTGYNQKFKGKATMTVDKSSSTAYLELARLTSEDSAIYYCAREGHYDDAMD | sequence |
| | YWGQGTSVTVSS | |
| 16 | EVQLQQSGPELVKPGASVKMSCKASGYTFTSYVMHWVKQKPGQGLEWIGYINP | 26D2 VH amino acid |
| | YTDGTKYNEKFKGKATLTSDKSSSTAYMDLSSLTSEDSAVYYCARGEVRRYAL | sequence |
| | DYWGQGTSVTVSS | |
| 17 | QVHLQQSGSELRSPGSSVKLSCKDFDSEVFPISYMSWIRQKPGHGFEWIGDIL | 26E2.A3 VH amino acid |
| | PSIGGRIYGVKFEDRATLDADTVSNTAYLELNSLTSEDSAIYYCARKDYGSLA | sequence; 24B4.A1 VH |
| | YWGQGTLVTVSA | amino acid sequence |
| 18 | EVQLQQSGPELVKPGASVKISCKTSGYTLSEYTMHWVIQSHGKSLEWIGGVIP | 3D3 A1 VH amino acid |
| | NSGGTSYNQKFRDKASLTVDKSSSTAYLELRSLTSEDSAVYYCARGDDSYRRG | sequence |
| | YALDYWGQGTSVTVSS | |
| 19 | EVQLQQSGAEVVKPGASVKLSCTASGFNIKDTYMHWVKQRPEQGLEWIGRIDP | 40H3.A4 VH amino acid |
| | ANGNTKYDPKFQGKATITADTSSNTAYLQLSSLTSEDTAVYYCATLFAYWGQG | sequence |
| | TLVTVSA | |
| 20 | DVQLQESGPGLVKPSQSLSLTCTVTGYSITSDYAWNWIRQFPGNKLEWMGYIN | 42E8.H1 VH amino acid |
| | YSGRTIYNPSLKSRISITRDTSKNHFFLQLISVTTEDTATYYCARWNGNYGFA | sequence |
| | YWGQGTLVTVSA | |
| 21 | DVQLQESGPGLVKPSQSLSLTCTVTGYSITSDYAWNWIRQFPGNRLEWMGYIS | 49H11.B1 VH amino acid |
| | FSGSTSYNPSLKSRISITRDTSKNQFFLQLNSVTTEDTATYYCARWNGNYGFA | sequence |
| | YWGQGTLVTVSA | |
| 22 | QVHLQQSGSELRSPGSSVKLSCKDFDSEVFPIAYMSWVRQKPGHGFEWIGDIL | 54C2.A1 VH amino acid |
| | PSIGRRIYGVKFEDKATLDADTVSNTAYLELNSLTSEDSAIYYCTRKDYGSLA | sequence |
| | YWGQGTLVTVSA | |
| 23 | QVQLKESGPGLVAPSQSLSITCTVSGFSLSRYSVYWVRQPPGKGLEWLGMIWG | 57D7.A1 VH amino acid |
| | GGNTDYNSALKSRLSISKDNSKSQVFLKMNSLQTDDSAMYYCVQYGGMDYWGQ | sequence |
| | GTSVTVSS | |
| 24 | QVQLQQPGAELVKPGASVKLSCKASGYTFTSYWMHWVKQSPGRGLEWIGRSDP | RS9.F6 VH amino acid |
| | TTGGTNYNEKFKTKATLTVDKPSSTAYMQLSSLTSDDSAVYYCVRTSGTGDYW | sequence; RS.F10 VH amino |
| | GQGTSLTVSS | acid sequence |
| 25 | DIQMTQSPASLSVSVGETVTITCRASENIYSNLAWYQQKQGRSPQLLVYAATN | 21D11 VL amino acid |
| | LADGVPSRFSGSGSGTQYSLKINSLQSEDFGYYYCQHFWGTPYTFGGGTKVEI | sequence |
| | K | |
| 26 | DVVMTQTPLTLSVTIGQPASFSCKSSQSLLDSDGKTYLNWLLRRPGQSPKRLI | 21D4.D1 VL amino acid |
| | YVVSKLDSGVPDRFTGSGSGTDFTLKISRVEAEDLGVYYCWQGTHFPYTFGGG | sequence |
| | TKLEIK | |
| 27 | DIQMTQSSSSFSVSLGDRVTITCKASEDIYNRLAWYQQKPGNAPRLLISGATS | 26D2 VL amino acid |
| | LETGVPSRFSGSGSGKDYTLSITSLQTEDVATYYCQQYWSTPWTFGGGTKLEI | sequence |
| | K | |
| 28 | DVVMTQTPLSLPVSLGDQASISCRSSQSLVHINGNTYLQWFLQKPGQSPKLLI | 26E2.A3 VL amino acid |
| | YKVSNRFSGVPDRFSGSGSGTAFTLKISRVEAEDLGVYFCSQSTHVPYTFGGG | sequence; 24B4.A1 VL |
| | TKLEIK | amino acid sequence |
| 29 | DIVMSQSPSSLAVSVGEKVTMSCKSSQSLLYSSNQKSYLAWYQQKPGQSPKLL | 3D3.A1 VL amino acid |
| | IYWASTRESGVPDRFRGSGSGTDFTLTISSVKAEDLAVYYCQQYFSYPPTFGG | sequence |
| | GTKLEIK | |
| 30 | DIVMTQAAFSNPVTLGTSASISCRSSKSLLHSNGITYLYWYLQKPGQSPQLLI | 40H3.A4 VL amino acid |
| | YQMSNLASGVPDRFSSSGSGIDFTLRINRVEAEDVGVYYCAQNLELPTFGSGT | sequence |
| | KLEIK | |
| 31 | DVVMTQNPLSLPVSLGDQASISCRSSQSLVHINGNTYLHWYLQKPGQSPKLLI | 42E8.H1 VL amino acid |
| | YKVSNRFSGVPDRFSGSGSGTDFTLKISRVEAEDLGVYFCSQTTHALFTFGSG | sequence |
| | TKLEIK | |
| 32 | DVVMTQTPLSLPVSLGDQASISCRSSQSLVHINGNTYLHWYLQKPGQSPKLLI | 49H11.B1 VL amino acid |
| | YKVSNRFSGVPDRFSGSGSGTDFTLKISRVEAEDLGVYFCSQSTHVTFTFGSG | sequence |
| | TKLEIK | |
| 33 | DVVMTQTPLSLPVSLGDQASISCRSSQSLVHINGNTYLQWYLQKPGQSPKLLI | 54C2.A1 VL amino acid |
| | YKVSNRFSGVPDRFSGSGSGTDFTLRISRVEAEDLGVYFCSQSTHLPYTFGGG | sequence |
| | TKLEIK | |
| 34 | DVLMTQTPLSLPVSLGDQASISCRSSQSIVHSNGNTYLEWYLQKPGQSPKLLI | 57D7.A1 VL amino acid |
| | YKVSNRFSGVPDRFSGSGSGTDFTLKISRVEAEDLGVYYCFQGSHVPYTFGGG | sequence |
| | TKLEIK | |
| 35 | DVVMTQTPLSLPVSLGDQASISCRSSQSLVHNNGNTFLHWYLQKPGQSPKLLI | RS9.F6 VL amino acid |
| | YKVSNRFSGVPDRFSGSGSGTDFTLKISRVEAEDLGVYFCSQTTHVPPTFGGG | sequence; RS.F10 VL amino |
| | TKLEIK | acid sequence |
| 36 | GYTFTSYWMH | RS9.F6 and RS.F10 CDR-H1 |
| 37 | RSDPTTGGTNYNEKFKT | RS9.F6 and RS.F10 CDR-H2 |
| 38 | KVSNRFS | RS9.F6, RS.F10, 26E2.A3, |
| | | 24B4.A1, 42E8.H1, |
| | | 49H11.B1, 54C2.A1, and |
| | | 57D7.A1 CDR-L2 |
| 39 | GYTFTSYWIQ | 21D11 CDR-H1 |
| 40 | TIYPGDGDARYTQKFKG | 21D11 CDR-H2 |
| 41 | ARNGITTAGYYAMDY | 21D11 CDR-H3 |
| 42 | RASENIYSNLA | 21D11 CDR-L1 |
| 43 | AATNLAD | 21D11 CDR-L2 |
| 44 | QHFWGTPYT | 21D11 CDR-L3 |
| 45 | GYTFTDHAMH | 21D4.D1 CDR-H1 |
| 46 | VISTYSGDTGYNQKFKG | 21D4.D1 CDR-H2 |
| 47 | AREGHYDDAMDY | 21D4.D1 CDR-H3 |
| 48 | KSSQSLLDSDGKTYLN | 21D4.D1 and 51D4 CDR-L1 |
| 49 | VVSKLDS | 21D4.D1 CDR-L2 |
| 50 | WQGTHFPYT | 21D4.D1 and 51D4 CDR-L3 |
| 51 | GYTFTSYVMH | 26D2 CDR-H1 |
| 52 | YINPYTDGTKYNEKFKG | 26D2 CDR-H2 |
| 53 | ARGEVRRYALDY | 26D2 CDR-H3 |
| 54 | KASEDIYNRLA | 26D2 CDR-L1 |
| 55 | GATSLET | 26D2 CDR-L2 |
| 56 | QQYWSTPWT | 26D2 CDR-L3 |
| 57 | DSEVFPISYMS | 26E2.A3 and 24B4.A1 CDR-H1 |
| 58 | DILPSIGGRIYGVKF | 26E2.A3 and 24B4.A1 CDR-H2 |
| 59 | ARKDYGSLAY | 26E2.A3 and 24B4.A1 CDR-H3 |
| 60 | RSSQSLVHINGNTYLQ | 26E2.A3, 24B4.A1, and |
| | | 54C2.A1 CDR-L1 |
| 61 | SQSTHVPYT | 26E2.A3 and 24B4.A1 CDR-L3 |
| 62 | GYTLSEYTMH | 3D3.A1 CDR-H1 |
| 63 | GVIPNSGGTSYNQKFRD | 3D3.A1 CDR-H2 |
| 64 | ARGDDSYRRGYALDY | 3D3.A1 CDR-H3 |
| 65 | KSSQSLLYSSNQKSYLA | 3D3.A1 CDR-L1 |
| 66 | WASTRES | 3D3.A1 CDR-L2 |
| 67 | QQYFSYPPT | 3D3.A1 CDR-L3 |
| 68 | GFNIKDTYMH | 40H3.A4 CDR-H1 |
| 69 | RIDPANGNTKYDPKFQG | 40H3.A4 CDR-H2 |
| 70 | ATLFAY | 40H3.A4 CDR-H3 |
| 71 | RSSKSLLHSNGITYLY | 40H3.A4 CDR-L1 |
| 72 | QMSNLAS | 40H3.A4 CDR-L2 |
| 73 | AQNLELPT | 40H3.A4 CDR-L3 |
| 74 | GYSITSDYAWN | 42E8.H1 and 49H11.B1 |
| | | CDR-H1 |
| 75 | YINYSGRTIYNPSLKS | 42E8.H1 CDR-H2 |
| 76 | ARWNGNYGFAY | 42E8.H1 and 49H11.B1 |
| | | CDR-H3 |
| 77 | RSSQSLVHINGNTYLH | 42E8.H1 and 49H11.B1 |
| | | CDR-L1 |
| 78 | SQTTHALFT | 42E8.H1 CDR-L3 |
| 79 | YISFSGSTSYNPSLKS | 49H11.B1 CDR-H2 |
| 80 | SQSTHVTFT | 49H11.B1 CDR-L3 |
| 81 | DSEVFPIAYMS | 54C2.A1 CDR-H1 |
| 82 | DILPSIGRRIYGVKFED | 54C2.A1 CDR-H2 |
| 83 | KDYGSLAY | 54C2.A1 CDR-H3 |
| 84 | SQSTHLPYT | 54C2.A1 CDR-L3 |
| 85 | GFSLSRYSVY | 57D7.A1 CDR-H1 |
| 86 | MIWGGGNTDYNSALKS | 57D7.A1 CDR-H2 |
| 87 | YGGMDY | 57D7.A1 CDR-H3 |
| 88 | RSSQSIVHSNGNTYLE | 57D7.A1 CDR-L1 |
| 89 | FQGSHVPYT | 57D7.A1 CDR-L3 |
| 90 | QVQLQQPGAELVKPGASVKLSCKASGYTFTSYWMHWVKQSPGRGLEWIGRSDP | RS9.F6-Fd |
| | TTGGTNYNEKFKTKATLTVDKPSSTAYMQLSSLTSDDSAVYYCVRTSGTGDYW | |
| | GQGTSLTVSSASTKGPSVFPLAPSSKSTSGGTAALGCLVKDYFPEPVTVSWNS | |
| | GALTSGVHTFPAVLQSSGLYSLSSVVTVPSSSLGTQTYICNVNHKPSNTKVDK | |
| | KVEPKSCDKTH | |
| 91 | QVQLQQPGAELVKPGASVKLSCKASGYTFTSYWMHWVKQSPGRGLEWIGRSDP | RS9.F6-Fd fused to Fc with |
| | TTGGTNYNEKFKTKATLTVDKPSSTAYMQLSSLTSDDSAVYYCVRTSGTGDYW | LALAPG, TfR binding, and |
| | GQGTSLTVSSASTKGPSVFPLAPSSKSTSGGTAALGCLVKDYFPEPVTVSWNS | knob mutations |
| | GALTSGVHTFPAVLQSSGLYSLSSVVTVPSSSLGTQTYICNVNHKPSNTKVDK | |
| | KVEPKSCDKTHTCPPCPAPEAAGGPSVFLFPPKPKDTLMISRTPEVTCVVVDV | |
| | SHEDPEVKFNWYVDGVEVHNAKTKPREEQYNSTYRVVSVLTVLHQDWLNGKEY | |
| | KCKVSNKALGAPIEKTISKAKGQPREPQVYTLPPSRDELTKNQVSLWCLVKGF | |
| | YPSDIAVLWESYGTEWASYKTTPPVLDSDGSFFLYSKLTVTKEEWQQGFVFSC | |
| | SVMHEALHNHYTQKSLSLSPGK | |
| 92 | QVQLQQPGAELVKPGASVKLSCKASGYTFTSYWMHWVKQSPGRGLEWIGRSDP | RS9.F6-Fd fused to Fc with |
| | TTGGTNYNEKFKTKATLTVDKPSSTAYMQLSSLTSDDSAVYYCVRTSGTGDYW | LALAPG and hole mutations |
| | GQGTSLTVSSASTKGPSVFPLAPSSKSTSGGTAALGCLVKDYFPEPVTVSWNS | |
| | GALTSGVHTFPAVLQSSGLYSLSSVVTVPSSSLGTQTYICNVNHKPSNTKVDK | |
| | KVEPKSCDKTHTCPPCPAPEAAGGPSVFLFPPKPKDTLMISRTPEVTCVVVDV | |
| | SHEDPEVKFNWYVDGVEVHNAKTKPREEQYNSTYRVVSVLTVLHQDWLNGKEY | |
| | KCKVSNKALGAPIEKTISKAKGQPREPQVYTLPPSRDELTKNQVSLSCAVKGF | |
| | YPSDIAVEWESNGQPENNYKTTPPVLDSDGSFFLVSKLTVDKSRWQQGNVFSC | |
| | SVMHEALHNHYTQKSLSLSPGK | |
| 93 | EVQLQQSGPELVKPGASVKISCKTSGYTLSEYTMHWVIQSHGKSLEWIGGVIP | 3D3.A1-Fd |
| | NSGGTSYNQKFRDKASLTVDKSSSTAYLELRSLTSEDSAVYYCARGDDSYRRG | |
| | YALDYWGQGTSVTVSSASTKGPSVFPLAPSSKSTSGGTAALGCLVKDYFPEPV | |
| | TVSWNSGALTSGVHTFPAVLQSSGLYSLSSVVTVPSSSLGTQTYICNVNHKPS | |
| | NTKVDKKVEPKSCDKTH | |
| 94 | EVQLQQSGPELVKPGASVKISCKTSGYTLSEYTMHWVIQSHGKSLEWIGGVIP | 3D3.A1-Fd fused to Fc with |
| | NSGGTSYNQKFRDKASLTVDKSSSTAYLELRSLTSEDSAVYYCARGDDSYRRG | LALAPG, TfR binding, and |
| | YALDYWGQGTSVTVSSASTKGPSVFPLAPSSKSTSGGTAALGCLVKDYFPEPV | knob mutations |
| | TVSWNSGALTSGVHTFPAVLQSSGLYSLSSVVTVPSSSLGTQTYICNVNHKPS | |
| | NTKVDKKVEPKSCDKTHTCPPCPAPEAAGGPSVFLFPPKPKDTLMISRTPEVT | |
| | CVVVDVSHEDPEVKFNWYVDGVEVHNAKTKPREEQYNSTYRVVSVLTVLHQDW | |
| | LNGKEYKCKVSNKALGAPIEKTISKAKGQPREPQVYTLPPSRDELTKNQVSLW | |
| | CLVKGFYPSDIAVLWESYGTEWASYKTTPPVLDSDGSFFLYSKLTVTKEEWQQ | |
| | GFVFSCSVMHEALHNHYTQKSLSLSPGK | |
| 95 | EVQLQQSGPELVKPGASVKISCKTSGYTLSEYTMHWVIQSHGKSLEWIGGVIP | 3D3.A1-Fd fused to Fc with |
| | PNSGGTSYNQKFRDKASLTVDKSSSTAYLELRSLTSEDSAVYYCARGDDSYRR | LALAPG and hole mutations |
| | GYALDYWGQGTSVTVSSASTKGPSVFPLAPSSKSTSGGTAALGCLVKDYFPEP | |
| | VTVSWNSGALTSGVHTFPAVLQSSGLYSLSSVVTVPSSSLGTQTYICNVNHKP | |
| | SNTKVDKKVEPKSCDKTHTCPPCPAPEAAGGPSVFLFPPKPKDTLMISRTPEV | |
| | TCVVVDVSHEDPEVKFNWYVDGVEVHNAKTKPREEQYNSTYRVVSVLTVLHQD | |
| | WLNGKEYKCKVSNKALGAPIEKTISKAKGQPREPQVYTLPPSRDELTKNQVSL | |
| | SCAVKGFYPSDIAVEWESNGQPENNYKTTPPVLDSDGSFFLVSKLTVDKSRWQ | |
| | QGNVFSCSVMHEALHNHYTQKSLSLSPGK | |
| 96 | MEPLRLLILLFVGELSGAHNTTVFQGVAGQSLQVSCPYDSMKHWGRRKAWCRQ | Human TREM2 protein |
| | LGEKGPCQRVVSTHNLWLLSFLRRWNGSTAITDDTLGGTLTITLRNLQPHDAG | |
| | LYQCQSLHGSEADTLRKVLVEVLADPLDHRDAGDLWFPGESESFEDAHVEHSI | |
| | SRSLLEGEIPFPPTSILLLLACIFLIKILAASALWAAAWHGQKPGTHPPSELD | |
| | CGHDPGYQLQTLPGLRDT | |
| 97 | MMDQARSAFSNLFGGEPLSYTRFSLARQVDGDNSHVEMKLAVDEEENADNNTK | Human transferrin receptor |
| | ANVTKPKRCSGSICYGTIAVIVFFLIGFMIGYLGYCKGVEPKTECERLAGTES | protein 1 (TFR1) |
| | PVREEPGEDFPAARRLYWDDLKRKLSEKLDSTDFTGTIKLLNENSYVPREAGS | |
| | QKDENLALYVENQFREFKLSKVWRDQHFVKIQVKDSAQNSVIIVDKNGRLVYL | |
| | VENPGGYVAYSKAATVTGKLVHANFGTKKDFEDLYTPVNGSIVIVRAGKITFA | |
| | EKVANAESLNAIGVLIYMDQTKFPIVNAELSFFGHAHLGTGDPYTPGFPSFNH | |
| | TQFPPSRSSGLPNIPVQTISRAAAEKLFGNMEGDCPSDWKTDSTCRMVTSESK | |
| | NVKLTVSNVLKEIKILNIFGVIKGFVEPDHYVVVGAQRDAWGPGAAKSGVGTA | |
| | LLLKLAQMFSDMVLKDGFQPSRSIIFASWSAGDFGSVGATEWLEGYLSSLHLK | |
| | AFTYINLDKAVLGTSNFKVSASPLLYTLIEKTMQNVKHPVTGQFLYQDSNWAS | |
| | KVEKLTLDNAAFPFLAYSGIPAVSFCFCEDTDYPYLGTTMDTYKELIERIPEL | |
| | NKVARAAAEVAGQFVIKLTHDVELNLDYERYNSQLLSFVRDLNQYRADIKEMG | |
| | LSLQWLYSARGDFFRATSRLTTDFGNAEKTDRFVMKKLNDRVMRVEYHFLSPY | |
| | VSPKESPFRHVFWGSGSHTLPALLENLKLRKQNNGAFNETLFRNQLALATWTI | |
| | QGAANALSGDVWDIDNEF | |
| 98 | APELLGGPSVFLFPPKPKDTLMISRTPEVTCVVVDVSHEDPEVKFNWYVDGVE | Wild-type human Fc |
| | VHNAKTKPREEQYNSTYRVVSVLTVLHQDWLNGKEYKCKVSNKALPAPIEKTI | sequence |
| | SKAKGQPREPQVYTLPPSRDELTKNQVSLTCLVKGFYPSDIAVEWESNGQPEN | positions 231-447 EU index |
| | NYKTTPPVLDSDGSFFLYSKLTVDKSRWQQGNVFSCSVMHEALHNHYTQKSLS | numbering |
| | LSPGK | |
| 99 | EPKSCDKTHTCPPCP | Human IgG1 hinge sequence |
| 100 | APELLGGPSVFLFPPKPKDTLMISRTPEVTCVVVDVSHEDPEVKFNWYVDGVE | Clone CH3C.35.20 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESYGTEWS | |
| | SYKTTPPVLDSDGSFFLYSKLTVTKEEWQQGFVFSCSVMHEALHNHYTQKSLS | |
| | LSPGK | |
| 101 | APELLGGPSVFLFPPKPKDTLMISRTPEVTCVVVDVSHEDPEVKFNWYVDGVE | Clone CH3C.35.21 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVWWESYGTEWS | |
| | SYKTTPPVLDSDGSFFLYSKLTVTKEEWQQGFVFSCSVMHEALHNHYTQKSLS | |
| | LSPGK | |
| 102 | APELLGGPSVFLFPPKPKDTLMISRTPEVTCVVVDVSHEDPEVKFNWYVDGVE | Clone CH3C.35.22 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVWWESYGTEWS | |
| | NYKTTPPVLDSDGSFFLYSKLTVTKSEWQQGFVFSCSVMHEALHNHYTQKSLS | |
| | LSPGK | |
| 103 | APELLGGPSVFLFPPKPKDTLMISRTPEVTCVVVDVSHEDPEVKFNWYVDGVE | Clone CH3C.35.23 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESYGTEWS | |
| | NYKTTPPVLDSDGSFFLYSKLTVTKEEWQQGFVFSCSVMHEALHNHYTQKSLS | |
| | LSPGK | |
| 104 | APELLGGPSVFLFPPKPKDTLMISRTPEVTCVVVDVSHEDPEVKFNWYVDGVE | Clone CH3C.35.24 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVWWESYGTEWS | |
| | NYKTTPPVLDSDGSFFLYSKLTVTKEEWQQGFVFSCSVMHEALHNHYTQKSLS | |
| | LSPGK | |
| 105 | APELLGGPSVFLFPPKPKDTLMISRTPEVTCVVVDVSHEDPEVKFNWYVDGVE | Clone CH3C.35.21.17 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVLWESYGTEWS | |
| | SYKTTPPVLDSDGSFFLYSKLTVTKEEWQQGFVFSCSVMHEALHNHYTQKSLS | |
| | LSPGK | |
| 106 | APELLGGPSVFLFPPKPKDTLMISRTPEVTCVVVDVSHEDPEVKFNWYVDGVE | Clone CH3C.35.20.1 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESFGTEWS | |
| | SYKTTPPVLDSDGSFFLYSKLTVTKEEWQQGFVFSCSVMHEALHNHYTQKSLS | |
| | LSPGK | |
| 107 | APELLGGPSVFLFPPKPKDTLMISRTPEVTCVVVDVSHEDPEVKFNWYVDGVE | Clone CH3C.35.20.2 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESYGTEWA | |
| | SYKTTPPVLDSDGSFFLYSKLTVTKEEWQQGFVFSCSVMHEALHNHYTQKSLS | |
| | LSPGK | |
| 108 | APELLGGPSVFLFPPKPKDTLMISRTPEVTCVVVDVSHEDPEVKFNWYVDGVE | Clone CH3C.35.20.3 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESYGTEWV | |
| | SYKTTPPVLDSDGSFFLYSKLTVTKEEWQQGFVFSCSVMHEALHNHYTQKSLS | |
| | LSPGK | |
| 109 | APELLGGPSVFLFPPKPKDTLMISRTPEVTCVVVDVSHEDPEVKFNWYVDGVE | Clone CH3C.35.20.4 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESYGTEWS | |
| | SYKTTPPVLDSDGSFFLYSKLTVSKEEWQQGFVFSCSVMHEALHNHYTQKSLS | |
| | LSPGK | |
| 110 | APELLGGPSVFLFPPKPKDTLMISRTPEVTCVVVDVSHEDPEVKFNWYVDGVE | Clone CH3C.35.20.5 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESFGTEWA | |
| | SYKTTPPVLDSDGSFFLYSKLTVTKEEWQQGFVFSCSVMHEALHNHYTQKSLS | |
| | LSPGK | |
| 111 | APELLGGPSVFLFPPKPKDTLMISRTPEVTCVVVDVSHEDPEVKFNWYVDGVE | Clone CH3C.35.20.6 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESFGTEWV | |
| | SYKTTPPVLDSDGSFFLYSKLTVTKEEWQQGFVFSCSVMHEALHNHYTQKSLS | |
| | LSPGK | |
| 112 | APELLGGPSVFLFPPKPKDTLMISRTPEVTCVVVDVSHEDPEVKFNWYVDGVE | Clone CH3C.35.21.a.1 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVWWESFGTEWS | |
| | SYKTTPPVLDSDGSFFLYSKLTVTKEEWQQGFVFSCSVMHEALHNHYTQKSLS | |
| | LSPGK | |
| 113 | APELLGGPSVFLFPPKPKDTLMISRTPEVTCVVVDVSHEDPEVKFNWYVDGVE | Clone CH3C.35.21.a.2 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVWWESYGTEWA | |
| | SYKTTPPVLDSDGSFFLYSKLTVTKEEWQQGFVFSCSVMHEALHNHYTQKSLS | |
| | LSPGK | |
| 114 | APELLGGPSVFLFPPKPKDTLMISRTPEVTCVVVDVSHEDPEVKFNWYVDGVE | Clone CH3C.35.21.a.3 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVWWESYGTEWV | |
| | SYKTTPPVLDSDGSFFLYSKLTVTKEEWQQGFVFSCSVMHEALHNHYTQKSLS | |
| | LSPGK | |
| 115 | APELLGGPSVFLFPPKPKDTLMISRTPEVTCVVVDVSHEDPEVKFNWYVDGVE | Clone CH3C.35.21.a.4 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVWWESYGTEWS | |
| | SYKTTPPVLDSDGSFFLYSKLTVSKEEWQQGFVFSCSVMHEALHNHYTQKSLS | |
| | LSPGK | |
| 116 | APELLGGPSVFLFPPKPKDTLMISRTPEVTCVVVDVSHEDPEVKFNWYVDGVE | Clone CH3C.35.21.a.5 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVWWESFGTEWA | |
| | SYKTTPPVLDSDGSFFLYSKLTVTKEEWQQGFVFSCSVMHEALHNHYTQKSLS | |
| | LSPGK | |
| 117 | APELLGGPSVFLFPPKPKDTLMISRTPEVTCVVVDVSHEDPEVKFNWYVDGVE | Clone CH3C.35.21.a.6 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVWWESFGTEWV | |
| | SYKTTPPVLDSDGSFFLYSKLTVTKEEWQQGFVFSCSVMHEALHNHYTQKSLS | |
| | LSPGK | |
| 118 | APELLGGPSVFLFPPKPKDTLMISRTPEVTCVVVDVSHEDPEVKFNWYVDGVE | Clone CH3C.35.23.1 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESFGTEWS | |
| | NYKTTPPVLDSDGSFFLYSKLTVTKEEWQQGFVFSCSVMHEALHNHYTQKSLS | |
| | LSPGK | |
| 119 | APELLGGPSVFLFPPKPKDTLMISRTPEVTCVVVDVSHEDPEVKFNWYVDGVE | Clone CH3C.35.23.2 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESYGTEWA | |
| | NYKTTPPVLDSDGSFFLYSKLTVTKEEWQQGFVFSCSVMHEALHNHYTQKSLS | |
| | LSPGK | |
| 120 | APELLGGPSVFLFPPKPKDTLMISRTPEVTCVVVDVSHEDPEVKFNWYVDGVE | Clone CH3C.35.23.3 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESYGTEWV | |
| | NYKTTPPVLDSDGSFFLYSKLTVTKEEWQQGFVFSCSVMHEALHNHYTQKSLS | |
| | LSPGK | |
| 121 | APELLGGPSVFLFPPKPKDTLMISRTPEVTCVVVDVSHEDPEVKFNWYVDGVE | Clone CH3C.35.23.4 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESYGTEWS | |
| | NYKTTPPVLDSDGSFFLYSKLTVSKEEWQQGFVFSCSVMHEALHNHYTQKSLS | |
| | LSPGK | |
| 122 | APELLGGPSVFLFPPKPKDTLMISRTPEVTCVVVDVSHEDPEVKFNWYVDGVE | Clone CH3C.35.23.5 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESFGTEWA | |
| | NYKTTPPVLDSDGSFFLYSKLTVTKEEWQQGFVFSCSVMHEALHNHYTQKSLS | |
| | LSPGK | |
| 123 | APELLGGPSVFLFPPKPKDTLMISRTPEVTCVVVDVSHEDPEVKFNWYVDGVE | Clone CH3C.35.23.6 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESFGTEWV | |
| | NYKTTPPVLDSDGSFFLYSKLTVTKEEWQQGFVFSCSVMHEALHNHYTQKSLS | |
| | LSPGK | |
| 124 | APELLGGPSVFLFPPKPKDTLMISRTPEVTCVVVDVSHEDPEVKFNWYVDGVE | Clone CH3C.35.24.1 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVWWESFGTEWS | |
| | NYKTTPPVLDSDGSFFLYSKLTVTKEEWQQGFVFSCSVMHEALHNHYTQKSLS | |
| | LSPGK | |
| 125 | APELLGGPSVFLFPPKPKDTLMISRTPEVTCVVVDVSHEDPEVKFNWYVDGVE | Clone CH3C.35.24.2 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVWWESYGTEWA | |
| | NYKTTPPVLDSDGSFFLYSKLTVTKEEWQQGFVFSCSVMHEALHNHYTQKSLS | |
| | LSPGK | |
| 126 | APELLGGPSVFLFPPKPKDTLMISRTPEVTCVVVDVSHEDPEVKFNWYVDGVE | Clone CH3C.35.24.3 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVWWESYGTEWV | |
| | NYKTTPPVLDSDGSFFLYSKLTVTKEEWQQGFVFSCSVMHEALHNHYTQKSLS | |
| | LSPGK | |
| 127 | APELLGGPSVFLFPPKPKDTLMISRTPEVTCVVVDVSHEDPEVKFNWYVDGVE | Clone CH3C.35.24.4 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVWWESYGTEWS | |
| | NYKTTPPVLDSDGSFFLYSKLTVSKEEWQQGFVFSCSVMHEALHNHYTQKSLS | |
| | LSPGK | |
| 128 | APELLGGPSVFLFPPKPKDTLMISRTPEVTCVVVDVSHEDPEVKFNWYVDGVE | Clone CH3C.35.24.5 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVWWESFGTEWA | |
| | NYKTTPPVLDSDGSFFLYSKLTVTKEEWQQGFVFSCSVMHEALHNHYTQKSLS | |
| | LSPGK | |
| 129 | APELLGGPSVFLFPPKPKDTLMISRTPEVTCVVVDVSHEDPEVKFNWYVDGVE | Clone CH3C.35.24.6 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVWWESFGTEWV | |
| | NYKTTPPVLDSDGSFFLYSKLTVTKEEWQQGFVFSCSVMHEALHNHYTQKSLS | |
| | LSPGK | |
| 130 | APELLGGPSVFLFPPKPKDTLMISRTPEVTCVVVDVSHEDPEVKFNWYVDGVE | Clone CH3C.35.21.17.1 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVLWESFGTEWS | |
| | SYKTTPPVLDSDGSFFLYSKLTVTKEEWQQGFVFSCSVMHEALHNHYTQKSLS | |
| | LSPGK | |
| 131 | APELLGGPSVFLFPPKPKDTLMISRTPEVTCVVVDVSHEDPEVKFNWYVDGVE | Clone CH3C.35.21.17.2 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVLWESYGTEWA | |
| | SYKTTPPVLDSDGSFFLYSKLTVTKEEWQQGFVFSCSVMHEALHNHYTQKSLS | |
| | LSPGK | |
| 132 | APELLGGPSVFLFPPKPKDTLMISRTPEVTCVVVDVSHEDPEVKFNWYVDGVE | Clone CH3C.35.21.17.3 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVLWESYGTEWV | |
| | SYKTTPPVLDSDGSFFLYSKLTVTKEEWQQGFVFSCSVMHEALHNHYTQKSLS | |
| | LSPGK | |
| 133 | APELLGGPSVFLFPPKPKDTLMISRTPEVTCVVVDVSHEDPEVKFNWYVDGVE | Clone CH3C.35.21.17.4 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVLWESYGTEWS | |
| | SYKTTPPVLDSDGSFFLYSKLTVSKEEWQQGFVFSCSVMHEALHNHYTQKSLS | |
| | LSPGK | |
| 134 | APELLGGPSVFLFPPKPKDTLMISRTPEVTCVVVDVSHEDPEVKFNWYVDGVE | Clone CH3C.35.21.17.5 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVLWESFGTEWA | |
| | SYKTTPPVLDSDGSFFLYSKLTVTKEEWQQGFVFSCSVMHEALHNHYTQKSLS | |
| | LSPGK | |
| 135 | APELLGGPSVFLFPPKPKDTLMISRTPEVTCVVVDVSHEDPEVKFNWYVDGVE | Clone CH3C.35.21.17.6 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVLWESFGTEWV | |
| | SYKTTPPVLDSDGSFFLYSKLTVTKEEWQQGFVFSCSVMHEALHNHYTQKSLS | |
| | LSPGK | |
| 136 | APELLGGPSVFLFPPKPKDTLMISRTPEVTCVVVDVSHEDPEVKFNWYVDGVE | Clones CH3C.35.N390 and |
| | VHNAKTKPREEQYNSTYRVVSVLTVLHQDWLNGKEYKCKVSNKALPAPIEKTI | CH3C.35.N163 |
| | SKAKGQPREPQVYTLPPSRDELTKNQVSLTCLVKGFYPSDIAVEWESYGTEWS | |
| | NYKTTPPVLDSDGSFFLYSKLTVTKSEWQQGFVFSCSVMHEALHNHYTQKSLS | |
| | LSPGK | |
| 137 | APELLGGPSVFLFPPKPKDTLMISRTPEVTCVVVDVSHEDPEVKFNWYVDGVE | Clone CH3C.1 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESLGLVWV | |
| | YKTTPPVLDSDGSFFLYSKLTVAKSTWQQGWVFSCSVMHEALHNHYTQKSLS | |
| | LSPGK | |
| 138 | APELLGGPSVFLFPPKPKDTLMISRTPEVTCVVVDVSHEDPEVKFNWYVDGVE | Clone CH3C.2 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESYGTVWS | |
| | HYKTTPPVLDSDGSFFLYSKLTVSKSEWQQGYVFSCSVMHEALHNHYTQKSLS | |
| | LSPGK | |
| 139 | APELLGGPSVFLFPPKPKDTLMISRTPEVTCVVVDVSHEDPEVKFNWYVDGVE | Clone CH3C.3 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESYGTEWS | |
| | QYKTTPPVLDSDGSFFLYSKLTVEKSDWQQGHVFSCSVMHEALHNHYTQKSLS | |
| | LSPGK | |
| 140 | APELLGGPSVFLFPPKPKDTLMISRTPEVTCVVVDVSHEDPEVKFNWYVDGVE | Clone CH3C.4 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESVGTPWA | |
| | LYKTTPPVLDSDGSFFLYSKLTVLKSEWQQGWVFSCSVMHEALHNHYTQKSLS | |
| | LSPGK | |
| 141 | APELLGGPSVFLFPPKPKDTLMISRTPEVTCVVVDVSHEDPEVKFNWYVDGVE | Clone CH3C.17 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESYGTVWS | |
| | KYKTTPPVLDSDGSFFLYSKLTVSKSEWQQGFVFSCSVMHEALHNHYTQKSLS | |
| | LSPGK | |
| 142 | APELLGGPSVFLFPPKPKDTLMISRTPEVTCVVVDVSHEDPEVKFNWYVDGVE | Clone CH3C.18 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESLGHVWA | |
| | VYKTTPPVLDSDGSFFLYSKLTVPKSTWQQGWVFSCSVMHEALHNHYTQKSLS | |
| | LSPGK | |
| 143 | APELLGGPSVFLFPPKPKDTLMISRTPEVTCVVVDVSHEDPEVKFNWYVDGVE | Clone CH3C.21 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESLGLVWV | |
| | YKTTPPVLDSDGSFFLYSKLTVPKSTWQQGWVFSCSVMHEALHNHYTQKSLS | |
| | LSPGK | |
| 144 | APELLGGPSVFLFPPKPKDTLMISRTPEVTCVVVDVSHEDPEVKFNWYVDGVE | Clone CH3C.25 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESMGHVWV | |
| | GYKTTPPVLDSDGSFFLYSKLTVDKSTWQQGWVFSCSVMHEALHNHYTQKSLS | |
| | LSPGK | |
| 145 | APELLGGPSVFLFPPKPKDTLMISRTPEVTCVVVDVSHEDPEVKFNWYVDGVE | Clone CH3C.34 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESLGLVWV | |
| | FSKTTPPVLDSDGSFFLYSKLTVPKSTWQQGWVFSCSVMHEALHNHYTQKSLS | |
| | LSPGK | |
| 146 | APELLGGPSVFLFPPKPKDTLMISRTPEVTCVVVDVSHEDPEVKFNWYVDGVE | Clone CH3C.35 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESYGTEWS | |
| | SYKTTPPVLDSDGSFFLYSKLTVTKSEWQQGFVFSCSVMHEALHNHYTQKSLS | |
| | LSPGK | |
| 147 | APELLGGPSVFLFPPKPKDTLMISRTPEVTCVVVDVSHEDPEVKFNWYVDGVE | Clone CH3C.44 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESYGTEWS | |
| | NYKTTPPVLDSDGSFFLYSKLTVSKSEWQQGFVFSCSVMHEALHNHYTQKSLS | |
| | LSPGK | |
| 148 | APELLGGPSVFLFPPKPKDTLMISRTPEVTCVVVDVSHEDPEVKFNWYVDGVE | Clone CH3C.51 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESLGHVWV | |
| | GYKTTPPVLDSDGSFFLYSKLTVSKSEWQQGWVFSCSVMHEALHNHYTQKSLS | |
| | LSPGK | |
| 149 | APELLGGPSVFLFPPKPKDTLMISRTPEVTCVVVDVSHEDPEVKFNWYVDGVE | Clone CH3C.3.1-3 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESLGHVWV | |
| | ATKTTPPVLDSDGSFFLYSKLTVPKSTWQQGWVFSCSVMHEALHNHYTQKSLS | |
| | LSPGK | |
| 150 | APELLGGPSVFLFPPKPKDTLMISRTPEVTCVVVDVSHEDPEVKFNWYVDGVE | Clone CH3C.3.1-9 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESLGPVWV | |
| | HTKTTPPVLDSDGSFFLYSKLTVPKSTWQQGWVFSCSVMHEALHNHYTQKSLS | |
| | LSPGK | |
| 151 | APELLGGPSVFLFPPKPKDTLMISRTPEVTCVVVDVSHEDPEVKFNWYVDGVE | Clone CH3C.3.2-5 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESLGHVWV | |
| | DQKTTPPVLDSDGSFFLYSKLTVPKSTWQQGWVFSCSVMHEALHNHYTQKSLS | |
| | LSPGK | |
| 152 | APELLGGPSVFLFPPKPKDTLMISRTPEVTCVVVDVSHEDPEVKFNWYVDGVE | Clone CH3C.3.2-19 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESLGHVWV | |
| | NQKTTPPVLDSDGSFFLYSKLTVPKSTWQQGWVFSCSVMHEALHNHYTQKSLS | |
| | LSPGK | |
| 153 | APELLGGPSVFLFPPKPKDTLMISRTPEVTCVVVDVSHEDPEVKFNWYVDGVE | Clone CH3C.3.2-1 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESLGHVWV | |
| | NFKTTPPVLDSDGSFFLYSKLTVPKSTWQQGWVFSCSVMHEALHNHYTQKSLS | |
| | LSPGK | |
| 154 | APELLGGPSVFLFPPKPKDTLMISRTPEVTCVVVDVSHEDPEVKFNWYVDGVE | Clone CH3C.18 variant |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVWWESLGHVWA | |
| | VYKTTPPVLDSDGSFFLYSKLTVPKSTWQQGWVFSCSVMHEALHNHYTQKSLS | |
| | LSPGK | |
| 155 | APELLGGPSVFLFPPKPKDTLMISRTPEVTCVVVDVSHEDPEVKFNWYVDGVE | Clone CH3C.18 variant |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVLWESLGHVWA | |
| | VYKTTPPVLDSDGSFFLYSKLTVPKSTWQQGWVFSCSVMHEALHNHYTQKSLS | |
| | LSPGK | |
| 156 | APELLGGPSVFLFPPKPKDTLMISRTPEVTCVVVDVSHEDPEVKFNWYVDGVE | Clone CH3C.18 variant |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVYWESLGHVWA | |
| | VYKTTPPVLDSDGSFFLYSKLTVPKSTWQQGWVFSCSVMHEALHNHYTQKSLS | |
| | LSPGK | |
| 157 | APELLGGPSVFLFPPKPKDTLMISRTPEVTCVVVDVSHEDPEVKFNWYVDGVE | Clone CH3C.18 variant |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESLGHVWA | |
| | VYQTTPPVLDSDGSFFLYSKLTVPKSTWQQGWVFSCSVMHEALHNHYTQKSLS | |
| | LSPGK | |
| 158 | APELLGGPSVFLFPPKPKDTLMISRTPEVTCVVVDVSHEDPEVKFNWYVDGVE | Clone CH3C.18 variant |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESLGHVWA | |
| | VYFTTPPVLDSDGSFFLYSKLTVPKSTWQQGWVFSCSVMHEALHNHYTQKSLS | |
| | LSPGK | |
| 159 | APELLGGPSVFLFPPKPKDTLMISRTPEVTCVVVDVSHEDPEVKFNWYVDGVE | Clone CH3C.18 variant |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESLGHVWA | |
| | VYHTTPPVLDSDGSFFLYSKLTVPKSTWQQGWVFSCSVMHEALHNHYTQKSLS | |
| | LSPGK | |
| 160 | APELLGGPSVFLFPPKPKDTLMISRTPEVTCVVVDVSHEDPEVKFNWYVDGVE | Clone CH3C.35.13 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVWWESLGHVWA | |
| | VYKTTPPVLDSDGSFFLYSKLTVPKSTWQQGWVFSCSVMHEALHNHYTQKSLS | |
| | LSPGK | |
| 161 | APELLGGPSVFLFPPKPKDTLMISRTPEVTCVVVDVSHEDPEVKFNWYVDGVE | Clone CH3C.35.14 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESLGHVWA | |
| | VYQTTPPVLDSDGSFFLYSKLTVPKSTWQQGWVFSCSVMHEALHNHYTQKSLS | |
| | LSPGK | |
| 162 | APELLGGPSVFLFPPKPKDTLMISRTPEVTCVVVDVSHEDPEVKFNWYVDGVE | Clone CH3C.35.15 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVWWESLGHVWA | |
| | VYQTTPPVLDSDGSFFLYSKLTVPKSTWQQGWVFSCSVMHEALHNHYTQKSLS | |
| | LSPGK | |
| 163 | APELLGGPSVFLFPPKPKDTLMISRTPEVTCVVVDVSHEDPEVKFNWYVDGVE | Clone CH3C.35.16 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVWWESLGHVWV | |
| | NQKTTPPVLDSDGSFFLYSKLTVPKSTWQQGWVFSCSVMHEALHNHYTQKSLS | |
| | LSPGK | |
| 164 | APELLGGPSVFLFPPKPKDTLMISRTPEVTCVVVDVSHEDPEVKFNWYVDGVE | Clone CH3C.35.17 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESLGHVWV | |
| | NQQTTPPVLDSDGSFFLYSKLTVPKSTWQQGWVFSCSVMHEALHNHYTQKSLS | |
| | LSPGK | |
| 165 | APELLGGPSVFLFPPKPKDTLMISRTPEVTCVVVDVSHEDPEVKFNWYVDGVE | Clone CH3C.35.18 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVWWESLGHVWV | |
| | NQQTTPPVLDSDGSFFLYSKLTVPKSTWQQGWVFSCSVMHEALHNHYTQKSLS | |
| | LSPGK | |
| 166 | APELLGGPSVFLFPPKPKDTLMISRTPEVTCVVVDVSHEDPEVKFNWYVDGVE | Clone CH3C.35.19 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVWWESYGTEWS | |
| | SYKTTPPVLDSDGSFFLYSKLTVTKSEWQQGFVFSCSVMHEALHNHYTQKSLS | |
| | LSPGK | |
| 167 | APELLGGPSVFLFPPKPKDTLMISRTPEVTCVVVDVSHEDPEVKFNWYVDGVE | Clone CH3C.35.K165Q |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESYGTEWS | |
| | SYQTTPPVLDSDGSFFLYSKLTVTKSEWQQGFVFSCSVMHEALHNHYTQKSLS | |
| | LSPGK | |
| 168 | APELLGGPSVFLFPPKPKDTLMISRTPEVTCVVVDVSHEDPEVKFNWYVDGVE | Clone CH3C.35.N163. |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESYGTEWS | K165Q |
| | NYQTTPPVLDSDGSFFLYSKLTVTKSEWQQGFVFSCSVMHEALHNHYTQKSLS | |
| | LSPGK | |
| 169 | APELLGGPSVFLFPPKPKDTLMISRTPEVTCVVVDVSHEDPEVKFNWYVDGVE | Clone CH3C.35.21.1 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVLWESYGTEWS | |
| | SYKTTPPVLDSDGSFFLYSKLTVTKSEWQQGFVFSCSVMHEALHNHYTQKSLS | |
| | LSPGK | |
| 170 | APELLGGPSVFLFPPKPKDTLMISRTPEVTCVVVDVSHEDPEVKFNWYVDGVE | Clone CH3C.35.21.2 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVLWESYGTEWS | |
| | SYRTTPPVLDSDGSFFLYSKLTVTKSEWQQGFVFSCSVMHEALHNHYTQKSLS | |
| | LSPGK | |
| 171 | APELLGGPSVFLFPPKPKDTLMISRTPEVTCVVVDVSHEDPEVKFNWYVDGVE | Clone CH3C.35.21.3 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVLWESYGTEWS | |
| | SYRTTPPVLDSDGSFFLYSKLTVTREEWQQGFVFSCSVMHEALHNHYTQKSLS | |
| | LSPGK | |
| 172 | APELLGGPSVFLFPPKPKDTLMISRTPEVTCVVVDVSHEDPEVKFNWYVDGVE | Clone CH3C.35.21. 4 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVLWESYGTEWS | |
| | SYRTTPPVLDSDGSFFLYSKLTVTGEEWQQGFVFSCSVMHEALHNHYTQKSLS | |
| | LSPGK | |
| 173 | APELLGGPSVFLFPPKPKDTLMISRTPEVTCVVVDVSHEDPEVKFNWYVDGVE | Clone CH3C.35.21. 5 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVLWESYGTEWS | |
| | SYRTTPPVLDSDGSFFLYSKLTVTREEWQQGFVFSCWVMHEALHNHYTQKSLS | |
| | LSPGK | |
| 174 | APELLGGPSVFLFPPKPKDTLMISRTPEVTCVVVDVSHEDPEVKFNWYVDGVE | Clone CH3C.35.21. 6 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVLWESYGTEWS | |
| | SYRTTPPVLDSDGSFFLYSKLTVTKEEWQQGFVFSCWVMHEALHNHYTQKSLS | |
| | LSPGK | |
| 175 | APELLGGPSVFLFPPKPKDTLMISRTPEVTCVVVDVSHEDPEVKFNWYVDGVE | Clone CH3C.35.21. 7 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVLWESYGTEWS | |
| | SYRTTPPVLDSDGSFFLYSKLTVTREEWQQGFVFTCWVMHEALHNHYTQKSLS | |
| | LSPGK | |
| 176 | APELLGGPSVFLFPPKPKDTLMISRTPEVTCVVVDVSHEDPEVKFNWYVDGVE | Clone CH3C.35.21.8 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVLWESYGTEWS | |
| | SYRTTPPVLDSDGSFFLYSKLTVTREEWQQGFVFTCGVMHEALHNHYTQKSLS | |
| | LSPGK | |
| 177 | APELLGGPSVFLFPPKPKDTLMISRTPEVTCVVVDVSHEDPEVKFNWYVDGVE | Clone CH3C.35.21.9 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVLWESYGTEWS | |
| | SYRTTPPVLDSDGSFFLYSKLTVTREEWQQGFVFECWVMHEALHNHYTQKSLS | |
| | LSPGK | |
| 178 | APELLGGPSVFLFPPKPKDTLMISRTPEVTCVVVDVSHEDPEVKFNWYVDGVE | Clone CH3C.35.21.10 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVLWESYGTEWS | |
| | SYRTTPPVLDSDGSFFLYSKLTVTREEWQQGFVFKCWVMHEALHNHYTQKSLS | |
| | LSPGK | |
| 179 | APELLGGPSVFLFPPKPKDTLMISRTPEVTCVVVDVSHEDPEVKFNWYVDGVE | Clone CH3C.35.21.11 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVLWESYGTEWS | |
| | SYRTTPPVLDSDGSFFLYSKLTVTPEEWQQGFVFKCWVMHEALHNHYTQKSLS | |
| | LSPGK | |
| 180 | APELLGGPSVFLFPPKPKDTLMISRTPEVTCVVVDVSHEDPEVKFNWYVDGVE | Clone CH3C.35.21.12 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVWWESYGTEWS | |
| | SYRTTPPVLDSDGSFFLYSKLTVTREEWQQGFVFSCSVMHEALHNHYTQKSLS | |
| | LSPGK | |
| 181 | APELLGGPSVFLFPPKPKDTLMISRTPEVTCVVVDVSHEDPEVKFNWYVDGVE | Clone CH3C.35.21.13 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVWWESYGTEWS | |
| | SYRTTPPVLDSDGSFFLYSKLTVTGEEWQQGFVFSCSVMHEALHNHYTQKSLS | |
| | LSPGK | |
| 182 | APELLGGPSVFLFPPKPKDTLMISRTPEVTCVVVDVSHEDPEVKFNWYVDGVE | Clone CH3C.35.21.14 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVWWESYGTEWS | |
| | SYRTTPPVLDSDGSFFLYSKLTVTREEWQQGFVFTCWVMHEALHNHYTQKSLS | |
| | LSPGK | |
| 183 | APELLGGPSVFLFPPKPKDTLMISRTPEVTCVVVDVSHEDPEVKFNWYVDGVE | Clone CH3C.35.21.15 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVWWESYGTEWS | |
| | SYRTTPPVLDSDGSFFLYSKLTVTGEEWQQGFVFTCWVMHEALHNHYTQKSLS | |
| | LSPGK | |
| 184 | APELLGGPSVFLFPPKPKDTLMISRTPEVTCVVVDVSHEDPEVKFNWYVDGVE | Clone CH3C.35.21.16 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVWWESYGTEWS | |
| | SYRTTPPVLDSDGSFFLYSKLTVTREEWQQGFVFTCGVMHEALHNHYTQKSLS | |
| | LSPGK | |
| 185 | APELLGGPSVFLFPPKPKDTLMISRTPEVTCVVVDVSHEDPEVKFNWYVDGVE | Clone CH3C.35.21.18 |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVLWESYGTEWS | |
| | SYRTTPPVLDSDGSFFLYSKLTVTKEEWQQGFVFSCSVMHEALHNHYTQKSLS | |
| | LSPGK | |
| 186 | APELLGGPSVFLFPPKPKDTLMISRTPEVTCVVVDVSHEDPEVKFNWYVDGVE | Clone CH3B.1 |
| | VHNAKTKPREEQYNSTYRVVSVLTVLHQDWLNGKEYKCKVSNKALPAPIEKTI | |
| | SKAKGQPRFDYVTTLPPSRDELTKNQVSLTCLVKGFYPSDIAVEWESNGQPEN | |
| | NYKTTPPVLDSDGSFFLYSKLTVDKSRWQQGNVFSCSVMHEALHNHYGFHDLS | |
| | LSPGK | |
| 187 | APELLGGPSVFLFPPKPKDTLMISRTPEVTCVVVDVSHEDPEVKFNWYVDGVE | Clone CH3B.2 |
| | VHNAKTKPREEQYNSTYRVVSVLTVLHQDWLNGKEYKCKVSNKALPAPIEKTI | |
| | SKAKGQPRFDMVTTLPPSRDELTKNQVSLTCLVKGFYPSDIAVEWESNGQPEN | |
| | NYKTTPPVLDSDGSFFLYSKLTVDKSRWQQGNVFSCSVMHEALHNHYGFHDLS | |
| | LSPGK | |
| 188 | APELLGGPSVFLFPPKPKDTLMISRTPEVTCVVVDVSHEDPEVKFNWYVDGVE | Clone CH3B.3 |
| | VHNAKTKPREEQYNSTYRVVSVLTVLHQDWLNGKEYKCKVSNKALPAPIEKTI | |
| | SKAKGQPRFEYVTTLPPSRDELTKNQVSLTCLVKGFYPSDIAVEWESNGQPEN | |
| | NYKTTPPVLDSDGSFFLYSKLTVDKSRWQQGNVFSCSVMHEALHNHYGFHDLS | |
| | LSPGK | |
| 189 | APELLGGPSVFLFPPKPKDTLMISRTPEVTCVVVDVSHEDPEVKFNWYVDGVE | Clone CH3B.4 |
| | VHNAKTKPREEQYNSTYRVVSVLTVLHQDWLNGKEYKCKVSNKALPAPIEKTI | |
| | SKAKGQPRFEMVTTLPPSRDELTKNQVSLTCLVKGFYPSDIAVEWESNGQPEN | |
| | NYKTTPPVLDSDGSFFLYSKLTVDKSRWQQGNVFSCSVMHEALHNHYGFHDLS | |
| | LSPGK | |
| 190 | APELLGGPSVFLFPPKPKDTLMISRTPEVTCVVVDVSHEDPEVKFNWYVDGVE | Clone CH3B.5 |
| | VHNAKTKPREEQYNSTYRVVSVLTVLHQDWLNGKEYKCKVSNKALPAPIEKTI | |
| | SKAKGQPRFELVTTLPPSRDELTKNQVSLTCLVKGFYPSDIAVEWESNGQPEN | |
| | NYKTTPPVLDSDGSFFLYSKLTVDKSRWQQGNVFSCSVMHEALHNHYGFHDLS | |
| | LSPGK | |
| 191 | APELLGGPSVFLFPPKPKDTLMISRTPEVTCVVVDVSHEDPEVEFIWYVDGVD | Clone CH2A2.1 |
| | VRYEWQLPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESNGQPEN | |
| | NYKTTPPVLDSDGSFFLYSKLTVDKSRWQQGNVFSCSVMHEALHNHYTQKSLS | |
| | LSPGK | |
| 192 | APELLGGPSVFLFPPKPKDTLMISRTPEVTCVVVDVSHEDPEVGFVWYVDGVP | Clone CH2A2.2 |
| | VSWEWYWPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESNGQPEN | |
| | NYKTTPPVLDSDGSFFLYSKLTVDKSRWQQGNVFSCSVMHEALHNHYTQKSLS | |
| | LSPGK | |
| 193 | APELLGGPSVFLFPPKPKDTLMISRTPEVTCVVVDVSHEDPEVQFDWYVDGVM | Clone CH2A2.3 |
| | VRREWHRPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESNGQPEN | |
| | NYKTTPPVLDSDGSFFLYSKLTVDKSRWQQGNVFSCSVMHEALHNHYTQKSLS | |
| | LSPGK | |
| 194 | APELLGGPSVFLFPPKPKDTLMISRTPEVTCVVVDVSHEDPEVSFEWYVDGVP | Clone CH2A2.4 |
| | VRWEWQWPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESNGQPEN | |
| | NYKTTPPVLDSDGSFFLYSKLTVDKSRWQQGNVFSCSVMHEALHNHYTQKSLS | |
| | LSPGK | |
| 195 | APELLGGPSVFLFPPKPKDTLMISRTPEVTCVVVDVSHEDPEVAFTWYVDGVP | Clone CH2A2.5 |
| | VRWEWQNPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESNGQPEN | |
| | NYKTTPPVLDSDGSFFLYSKLTVDKSRWQQGNVFSCSVMHEALHNHYTQKSLS | |
| | LSPGK | |
| 196 | APELLGGPSVFLFPPKPKDTLMISRTPEVTCVVVDPQTPPWEVKFNWYVDGVE | Clone CH2C.1 |
| | VHNAKTKPREEEYYTYYRVVSVLTVLHQDWLNGKEYKCKVSNKALPAPIEKTI | |
| | SKAKGQPREPQVYTLPPSRDELTKNQVSLTCLVKGFYPSDIAVEWESNGQPEN | |
| | NYKTTPPVLDSDGSFFLYSKLTVDKSRWQQGNVFSCSVMHEALHNHYTQKSLS | |
| | LSPGK | |
| 197 | APELLGGPSVFLFPPKPKDTLMISRTPEVTCVVVDPPSPPWEVKFNWYVDGVE | Clone CH2C.2 |
| | VHNAKTKPREEEYYSNYRVVSVLTVLHQDWLNGKEYKCKVSNKALPAPIEKTI | |
| | SKAKGQPREPQVYTLPPSRDELTKNQVSLTCLVKGFYPSDIAVEWESNGQPEN | |
| | NYKTTPPVLDSDGSFFLYSKLTVDKSRWQQGNVFSCSVMHEALHNHYTQKSLS | |
| | LSPGK | |
| 198 | APELLGGPSVFLFPPKPKDTLMISRTPEVTCVVVDPQTPPWEVKFNWYVDGVE | Clone CH2C.3 |
| | VHNAKTKPREEEYYSNYRVVSVLTVLHQDWLNGKEYKCKVSNKALPAPIEKTI | |
| | SKAKGQPREPQVYTLPPSRDELTKNQVSLTCLVKGFYPSDIAVEWESNGQPEN | |
| | NYKTTPPVLDSDGSFFLYSKLTVDKSRWQQGNVFSCSVMHEALHNHYTQKSLS | |
| | LSPGK | |
| 199 | APELLGGPSVFLFPPKPKDTLMISRTPEVTCVVVDFRGPPWEVKFNWYVDGVE | Clone CH2C.4 |
| | VHNAKTKPREEEYYHDYRVVSVLTVLHQDWLNGKEYKCKVSNKALPAPIEKTI | |
| | SKAKGQPREPQVYTLPPSRDELTKNQVSLTCLVKGFYPSDIAVEWESNGQPEN | |
| | NYKTTPPVLDSDGSFFLYSKLTVDKSRWQQGNVFSCSVMHEALHNHYTQKSLS | |
| | LSPGK | |
| 200 | APELLGGPSVFLFPPKPKDTLMISRTPEVTCVVVDPQTVPWEVKFNWYVDGVE | Clone CH2C.5 |
| | VHNAKTKPREEEYYSNYRVVSVLTVLHQDWLNGKEYKCKVSNKALPAPIEKTI | |
| | SKAKGQPREPQVYTLPPSRDELTKNQSLTCLVKGFYPSDIAVEWESNGQPEN | |
| | NYKTTPPVLDSDGSFFLYSKLTVDKSRWQQGNVFSCSVMHEALHNHYTQKSLS | |
| | LSPGK | |
| 201 | APELLGGPSVFLFPPKPKDTLMISRTPEVTCVVVDVSVPPRMVKFNWYVDGVE | Clone CH2D.1 |
| | VHNAKTKSLTSQHNSTVRVVSVLTVLHQDWLNGKEYKCKVSNKALPAPIEKTI | |
| | SKAKGQPREPQVYTLPPSRDELTKNQVSLTCLVKGFYPSDIAVEWESNGQPEN | |
| | NYKTTPPVLDSDGSFFLYSKLTVDKSRWQQGNVFSCSVMHEALHNHYTQKSLS | |
| | LSPGK | |
| 202 | APELLGGPSVFLFPPKPKDTLMISRTPEVTCVVVDVSVPPWMVKFNWYVDGVE | Clone CH2D.2 |
| | VHNAKTKSLTSQHNSTVRVVSVLTVLHQDWLNGKEYKCKVSNKALPAPIEKTI | |
| | SKAKGQPREPQVYTLPPSRDELTKNQVSLTCLVKGFYPSDIAVEWESNGQPEN | |
| | NYKTTPPVLDSDGSFFLYSKLTVDKSRWQQGNVFSCSVMHEALHNHYTQKSLS | |
| | LSPGK | |
| 203 | APELLGGPSVFLFPPKPKDTLMISRTPEVTCVVVDVSDMWEYVKFNWYVDGVE | Clone CH2D.3 |
| | VHNAKTKPWVKQLNSTWRVVSVLTVLHQDWLNG KEYKCKVSNKALPAPIEKTI | |
| | SKAKGQPREPQVYTLPPSRDELTKNQVSLTCLVKGFYPSDIAVEWESNGQPEN | |
| | NYKTTPPVLDSDGSFFLYSKLTVDKSRWQQGNVFSCSVMHEALHNHYTQKSLS | |
| | LSPGK | |
| 204 | APELLGGPSVFLFPPKPKDTLMISRTPEVTCVVVDVSDDWTWVKFNWYVDGVE | Clone CH2D.4 |
| | VHNAKTKPWIAQPNSTWRVVSVLTVLHQDWLNGKEYKCKVSNKALPAPIEKTI | |
| | SKAKGQPREPQVYTLPPSRDELTKNQVSLTCLVKGFYPSDIAVEWESNGQPEN | |
| | NYKTTPPVLDSDGSFFLYSKLTVDKSRWQQGNVFSCSVMHEALHNHYTQKSLS | |
| | LSPGK | |
| 205 | APELLGGPSVFLFPPKPKDTLMISRTPEVTCVVVDVSDDWEWVKFNWYVDGVE | Clone CH2D.5 |
| | VHNAKTKPWKLQLNSTWRVVSVLTVLHQDWLNGKEYKCKVSNKALPAPIEKTI | |
| | SKAKGQPREPQVYTLPPSRDELTKNQVSLTCLVKGFYPSDIAVEWESNGQPEN | |
| | NYKTTPPVLDSDGSFFLYSKLTVDKSRWQQGNVFSCSVMHEALHNHYTQKSLS | |
| | LSPGK | |
| 206 | APELLGGPSVFLFPPKPKDTLMISRTPEVTCVVVDVSHEDPWVWFYWYVDGVE | Clone CH2E3.1 |
| | VHNAKTKPREEQYNSTYRVVSVLTVLHQDWLNGKEYKCSVVNIALWWSIEKTI | |
| | SKAKGQPREPQVYTLPPSRDELTKNQVSLTCLVKGFYPSDIAVEWESNGQPEN | |
| | NYKTTPPVLDSDGSFFLYSKLTVDKSRWQQGNVFSCSVMHEALHNHYTQKSLS | |
| | LSPGK | |
| 207 | APELLGGPSVFLFPPKPKDTLMISRTPEVTCVVVDVSHEDPVVGFRWYVDGVE | Clone CH2E3.2 |
| | VHNAKTKPREEQYNSTYRVVSVLTVLHQDWLNGKEYKCRVSNSALTWKIEKTI | |
| | SKAKGQPREPQVYTLPPSRDELTKNQVSLTCLVKGFYPSDIAVEWESNGQPEN | |
| | NYKTTPPVLDSDGSFFLYSKLTVDKSRWQQGNVFSCSVMHEALHNHYTQKSLS | |
| | LSPGK | |
| 208 | APELLGGPSVFLFPPKPKDTLMISRTPEVTCVVVDVSHEDPVVGFRWYVDGVE | Clone CH2E3.3 |
| | VHNAKTKPREEQYNSTYRVVSVLTVLHQDWLNGKEYKCRVSNSALSWRIEKTI | |
| | SKAKGQPREPQVYTLPPSRDELTKNQVSLTCLVKGFYPSDIAVEWESNGQPEN | |
| | NYKTTPPVLDSDGSFFLYSKLTVDKSRWQQGNVFSCSVMHEALHNHYTQKSLS | |
| | LSPGK | |
| 209 | APELLGGPSVFLFPPKPKDTLMISRTPEVTCVVVDVSHEDPIVGFRWYVDGVE | Clone CH2E3.4 |
| | VHNAKTKPREEQYNSTYRVVSVLTVLHQDWLNGKEYKCRVSNSALRWRIEKTI | |
| | SKAKGQPREPQVYTLPPSRDELTKNQVSLTCLVKGFYPSDIAVEWESNGQPEN | |
| | NYKTTPPVLDSDGSFFLYSKLTVDKSRWQQGNVFSCSVMHEALHNHYTQKSLS | |
| | LSPGK | |
| 210 | APELLGGPSVFLFPPKPKDTLMISRTPEVTCVVVDVSHEDPAVGFEWYVDGVE | Clone CH2E3.5 |
| | VHNAKTKPREEQYNSTYRVVSVLTVLHQDWLNGKEYKCQVFNWALDWVIEKTI | |
| | SKAKGQPREPQVYTLPPSRDELTKNQVSLTCLVKGFYPSDIAVEWESNGQPEN | |
| | NYKTTPPVLDSDGSFFLYSKLTVDKSRWQQGNVFSCSVMHEALHNHYTQKSLS | |
| | LSPGK | |
| 211 | APELLGGPSVFLFPPKPKDTLMISRTPEVTCVVVDVSHEDPEVKFNWYVDGVE | Fc sequence with hole |
| | VHNAKTKPREEQYNSTYRVVSVLTVLHQDWLNGKEYKCKVSNKALPAPIEKTI | mutations |
| | SKAKGQPREPQVYTLPPSRDELTKNQVSLSCAVKGFYPSDIAVEWESNGQPEN | |
| | NYKTTPPVLDSDGSFFLVSKLTVDKSRWQQGNVFSCSVMHEALHNHYTQKSLS | |
| | LSPGK | |
| 212 | APEAAGGPSVFLFPPKPKDTLMISRTPEVTCVVVDVSHEDPEVKFNWYVDGVE | Fc sequence with hole and |
| | VHNAKTKPREEQYNSTYRVVSVLTVLHQDWLNGKEYKCKVSNKALPAPIEKTI | LALA mutations |
| | SKAKGQPREPQVYTLPPSRDELTKNQVSLSCAVKGFYPSDIAVEWESNGQPEN | |
| | NYKTTPPVLDSDGSFFLVSKLTVDKSRWQQGNVFSCSVMHEALHNHYTQKSLS | |
| | LSPGK | |
| 213 | APELLGGPSVFLFPPKPKDTLYITREPEVTCVVVDVSHEDPEVKFNWYVDGVE | Fc sequence with hole and |
| | VHNAKTKPREEQYNSTYRVVSVLTVLHQDWLNGKEYKCKVSNKALPAPIEKTI | YTE mutations |
| | SKAKGQPREPQVYTLPPSRDELTKNQVSLSCAVKGFYPSDIAVEWESNGQPEN | |
| | NYKTTPPVLDSDGSFFLVSKLTVDKSRWQQGNVFSCSVMHEALHNHYTQKSLS | |
| | LSPGK | |
| 214 | APEAAGGPSVFLFPPKPKDTLYITREPEVTCVVVDVSHEDPEVKFNWYVDGVE | Fc sequence with hole, |
| | VHNAKTKPREEQYNSTYRVVSVLTVLHQDWLNGKEYKCKVSNKALPAPIEKTI | LALA, and YTE mutations |
| | SKAKGQPREPQVYTLPPSRDELTKNQVSLSCAVKGFYPSDIAVEWESNGQPEN | |
| | NYKTTPPVLDSDGSFFLVSKLTVDKSRWQQGNVFSCSVMHEALHNHYTQKSLS | |
| | LSPGK | |
| 215 | APELLGGPSVFLFPPKPKDTLMISRTPEVTCVVVDVSHEDPEVKFNWYVDGVE | Fc sequence with knob |
| | VHNAKTKPREEQYNSTYRVVSVLTVLHQDWLNGKEYKCKVSNKALPAPIEKTI | mutation |
| | SKAKGQPREPQVYTLPPSRDELTKNQVSLWCLVKGFYPSDIAVEWESNGQPEN | |
| | NYKTTPPVLDSDGSFFLYSKLTVDKSRWQQGNVFSCSVMHEALHNHYTQKSLS | |
| | LSPGK | |
| 216 | APEAAGGPSVFLFPPKPKDTLMISRTPEVTCVVVDVSHEDPEVKFNWYVDGVE | Fc sequence with knob and |
| | VHNAKTKPREEQYNSTYRVVSVLTVLHQDWLNGKEYKCKVSNKALPAPIEKTI | LALA mutations |
| | SKAKGQPREPQVYTLPPSRDELTKNQVSLWCLVKGFYPSDIAVEWESNGQPEN | |
| | NYKTTPPVLDSDGSFFLYSKLTVDKSRWQQGNVFSCSVMHEALHNHYTQKSLS | |
| | LSPGK | |
| 217 | APELLGGPSVFLFPPKPKDTLYITREPEVTCVVVDVSHEDPEVKFNWYVDGVE | Fc sequence with knob and |
| | VHNAKTKPREEQYNSTYRVVSVLTVLHQDWLNGKEYKCKVSNKALPAPIEKTI | YTE mutations |
| | SKAKGQPREPQVYTLPPSRDELTKNQVSLWCLVKGFYPSDIAVEWESNGQPEN | |
| | NYKTTPPVLDSDGSFFLYSKLTVDKSRWQQGNVFSCSVMHEALHNHYTQKSLS | |
| | LSPGK | |
| 218 | APEAAGGPSVFLFPPKPKDTLYITREPEVTCVVVDVSHEDPEVKFNWYVDGVE | Fc sequence with knob, |
| | VHNAKTKPREEQYNSTYRVVSVLTVLHQDWLNGKEYKCKVSNKALPAPIEKTI | LALA, and YTE mutations |
| | SKAKGQPREPQVYTLPPSRDELTKNQVSLWCLVKGFYPSDIAVEWESNGQPEN | |
| | NYKTTPPVLDSDGSFFLYSKLTVDKSRWQQGNVFSCSVMHEALHNHYTQKSLS | |
| | LSPGK | |
| 219 | APELLGGPSVFLFPPKPKDTLMISRTPEVTCVVVDVSHEDPEVKFNWYVDGVE | Clone CH3C.35.21 with knob |
| | VHNAKTKPREEQYNSTYRVVSVLTVLHQDWLNGKEYKCKVSNKALPAPIEKTI | mutation |
| | SKAKGQPREPQVYTLPPSRDELTKNQVSLWCLVKGFYPSDIAVWWESYGTEWS | |
| | SYKTTPPVLDSDGSFFLYSKLTVTKEEWQQGFVFSCSVMHEALHNHYTQKSLS | |
| | LSPGK | |
| 220 | APEAAGGPSVFLFPPKPKDTLMISRTPEVTCVVVDVSHEDPEVKFNWYVDGVE | Clone CH3C.35.21 with knob |
| | VHNAKTKPREEQYNSTYRVVSVLTVLHQDWLNGKEYKCKVSNKALPAPIEKTI | and LALA mutations |
| | SKAKGQPREPQVYTLPPSRDELTKNQVSLWCLVKGFYPSDIAVWWESYGTEWS | |
| | SYKTTPPVLDSDGSFFLYSKLTVTKEEWQQGFVFSCSVMHEALHNHYTQKSLS | |
| | LSPGK | |
| 221 | APELLGGPSVFLFPPKPKDTLYITREPEVTCVVVDVSHEDPEVKFNWYVDGVE | Clone CH3C.35.21 with knob |
| | VHNAKTKPREEQYNSTYRVVSVLTVLHQDWLNGKEYKCKVSNKALPAPIEKTI | and YTE mutations |
| | SKAKGQPREPQVYTLPPSRDELTKNQVSLWCLVKGFYPSDIAVWWESYGTEWS | |
| | SYKTTPPVLDSDGSFFLYSKLTVTKEEWQQGFVFSCSVMHEALHNHYTQKSLS | |
| | LSPGK | |
| 222 | APEAAGGPSVFLFPPKPKDTLYITREPEVTCVVVDVSHEDPEVKFNWYVDGVE | Clone CH3C.35.21 with |
| | VHNAKTKPREEQYNSTYRVVSVLTVLHQDWLNGKEYKCKVSNKALPAPIEKTI | knob, LALA, and YTE |
| | SKAKGQPREPQVYTLPPSRDELTKNQVSLWCLVKGFYPSDIAVWWESYGTEWS | mutations |
| | SYKTTPPVLDSDGSFFLYSKLTVTKEEWQQGFVFSCSVMHEALHNHYTQKSLS | |
| | LSPGK | |
| 223 | APELLGGPSVFLFPPKPKDTLMISRTPEVTCVVVDVSHEDPEVKFNWYVDGVE | Clone CH3C.35.21 with hole |
| | VHNAKTKPREEQYNSTYRVVSVLTVLHQDWLNGKEYKCKVSNKALPAPIEKTI | mutations |
| | SKAKGQPREPQVYTLPPSRDELTKNQVSLSCAVKGFYPSDIAVWWESYGTEWS | |
| | SYKTTPPVLDSDGSFFLVSKLTVTKEEWQQGFVFSCSVMHEALHNHYTQKSLS | |
| | LSPGK | |
| 224 | APEAAGGPSVFLFPPKPKDTLMISRTPEVTCVVVDVSHEDPEVKFNWYVDGVE | Clone CH3C.35.21 with hole |
| | VHNAKTKPREEQYNSTYRVVSVLTVLHQDWLNGKEYKCKVSNKALPAPIEKTI | and LALA mutations |
| | SKAKGQPREPQVYTLPPSRDELTKNQVSLSCAVKGFYPSDIAVWWESYGTEWS | |
| | SYKTTPPVLDSDGSFFLVSKLTVTKEEWQQGFVFSCSVMHEALHNHYTQKSLS | |
| | LSPGK | |
| 225 | APELLGGPSVFLFPPKPKDTLYITREPEVTCVVVDVSHEDPEVKFNWYVDGVE | Clone CH3C.35.21 with hole |
| | VHNAKTKPREEQYNSTYRVVSVLTVLHQDWLNGKEYKCKVSNKALPAPIEKTI | and YTE mutations |
| | SKAKGQPREPQVYTLPPSRDELTKNQVSLSCAVKGFYPSDIAVWWESYGTEWS | |
| | SYKTTPPVLDSDGSFFLVSKLTVTKEEWQQGFVFSCSVMHEALHNHYTQKSLS | |
| | LSPGK | |
| 226 | APEAAGGPSVFLFPPKPKDTLYITREPEVTCVVVDVSHEDPEVKFNWYVDGVE | Clone CH3C.35.21 with hole, |
| | VHNAKTKPREEQYNSTYRVVSVLTVLHQDWLNGKEYKCKVSNKALPAPIEKTI | LALA, and YTE mutations |
| | SKAKGQPREPQVYTLPPSRDELTKNQVSLSCAVKGFYPSDIAVWWESYGTEWS | |
| | SYKTTPPVLDSDGSFFLVSKLTVTKEEWQQGFVFSCSVMHEALHNHYTQKSLS | |
| | LSPGK | |
| 227 | APELLGGPSVFLFPPKPKDTLMISRTPEVTCVVVDVSHEDPEVKFNWYVDGVE | Clone CH3C.35.20.1 with |
| | VHNAKTKPREEQYNSTYRVVSVLTVLHQDWLNGKEYKCKVSNKALPAPIEKTI | knob mutation |
| | SKAKGQPREPQVYTLPPSRDELTKNQVSLWCLVKGFYPSDIAVEWESFGTEWS | |
| | SYKTTPPVLDSDGSFFLYSKLTVTKEEWQQGFVFSCSVMHEALHNHYTQKSLS | |
| | LSPGK | |
| 228 | APEAAGGPSVFLFPPKPKDTLMISRTPEVTCVVVDVSHEDPEVKFNWYVDGVE | Clone CH3C.35.20.1 with |
| | VHNAKTKPREEQYNSTYRVVSVLTVLHQDWLNGKEYKCKVSNKALPAPIEKTI | knob and LALA mutations |
| | SKAKGQPREPQVYTLPPSRDELTKNQVSLWCLVKGFYPSDIAVEWESFGTEWS | |
| | SYKTTPPVLDSDGSFFLYSKLTVTKEEWQQGFVFSCSVMHEALHNHYTQKSLS | |
| | LSPGK | |
| 229 | APEAAGGPSVFLFPPKPKDTLMISRTPEVTCVVVDVSHEDPEVKFNWYVDGVE | Clone CH3C.35.20.1 with |
| | VHNAKTKPREEQYNSTYRVVSVLTVLHQDWLNGKEYKCKVSNKALGAPIEKTI | knob and LALAPG |
| | SKAKGQPREPQVYTLPPSRDELTKNQVSLWCLVKGFYPSDIAVEWESFGTEWS | mutations |
| | SYKTTPPVLDSDGSFFLYSKLTVTKEEWQQGFVFSCSVMHEALHNHYTQKSLS | |
| | LSPGK | |
| 230 | APELLGGPSVFLFPPKPKDTLYITREPEVTCVVVDVSHEDPEVKFNWYVDGVE | Clone CH3C.35.20.1 with |
| | VHNAKTKPREEQYNSTYRVVSVLTVLHQDWLNGKEYKCKVSNKALPAPIEKTI | knob and YTE mutations |
| | SKAKGQPREPQVYTLPPSRDELTKNQVSLWCLVKGFYPSDIAVEWESFGTEWS | |
| | SYKTTPPVLDSDGSFFLYSKLTVTKEEWQQGFVFSCSVMHEALHNHYTQKSLS | |
| | LSPGK | |
| 231 | APEAAGGPSVFLFPPKPKDTLYITREPEVTCVVVDVSHEDPEVKFNWYVDGVE | Clone CH3C.35.20.1 with |
| | VHNAKTKPREEQYNSTYRVVSVLTVLHQDWLNGKEYKCKVSNKALPAPIEKTI | knob, LALA, and YTE |
| | SKAKGQPREPQVYTLPPSRDELTKNQVSLWCLVKGFYPSDIAVEWESFGTEWS | mutations |
| | SYKTTPPVLDSDGSFFLYSKLTVTKEEWQQGFVFSCSVMHEALHNHYTQKSLS | |
| | LSPGK | |
| 232 | APEAAGGPSVFLFPPKPKDTLYITREPEVTCVVVDVSHEDPEVKFNWYVDGVE | Clone CH3C.35.20.1 with |
| | VHNAKTKPREEQYNSTYRVVSVLTVLHQDWLNGKEYKCKVSNKALGAPIEKTI | knob, LALAPG, and YTE |
| | SKAKGQPREPQVYTLPPSRDELTKNQVSLWCLVKGFYPSDIAVEWESFGTEWS | mutations |
| | SYKTTPPVLDSDGSFFLYSKLTVTKEEWQQGFVFSCSVMHEALHNHYTQKSLS | |
| | LSPGK | |
| 233 | APELLGGPSVFLFPPKPKDTLMISRTPEVTCVVVDVSHEDPEVKFNWYVDGVE | Clone CH3C.35.20.1 with |
| | VHNAKTKPREEQYNSTYRVVSVLTVLHQDWLNGKEYKCKVSNKALPAPIEKTI | hole mutations |
| | SKAKGQPREPQVYTLPPSRDELTKNQVSLSCAVKGFYPSDIAVEWESFGTEWS | |
| | SYKTTPPVLDSDGSFFLVSKLTVTKEEWQQGFVFSCSVMHEALHNHYTQKSLS | |
| | LSPGK | |
| 234 | APEAAGGPSVFLFPPKPKDTLMISRTPEVTCVVVDVSHEDPEVKFNWYVDGVE | Clone CH3C.35.20.1 with |
| | VHNAKTKPREEQYNSTYRVVSVLTVLHQDWLNGKEYKCKVSNKALPAPIEKTI | hole and LALA mutations |
| | SKAKGQPREPQVYTLPPSRDELTKNQVSLSCAVKGFYPSDIAVEWESFGTEWS | |
| | SYKTTPPVLDSDGSFFLVSKLTVTKEEWQQGFVFSCSVMHEALHNHYTQKSLS | |
| | LSPGK | |
| 235 | APEAAGGPSVFLFPPKPKDTLMISRTPEVTCVVVDVSHEDPEVKFNWYVDGVE | Clone CH3C.35.20.1 with |
| | VHNAKTKPREEQYNSTYRVVSVLTVLHQDWLNGKEYKCKVSNKALGAPIEKTI | hole and LALAPG mutations |
| | SKAKGQPREPQVYTLPPSRDELTKNQVSLSCAVKGFYPSDIAVEWESFGTEWS | |
| | SYKTTPPVLDSDGSFFLVSKLTVTKEEWQQGFVFSCSVMHEALHNHYTQKSLS | |
| | LSPGK | |
| 236 | APELLGGPSVFLFPPKPKDTLYITREPEVTCVVVDVSHEDPEVKFNWYVDGVE | Clone CH3C.35.20.1 with |
| | VHNAKTKPREEQYNSTYRVVSVLTVLHQDWLNGKEYKCKVSNKALPAPIEKTI | hole and YTE mutations |
| | SKAKGQPREPQVYTLPPSRDELTKNQVSLSCAVKGFYPSDIAVEWESFGTEWS | |
| | SYKTTPPVLDSDGSFFLVSKLTVTKEEWQQGFVFSCSVMHEALHNHYTQKSLS | |
| | LSPGK | |
| 237 | APEAAGGPSVFLFPPKPKDTLYITREPEVTCVVVDVSHEDPEVKFNWYVDGVE | Clone CH3C.35.20.1 with |
| | VHNAKTKPREEQYNSTYRVVSVLTVLHQDWLNGKEYKCKVSNKALPAPIEKTI | hole, LALA, and Y1E |
| | SKAKGQPREPQVYTLPPSRDELTKNQVSLSCAVKGFYPSDIAVEWESFGTEWS | mutations |
| | SYKTTPPVLDSDGSFFLVSKLTVTKEEWQQGFVFSCSVMHEALHNHYTQKSLS | |
| | LSPGK | |
| 238 | APEAAGGPSVFLFPPKPKDTLYITREPEVTCVVVDVSHEDPEVKFNWYVDGVE | Clone CH3C.35.20.1 with |
| | VHNAKTKPREEQYNSTYRVVSVLTVLHQDWLNGKEYKCKVSNKALGAPIEKTI | hole, LALAPG, and YTE |
| | SKAKGQPREPQVYTLPPSRDELTKNQVSLSCAVKGFYPSDIAVEWESFGTEWS | mutations |
| | SYKTTPPVLDSDGSFFLVSKLTVTKEEWQQGFVFSCSVMHEALHNHYTQKSLS | |
| | LSPGK | |
| 239 | APELLGGPSVFLFPPKPKDTLMISRTPEVTCVVVDVSHEDPEVKFNWYVDGVE | Clone CH3C.35.23.2 with |
| | VHNAKTKPREEQYNSTYRVVSVLTVLHQDWLNGKEYKCKVSNKALPAPIEKTI | knob mutation |
| | SKAKGQPREPQVYTLPPSRDELTKNQVSLWCLVKGFYPSDIAVEWESYGTEWA | |
| | NYKTTPPVLDSDGSFFLYSKLTVTKEEWQQGFVFSCSVMHEALHNHYTQKSLS | |
| | LSPGK | |
| 240 | APEAAGGPSVFLFPPKPKDTLMISRTPEVTCVVVDVSHEDPEVKFNWYVDGVE | Clone CH3C.35.23.2 with |
| | VHNAKTKPREEQYNSTYRVVSVLTVLHQDWLNGKEYKCKVSNKALPAPIEKTI | knob and LALA mutations |
| | SKAKGQPREPQVYTLPPSRDELTKNQVSLWCLVKGFYPSDIAVEWESYGTEWA | |
| | NYKTTPPVLDSDGSFFLYSKLTVTKEEWQQGFVFSCSVMHEALHNHYTQKSLS | |
| | LSPGK | |
| 241 | APEAAGGPSVFLFPPKPKDTLMISRTPEVTCVVVDVSHEDPEVKFNWYVDGVE | Clone CH3C.35.23.2 with |
| | VHNAKTKPREEQYNSTYRVVSVLTVLHQDWLNGKEYKCKVSNKALGAPIEKTI | knob and LALAPG |
| | SKAKGQPREPQVYTLPPSRDELTKNQVSLWCLVKGFYPSDIAVEWESYGTEWA | mutations |
| | NYKTTPPVLDSDGSFFLYSKLTVTKEEWQQGFVFSCSVMHEALHNHYTQKSLS | |
| | LSPGK | |
| 242 | APELLGGPSVFLFPPKPKDTLYITREPEVTCVVVDVSHEDPEVKFNWYVDGVE | Clone CH3C.35.23.2 with |
| | VHNAKTKPREEQYNSTYRVVSVLTVLHQDWLNGKEYKCKVSNKALPAPIEKTI | knob and YTE mutations |
| | SKAKGQPREPQVYTLPPSRDELTKNQVSLWCLVKGFYPSDIAVEWESYGTEWA | |
| | NYKTTPPVLDSDGSFFLYSKLTVTKEEWQQGFVFSCSVMHEALHNHYTQKSLS | |
| | LSPGK | |
| 243 | APEAAGGPSVFLFPPKPKDTLYITREPEVTCVVVDVSHEDPEVKFNWYVDGVE | Clone CH3C.35.23.2 with |
| | VHNAKTKPREEQYNSTYRVVSVLTVLHQDWLNGKEYKCKVSNKALPAPIEKTI | knob, LALA, and YTE |
| | SKAKGQPREPQVYTLPPSRDELTKNQVSLWCLVKGFYPSDIAVEWESYGTEWA | mutations |
| | NYKTTPPVLDSDGSFFLYSKLTVTKEEWQQGFVFSCSVMHEALHNHYTQKSLS | |
| | LSPGK | |
| 244 | APEAAGGPSVFLFPPKPKDTLYITREPEVTCVVVDVSHEDPEVKFNWYVDGVE | Clone CH3C.35.23.2 with |
| | VHNAKTKPREEQYNSTYRVVSVLTVLHQDWLNGKEYKCKVSNKALGAPIEKTI | knob, LALAPG, and YTE |
| | SKAKGQPREPQVYTLPPSRDELTKNQVSLWCLVKGFYPSDIAVEWESYGTEWA | mutations |
| | NYKTTPPVLDSDGSFFLYSKLTVTKEEWQQGFVFSCSVMHEALHNHYTQKSLS | |
| | LSPGK | |
| 245 | APELLGGPSVFLFPPKPKDTLMISRTPEVTCVVVDVSHEDPEVKFNWYVDGVE | Clone CH3C.35.23.2 with |
| | VHNAKTKPREEQYNSTYRVVSVLTVLHQDWLNGKEYKCKVSNKALPAPIEKTI | hole mutations |
| | SKAKGQPREPQVYTLPPSRDELTKNQVSLSCAVKGFYPSDIAVEWESYGTEWA | |
| | NYKTTPPVLDSDGSFFLVSKLTVTKEEWQQGFVFSCSVMHEALHNHYTQKSLS | |
| | LSPGK | |
| 246 | APEAAGGPSVFLFPPKPKDTLMISRTPEVTCVVVDVSHEDPEVKFNWYVDGVE | Clone CH3C.35.23.2 with |
| | VHNAKTKPREEQYNSTYRVVSVLTVLHQDWLNGKEYKCKVSNKALPAPIEKTI | hole and LALA mutations |
| | SKAKGQPREPQVYTLPPSRDELTKNQVSLSCAVKGFYPSDIAVEWESYGTEWA | |
| | NYKTTPPVLDSDGSFFLVSKLTVTKEEWQQGFVFSCSVMHEALHNHYTQKSLS | |
| | LSPGK | |
| 247 | APEAAGGPSVFLFPPKPKDTLMISRTPEVTCVVVDVSHEDPEVKFNWYVDGVE | Clone CH3C.35.23.2 with |
| | VHNAKTKPREEQYNSTYRVVSVLTVLHQDWLNGKEYKCKVSNKALGAPIEKTI | hole and LALAPG mutations |
| | SKAKGQPREPQVYTLPPSRDELTKNQVSLSCAVKGFYPSDIAVEWESYGTEWA | |
| | NYKTTPPVLDSDGSFFLVSKLTVTKEEWQQGFVFSCSVMHEALHNHYTQKSLS | |
| | LSPGK | |
| 248 | APELLGGPSVFLFPPKPKDTLYITREPEVTCVVVDVSHEDPEVKFNWYVDGVE | Clone CH3C.35.23.2 with |
| | VHNAKTKPREEQYNSTYRVVSVLTVLHQDWLNGKEYKCKVSNKALPAPIEKTI | hole and YTE mutations |
| | SKAKGQPREPQVYTLPPSRDELTKNQVSLSCAVKGFYPSDIAVEWESYGTEWA | |
| | NYKTTPPVLDSDGSFFLVSKLTVTKEEWQQGFVFSCSVMHEALHNHYTQKSLS | |
| | LSPGK | |
| 249 | APEAAGGPSVFLFPPKPKDTLYITREPEVTCVVVDVSHEDPEVKFNWYVDGVE | Clone CH3C.35.23.2 with |
| | VHNAKTKPREEQYNSTYRVVSVLTVLHQDWLNGKEYKCKVSNKALPAPIEKTI | hole, LALA, and Y1E |
| | SKAKGQPREPQVYTLPPSRDELTKNQVSLSCAVKGFYPSDIAVEWESYGTEWA | mutations |
| | NYKTTPPVLDSDGSFFLVSKLTVTKEEWQQGFVFSCSVMHEALHNHYTQKSLS | |
| | LSPGK | |
| 250 | APEAAGGPSVFLFPPKPKDTLYITREPEVTCVVVDVSHEDPEVKFNWYVDGVE | Clone CH3C.35.23.2 with |
| | VHNAKTKPREEQYNSTYRVVSVLTVLHQDWLNGKEYKCKVSNKALGAPIEKTI | hole, LALAPG, and YTE |
| | SKAKGQPREPQVYTLPPSRDELTKNQVSLSCAVKGFYPSDIAVEWESYGTEWA | mutations |
| | NYKTTPPVLDSDGSFFLVSKLTVTKEEWQQGFVFSCSVMHEALHNHYTQKSLS | |
| | LSPGK | |
| 251 | APELLGGPSVFLFPPKPKDTLMISRTPEVTCVVVDVSHEDPEVKFNWYVDGVE | Clone CH3C.35.23.3 with |
| | VHNAKTKPREEQYNSTYRVVSVLTVLHQDWLNGKEYKCKVSNKALPAPIEKTI | knob mutation |
| | SKAKGQPREPQVYTLPPSRDELTKNQVSLWCLVKGFYPSDIAVEWESYGTEWV | |
| | NYKTTPPVLDSDGSFFLYSKLTVTKEEWQQGFVFSCSVMHEALHNHYTQKSLS | |
| | LSPGK | |
| 252 | APEAAGGPSVFLFPPKPKDTLMISRTPEVTCVVVDVSHEDPEVKFNWYVDGVE | Clone CH3C.35.23.3 with |
| | VHNAKTKPREEQYNSTYRVVSVLTVLHQDWLNGKEYKCKVSNKALPAPIEKTI | knob and LALA mutations |
| | SKAKGQPREPQVYTLPPSRDELTKNQVSLWCLVKGFYPSDIAVEWESYGTEWV | |
| | NYKTTPPVLDSDGSFFLYSKLTVTKEEWQQGFVFSCSVMHEALHNHYTQKSLS | |
| | LSPGK | |
| 253 | APEAAGGPSVFLFPPKPKDTLMISRTPEVTCVVVDVSHEDPEVKFNWYVDGVE | Clone CH3C.35.23.3 with |
| | VHNAKTKPREEQYNSTYRVVSVLTVLHQDWLNGKEYKCKVSNKALGAPIEKTI | knob and LALAPG |
| | SKAKGQPREPQVYTLPPSRDELTKNQVSLWCLVKGFYPSDIAVEWESYGTEWV | mutations |
| | NYKTTPPVLDSDGSFFLYSKLTVTKEEWQQGFVFSCSVMHEALHNHYTQKSLS | |
| | LSPGK | |
| 254 | APELLGGPSVFLFPPKPKDTLYITREPEVTCVVVDVSHEDPEVKFNWYVDGVE | Clone CH3C.35.23.3 with |
| | VHNAKTKPREEQYNSTYRVVSVLTVLHQDWLNGKEYKCKVSNKALPAPIEKTI | knob and YTE mutations |
| | SKAKGQPREPQVYTLPPSRDELTKNQVSLWCLVKGFYPSDIAVEWESYGTEWV | |
| | NYKTTPPVLDSDGSFFLYSKLTVTKEEWQQGFVFSCSVMHEALHNHYTQKSLS | |
| | LSPGK | |
| 255 | APEAAGGPSVFLFPPKPKDTLYITREPEVTCVVVDVSHEDPEVKFNWYVDGVE | Clone CH3C.35.23.3 with |
| | VHNAKTKPREEQYNSTYRVVSVLTVLHQDWLNGKEYKCKVSNKALPAPIEKTI | knob, LALA, and YTE |
| | SKAKGQPREPQVYTLPPSRDELTKNQVSLWCLVKGFYPSDIAVEWESYGTEWV | mutations |
| | NYKTTPPVLDSDGSFFLYSKLTVTKEEWQQGFVFSCSVMHEALHNHYTQKSLS | |
| | LSPGK | |
| 256 | APEAAGGPSVFLFPPKPKDTLYITREPEVTCVVVDVSHEDPEVKFNWYVDGVE | Clone CH3C.35.23.3 with |
| | VHNAKTKPREEQYNSTYRVVSVLTVLHQDWLNGKEYKCKVSNKALGAPIEKTI | knob, LALAPG, and YTE |
| | SKAKGQPREPQVYTLPPSRDELTKNQVSLWCLVKGFYPSDIAVEWESYGTEWV | mutations |
| | NYKTTPPVLDSDGSFFLYSKLTVTKEEWQQGFVFSCSVMHEALHNHYTQKSLS | |
| | LSPGK | |
| 257 | APELLGGPSVFLFPPKPKDTLMISRTPEVTCVVVDVSHEDPEVKFNWYVDGVE | Clone CH3C.35.23.3 with |
| | VHNAKTKPREEQYNSTYRVVSVLTVLHQDWLNGKEYKCKVSNKALPAPIEKTI | hole mutations |
| | SKAKGQPREPQVYTLPPSRDELTKNQVSLSCAVKGFYPSDIAVEWESYGTEWV | |
| | NYKTTPPVLDSDGSFFLVSKLTVTKEEWQQGFVFSCSVMHEALHNHYTQKSLS | |
| | LSPGK | |
| 258 | APEAAGGPSVFLFPPKPKDTLMISRTPEVTCVVVDVSHEDPEVKFNWYVDGVE | Clone CH3C.35.23.3 with |
| | VHNAKTKPREEQYNSTYRVVSVLTVLHQDWLNGKEYKCKVSNKALPAPIEKTI | hole and LALA mutations |
| | SKAKGQPREPQVYTLPPSRDELTKNQVSLSCAVKGFYPSDIAVEWESYGTEWV | |
| | NYKTTPPVLDSDGSFFLVSKLTVTKEEWQQGFVFSCSVMHEALHNHYTQKSLS | |
| | LSPGK | |
| 259 | APEAAGGPSVFLFPPKPKDTLMISRTPEVTCVVVDVSHEDPEVKFNWYVDGVE | Clone CH3C.35.23.3 with |
| | VHNAKTKPREEQYNSTYRVVSVLTVLHQDWLNGKEYKCKVSNKALGAPIEKTI | hole and LALAPG mutations |
| | SKAKGQPREPQVYTLPPSRDELTKNQVSLSCAVKGFYPSDIAVEWESYGTEWV | |
| | NYKTTPPVLDSDGSFFLVSKLTVTKEEWQQGFVFSCSVMHEALHNHYTQKSLS | |
| | LSPGK | |
| 260 | APELLGGPSVFLFPPKPKDTLYITREPEVTCVVVDVSHEDPEVKFNWYVDGVE | Clone CH3C.35.23.3 with |
| | VHNAKTKPREEQYNSTYRVVSVLTVLHQDWLNGKEYKCKVSNKALPAPIEKTI | hole and YTE mutations |
| | SKAKGQPREPQVYTLPPSRDELTKNQVSLSCAVKGFYPSDIAVEWESYGTEWV | |
| | NYKTTPPVLDSDGSFFLVSKLTVTKEEWQQGFVFSCSVMHEALHNHYTQKSLS | |
| | LSPGK | |
| 261 | APEAAGGPSVFLFPPKPKDTLYITREPEVTCVVVDVSHEDPEVKFNWYVDGVE | Clone CH3C.35.23.3 with |
| | VHNAKTKPREEQYNSTYRVVSVLTVLHQDWLNGKEYKCKVSNKALPAPIEKTI | hole, LALA, and Y1E |
| | SKAKGQPREPQVYTLPPSRDELTKNQVSLSCAVKGFYPSDIAVEWESYGTEWV | mutations |
| | NYKTTPPVLDSDGSFFLVSKLTVTKEEWQQGFVFSCSVMHEALHNHYTQKSLS | |
| | LSPGK | |
| 262 | APEAAGGPSVFLFPPKPKDTLYITREPEVTCVVVDVSHEDPEVKFNWYVDGVE | Clone CH3C.35.23.3 with |
| | VHNAKTKPREEQYNSTYRVVSVLTVLHQDWLNGKEYKCKVSNKALGAPIEKTI | hole, LALAPG, and YTE |
| | SKAKGQPREPQVYTLPPSRDELTKNQVSLSCAVKGFYPSDIAVEWESYGTEWV | mutations |
| | NYKTTPPVLDSDGSFFLVSKLTVTKEEWQQGFVFSCSVMHEALHNHYTQKSLS | |
| | LSPGK | |
| 263 | APELLGGPSVFLFPPKPKDTLMISRTPEVTCVVVDVSHEDPEVKFNWYVDGVE | Clone CH3C.35.23.4 with |
| | VHNAKTKPREEQYNSTYRVVSVLTVLHQDWLNGKEYKCKVSNKALPAPIEKTI | knob mutation |
| | SKAKGQPREPQVYTLPPSRDELTKNQVSLWCLVKGFYPSDIAVEWESYGTEWS | |
| | NYKTTPPVLDSDGSFFLYSKLTVSKEEWQQGFVFSCSVMHEALHNHYTQKSLS | |
| | LSPGK | |
| 264 | APEAAGGPSVFLFPPKPKDTLMISRTPEVTCVVVDVSHEDPEVKFNWYVDGVE | Clone CH3C.35.23.4 with |
| | VHNAKTKPREEQYNSTYRVVSVLTVLHQDWLNGKEYKCKVSNKALPAPIEKTI | knob and LALA mutations |
| | SKAKGQPREPQVYTLPPSRDELTKNQVSLWCLVKGFYPSDIAVEWESYGTEWS | |
| | NYKTTPPVLDSDGSFFLYSKLTVSKEEWQQGFVFSCSVMHEALHNHYTQKSLS | |
| | LSPGK | |
| 265 | APEAAGGPSVFLFPPKPKDTLMISRTPEVTCVVVDVSHEDPEVKFNWYVDGVE | Clone CH3C.35.23.4 with |
| | VHNAKTKPREEQYNSTYRVVSVLTVLHQDWLNGKEYKCKVSNKALGAPIEKTI | knob and LALAPG |
| | SKAKGQPREPQVYTLPPSRDELTKNQVSLWCLVKGFYPSDIAVEWESYGTEWS | mutations |
| | NYKTTPPVLDSDGSFFLYSKLTVSKEEWQQGFVFSCSVMHEALHNHYTQKSLS | |
| | LSPGK | |
| 266 | APELLGGPSVFLFPPKPKDTLYITREPEVTCVVVDVSHEDPEVKFNWYVDGVE | Clone CH3C.35.23.4 with |
| | VHNAKTKPREEQYNSTYRVVSVLTVLHQDWLNGKEYKCKVSNKALPAPIEKTI | knob and YTE mutations |
| | SKAKGQPREPQVYTLPPSRDELTKNQVSLWCLVKGFYPSDIAVEWESYGTEWS | |
| | NYKTTPPVLDSDGSFFLYSKLTVSKEEWQQGFVFSCSVMHEALHNHYTQKSLS | |
| | LSPGK | |
| 267 | APEAAGGPSVFLFPPKPKDTLYITREPEVTCVVVDVSHEDPEVKFNWYVDGVE | Clone CH3C.35.23.4 with |
| | VHNAKTKPREEQYNSTYRVVSVLTVLHQDWLNGKEYKCKVSNKALPAPIEKTI | knob, LALA, and YTE |
| | SKAKGQPREPQVYTLPPSRDELTKNQVSLWCLVKGFYPSDIAVEWESYGTEWS | mutations |
| | NYKTTPPVLDSDGSFFLYSKLTVSKEEWQQGFVFSCSVMHEALHNHYTQKSLS | |
| | LSPGK | |
| 268 | APEAAGGPSVFLFPPKPKDTLYITREPEVTCVVVDVSHEDPEVKFNWYVDGVE | Clone CH3C.35.23.4 with |
| | VHNAKTKPREEQYNSTYRVVSVLTVLHQDWLNGKEYKCKVSNKALGAPIEKTI | knob, LALAPG, and YTE |
| | SKAKGQPREPQVYTLPPSRDELTKNQVSLWCLVKGFYPSDIAVEWESYGTEWS | mutations |
| | NYKTTPPVLDSDGSFFLYSKLTVSKEEWQQGFVFSCSVMHEALHNHYTQKSLS | |
| | LSPGK | |
| 269 | APELLGGPSVFLFPPKPKDTLMISRTPEVTCVVVDVSHEDPEVKFNWYVDGVE | Clone CH3C.35.23.4 with |
| | VHNAKTKPREEQYNSTYRVVSVLTVLHQDWLNGKEYKCKVSNKALPAPIEKTI | hole mutations |
| | SKAKGQPREPQVYTLPPSRDELTKNQVSLSCAVKGFYPSDIAVEWESYGTEWS | |
| | NYKTTPPVLDSDGSFFLVSKLTVSKEEWQQGFVFSCSVMHEALHNHYTQKSLS | |
| | LSPGK | |
| 270 | APEAAGGPSVFLFPPKPKDTLMISRTPEVTCVVVDVSHEDPEVKFNWYVDGVE | Clone CH3C.35.23.4 with |
| | VHNAKTKPREEQYNSTYRVVSVLTVLHQDWLNGKEYKCKVSNKALPAPIEKTI | hole and LALA mutations |
| | SKAKGQPREPQVYTLPPSRDELTKNQVSLSCAVKGFYPSDIAVEWESYGTEWS | |
| | NYKTTPPVLDSDGSFFLVSKLTVSKEEWQQGFVFSCSVMHEALHNHYTQKSLS | |
| | LSPGK | |
| 271 | APEAAGGPSVFLFPPKPKDTLMISRTPEVTCVVVDVSHEDPEVKFNWYVDGVE | Clone CH3C.35.23.4 with |
| | VHNAKTKPREEQYNSTYRVVSVLTVLHQDWLNGKEYKCKVSNKALGAPIEKTI | hole and LALAPG mutations |
| | SKAKGQPREPQVYTLPPSRDELTKNQVSLSCAVKGFYPSDIAVEWESYGILWS | |
| | NYKTTPPVLDSDGSFFLVSKLTVSKEEWQQGFVFSCSVMHEALHNHYTQKSLS | |
| | LSPGK | |
| 272 | APELLGGPSVFLFPPKPKDTLYITREPEVTCVVVDVSHEDPEVKFNWYVDGVE | Clone CH3C.35.23.4 with |
| | VHNAKTKPREEQYNSTYRVVSVLTVLHQDWLNGKEYKCKVSNKALPAPIEKTI | hole and YTE mutations |
| | SKAKGQPREPQVYTLPPSRDELTKNQVSLSCAVKGFYPSDIAVEWESYGTEWS | |
| | NYKTTPPVLDSDGSFFLVSKLTVSKEEWQQGFVFSCSVMHEALHNHYTQKSLS | |
| | LSPGK | |
| 273 | APEAAGGPSVFLFPPKPKDTLYITREPEVTCVVVDVSHEDPEVKFNWYVDGVE | Clone CH3C.35.23.4 with |
| | VHNAKTKPREEQYNSTYRVVSVLTVLHQDWLNGKEYKCKVSNKALPAPIEKTI | hole, LALA, and Y1E |
| | SKAKGQPREPQVYTLPPSRDELTKNQVSLSCAVKGFYPSDIAVEWESYGTEWS | mutations |
| | NYKTTPPVLDSDGSFFLVSKLTVSKEEWQQGFVFSCSVMHEALHNHYTQKSLS | |
| | LSPGK | |
| 274 | APEAAGGPSVFLFPPKPKDTLYITREPEVTCVVVDVSHEDPEVKFNWYVDGVE | Clone CH3C.35.23.4 with |
| | VHNAKTKPREEQYNSTYRVVSVLTVLHQDWLNGKEYKCKVSNKALGAPIEKTI | hole, LALAPG, and YTE |
| | SKAKGQPREPQVYTLPPSRDELTKNQVSLSCAVKGFYPSDIAVEWESYGTEWS | mutations |
| | NYKTTPPVLDSDGSFFLVSKLTVSKEEWQQGFVFSCSVMHEALHNHYTQKSLS | |
| | LSPGK | |
| 275 | APELLGGPSVFLFPPKPKDTLMISRTPEVTCVVVDVSHEDPEVKFNWYVDGVE | Clone CH3C.35.21.17.2 with |
| | VHNAKTKPREEQYNSTYRVVSVLTVLHQDWLNGKEYKCKVSNKALPAPIEKTI | knob mutation |
| | SKAKGQPREPQVYTLPPSRDELTKNQVSLWCLVKGFYPSDIAVLWESYGTEWA | |
| | SYKTTPPVLDSDGSFFLYSKLTVTKEEWQQGFVFSCSVMHEALHNHYTQKSLS | |
| | LSPGK | |
| 276 | APEAAGGPSVFLFPPKPKDTLMISRTPEVTCVVVDVSHEDPEVKFNWYVDGVE | Clone CH3C.35.21.17.2 with |
| | VHNAKTKPREEQYNSTYRVVSVLTVLHQDWLNGKEYKCKVSNKALPAPIEKTI | knob and LALA mutations |
| | SKAKGQPREPQVYTLPPSRDELTKNQVSLWCLVKGFYPSDIAVLWESYGTEWA | |
| | SYKTTPPVLDSDGSFFLYSKLTVTKEEWQQGFVFSCSVMHEALHNHYTQKSLS | |
| | LSPGK | |
| 277 | APEAAGGPSVFLFPPKPKDTLMISRTPEVTCVVVDVSHEDPEVKFNWYVDGVE | Clone CH3C.35.21.17.2 with |
| | VHNAKTKPREEQYNSTYRVVSVLTVLHQDWLNGKEYKCKVSNKALGAPIEKTI | knob and LALAPG |
| | SKAKGQPREPQVYTLPPSRDELTKNQVSLWCLVKGFYPSDIAVLWESYGTEWA | mutations |
| | SYKTTPPVLDSDGSFFLYSKLTVTKEEWQQGFVFSCSVMHEALHNHYTQKSLS | |
| | LSPGK | |
| 278 | APELLGGPSVFLFPPKPKDTLYITREPEVTCVVVDVSHEDPEVKFNWYVDGVE | Clone CH3C.35.21.17.2 with |
| | VHNAKTKPREEQYNSTYRVVSVLTVLHQDWLNGKEYKCKVSNKALPAPIEKTI | knob and YTE mutations |
| | SKAKGQPREPQVYTLPPSRDELTKNQVSLWCLVKGFYPSDIAVLWESYGTEWA | |
| | SYKTTPPVLDSDGSFFLYSKLTVTKEEWQQGFVFSCSVMHEALHNHYTQKSLS | |
| | LSPGK | |
| 279 | APEAAGGPSVFLFPPKPKDTLYITREPEVTCVVVDVSHEDPEVKFNWYVDGVE | Clone CH3C.35.21.17.2 with |
| | VHNAKTKPREEQYNSTYRVVSVLTVLHQDWLNGKEYKCKVSNKALPAPIEKTI | knob, LALA, and YTE |
| | SKAKGQPREPQVYTLPPSRDELTKNQVSLWCLVKGFYPSDIAVLWESYGTEWA | mutations |
| | SYKTTPPVLDSDGSFFLYSKLTVTKEEWQQGFVFSCSVMHEALHNHYTQKSLS | |
| | LSPGK | |
| 280 | APEAAGGPSVFLFPPKPKDTLYITREPEVTCVVVDVSHEDPEVKFNWYVDGVE | Clone CH3C.35.21.17.2 with |
| | VHNAKTKPREEQYNSTYRVVSVLTVLHQDWLNGKEYKCKVSNKALGAPIEKTI | knob, LALAPG, and YTE |
| | SKAKGQPREPQVYTLPPSRDELTKNQVSLWCLVKGFYPSDIAVLWESYGTEWA | mutations |
| | SYKTTPPVLDSDGSFFLYSKLTVTKEEWQQGFVFSCSVMHEALHNHYTQKSLS | |
| | LSPGK | |
| 281 | APELLGGPSVFLFPPKPKDTLMISRTPEVTCVVVDVSHEDPEVKFNWYVDGVE | Clone CH3C.35.21.17.2 with |
| | VHNAKTKPREEQYNSTYRVVSVLTVLHQDWLNGKEYKCKVSNKALPAPIEKTI | hole mutations |
| | SKAKGQPREPQVYTLPPSRDELTKNQVSLSCAVKGFYPSDIAVLWESYGTEWA | |
| | SYKTTPPVLDSDGSFFLVSKLTVTKEEWQQGFVFSCSVMHEALHNHYTQKSLS | |
| | LSPGK | |
| 282 | APEAAGGPSVFLFPPKPKDTLMISRTPEVTCVVVDVSHEDPEVKFNWYVDGVE | Clone CH3C.35.21.17.2 with |
| | VHNAKTKPREEQYNSTYRVVSVLTVLHQDWLNGKEYKCKVSNKALPAPIEKTI | hole and LALA mutations |
| | SKAKGQPREPQVYTLPPSRDELTKNQVSLSCAVKGFYPSDIAVLWESYGTEWA | |
| | SYKTTPPVLDSDGSFFLVSKLTVTKEEWQQGFVFSCSVMHEALHNHYTQKSLS | |
| | LSPGK | |
| 283 | APEAAGGPSVFLFPPKPKDTLMISRTPEVTCVVVDVSHEDPEVKFNWYVDGVE | Clone CH3C.35.21.17.2 with |
| | VHNAKTKPREEQYNSTYRVVSVLTVLHQDWLNGKEYKCKVSNKALGAPIEKTI | hole and LALAPG mutations |
| | SKAKGQPREPQVYTLPPSRDELTKNQVSLSCAVKGFYPSDIAVLWESYGTEWA | |
| | SYKTTPPVLDSDGSFFLVSKLTVTKEEWQQGFVFSCSVMHEALHNHYTQKSLS | |
| | LSPGK | |
| 284 | APELLGGPSVFLFPPKPKDTLYITREPEVTCVVVDVSHEDPEVKFNWYVDGVE | Clone CH3C.35.21.17.2 with |
| | VHNAKTKPREEQYNSTYRVVSVLTVLHQDWLNGKEYKCKVSNKALPAPIEKTI | hole and YTE mutations |
| | SKAKGQPREPQVYTLPPSRDELTKNQVSLSCAVKGFYPSDIAVLWESYGTEWA | |
| | SYKTTPPVLDSDGSFFLVSKLTVTKEEWQQGFVFSCSVMHEALHNHYTQKSLS | |
| | LSPGK | |
| 285 | APEAAGGPSVFLFPPKPKDTLYITREPEVTCVVVDVSHEDPEVKFNWYVDGVE | Clone CH3C.35.21.17.2 with |
| | VHNAKTKPREEQYNSTYRVVSVLTVLHQDWLNGKEYKCKVSNKALPAPIEKTI | hole, LALA, and Y1E |
| | SKAKGQPREPQVYTLPPSRDELTKNQVSLSCAVKGFYPSDIAVLWESYGTEWA | mutations |
| | SYKTTPPVLDSDGSFFLVSKLTVTKEEWQQGFVFSCSVMHEALHNHYTQKSLS | |
| | LSPGK | |
| 286 | APEAAGGPSVFLFPPKPKDTLYITREPEVTCVVVDVSHEDPEVKFNWYVDGVE | Clone CH3C.35.21.17.2 with |
| | VHNAKTKPREEQYNSTYRVVSVLTVLHQDWLNGKEYKCKVSNKALGAPIEKTI | hole, LALAPG, and YTE |
| | SKAKGQPREPQVYTLPPSRDELTKNQVSLSCAVKGFYPSDIAVLWESYGTEWA | mutations |
| | SYKTTPPVLDSDGSFFLVSKLTVTKEEWQQGFVFSCSVMHEALHNHYTQKSLS | |
| | LSPGK | |
| 287 | APELLGGPSVFLFPPKPKDTLMISRTPEVTCVVVDVSHEDPEVKFNWYVDGVE | Clone CH3C.35.23 with knob |
| | VHNAKTKPREEQYNSTYRVVSVLTVLHQDWLNGKEYKCKVSNKALPAPIEKTI | mutation |
| | SKAKGQPREPQVYTLPPSRDELTKNQVSLWCLVKGFYPSDIAVEWESYGTEWS | |
| | NYKTTPPVLDSDGSFFLYSKLTVTKEEWQQGFVFSCSVMHEALHNHYTQKSLS | |
| | LSPGK | |
| 288 | APEAAGGPSVFLFPPKPKDTLMISRTPEVTCVVVDVSHEDPEVKFNWYVDGVE | Clone CH3C.35.23 with knob |
| | VHNAKTKPREEQYNSTYRVVSVLTVLHQDWLNGKEYKCKVSNKALPAPIEKTI | and LALA mutations |
| | SKAKGQPREPQVYTLPPSRDELTKNQVSLWCLVKGFYPSDIAVEWESYGTEWS | |
| | NYKTTPPVLDSDGSFFLYSKLTVTKEEWQQGFVFSCSVMHEALHNHYTQKSLS | |
| | LSPGK | |
| 289 | APEAAGGPSVFLFPPKPKDTLMISRTPEVTCVVVDVSHEDPEVKFNWYVDGVE | Clone CH3C.35.23 with knob |
| | VHNAKTKPREEQYNSTYRVVSVLTVLHQDWLNGKEYKCKVSNKALGAPIEKTI | and LALAPG mutations |
| | SKAKGQPREPQVYTLPPSRDELTKNQVSLWCLVKGFYPSDIAVEWESYGTEWS | |
| | NYKTTPPVLDSDGSFFLYSKLTVTKEEWQQGFVFSCSVMHEALHNHYTQKSLS | |
| | LSPGK | |
| 290 | APELLGGPSVFLFPPKPKDTLYITREPEVTCVVVDVSHEDPEVKFNWYVDGVE | Clone CH3C.35.23 with knob |
| | VHNAKTKPREEQYNSTYRVVSVLTVLHQDWLNGKEYKCKVSNKALPAPIEKTI | and YTE mutations |
| | SKAKGQPREPQVYTLPPSRDELTKNQVSLWCLVKGFYPSDIAVEWESYGTEWS | |
| | NYKTTPPVLDSDGSFFLYSKLTVTKEEWQQGFVFSCSVMHEALHNHYTQKSLS | |
| | LSPGK | |
| 291 | APEAAGGPSVFLFPPKPKDTLYITREPEVTCVVVDVSHEDPEVKFNWYVDGVE | Clone CH3C.35.23 with |
| | VHNAKTKPREEQYNSTYRVVSVLTVLHQDWLNGKEYKCKVSNKALPAPIEKTI | knob, LALA, and YTE |
| | SKAKGQPREPQVYTLPPSRDELTKNQVSLWCLVKGFYPSDIAVEWESYGTEWS | mutations |
| | NYKTTPPVLDSDGSFFLYSKLTVTKEEWQQGFVFSCSVMHEALHNHYTQKSLS | |
| | LSPGK | |
| 292 | APEAAGGPSVFLFPPKPKDTLYITREPEVTCVVVDVSHEDPEVKFNWYVDGVE | Clone CH3C.35.23 with |
| | VHNAKTKPREEQYNSTYRVVSVLTVLHQDWLNGKEYKCKVSNKALGAPIEKTI | knob, LALAPG, and Y1E |
| | SKAKGQPREPQVYTLPPSRDELTKNQVSLWCLVKGFYPSDIAVEWESYGTEWS | mutations |
| | NYKTTPPVLDSDGSFFLYSKLTVTKEEWQQGFVFSCSVMHEALHNHYTQKSLS | |
| | LSPGK | |
| 293 | APELLGGPSVFLFPPKPKDTLMISRTPEVTCVVVDVSHEDPEVKFNWYVDGVE | Clone CH3C.35.23 with hole |
| | VHNAKTKPREEQYNSTYRVVSVLTVLHQDWLNGKEYKCKVSNKALPAPIEKTI | mutations |
| | SKAKGQPREPQVYTLPPSRDELTKNQVSLSCAVKGFYPSDIAVEWESYGTEWS | |
| | NYKTTPPVLDSDGSFFLVSKLTVTKEEWQQGFVFSCSVMHEALHNHYTQKSLS | |
| | LSPGK | |
| 294 | APEAAGGPSVFLFPPKPKDTLMISRTPEVTCVVVDVSHEDPEVKFNWYVDGVE | Clone CH3C.35.23 with hole |
| | VHNAKTKPREEQYNSTYRVVSVLTVLHQDWLNGKEYKCKVSNKALPAPIEKTI | and LALA mutations |
| | SKAKGQPREPQVYTLPPSRDELTKNQVSLSCAVKGFYPSDIAVEWESYGTEWS | |
| | NYKTTPPVLDSDGSFFLVSKLTVTKEEWQQGFVFSCSVMHEALHNHYTQKSLS | |
| | LSPGK | |
| 295 | APEAAGGPSVFLFPPKPKDTLMISRTPEVTCVVVDVSHEDPEVKFNWYVDGVE | Clone CH3C.35.23 with hole |
| | VHNAKTKPREEQYNSTYRVVSVLTVLHQDWLNGKEYKCKVSNKALGAPIEKTI | and LALAPG mutations |
| | SKAKGQPREPQVYTLPPSRDELTKNQVSLSCAVKGFYPSDIAVEWESYGTEWS | |
| | NYKTTPPVLDSDGSFFLVSKLTVTKEEWQQGFVFSCSVMHEALHNHYTQKSLS | |
| | LSPGK | |
| 296 | APELLGGPSVFLFPPKPKDTLYITREPEVTCVVVDVSHEDPEVKFNWYVDGVE | Clone CH3C.35.23 with hole |
| | VHNAKTKPREEQYNSTYRVVSVLTVLHQDWLNGKEYKCKVSNKALPAPIEKTI | and YTE mutations |
| | SKAKGQPREPQVYTLPPSRDELTKNQVSLSCAVKGFYPSDIAVEWESYGTEWS | |
| | NYKTTPPVLDSDGSFFLVSKLTVTKEEWQQGFVFSCSVMHEALHNHYTQKSLS | |
| | LSPGK | |
| 297 | APEAAGGPSVFLFPPKPKDTLYITREPEVTCVVVDVSHEDPEVKFNWYVDGVE | Clone CH3C.35.23 with hole, |
| | VHNAKTKPREEQYNSTYRVVSVLTVLHQDWLNGKEYKCKVSNKALPAPIEKTI | LALA, and YTE mutations |
| | SKAKGQPREPQVYTLPPSRDELTKNQVSLSCAVKGFYPSDIAVEWESYGTEWS | |
| | NYKTTPPVLDSDGSFFLVSKLTVTKEEWQQGFVFSCSVMHEALHNHYTQKSLS | |
| | LSPGK | |
| 298 | APEAAGGPSVFLFPPKPKDTLYITREPEVTCVVVDVSHEDPEVKFNWYVDGVE | Clone CH3C.35.23 with hole, |
| | VHNAKTKPREEQYNSTYRVVSVLTVLHQDWLNGKEYKCKVSNKALGAPIEKTI | LALAPG, and YTE |
| | SKAKGQPREPQVYTLPPSRDELTKNQVSLSCAVKGFYPSDIAVEWESYGTEWS | mutations |
| | NYKTTPPVLDSDGSFFLVSKLTVTKEEWQQGFVFSCSVMHEALHNHYTQKSLS | |
| | LSPGK | |
| 299 | YxTEWSS | CH3C motif |
| 300 | TxxExxxxF | CH3C motif |
| 301 | GGACAGGGATCCAGAGTTCC | muIgG1 3′ VH PCR primer |
| 302 | AGCTGGGAAGGTGTGCACAC | muIgG2 3′ VH PCR primer |
| 303 | CAGGGGCCAGTGGATAGAC | muIgG3 3′ VH PCR primer |
| 304 | GACATTGATGTCTTTGGGGT | muCkappa.1 3′ VL PCR |
| | | primer |
| 305 | TTCACTGCCATCAATCTTCC | muCkappa.2 3′ VL PCR |
| | | primer |
| 306 | EVQLQQSGPELVKPGASVKMSCKASGYTFTDYNMHWVKQSHGKSLEWIGYINP | 7B10.A2 VH amino acid |
| | NNGGTTYNQKFKGKATLTVNKSSSTAYMELRSLTSEDSAVYYCATYNNHYFDS | sequence |
| | WGQGTTLTVSS | |
| 307 | GYTFTDYNMH | 7B10.A2 CDR-H1 |
| 308 | YINPNNGGTTYNQKFKG | 7B10.A2 CDR-H2 |
| 309 | ATYNNHYFDS | 7B10.A2 CDR-H3 |
| 310 | DIQMTQTTSSLSASLGDRVTISCSASQGISNYLNWYQQKPDGTVKLLIYYTSN | 7B10.A2 VL amino acid |
| | LHSGVPSRFSGSGSGTDYSLTISNLEPEDIATYYCQQYSNLPYTFGGGTKLEI | sequence |
| | K | |
| 311 | SASQGISNYLN | 7B10.A2 CDR-L1 |
| 312 | YTSNLHS | 7B10.A2 CDR-L2 |
| 313 | QQYSNLPYT | 7B10.A2 CDR-L3 |
| 314 | QVHLQQSGPEVVRPGVSVKISCKGSGYTFTDYGMHWVKQSHAKSLEWIGVIST | 51D4 VH amino acid |
| | YNGNTSYNQKYKGKATVTVDKPSSTAYMELVRLTSEDSAIYYCARDFGYVPFD | sequence |
| | YWGQGTTLTVSS | |
| 315 | GYTFTDYGMH | 51D4 CDR-H1 |
| 316 | VISTYNGNTSYNQKYKG | 51D4 CDR-H2 |
| 317 | ARDFGYVPFDY | 51D4 CDR-H3 |
| 318 | DVVMTQTPLTLSVTIGQPASISCKSSQSLLDSDGKTYLNWLLQRPGQSPKRLI | 51D4 VL amino acid |
| | YLVSYLDSGVPDRFTGSGSGTDFTLKISRVEADDLGVYYCWQGTHFPYTFGGG | sequence |
| | TKLEIK | |
| 319 | LVSYLDS | 51D4 CDR-L2 |
| 320 | GX₂X₃X₄X₅X₆X₇X₈X₉X₁₀X₁₁, wherein X₂ is Y or F; X₃ is T, N, | CDR-H1 consensus sequence |
| | or S; X₄ is F, L, or I; X₅ is T, S, or K; X₆ is D, S, | |
| | or E; X₇ is D or absent; X₈ is H, Y, or T; X₉ is A, N, | |
| | G, V, W, T, or Y; X₁₀ is M, I, or W; andX₁₁ is H, Q, | |
| | or N | |
| 321 | GYX₃X₄X₅X₆X₇X₈X₉X₁₀X₁₁, wherein X₃ is T or S; X₄ is F, L, | CDR-H1 consensus sequence |
| | or I; X₅ is T or S; X₆ is D, S, or E; X₇ is D or | |
| | absent; X₈ is H or Y; X₉ is A, N, G, V, W, T, or A; | |
| | X₁₀ is M, I, or W; and X₁₁ is H, Q, or N | |
| 322 | GX₂X₃X₄X₅X₆X₈X₉X₁₀X₁₁, wherein X₂ is Y or F; X₃ is T or N; | CDR-H1 consensus sequence |
| | X₄ is F, L, or I; X₅ is T, S, or K; X₆ is D, S, or E; | |
| | X₈ is H, Y, or T; X₉ is A, N, G, V, W, T, Y, or A;; | |
| | X₁₀ is M or I; and X₁₁ is H or Q | |
| 323 | GYX₄X₅X₆X₈X₉X₁₀X₁₁, wherein X₄ is F or L; X₅ is T or S; | CDR-H1 consensus sequence |
| | X₆ is D, S, or E; X₈ is H, Y; X₉ is A, N, G, V, W, T,; | |
| | X₁₀ is M or I; and X₁₁ is H or Q | |
| 324 | X₁X₂X₃X₄X₅X₆X₇X₈X₉X₁₀YX₁₂X₁₃X₁₄X₁₅X₁₆X₁₇, wherein X₁ is D, V, | CDR-H2 consensus sequence |
| | Y, R, G, or T; X₂ is I, S, or V; X₃ is L, S, N, D, I, | |
| | or Y; X₄ is P, T, or absent; X₅ is S, Y, N, T, A, G, | |
| | or F; X₆ is I, S, N, T, or D; X₇ is G or D; X₈ is G, | |
| | D, N, R, or S; X₉ is R, T, or A; X₁₀ is I, G, S, K, T, | |
| | N, or R; X₁₂ is G, N, D, or T; X₁₃ is V, Q, E, or P; | |
| | X₁₄ is K or S; X₁₅ is F, Y, or L; X₁₆ is K, R, Q, or | |
| | is absent; and X₁₇ is G, T, D, S, or is absent | |
| 325 | X₁X₂X₃X₄X₅X₆X₇X₈X₉X₁₀YX₁₂X₁₃X₁₄X₁₅X₁₆X₁₇, wherein X₁ is V, Y, | CDR-H2 consensus sequence |
| | R, G, or T; X₂ is I, S, or V; X₃ is S, N, D, I, or Y; | |
| | X₄ is P, T, or absent; X₅ is Y, N, T, A, G, or F; X₆ | |
| | is S, N, T, or D; X₇ is G or D; X₈ is G, D, N, R, or | |
| | s; X₉ is T, or A; X₁₀ is I, G, S, K, T, N, or R; X₁₂ | |
| | is N, D, or T; X₁₃ is Q, E, or P; X₁₄ is K or S; X₁₅ | |
| | is F, Y or L; X₁₆ is K, R, or Q; and X₁₇ is G, T, D, | |
| | or S | |
| 326 | X₁X₂X₃X₄X₅X₆X₇X₈X₉X₁₀YX₁₂X₁₃KX₁₅X₁₆X₁₇, wherein X₁ is V, Y, | CDR-H2 consensus sequence |
| | R, G, or T; X₂ is I, S, or V; X₃ is S, N, D, I, or Y; | |
| | X₄ is P or T; X₅ is Y, N, T, A, or G; X₆ is S, N, T, | |
| | or D; X₇ is G or D; X₈ is G, D, or N; X₉ is T, or A; | |
| | X₁₀ is G, S, K, T, N, or R; X₁₂ is N, D, or T; X₁₃ is | |
| | Q, E, or P; X₁₅ is F or Y; X₁₆ is K, R, or Q; and X₁₇ | |
| | is G, T, or D | |
| 327 | ARX₃X₄X₅X₆X₇X₈X₉X₁₀YAX₁₃DY, wherein X₃ is G or N; X₄ is | CDR-H3 consensus sequence |
| | D or G; X₅ is D or I; X₆ is S or T; X₇ is Y or T; X₈ | |
| | is R or A; X₉ is R or G; X₁₀ is G or Y; and X₁₃ is L | |
| | or M | |
| 328 | X₁SSX₄SLX₇X₈X₉X₁₀X₁₁X₁₂X₁₃X₁₄X₁₅LX₁₇, wherein X₁ is R or K; | CDR-L1 consensus sequence |
| | X₄ is Q or K; X₇ is V or L; X₈ is H, D, or Y; X₉ is | |
| | I, N, or S; X₁₀ is S or absent; X₁₁ is D or N; X₁₂ is | |
| | G or Q; X₁₃ is N, I, or K; X₁₄ is T or S; X₁₅ is Y or | |
| | F; and X₁₇ is Q, H, Y, N, or A | |
| 329 | X₁ASX₄X₅IX₇X₈X₉LX₁₁, wherein X₁ is R, K, or S; X₄ is E | CDR-L1 consensus sequence |
| | or Q; X₅ is N, D, or G; X₇ is Y or S; X₈ is S or N; | |
| | X₉ is N, R, or Y; and X₁₁ is A or N | |
| 330 | X₁X₂SX₄X₅X₆S, wherein X₁ is K, Q, Y, V, or L; X₂ is V, | CDR-L2 consensus sequence |
| | M, or T; X₄ is N, K, or Y; X₅ is R or L; and X₆ is F, | |
| | A, H, or D | |
| 331 | X₁X₂X₃X₄X₅X₆X₇X₈T, wherein X₁ is S, W, or Q; X₂ is Q or | CDR-L3 consensus sequence |
| | H; X₃ is S, T, G, Y, or F; X₄ is T, F, W, S; X₅ is H, | |
| | S, G, or N; X₆ is V, A, F, Y, T, or L; and X₈ is Y, | |
| | F, P, or W | |
| 332 | QX₂X₃X₄X₅X₆PX₈T, wherein X₂ is Q or H; X₃ is Y or F; X₄ | CDR-L3 consensus sequence |
| | is F, W, or S; X₅ is S, G, or N; X₆ is Y, T, or L; | |
| | and X₈ is P, Y, or W | |
| 333 | SGAHNTTVFQGVAGQSLQVSCPYDSMKHWGRRKAWCRQLGEKGPCQRVVSTHN | Human TREM2 extracellular |
| | LWLLSFLRRWNGSTAITDDTLGGTLTITLRNLQPHDAGLYQCQSLHGSEADTL | domain (ECD) amino acid |
| | RKVLVEVLADPLDHRDAGDLWFPGESESFEDAHVEHSISRSLLEGEIPFPPTS | sequence (without signal |
| | | peptide and His tag) |
| 334 | DLWFPGESES | Human TREM2 peptide |
| 335 | DLWFPGESE | Human TREM2 peptide 9- |
| | | mer amino acid sequence |
| 336 | DLWFP | Human TREM2 peptide |
| | | sequence (residues 140-144 |
| | | of full-length TREM2) |
| 337 | APELLGGPSVFLFPPKPKDTLMISRTPEVTCVVVDVSHEDPEVKFNWYVDGVE | Clone CH3C.18.3.4-1 |
| | VHNAKTKPREEQYNSTYRVVSVLTVLHQDWLNGKEYKCKVSNKALPAPIEKTI | (CH3C.3.4-1) |
| | SKAKGQPREPQVYTLPPSRDELTKNQVSLTCLVKGFYPSDIAVEWESWGFVWS | |
| | TYKTTPPVLDSDGSFFLYSKLTVPKSNWQQGFVFSCSVMHEALHNHYTQKSLS | |
| | LSPGK | |
| 338 | APELLGGPSVFLFPPKPKDTLMISRTPEVTCVVVDVSHEDPEVKFNWYVDGVE | Clone CH3C.18.3.4-19 |
| | VHNAKTKPREEQYNSTYRVVSVLTVLHQDWLNGKEYKCKVSNKALPAPIEKTI | (CH3C.3.4-19) |
| | SKAKGQPREPQVYTLPPSRDELTKNQVSLTCLVKGFYPSDIAVEWESWGHVWS | |
| | TYKTTPPVLDSDGSFFLYSKLTVPKSNWQQGYVFSCSVMHEALHNHYTQKSLS | |
| | LSPGK | |
| 339 | APELLGGPSVFLFPPKPKDTLMISRTPEVTCVVVDVSHEDPEVKFNWYVDGVE | Clone CH3C.18.3.2-3 |
| | VHNAKTKPREEQYNSTYRVVSVLTVLHQDWLNGKEYKCKVSNKALPAPIEKTI | (CH3C.3.2-3) |
| | SKAKGQPREPQVYTLPPSRDELTKNQVSLTCLVKGFYPSDIAVEWESLGHVWV | |
| | EQKTTPPVLDSDGSFFLYSKLTVPKSTWQQGWVFSCSVMHEALHNHYTQKSLS | |
| | LSPGK | |
| 340 | APELLGGPSVFLFPPKPKDTLMISRTPEVTCVVVDVSHEDPEVKFNWYVDGVE | Clone CH3C.18.3.2-14 |
| | VHNAKTKPREEQYNSTYRVVSVLTVLHQDWLNGKEYKCKVSNKALPAPIEKTI | (CH3C.3.2-14) |
| | SKAKGQPREPQVYTLPPSRDELTKNQVSLTCLVKGFYPSDIAVEWESLGHVWV | |
| | GVKTTPPVLDSDGSFFLYSKLTVPKSTWQQGWVFSCSVMHEALHNHYTQKSLS | |
| | LSPGK | |
| 341 | APELLGGPSVFLFPPKPKDTLMISRTPEVTCVVVDVSHEDPEVKFNWYVDGVE | Clone CH3C.18.3.2-24 |
| | VHNAKTKPREEQYNSTYRVVSVLTVLHQDWLNGKEYKCKVSNKALPAPIEKTI | (CH3C.3.2-24) |
| | SKAKGQPREPQVYTLPPSRDELTKNQVSLTCLVKGFYPSDIAVEWESLGHVWV | |
| | HTKTTPPVLDSDGSFFLYSKLTVPKSTWQQGWVFSCSVMHEALHNHYTQKSLS | |
| | LSPGK | |
| 342 | APELLGGPSVFLFPPKPKDTLMISRTPEVTCVVVDVSHEDPEVKFNWYVDGVE | Clone CH3C.18.3.4-26 |
| | VHNAKTKPREEQYNSTYRVVSVLTVLHQDWLNGKEYKCKVSNKALPAPIEKTI | (CH3C.3.4-26) |
| | SKAKGQPREPQVYTLPPSRDELTKNQVSLTCLVKGFYPSDIAVEWESWGTVWG | |
| | TYKTTPPVLDSDGSFFLYSKLTVPKSNWQQGYVFSCSVMHEALHNHYTQKSLS | |
| | LSPGK | |
| 343 | APELLGGPSVFLFPPKPKDTLMISRTPEVTCVVVDVSHEDPEVKFNWYVDGVE | Clone CH3C.18.3.2-27 |
| | VHNAKTKPREEQYNSTYRVVSVLTVLHQDWLNGKEYKCKVSNKALPAPIEKTI | (CH3C.3.2-27) |
| | SKAKGQPREPQVYTLPPSRDELTKNQVSLTCLVKGFYPSDIAVEWESLGHVWV | |
| | GTKTTPPVLDSDGSFFLYSKLTVPKSTWQQGWVFSCSVMHEALHNHYTQKSLS | |
| | LSPGK | |
| 344 | APELLGGPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESFGTEWS | Clone CH3C.35.20.1.1 |
| | SYKTTPPVLDSDGSFFLYSKLTVSKEEWQQGFVFSCSVMHEALHNHYTQKSLS | |
| | LSPGK | |
| 345 | APELLGGPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESYGTEWA | Clone CH3C.35.23.2.1 |
| | NYKTTPPVLDSDGSFFLYSKLTVSKSEWQQGFVFSCSVMHEALHNHYTQKSLS | |
| | LSPGK | |
| 346 | APELLGGPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESFGTEWS | Clone CH3C.35.23.1.1 |
| | NYKTTPPVLDSDGSFFLYSKLTVSKEEWQQGFVFSCSVMHEALHNHYTQKSLS | |
| | LSPGK | |
| 347 | APELLGGPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESYGTEWS | Clone CH3C.35.S413 |
| | SYKTTPPVLDSDGSFFLYSKLTVSKSEWQQGFVFSCSVMHEALHNHYTQKSLS | |
| | LSPGK | |
| 348 | APELLGGPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESYGTEWV | Clone CH3C.35.23.3.1 |
| | NYKTTPPVLDSDGSFFLYSKLTVSKEEWQQGFVFSCSVMHEALHNHYTQKSLS | |
| | LSPGK | |
| 349 | APELLGGPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESYGTEWS | Clone CH3C.35.N390.1 |
| | NYKTTPPVLDSDGSFFLYSKLTVSKSEWQQGFVFSCSVMHEALHNHYTQKSLS | |
| | LSPGK | |
| 350 | APELLGGPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALPAPIEKTI | |
| | SKAKGQPREPQVYTLPPSRDELTKNQVSLTCLVKGFYPSDIAVEWESFGTEWV | Clone CH3C.35.23.6.1 |
| | NYKTTPPVLDSDGSFFLYSKLTVSKEEWQQGFVFSCSVMHEALHNHYTQKSLS | |
| | LSPGK | |
| 351 | APEAAGGPSVFLFPPKPKDTLMISRTPEVTCVVVDVSHEDPEVKFNWYVDGVE | Clone CH3C.35.21 with knob |
| | VHNAKTKPREEQYNSTYRVVSVLTVLHQDWLNGKEYKCKVSNKALGAPIEKTI | and LALAPG mutations |
| | SKAKGQPREPQVYTLPPSRDELTKNQVSLWCLVKGFYPSDIAVWWESYGTEWS | |
| | SYKTTPPVLDSDGSFFLYSKLTVTKEEWQQGFVFSCSVMHEALHNHYTQKSLS | |
| | LSPGK | |
| 352 | APEAAGGPSVFLFPPKPKDTLYITREPEVTCVVVDVSHEDPEVKFNWYVDGVE | Clone CH3C.35.21 with |
| | VHNAKTKPREEQYNSTYRVVSVLTVLHQDWLNGKEYKCKVSNKALGAPIEKTI | knob, LALAPG, and YTE |
| | SKAKGQPREPQVYTLPPSRDELTKNQVSLWCLVKGFYPSDIAVWWESYGTEWS | mutations |
| | SYKTTPPVLDSDGSFFLYSKLTVTKEEWQQGFVFSCSVMHEALHNHYTQKSLS | |
| | LSPGK | |
| 353 | APEAAGGPSVFLFPPKPKDTLMISRTPEVTCVVVDVSHEDPEVKFNWYVDGVE | Clone CH3C.35.21 with hole |
| | VHNAKTKPREEQYNSTYRVVSVLTVLHQDWLNGKEYKCKVSNKALGAPIEKTI | and LALAPG mutations |
| | SKAKGQPREPQVYTLPPSRDELTKNQVSLSCAVKGFYPSDIAVWWESYGTEWS | |
| | SYKTTPPVLDSDGSFFLVSKLTVTKEEWQQGFVFSCSVMHEALHNHYTQKSLS | |
| | LSPGK | |
| 354 | APEAAGGPSVFLFPPKPKDTLYITREPEVTCVVVDVSHEDPEVKFNWYVDGVE | |
| | VHNAKTKPREEQYNSTYRVVSVLTVLHQDWLNGKEYKCKVSNKALGAPIEKTI | Clone CH3C.35.21 with hole, |
| | SKAKGQPREPQVYTLPPSRDELTKNQVSLSCAVKGFYPSDIAVWWESYGTEWS | LALAPG, and YTE |
| | SYKTTPPVLDSDGSFFLVSKLTVTKEEWQQGFVFSCSVMHEALHNHYTQKSLS | mutations |
| | LSPGK | |
| 355 | APELLGGPSVFLFPPKPKDTLMISRTPEVTCVVVDVSHEDPEVKFNWYVDGVE | Clone CH3C.35.20.1.1 with |
| | VHNAKTKPREEQYNSTYRVVSVLTVLHQDWLNGKEYKCKVSNKALPAPIEKTI | knob mutation |
| | SKAKGQPREPQVYTLPPSRDELTKNQVSLWCLVKGFYPSDIAVEWESFGTEWS | |
| | SYKTTPPVLDSDGSFFLYSKLTVSKEEWQQGFVFSCSVMHEALHNHYTQKSLS | |
| | LSPGK | |
| 356 | APEAAGGPSVFLFPPKPKDTLMISRTPEVTCVVVDVSHEDPEVKFNWYVDGVE | Clone CH3C.35.20.1.1 with |
| | VHNAKTKPREEQYNSTYRVVSVLTVLHQDWLNGKEYKCKVSNKALPAPIEKTI | knob and LALA mutations |
| | SKAKGQPREPQVYTLPPSRDELTKNQVSLWCLVKGFYPSDIAVEWESFGTEWS | |
| | SYKTTPPVLDSDGSFFLYSKLTVSKEEWQQGFVFSCSVMHEALHNHYTQKSLS | |
| | LSPGK | |
| 357 | APEAAGGPSVFLFPPKPKDTLMISRTPEVTCVVVDVSHEDPEVKFNWYVDGVE | Clone CH3C.35.20.1.1 with |
| | VHNAKTKPREEQYNSTYRVVSVLTVLHQDWLNGKEYKCKVSNKALGAPIEKTI | knob and LALAPG |
| | SKAKGQPREPQVYTLPPSRDELTKNQVSLWCLVKGFYPSDIAVEWESFGTEWS | mutations |
| | SYKTTPPVLDSDGSFFLYSKLTVSKEEWQQGFVFSCSVMHEALHNHYTQKSLS | |
| | LSPGK | |
| 358 | APELLGGPSVFLFPPKPKDTLYITREPEVTCVVVDVSHEDPEVKFNWYVDGVE | Clone CH3C.35.20.1.1 with |
| | VHNAKTKPREEQYNSTYRVVSVLTVLHQDWLNGKEYKCKVSNKALPAPIEKTI | knob and YTE mutations |
| | SKAKGQPREPQVYTLPPSRDELTKNQVSLWCLVKGFYPSDIAVEWESFGTEWS | |
| | SYKTTPPVLDSDGSFFLYSKLTVSKEEWQQGFVFSCSVMHEALHNHYTQKSLS | |
| | LSPGK | |
| 359 | APEAAGGPSVFLFPPKPKDTLYITREPEVTCVVVDVSHEDPEVKFNWYVDGVE | Clone CH3C.35.20.1.1 with |
| | VHNAKTKPREEQYNSTYRVVSVLTVLHQDWLNGKEYKCKVSNKALPAPIEKTI | knob, LALA, and YTE |
| | SKAKGQPREPQVYTLPPSRDELTKNQVSLWCLVKGFYPSDIAVEWESFGTEWS | mutations |
| | SYKTTPPVLDSDGSFFLYSKLTVSKEEWQQGFVFSCSVMHEALHNHYTQKSLS | |
| | LSPGK | |
| 360 | APEAAGGPSVFLFPPKPKDTLYITREPEVTCVVVDVSHEDPEVKFNWYVDGVE | Clone CH3C.35.20.1.1 with |
| | VHNAKTKPREEQYNSTYRVVSVLTVLHQDWLNGKEYKCKVSNKALGAPIEKTI | knob, LALAPG, and YTE |
| | SKAKGQPREPQVYTLPPSRDELTKNQVSLWCLVKGFYPSDIAVEWESFGTEWS | mutations |
| | SYKTTPPVLDSDGSFFLYSKLTVSKEEWQQGFVFSCSVMHEALHNHYTQKSLS | |
| | LSPGK | |
| 361 | APELLGGPSVFLFPPKPKDTLMISRTPEVTCVVVDVSHEDPEVKFNWYVDGVE | Clone CH3C.35.20.1.1 with |
| | VHNAKTKPREEQYNSTYRVVSVLTVLHQDWLNGKEYKCKVSNKALPAPIEKTI | hole mutations |
| | SKAKGQPREPQVYTLPPSRDELTKNQVSLSCAVKGFYPSDIAVEWESFGTEWS | |
| | SYKTTPPVLDSDGSFFLVSKLTVSKEEWQQGFVFSCSVMHEALHNHYTQKSLS | |
| | LSPGK | |
| 362 | APEAAGGPSVFLFPPKPKDTLMISRTPEVTCVVVDVSHEDPEVKFNWYVDGVE | Clone CH3C.35.20.1.1 with |
| | VHNAKTKPREEQYNSTYRVVSVLTVLHQDWLNGKEYKCKVSNKALPAPIEKTI | hole and LALA mutations |
| | SKAKGQPREPQVYTLPPSRDELTKNQVSLSCAVKGFYPSDIAVEWESFGTEWS | |
| | SYKTTPPVLDSDGSFFLVSKLTVSKEEWQQGFVFSCSVMHEALHNHYTQKSLS | |
| | LSPGK | |
| 363 | APEAAGGPSVFLFPPKPKDTLMISRTPEVTCVVVDVSHEDPEVKFNWYVDGVE | Clone CH3C.35.20.1.1 with |
| | VHNAKTKPREEQYNSTYRVVSVLTVLHQDWLNGKEYKCKVSNKALGAPIEKTI | hole and LALAPG mutations |
| | SKAKGQPREPQVYTLPPSRDELTKNQVSLSCAVKGFYPSDIAVEWESFGTEWS | |
| | SYKTTPPVLDSDGSFFLVSKLTVSKEEWQQGFVFSCSVMHEALHNHYTQKSLS | |
| | LSPGK | |
| 364 | APELLGGPSVFLFPPKPKDTLYITREPEVTCVVVDVSHEDPEVKFNWYVDGVE | Clone CH3C.35.20.1.1 with |
| | VHNAKTKPREEQYNSTYRVVSVLTVLHQDWLNGKEYKCKVSNKALPAPIEKTI | hole and YTE mutations |
| | SKAKGQPREPQVYTLPPSRDELTKNQVSLSCAVKGFYPSDIAVEWESFGTEWS | |
| | SYKTTPPVLDSDGSFFLVSKLTVSKEEWQQGFVFSCSVMHEALHNHYTQKSLS | |
| | LSPGK | |
| 365 | APEAAGGPSVFLFPPKPKDTLYITREPEVTCVVVDVSHEDPEVKFNWYVDGVE | Clone CH3C.35.20.1.1 with |
| | VHNAKTKPREEQYNSTYRVVSVLTVLHQDWLNGKEYKCKVSNKALPAPIEKTI | hole, LALA, and YTE |
| | SKAKGQPREPQVYTLPPSRDELTKNQVSLSCAVKGFYPSDIAVEWESFGTEWS | mutations |
| | SYKTTPPVLDSDGSFFLVSKLTVSKEEWQQGFVFSCSVMHEALHNHYTQKSLS | |
| | LSPGK | |
| 366 | APEAAGGPSVFLFPPKPKDTLYITREPEVTCVVVDVSHEDPEVKFNWYVDGVE | Clone CH3C.35.20.1.1 with |
| | VHNAKTKPREEQYNSTYRVVSVLTVLHQDWLNGKEYKCKVSNKALGAPIEKTI | hole, LALAPG, and YTE |
| | SKAKGQPREPQVYTLPPSRDELTKNQVSLSCAVKGFYPSDIAVEWESFGTEWS | mutations |
| | SYKTTPPVLDSDGSFFLVSKLTVSKEEWQQGFVFSCSVMHEALHNHYTQKSLS | |
| | LSPGK | |
| 367 | APELLGGPSVFLFPPKPKDTLMISRTPEVTCVVVDVSHEDPEVKFNWYVDGVE | Clone CH3C.35.23.2.1 with |
| | VHNAKTKPREEQYNSTYRVVSVLTVLHQDWLNGKEYKCKVSNKALPAPIEKTI | knob mutation |
| | SKAKGQPREPQVYTLPPSRDELTKNQVSLWCLVKGFYPSDIAVEWESYGTEWA | |
| | NYKTTPPVLDSDGSFFLYSKLTVSKSEWQQGFVFSCSVMHEALHNHYTQKSLS | |
| | LSPGK | |
| 368 | APEAAGGPSVFLFPPKPKDTLMISRTPEVTCVVVDVSHEDPEVKFNWYVDGVE | Clone CH3C.35.23.2.1 with |
| | VHNAKTKPREEQYNSTYRVVSVLTVLHQDWLNGKEYKCKVSNKALPAPIEKTI | knob and LALA mutations |
| | SKAKGQPREPQVYTLPPSRDELTKNQVSLWCLVKGFYPSDIAVEWESYGTEWA | |
| | NYKTTPPVLDSDGSFFLYSKLTVSKSEWQQGFVFSCSVMHEALHNHYTQKSLS | |
| | LSPGK | |
| 369 | APEAAGGPSVFLFPPKPKDTLMISRTPEVTCVVVDVSHEDPEVKFNWYVDGVE | Clone CH3C.35.23.2.1 with |
| | VHNAKTKPREEQYNSTYRVVSVLTVLHQDWLNGKEYKCKVSNKALGAPIEKTI | knob and LALAPG mutations |
| | SKAKGQPREPQVYTLPPSRDELTKNQVSLWCLVKGFYPSDIAVEWESYGTEWA | |
| | NYKTTPPVLDSDGSFFLYSKLTVSKSEWQQGFVFSCSVMHEALHNHYTQKSLS | |
| | LSPGK | |
| 370 | APELLGGPSVFLFPPKPKDTLYITREPEVTCVVVDVSHEDPEVKFNWYVDGVE | Clone CH3C.35.23.2.1 with |
| | VHNAKTKPREEQYNSTYRVVSVLTVLHQDWLNGKEYKCKVSNKALPAPIEKTI | knob and YTE mutations |
| | SKAKGQPREPQVYTLPPSRDELTKNQVSLWCLVKGFYPSDIAVEWESYGTEWA | |
| | NYKTTPPVLDSDGSFFLYSKLTVSKSEWQQGFVFSCSVMHEALHNHYTQKSLS | |
| | LSPGK | |
| 371 | APEAAGGPSVFLFPPKPKDTLYITREPEVTCVVVDVSHEDPEVKFNWYVDGVE | Clone CH3C.35.23.2.1 with |
| | VHNAKTKPREEQYNSTYRVVSVLTVLHQDWLNGKEYKCKVSNKALPAPIEKTI | knob, LALA, and YTE |
| | SKAKGQPREPQVYTLPPSRDELTKNQVSLWCLVKGFYPSDIAVEWESYGTEWA | mutations |
| | NYKTTPPVLDSDGSFFLYSKLTVSKSEWQQGFVFSCSVMHEALHNHYTQKSLS | |
| | LSPGK | |
| 372 | APEAAGGPSVFLFPPKPKDTLYITREPEVTCVVVDVSHEDPEVKFNWYVDGVE | Clone CH3C.35.23.2.1 with |
| | VHNAKTKPREEQYNSTYRVVSVLTVLHQDWLNGKEYKCKVSNKALGAPIEKTI | knob, LALAPG, and YTE |
| | SKAKGQPREPQVYTLPPSRDELTKNQVSLWCLVKGFYPSDIAVEWESYGTEWA | mutations |
| | NYKTTPPVLDSDGSFFLYSKLTVSKSEWQQGFVFSCSVMHEALHNHYTQKSLS | |
| | LSPGK | |
| 373 | APELLGGPSVFLFPPKPKDTLMISRTPEVTCVVVDVSHEDPEVKFNWYVDGVE | Clone CH3C.35.23.2.1 with |
| | VHNAKTKPREEQYNSTYRVVSVLTVLHQDWLNGKEYKCKVSNKALPAPIEKTI | hole mutations |
| | SKAKGQPREPQVYTLPPSRDELTKNQVSLSCAVKGFYPSDIAVEWESYGTEWA | |
| | NYKTTPPVLDSDGSFFLVSKLTVSKSEWQQGFVFSCSVMHEALHNHYTQKSLS | |
| | LSPGK | |
| 374 | APEAAGGPSVFLFPPKPKDTLMISRTPEVTCVVVDVSHEDPEVKFNWYVDGVE | Clone CH3C.35.23.2.1 with |
| | VHNAKTKPREEQYNSTYRVVSVLTVLHQDWLNGKEYKCKVSNKALPAPIEKTI | hole and LALA mutations |
| | SKAKGQPREPQVYTLPPSRDELTKNQVSLSCAVKGFYPSDIAVEWESYGTEWA | |
| | NYKTTPPVLDSDGSFFLVSKLTVSKSEWQQGFVFSCSVMHEALHNHYTQKSLS | |
| | LSPGK | |
| 375 | APEAAGGPSVFLFPPKPKDTLMISRTPEVTCVVVDVSHEDPEVKFNWYVDGVE | Clone CH3C.35.23.2.1 with |
| | VHNAKTKPREEQYNSTYRVVSVLTVLHQDWLNGKEYKCKVSNKALGAPIEKTI | hole and LALAPG mutations |
| | SKAKGQPREPQVYTLPPSRDELTKNQVSLSCAVKGFYPSDIAVEWESYGTEWA | |
| | NYKTTPPVLDSDGSFFLVSKLTVSKSEWQQGFVFSCSVMHEALHNHYTQKSLS | |
| | LSPGK | |
| 376 | APELLGGPSVFLFPPKPKDTLYITREPEVTCVVVDVSHEDPEVKFNWYVDGVE | Clone CH3C.35.23.2.1 with |
| | VHNAKTKPREEQYNSTYRVVSVLTVLHQDWLNGKEYKCKVSNKALPAPIEKTI | hole and YTE mutations |
| | SKAKGQPREPQVYTLPPSRDELTKNQVSLSCAVKGFYPSDIAVEWESYGTEWA | |
| | NYKTTPPVLDSDGSFFLVSKLTVSKSEWQQGFVFSCSVMHEALHNHYTQKSLS | |
| | LSPGK | |
| 377 | APEAAGGPSVFLFPPKPKDTLYITREPEVTCVVVDVSHEDPEVKFNWYVDGVE | Clone CH3C.35.23.2.1 with |
| | VHNAKTKPREEQYNSTYRVVSVLTVLHQDWLNGKEYKCKVSNKALPAPIEKTI | hole, LALA, and YTE |
| | SKAKGQPREPQVYTLPPSRDELTKNQVSLSCAVKGFYPSDIAVEWESYGTEWA | mutations |
| | NYKTTPPVLDSDGSFFLVSKLTVSKSEWQQGFVFSCSVMHEALHNHYTQKSLS | |
| | LSPGK | |
| 378 | APEAAGGPSVFLFPPKPKDTLYITREPEVTCVVVDVSHEDPEVKFNWYVDGVE | Clone CH3C.35.23.2.1 with |
| | VHNAKTKPREEQYNSTYRVVSVLTVLHQDWLNGKEYKCKVSNKALGAPIEKTI | hole, LALAPG, and YTE |
| | SKAKGQPREPQVYTLPPSRDELTKNQVSLSCAVKGFYPSDIAVEWESYGTEWA | SDGSFFLV mutations |
| | NYKTTPPVLDSKLTVSKSEWQQGFVFSCSVMHEALHNHYTQKSLS | |
| | LSPGK | |
| 379 | APELLGGPSVFLFPPKPKDTLMISRTPEVTCVVVDVSHEDPEVKFNWYVDGVE | Clone CH3C.35.23.1.1 with |
| | VHNAKTKPREEQYNSTYRVVSVLTVLHQDWLNGKEYKCKVSNKALPAPIEKTI | knob mutation |
| | SKAKGQPREPQVYTLPPSRDELTKNQVSLWCLVKGFYPSDIAVEWESFGTEWS | |
| | NYKTTPPVLDSDGSFFLYSKLTVSKEEWQQGFVFSCSVMHEALHNHYTQKSLS | |
| | LSPGK | |
| 380 | APEAAGGPSVFLFPPKPKDTLMISRTPEVTCVVVDVSHEDPEVKFNWYVDGVE | Clone CH3C.35.23.1.1 with |
| | VHNAKTKPREEQYNSTYRVVSVLTVLHQDWLNGKEYKCKVSNKALPAPIEKTI | knob and LALA mutations |
| | SKAKGQPREPQVYTLPPSRDELTKNQVSLWCLVKGFYPSDIAVEWESFGTEWS | |
| | NYKTTPPVLDSDGSFFLYSKLTVSKEEWQQGFVFSCSVMHEALHNHYTQKSLS | |
| | LSPGK | |
| 381 | APEAAGGPSVFLFPPKPKDTLMISRTPEVTCVVVDVSHEDPEVKFNWYVDGVE | Clone CH3C.35.23.1.1 with |
| | VHNAKTKPREEQYNSTYRVVSVLTVLHQDWLNGKEYKCKVSNKALGAPIEKTI | knob and LALAPG |
| | SKAKGQPREPQVYTLPPSRDELTKNQVSLWCLVKGFYPSDIAVEWESFGTEWS | mutations |
| | NYKTTPPVLDSDGSFFLYSKLTVSKEEWQQGFVFSCSVMHEALHNHYTQKSLS | |
| | LSPGK | |
| 382 | APELLGGPSVFLFPPKPKDTLYITREPEVTCVVVDVSHEDPEVKFNWYVDGVE | Clone CH3C.35.23.1.1 with |
| | VHNAKTKPREEQYNSTYRVVSVLTVLHQDWLNGKEYKCKVSNKALPAPIEKTI | knob and YI E mutations |
| | SKAKGQPREPQVYTLPPSRDELTKNQVSLWCLVKGFYPSDIAVEWESFGTEWS | |
| | NYKTTPPVLDSDGSFFLYSKLTVSKEEWQQGFVFSCSVMHEALHNHYTQKSLS | |
| | LSPGK | |
| 383 | APEAAGGPSVFLFPPKPKDTLYITREPEVTCVVVDVSHEDPEVKFNWYVDGVE | Clone CH3C.35.23.1.1 with |
| | VHNAKTKPREEQYNSTYRVVSVLTVLHQDWLNGKEYKCKVSNKALPAPIEKTI | knob, LALA, and YTE |
| | SKAKGQPREPQVYTLPPSRDELTKNQVSLWCLVKGFYPSDIAVEWESFGTEWS | mutations |
| | NYKTTPPVLDSDGSFFLYSKLTVSKEEWQQGFVFSCSVMHEALHNHYTQKSLS | |
| | LSPGK | |
| 384 | APEAAGGPSVFLFPPKPKDTLYITREPEVTCVVVDVSHEDPEVKFNWYVDGVE | Clone CH3C.35.23.1.1 with |
| | VHNAKTKPREEQYNSTYRVVSVLTVLHQDWLNGKEYKCKVSNKALGAPIEKTI | knob, LALAPG, and YTE |
| | SKAKGQPREPQVYTLPPSRDELTKNQVSLWCLVKGFYPSDIAVEWESFGTEWS | mutations |
| | NYKTTPPVLDSDGSFFLYSKLTVSKEEWQQGFVFSCSVMHEALHNHYTQKSLS | |
| | LSPGK | |
| 385 | APELLGGPSVFLFPPKPKDTLMISRTPEVTCVVVDVSHEDPEVKFNWYVDGVE | Clone CH3C.35.23.1.1 with |
| | VHNAKTKPREEQYNSTYRVVSVLTVLHQDWLNGKEYKCKVSNKALPAPIEKTI | hole mutations |
| | SKAKGQPREPQVYTLPPSRDELTKNQVSLSCAVKGFYPSDIAVEWESFGTEWS | |
| | NYKTTPPVLDSDGSFFLVSKLTVSKEEWQQGFVFSCSVMHEALHNHYTQKSLS | |
| | LSPGK | |
| 386 | APEAAGGPSVFLFPPKPKDTLMISRTPEVTCVVVDVSHEDPEVKFNWYVDGVE | Clone CH3C.35.23.1.1 with |
| | VHNAKTKPREEQYNSTYRVVSVLTVLHQDWLNGKEYKCKVSNKALPAPIEKTI | hole and LALA mutations |
| | SKAKGQPREPQVYTLPPSRDELTKNQVSLSCAVKGFYPSDIAVEWESFGTEWS | |
| | NYKTTPPVLDSDGSFFLVSKLTVSKEEWQQGFVFSCSVMHEALHNHYTQKSLS | |
| | LSPGK | |
| 387 | APEAAGGPSVFLFPPKPKDTLMISRTPEVTCVVVDVSHEDPEVKFNWYVDGVE | Clone CH3C.35.23.1.1 with |
| | VHNAKTKPREEQYNSTYRVVSVLTVLHQDWLNGKEYKCKVSNKALGAPIEKTI | hole and LALAPG mutations |
| | SKAKGQPREPQVYTLPPSRDELTKNQVSLSCAVKGFYPSDIAVEWESFGTEWS | |
| | NYKTTPPVLDSDGSFFLVSKLTVSKEEWQQGFVFSCSVMHEALHNHYTQKSLS | |
| | LSPGK | |
| 388 | APELLGGPSVFLFPPKPKDTLYITREPEVTCVVVDVSHEDPEVKFNWYVDGVE | Clone CH3C.35.23.1.1 with |
| | VHNAKTKPREEQYNSTYRVVSVLTVLHQDWLNGKEYKCKVSNKALPAPIEKTI | hole and YTE mutations |
| | SKAKGQPREPQVYTLPPSRDELTKNQVSLSCAVKGFYPSDIAVEWESFGTEWS | |
| | NYKTTPPVLDSDGSFFLVSKLTVSKEEWQQGFVFSCSVMHEALHNHYTQKSLS | |
| | LSPGK | |
| 389 | APEAAGGPSVFLFPPKPKDTLYITREPEVTCVVVDVSHEDPEVKFNWYVDGVE | Clone CH3C.35.23.1.1 with |
| | VHNAKTKPREEQYNSTYRVVSVLTVLHQDWLNGKEYKCKVSNKALPAPIEKTI | hole, LALA, and YTE |
| | SKAKGQPREPQVYTLPPSRDELTKNQVSLSCAVKGFYPSDIAVEWESFGTEWS | mutations |
| | NYKTTPPVLDSDGSFFLVSKLTVSKEEWQQGFVFSCSVMHEALHNHYTQKSLS | |
| | LSPGK | |
| 390 | APEAAGGPSVFLFPPKPKDTLYITREPEVTCVVVDVSHEDPEVKFNWYVDGVE | Clone CH3C.35.23.1.1 with |
| | VHNAKTKPREEQYNSTYRVVSVLTVLHQDWLNGKEYKCKVSNKALGAPIEKTI | hole, LALAPG, and YTE |
| | SKAKGQPREPQVYTLPPSRDELTKNQVSLSCAVKGFYPSDIAVEWESFGTEWS | mutations |
| | NYKTTPPVLDSDGSFFLVSKLTVSKEEWQQGFVFSCSVMHEALHNHYTQKSLS | |
| | LSPGK | |
| 391 | APELLGGPSVFLFPPKPKDTLMISRTPEVTCVVVDVSHEDPEVKFNWYVDGVE | Clone CH3C.35.20.1 M428L |
| | VHNAKTKPREEQYNSTYRVVSVLTVLHQDWLNGKEYKCKVSNKALPAPIEKTI | and N434S mutations |
| | SKAKGQPREPQVYTLPPSRDELTKNQVSLTCLVKGFYPSDIAVEWESFGTEWS | |
| | SYKTTPPVLDSDGSFFLYSKLTVTKEEWQQGFVFSCSVLHEALHSHYTQKSLS | |
| | LSPGK | |
| 392 | APELLGGPSVFLFPPKPKDTLMISRTPEVTCVVVDVSHEDPEVKFNWYVDGVE | Clone CH3C.35.20.1 with |
| | VHNAKTKPREEQYNSTYRVVSVLTVLHQDWLNGKEYKCKVSNKALPAPIEKTI | knob and M428L and N4345 |
| | SKAKGQPREPQVYTLPPSRDELTKNQVSLWCLVKGFYPSDIAVEWESFGTEWS | mutations |
| | SYKTTPPVLDSDGSFFLYSKLTVTKEEWQQGFVFSCSVLHEALHSHYTQKSLS | |
| | LSPGK | |
| 393 | APEAAGGPSVFLFPPKPKDTLMISRTPEVTCVVVDVSHEDPEVKFNWYVDGVE | Clone CH3C.35.20.1 with |
| | VHNAKTKPREEQYNSTYRVVSVLTVLHQDWLNGKEYKCKVSNKALPAPIEKTI | knob, LALA, and M428L and |
| | SKAKGQPREPQVYTLPPSRDELTKNQVSLWCLVKGFYPSDIAVEWESFGTEWS | N4345 mutations |
| | SYKTTPPVLDSDGSFFLYSKLTVTKEEWQQGFVFSCSVLHEALHSHYTQKSLS | |
| | LSPGK | |
| 394 | APEAAGGPSVFLFPPKPKDTLMISRTPEVTCVVVDVSHEDPEVKFNWYVDGVE | Clone CH3C.35.20.1 with |
| | VHNAKTKPREEQYNSTYRVVSVLTVLHQDWLNGKEYKCKVSNKALGAPIEKTI | knob, LALAPG, and M428L |
| | SKAKGQPREPQVYTLPPSRDELTKNQVSLWCLVKGFYPSDIAVEWESFGTEWS | and N4345 mutations |
| | SYKTTPPVLDSDGSFFLYSKLTVTKEEWQQGFVFSCSVLHEALHSHYTQKSLS | |
| | LSPGK | |
| 395 | APELLGGPSVFLFPPKPKDTLMISRTPEVTCVVVDVSHEDPEVKFNWYVDGVE | Clone CH3C.35.20.1 with |
| | VHNAKTKPREEQYNSTYRVVSVLTVLHQDWLNGKEYKCKVSNKALPAPIEKTI | hole and M428L and N4345 |
| | SKAKGQPREPQVYTLPPSRDELTKNQVSLSCAVKGFYPSDIAVEWESFGTEWS | mutations |
| | SYKTTPPVLDSDGSFFLVSKLTVTKEEWQQGFVFSCSVLHEALHSHYTQKSLS | |
| | LSPGK | |
| 396 | APEAAGGPSVFLFPPKPKDTLMISRTPEVTCVVVDVSHEDPEVKFNWYVDGVE | Clone CH3C.35.20.1 with |
| | VHNAKTKPREEQYNSTYRVVSVLTVLHQDWLNGKEYKCKVSNKALPAPIEKTI | hole, LALA, and M428L and |
| | SKAKGQPREPQVYTLPPSRDELTKNQVSLSCAVKGFYPSDIAVEWESFGTEWS | N4345 mutations |
| | SYKTTPPVLDSDGSFFLVSKLTVTKEEWQQGFVFSCSVLHEALHSHYTQKSLS | |
| | LSPGK | |
| 397 | APEAAGGPSVFLFPPKPKDTLMISRTPEVTCVVVDVSHEDPEVKFNWYVDGVE | Clone CH3C.35.20.1 with |
| | VHNAKTKPREEQYNSTYRVVSVLTVLHQDWLNGKEYKCKVSNKALGAPIEKTI | hole, LALAPG, and M428L |
| | SKAKGQPREPQVYTLPPSRDELTKNQVSLSCAVKGFYPSDIAVEWESFGTEWS | and N4345 mutations |
| | SYKTTPPVLDSDGSFFLVSKLTVTKEEWQQGFVFSCSVLHEALHSHYTQKSLS | |
| | LSPGK | |
| 398 | APELLGGPSVFLFPPKPKDTLMISRTPEVTCVVVDVSHEDPEVKFNWYVDGVE | Clone CH3C.35.20.1.1 with |
| | VHNAKTKPREEQYNSTYRVVSVLTVLHQDWLNGKEYKCKVSNKALPAPIEKTI | M428L and N4345 mutations |
| | SKAKGQPREPQVYTLPPSRDELTKNQVSLTCLVKGFYPSDIAVEWESFGTEWS | |
| | SYKTTPPVLDSDGSFFLYSKLTVSKEEWQQGFVFSCSVLHEALHSHYTQKSLS | |
| | LSPGK | |
| 399 | APELLGGPSVFLFPPKPKDTLMISRTPEVTCVVVDVSHEDPEVKFNWYVDGVE | Clone CH3C.35.20.1.1 with |
| | VHNAKTKPREEQYNSTYRVVSVLTVLHQDWLNGKEYKCKVSNKALPAPIEKTI | knob and M428L and N4345 |
| | SKAKGQPREPQVYTLPPSRDELTKNQVSLWCLVKGFYPSDIAVEWESFGTEWS | mutations |
| | SYKTTPPVLDSDGSFFLYSKLTVSKEEWQQGFVFSCSVLHEALHSHYTQKSLS | |
| | LSPGK | |
| 400 | APEAAGGPSVFLFPPKPKDTLMISRTPEVTCVVVDVSHEDPEVKFNWYVDGVE | Clone CH3C.35.20.1.1 with |
| | VHNAKTKPREEQYNSTYRVVSVLTVLHQDWLNGKEYKCKVSNKALPAPIEKTI | knob, LALA, and M428L and |
| | SKAKGQPREPQVYTLPPSRDELTKNQVSLWCLVKGFYPSDIAVEWESFGTEWS | N434S mutations |
| | SYKTTPPVLDSDGSFFLYSKLTVSKEEWQQGFVFSCSVLHEALHSHYTQKSLS | |
| | LSPGK | |
| 401 | APEAAGGPSVFLFPPKPKDTLMISRTPEVTCVVVDVSHEDPEVKFNWYVDGVE | Clone CH3C.35.20.1.1 with |
| | VHNAKTKPREEQYNSTYRVVSVLTVLHQDWLNGKEYKCKVSNKALGAPIEKTI | knob, LALAPG, and M428L |
| | SKAKGQPREPQVYTLPPSRDELTKNQVSLWCLVKGFYPSDIAVEWESFGTEWS | and N434S mutations |
| | SYKTTPPVLDSDGSFFLYSKLTVSKEEWQQGFVFSCSVLHEALHSHYTQKSLS | |
| | LSPGK | |
| 402 | APELLGGPSVFLFPPKPKDTLMISRTPEVTCVVVDVSHEDPEVKFNWYVDGVE | Clone CH3C.35.20.1.1 with |
| | VHNAKTKPREEQYNSTYRVVSVLTVLHQDWLNGKEYKCKVSNKALPAPIEKTI | hole and M428L and N434S |
| | SKAKGQPREPQVYTLPPSRDELTKNQVSLSCAVKGFYPSDIAVEWESFGTEWS | mutations |
| | SYKTTPPVLDSDGSFFLVSKLTVSKEEWQQGFVFSCSVLHEALHSHYTQKSLS | |
| | LSPGK | |
| 403 | APEAAGGPSVFLFPPKPKDTLMISRTPEVTCVVVDVSHEDPEVKFNWYVDGVE | Clone CH3C.35.20.1.1 with |
| | VHNAKTKPREEQYNSTYRVVSVLTVLHQDWLNGKEYKCKVSNKALPAPIEKTI | hole, LALA, and M428L and |
| | SKAKGQPREPQVYTLPPSRDELTKNQVSLSCAVKGFYPSDIAVEWESFGTEWS | N434S mutations |
| | SYKTTPPVLDSDGSFFLVSKLTVSKEEWQQGFVFSCSVLHEALHSHYTQKSLS | |
| | LSPGK | |
| 404 | APEAAGGPSVFLFPPKPKDTLMISRTPEVTCVVVDVSHEDPEVKFNWYVDGVE | Clone CH3C.35.20.1.1 with |
| | VHNAKTKPREEQYNSTYRVVSVLTVLHQDWLNGKEYKCKVSNKALGAPIEKTI | hole, LALAPG, and M428L |
| | SKAKGQPREPQVYTLPPSRDELTKNQVSLSCAVKGFYPSDIAVEWESFGTEWS | and N434S mutations |
| | SYKTTPPVLDSDGSFFLVSKLTVSKEEWQQGFVFSCSVLHEALHSHYTQKSLS | |
| | LSPGK | |
| 405 | APELLGGPSVFLFPPKPKDTLMISRTPEVTCVVVDVSHEDPEVKFNWYVDGVE | Clone CH3C.35.21 with |
| | VHNAKTKPREEQYNSTYRVVSVLTVLHQDWLNGKEYKCKVSNKALPAPIEKTI | M428L and N434S mutations |
| | SKAKGQPREPQVYTLPPSRDELTKNQVSLTCLVKGFYPSDIAVWWESYGTEWS | |
| | SYKTTPPVLDSDGSFFLYSKLTVTKEEWQQGFVFSCSVLHEALHSHYTQKSLS | |
| | LSPGK | |
| 406 | APELLGGPSVFLFPPKPKDTLMISRTPEVTCVVVDVSHEDPEVKFNWYVDGVE | Clone CH3C.35.21 with knob |
| | VHNAKTKPREEQYNSTYRVVSVLTVLHQDWLNGKEYKCKVSNKALPAPIEKTI | and M428L and N434S |
| | SKAKGQPREPQVYTLPPSRDELTKNQVSLWCLVKGFYPSDIAVWWESYGTEWS | mutations |
| | SYKTTPPVLDSDGSFFLYSKLTVTKEEWQQGFVFSCSVLHEALHSHYTQKSLS | |
| | LSPGK | |
| 407 | APEAAGGPSVFLFPPKPKDTLMISRTPEVTCVVVDVSHEDPEVKFNWYVDGVE | Clone CH3C.35.21 with |
| | VHNAKTKPREEQYNSTYRVVSVLTVLHQDWLNGKEYKCKVSNKALPAPIEKTI | knob, LALA, and M428L and |
| | SKAKGQPREPQVYTLPPSRDELTKNQVSLWCLVKGFYPSDIAVWWESYGTEWS | N434S mutations |
| | SYKTTPPVLDSDGSFFLYSKLTVTKEEWQQGFVFSCSVLHEALHSHYTQKSLS | |
| | LSPGK | |
| 408 | APEAAGGPSVFLFPPKPKDTLMISRTPEVTCVVVDVSHEDPEVKFNWYVDGVE | Clone CH3C.35.21 with |
| | VHNAKTKPREEQYNSTYRVVSVLTVLHQDWLNGKEYKCKVSNKALGAPIEKTI | knob, LALAPG, and M428L |
| | SKAKGQPREPQVYTLPPSRDELTKNQVSLWCLVKGFYPSDIAVWWESYGTEWS | and N434S mutations |
| | SYKTTPPVLDSDGSFFLYSKLTVTKEEWQQGFVFSCSVLHEALHSHYTQKSLS | |
| | LSPGK | |
| 409 | APELLGGPSVFLFPPKPKDTLMISRTPEVTCVVVDVSHEDPEVKFNWYVDGVE | Clone CH3C.35.21 with hole |
| | VHNAKTKPREEQYNSTYRVVSVLTVLHQDWLNGKEYKCKVSNKALPAPIEKTI | and M428L and N434S |
| | SKAKGQPREPQVYTLPPSRDELTKNQVSLSCAVKGFYPSDIAVWWESYGTEWS | mutations |
| | SYKTTPPVLDSDGSFFLVSKLTVTKEEWQQGFVFSCSVLHEALHSHYTQKSLS | |
| | LSPGK | |
| 410 | APEAAGGPSVFLFPPKPKDTLMISRTPEVTCVVVDVSHEDPEVKFNWYVDGVE | Clone CH3C.35.21 with hole, |
| | VHNAKTKPREEQYNSTYRVVSVLTVLHQDWLNGKEYKCKVSNKALPAPIEKTI | LALA, and M428L and |
| | SKAKGQPREPQVYTLPPSRDELTKNQVSLSCAVKGFYPSDIAVWWESYGTEWS | N434S mutations |
| | SYKTTPPVLDSDGSFFLVSKLTVTKEEWQQGFVFSCSVLHEALHSHYTQKSLS | |
| | LSPGK | |
| 411 | APEAAGGPSVFLFPPKPKDTLMISRTPEVTCVVVDVSHEDPEVKFNWYVDGVE | Clone CH3C.35.21 with hole, |
| | VHNAKTKPREEQYNSTYRVVSVLTVLHQDWLNGKEYKCKVSNKALGAPIEKTI | LALAPG, and M428L and |
| | SKAKGQPREPQVYTLPPSRDELTKNQVSLSCAVKGFYPSDIAVWWESYGTEWS | N434S mutations |
| | SYKTTPPVLDSDGSFFLVSKLTVTKEEWQQGFVFSCSVLHEALHSHYTQKSLS | |
| | LSPGK | |
| 412 | APELLGGPSVFLFPPKPKDTLMISRTPEVTCVVVDVSHEDPEVKFNWYVDGVE | Clone CH3C.35.21.17.2 with |
| | VHNAKTKPREEQYNSTYRVVSVLTVLHQDWLNGKEYKCKVSNKALPAPIEKTI | M428L and N434S mutations |
| | SKAKGQPREPQVYTLPPSRDELTKNQVSLTCLVKGFYPSDIAVLWESYGTEWA | |
| | SYKTTPPVLDSDGSFFLYSKLTVTKEEWQQGFVFSCSVLHEALHSHYTQKSLS | |
| | LSPGK | |
| 413 | APELLGGPSVFLFPPKPKDTLMISRTPEVTCVVVDVSHEDPEVKFNWYVDGVE | Clone CH3C.35.21.17.2 with |
| | VHNAKTKPREEQYNSTYRVVSVLTVLHQDWLNGKEYKCKVSNKALPAPIEKTI | knob and M428L and N434S |
| | SKAKGQPREPQVYTLPPSRDELTKNQVSLWCLVKGFYPSDIAVLWESYGTEWA | mutations |
| | SYKTTPPVLDSDGSFFLYSKLTVTKEEWQQGFVFSCSVLHEALHSHYTQKSLS | |
| | LSPGK | |
| 414 | APEAAGGPSVFLFPPKPKDTLMISRTPEVTCVVVDVSHEDPEVKFNWYVDGVE | Clone CH3C.35.21.17.2 with |
| | VHNAKTKPREEQYNSTYRVVSVLTVLHQDWLNGKEYKCKVSNKALPAPIEKTI | knob, LALA, and M428L and |
| | SKAKGQPREPQVYTLPPSRDELTKNQVSLWCLVKGFYPSDIAVLWESYGTEWA | N434S mutations |
| | SYKTTPPVLDSDGSFFLYSKLTVTKEEWQQGFVFSCSVLHEALHSHYTQKSLS | |
| | LSPGK | |
| 415 | APEAAGGPSVFLFPPKPKDTLMISRTPEVTCVVVDVSHEDPEVKFNWYVDGVE | Clone CH3C.35.21.17.2 with |
| | VHNAKTKPREEQYNSTYRVVSVLTVLHQDWLNGKEYKCKVSNKALGAPIEKTI | knob, LALAPG, and M428L |
| | SKAKGQPREPQVYTLPPSRDELTKNQVSLWCLVKGFYPSDIAVLWESYGTEWA | and N434S mutations |
| | SYKTTPPVLDSDGSFFLYSKLTVTKEEWQQGFVFSCSVLHEALHSHYTQKSLS | |
| | LSPGK | |
| 416 | APELLGGPSVFLFPPKPKDTLMISRTPEVTCVVVDVSHEDPEVKFNWYVDGVE | Clone CH3C.35.21.17.2 with |
| | VHNAKTKPREEQYNSTYRVVSVLTVLHQDWLNGKEYKCKVSNKALPAPIEKTI | hole and M428L and N434S |
| | SKAKGQPREPQVYTLPPSRDELTKNQVSLSCAVKGFYPSDIAVLWESYGTEWA | mutations |
| | SYKTTPPVLDSDGSFFLVSKLTVTKEEWQQGFVFSCSVLHEALHSHYTQKSLS | |
| | LSPGK | |
| 417 | APEAAGGPSVFLFPPKPKDTLMISRTPEVTCVVVDVSHEDPEVKFNWYVDGVE | Clone CH3C.35.21.17.2 with |
| | VHNAKTKPREEQYNSTYRVVSVLTVLHQDWLNGKEYKCKVSNKALPAPIEKTI | hole, LALA, and M428L and |
| | SKAKGQPREPQVYTLPPSRDELTKNQVSLSCAVKGFYPSDIAVLWESYGTEWA | N434S mutations |
| | SYKTTPPVLDSDGSFFLVSKLTVTKEEWQQGFVFSCSVLHEALHSHYTQKSLS | |
| | LSPGK | |
| 418 | APEAAGGPSVFLFPPKPKDTLMISRTPEVTCVVVDVSHEDPEVKFNWYVDGVE | Clone CH3C.35.21.17.2 with |
| | VHNAKTKPREEQYNSTYRVVSVLTVLHQDWLNGKEYKCKVSNKALGAPIEKTI | hole, LALAPG, and M428L |
| | SKAKGQPREPQVYTLPPSRDELTKNQVSLSCAVKGFYPSDIAVLWESYGTEWA | and N434S mutations |
| | SYKTTPPVLDSDGSFFLVSKLTVTKEEWQQGFVFSCSVLHEALHSHYTQKSLS | |
| | LSPGK | |
| 419 | APELLGGPSVFLFPPKPKDTLMISRTPEVTCVVVDVSHEDPEVKFNWYVDGVE | Clone CH3C.35.23 with |
| | VHNAKTKPREEQYNSTYRVVSVLTVLHQDWLNGKEYKCKVSNKALPAPIEKTI | M428L and N434S mutations |
| | SKAKGQPREPQVYTLPPSRDELTKNQVSLTCLVKGFYPSDIAVEWESYGTEWS | SDGSFFLYS |
| | NYKTTPPVLDKLTVTKEEWQQGFVFSCSVLHEALHSHYTQKSLS | |
| | LSPGK | |
| 420 | APELLGGPSVFLFPPKPKDTLMISRTPEVTCVVVDVSHEDPEVKFNWYVDGVE | Clone CH3C.35.23 with knob |
| | VHNAKTKPREEQYNSTYRVVSVLTVLHQDWLNGKEYKCKVSNKALPAPIEKTI | and M428L and N434S |
| | SKAKGQPREPQVYTLPPSRDELTKNQVSLWCLVKGFYPSDIAVEWESYGTEWS | mutations |
| | NYKTTPPVLDSDGSFFLYSKLTVTKEEWQQGFVFSCSVLHEALHSHYTQKSLS | |
| | LSPGK | |
| 421 | APEAAGGPSVFLFPPKPKDTLMISRTPEVTCVVVDVSHEDPEVKFNWYVDGVE | Clone CH3C.35.23 with |
| | VHNAKTKPREEQYNSTYRVVSVLTVLHQDWLNGKEYKCKVSNKALPAPIEKTI | knob, LALA, and M428L and |
| | SKAKGQPREPQVYTLPPSRDELTKNQVSLWCLVKGFYPSDIAVEWESYGTEWS | N434S mutations |
| | NYKTTPPVLDSDGSFFLYSKLTVTKEEWQQGFVFSCSVLHEALHSHYTQKSLS | |
| | LSPGK | |
| 422 | APEAAGGPSVFLFPPKPKDTLMISRTPEVTCVVVDVSHEDPEVKFNWYVDGVE | Clone CH3C.35.23 with |
| | VHNAKTKPREEQYNSTYRVVSVLTVLHQDWLNGKEYKCKVSNKALGAPIEKTI | knob, LALAPG, and M428L |
| | SKAKGQPREPQVYTLPPSRDELTKNQVSLWCLVKGFYPSDIAVEWESYGTEWS | and N434S mutations |
| | NYKTTPPVLDSDGSFFLYSKLTVTKEEWQQGFVFSCSVLHEALHSHYTQKSLS | |
| | LSPGK | |
| 423 | APELLGGPSVFLFPPKPKDTLMISRTPEVTCVVVDVSHEDPEVKFNWYVDGVE | Clone CH3C.35.23 with hole |
| | VHNAKTKPREEQYNSTYRVVSVLTVLHQDWLNGKEYKCKVSNKALPAPIEKTI | and M428L and N434S |
| | SKAKGQPREPQVYTLPPSRDELTKNQVSLSCAVKGFYPSDIAVEWESYGTEWS | mutations |
| | NYKTTPPVLDSDGSFFLVSKLTVTKEEWQQGFVFSCSVLHEALHSHYTQKSLS | |
| | LSPGK | |
| 424 | APEAAGGPSVFLFPPKPKDTLMISRTPEVTCVVVDVSHEDPEVKFNWYVDGVE | Clone CH3C.35.23 with hole, |
| | VHNAKTKPREEQYNSTYRVVSVLTVLHQDWLNGKEYKCKVSNKALPAPIEKTI | LALA, and M428L and |
| | SKAKGQPREPQVYTLPPSRDELTKNQVSLSCAVKGFYPSDIAVEWESYGTEWS | N434S mutations |
| | NYKTTPPVLDSDGSFFLVSKLTVTKEEWQQGFVFSCSVLHEALHSHYTQKSLS | |
| | LSPGK | |
| 425 | APEAAGGPSVFLFPPKPKDTLMISRTPEVTCVVVDVSHEDPEVKFNWYVDGVE | Clone CH3C.35.23 with hole, |
| | VHNAKTKPREEQYNSTYRVVSVLTVLHQDWLNGKEYKCKVSNKALGAPIEKTI | LALAPG, and M428L and |
| | SKAKGQPREPQVYTLPPSRDELTKNQVSLSCAVKGFYPSDIAVEWESYGTEWS | N434S mutations |
| | NYKTTPPVLDSDGSFFLVSKLTVTKEEWQQGFVFSCSVLHEALHSHYTQKSLS | |
| | LSPGK | |
| 426 | APELLGGPSVFLFPPKPKDTLMISRTPEVTCVVVDVSHEDPEVKFNWYVDGVE | Clone CH3C.35.23.1.1 with |
| | VHNAKTKPREEQYNSTYRVVSVLTVLHQDWLNGKEYKCKVSNKALPAPIEKTI | M428L and N434S mutations |
| | SKAKGQPREPQVYTLPPSRDELTKNQVSLTCLVKGFYPSDIAVEWESFGTEWS | |
| | NYKTTPPVLDSDGSFFLYSKLTVSKEEWQQGFVFSCSVLHEALHSHYTQKSLS | |
| | LSPGK | |
| 427 | APELLGGPSVFLFPPKPKDTLMISRTPEVTCVVVDVSHEDPEVKFNWYVDGVE | |
| | VHNAKTKPREEQYNSTYRVVSVLTVLHQDWLNGKEYKCKVSNKALPAPIEKTI | Clone CH3C.35.23.1.1 with |
| | SKAKGQPREPQVYTLPPSRDELTKNQVSLWCLVKGFYPSDIAVEWESFGTEWS | knob and M428L and N434S |
| | NYKTTPPVLDSDGSFFLYSKLTVSKEEWQQGFVFSCSVLHEALHSHYTQKSLS | mutations |
| | LSPGK | |
| 428 | APEAAGGPSVFLFPPKPKDTLMISRTPEVTCVVVDVSHEDPEVKFNWYVDGVE | Clone CH3C.35.23.1.1 with |
| | VHNAKTKPREEQYNSTYRVVSVLTVLHQDWLNGKEYKCKVSNKALPAPIEKTI | knob, LALA, and M428L and |
| | SKAKGQPREPQVYTLPPSRDELTKNQVSLWCLVKGFYPSDIAVEWESFGTEWS | N434S mutations |
| | NYKTTPPVLDSDGSFFLYSKLTVSKEEWQQGFVFSCSVLHEALHSHYTQKSLS | |
| | LSPGK | |
| 429 | APEAAGGPSVFLFPPKPKDTLMISRTPEVTCVVVDVSHEDPEVKFNWYVDGVE | Clone CH3C.35.23.1.1 with |
| | VHNAKTKPREEQYNSTYRVVSVLTVLHQDWLNGKEYKCKVSNKALGAPIEKTI | knob, LALAPG, and M428L |
| | SKAKGQPREPQVYTLPPSRDELTKNQVSLWCLVKGFYPSDIAVEWESFGTEWS | and N434S mutations |
| | NYKTTPPVLDSDGSFFLYSKLTVSKEEWQQGFVFSCSVLHEALHSHYTQKSLS | |
| | LSPGK | |
| 430 | APELLGGPSVFLFPPKPKDTLMISRTPEVTCVVVDVSHEDPEVKFNWYVDGVE | Clone CH3C.35.23.1.1 with |
| | VHNAKTKPREEQYNSTYRVVSVLTVLHQDWLNGKEYKCKVSNKALPAPIEKTI | hole and M428L and N434S |
| | SKAKGQPREPQVYTLPPSRDELTKNQVSLSCAVKGFYPSDIAVEWESFGTEWS | mutations |
| | NYKTTPPVLDSDGSFFLVSKLTVSKEEWQQGFVFSCSVLHEALHSHYTQKSLS | |
| | LSPGK | |
| 431 | APEAAGGPSVFLFPPKPKDTLMISRTPEVTCVVVDVSHEDPEVKFNWYVDGVE | Clone CH3C.35.23.1.1 with |
| | VHNAKTKPREEQYNSTYRVVSVLTVLHQDWLNGKEYKCKVSNKALPAPIEKTI | hole, LALA, and M428L and |
| | SKAKGQPREPQVYTLPPSRDELTKNQVSLSCAVKGFYPSDIAVEWESFGTEWS | N434S mutations |
| | NYKTTPPVLDSDGSFFLVSKLTVSKEEWQQGFVFSCSVLHEALHSHYTQKSLS | |
| | LSPGK | |
| 432 | APEAAGGPSVFLFPPKPKDTLMISRTPEVTCVVVDVSHEDPEVKFNWYVDGVE | Clone CH3C.35.23.1.1 with |
| | VHNAKTKPREEQYNSTYRVVSVLTVLHQDWLNGKEYKCKVSNKALGAPIEKTI | hole, LALAPG, and M428L |
| | SKAKGQPREPQVYTLPPSRDELTKNQVSLSCAVKGFYPSDIAVEWESFGTEWS | and N434S mutations |
| | NYKTTPPVLDSDGSFFLVSKLTVSKEEWQQGFVFSCSVLHEALHSHYTQKSLS | |
| | LSPGK | |
| 433 | APELLGGPSVFLFPPKPKDTLMISRTPEVTCVVVDVSHEDPEVKFNWYVDGVE | Clone CH3C.35.23.2 with |
| | VHNAKTKPREEQYNSTYRVVSVLTVLHQDWLNGKEYKCKVSNKALPAPIEKTI | M428L and N434S mutations |
| | SKAKGQPREPQVYTLPPSRDELTKNQVSLTCLVKGFYPSDIAVEWESYGTEWA | |
| | NYKTTPPVLDSDGSFFLYSKLTVTKEEWQQGFVFSCSVLHEALHSHYTQKSLS | |
| | LSPGK | |
| 434 | APELLGGPSVFLFPPKPKDTLMISRTPEVTCVVVDVSHEDPEVKFNWYVDGVE | Clone CH3C.35.23.2 with |
| | VHNAKTKPREEQYNSTYRVVSVLTVLHQDWLNGKEYKCKVSNKALPAPIEKTI | knob and M428L and N434S |
| | SKAKGQPREPQVYTLPPSRDELTKNQVSLWCLVKGFYPSDIAVEWESYGTEWA | mutations |
| | NYKTTPPVLDSDGSFFLYSKLTVTKEEWQQGFVFSCSVLHEALHSHYTQKSLS | |
| | LSPGK | |
| 435 | APEAAGGPSVFLFPPKPKDTLMISRTPEVTCVVVDVSHEDPEVKFNWYVDGVE | Clone CH3C.35.23.2 with |
| | VHNAKTKPREEQYNSTYRVVSVLTVLHQDWLNGKEYKCKVSNKALPAPIEKTI | knob, LALA, and M428L and |
| | SKAKGQPREPQVYTLPPSRDELTKNQVSLWCLVKGFYPSDIAVEWESYGTEWA | N434S mutations |
| | NYKTTPPVLDSDGSFFLYSKLTVTKEEWQQGFVFSCSVLHEALHSHYTQKSLS | |
| | LSPGK | |
| 436 | APEAAGGPSVFLFPPKPKDTLMISRTPEVTCVVVDVSHEDPEVKFNWYVDGVE | Clone CH3C.35.23.2 with |
| | VHNAKTKPREEQYNSTYRVVSVLTVLHQDWLNGKEYKCKVSNKALGAPIEKTI | knob, LALAPG, and M428L |
| | SKAKGQPREPQVYTLPPSRDELTKNQVSLWCLVKGFYPSDIAVEWESYGTEWA | and N434S mutations |
| | NYKTTPPVLDSDGSFFLYSKLTVTKEEWQQGFVFSCSVLHEALHSHYTQKSLS | |
| | LSPGK | |
| 437 | APELLGGPSVFLFPPKPKDTLMISRTPEVTCVVVDVSHEDPEVKFNWYVDGVE | Clone CH3C.35.23.2 with |
| | VHNAKTKPREEQYNSTYRVVSVLTVLHQDWLNGKEYKCKVSNKALPAPIEKTI | hole and M428L and N434S |
| | SKAKGQPREPQVYTLPPSRDELTKNQVSLSCAVKGFYPSDIAVEWESYGTEWA | mutations |
| | NYKTTPPVLDSDGSFFLVSKLTVTKEEWQQGFVFSCSVLHEALHSHYTQKSLS | |
| | LSPGK | |
| 438 | APEAAGGPSVFLFPPKPKDTLMISRTPEVTCVVVDVSHEDPEVKFNWYVDGVE | Clone CH3C.35.23.2 with |
| | VHNAKTKPREEQYNSTYRVVSVLTVLHQDWLNGKEYKCKVSNKALPAPIEKTI | hole, LALA, and M428L and |
| | SKAKGQPREPQVYTLPPSRDELTKNQVSLSCAVKGFYPSDIAVEWESYGTEWA | N434S mutations |
| | NYKTTPPVLDSDGSFFLVSKLTVTKEEWQQGFVFSCSVLHEALHSHYTQKSLS | |
| | LSPGK | |
| 439 | APEAAGGPSVFLFPPKPKDTLMISRTPEVTCVVVDVSHEDPEVKFNWYVDGVE | Clone CH3C.35.23.2 with |
| | VHNAKTKPREEQYNSTYRVVSVLTVLHQDWLNGKEYKCKVSNKALGAPIEKTI | hole, LALAPG, and M428L |
| | SKAKGQPREPQVYTLPPSRDELTKNQVSLSCAVKGFYPSDIAVEWESYGTEWA | and N434S mutations |
| | NYKTTPPVLDSDGSFFLVSKLTVTKEEWQQGFVFSCSVLHEALHSHYTQKSLS | |
| | LSPGK | |
| 440 | APELLGGPSVFLFPPKPKDTLMISRTPEVTCVVVDVSHEDPEVKFNWYVDGVE | Clone CH3C.35.23.2.1 with |
| | VHNAKTKPREEQYNSTYRVVSVLTVLHQDWLNGKEYKCKVSNKALPAPIEKTI | M428L and N434S mutations |
| | SKAKGQPREPQVYTLPPSRDELTKNQVSLTCLVKGFYPSDIAVEWESYGTEWA | |
| | NYKTTPPVLDSDGSFFLYSKLTVSKSEWQQGFVFSCSVLHEALHSHYTQKSLS | |
| | LSPGK | |
| 441 | APELLGGPSVFLFPPKPKDTLMISRTPEVTCVVVDVSHEDPEVKFNWYVDGVE | Clone CH3C.35.23.2.1 with |
| | VHNAKTKPREEQYNSTYRVVSVLTVLHQDWLNGKEYKCKVSNKALPAPIEKTI | knob and M428L and N434S |
| | SKAKGQPREPQVYTLPPSRDELTKNQVSLWCLVKGFYPSDIAVEWESYGTEWA | mutations |
| | NYKTTPPVLDSDGSFFLYSKLTVSKSEWQQGFVFSCSVLHEALHSHYTQKSLS | |
| | LSPGK | |
| 442 | APEAAGGPSVFLFPPKPKDTLMISRTPEVTCVVVDVSHEDPEVKFNWYVDGVE | Clone CH3C.35.23.2.1 with |
| | VHNAKTKPREEQYNSTYRVVSVLTVLHQDWLNGKEYKCKVSNKALPAPIEKTI | knob, LALA, and M428L and |
| | SKAKGQPREPQVYTLPPSRDELTKNQVSLWCLVKGFYPSDIAVEWESYGTEWA | N434S mutations |
| | NYKTTPPVLDSDGSFFLYSKLTVSKSEWQQGFVFSCSVLHEALHSHYTQKSLS | |
| | LSPGK | |
| 443 | APEAAGGPSVFLFPPKPKDTLMISRTPEVTCVVVDVSHEDPEVKFNWYVDGVE | Clone CH3C.35.23.2.1 with |
| | VHNAKTKPREEQYNSTYRVVSVLTVLHQDWLNGKEYKCKVSNKALGAPIEKTI | knob, LALAPG, and M428L |
| | SKAKGQPREPQVYTLPPSRDELTKNQVSLWCLVKGFYPSDIAVEWESYGTEWA | and N434S mutations |
| | NYKTTPPVLDSDGSFFLYSKLTVSKSEWQQGFVFSCSVLHEALHSHYTQKSLS | |
| | LSPGK | |
| 444 | APELLGGPSVFLFPPKPKDTLMISRTPEVTCVVVDVSHEDPEVKFNWYVDGVE | Clone CH3C.35.23.2.1 with |
| | VHNAKTKPREEQYNSTYRVVSVLTVLHQDWLNGKEYKCKVSNKALPAPIEKTI | hole and M428L and N434S |
| | SKAKGQPREPQVYTLPPSRDELTKNQVSLSCAVKGFYPSDIAVEWESYGTEWA | mutations |
| | NYKTTPPVLDSDGSFFLVSKLTVSKSEWQQGFVFSCSVLHEALHSHYTQKSLS | |
| | LSPGK | |
| 445 | APEAAGGPSVFLFPPKPKDTLMISRTPEVTCVVVDVSHEDPEVKFNWYVDGVE | Clone CH3C.35.23.2.1 with |
| | VHNAKTKPREEQYNSTYRVVSVLTVLHQDWLNGKEYKCKVSNKALPAPIEKTI | hole, LALA, and M428L and |
| | SKAKGQPREPQVYTLPPSRDELTKNQVSLSCAVKGFYPSDIAVEWESYGTEWA | N434S mutations |
| | NYKTTPPVLDSDGSFFLVSKLTVSKSEWQQGFVFSCSVLHEALHSHYTQKSLS | |
| | LSPGK | |
| 446 | APEAAGGPSVFLFPPKPKDTLMISRTPEVTCVVVDVSHEDPEVKFNWYVDGVE | Clone CH3C.35.23.2.1 with |
| | VHNAKTKPREEQYNSTYRVVSVLTVLHQDWLNGKEYKCKVSNKALGAPIEKTI | hole, LALAPG, and M428L |
| | SKAKGQPREPQVYTLPPSRDELTKNQVSLSCAVKGFYPSDIAVEWESYGTEWA | and N434S mutations |
| | NYKTTPPVLDSDGSFFLVSKLTVSKSEWQQGFVFSCSVLHEALHSHYTQKSLS | |
| | LSPGK | |
| 447 | APELLGGPSVFLFPPKPKDTLMISRTPEVTCVVVDVSHEDPEVKFNWYVDGVE | Clone CH3C.35.23.3 with |
| | VHNAKTKPREEQYNSTYRVVSVLTVLHQDWLNGKEYKCKVSNKALPAPIEKTI | M428L and N434S mutations |
| | SKAKGQPREPQVYTLPPSRDELTKNQVSLTCLVKGFYPSDIAVEWESYGTEWV | |
| | NYKTTPPVLDSDGSFFLYSKLTVTKEEWQQGFVFSCSVLHEALHSHYTQKSLS | |
| | LSPGK | |
| 448 | APELLGGPSVFLFPPKPKDTLMISRTPEVTCVVVDVSHEDPEVKFNWYVDGVE | Clone CH3C.35.23.3 with |
| | VHNAKTKPREEQYNSTYRVVSVLTVLHQDWLNGKEYKCKVSNKALPAPIEKTI | knob and M428L and N434S |
| | SKAKGQPREPQVYTLPPSRDELTKNQVSLWCLVKGFYPSDIAVEWESYGTEWV | mutations |
| | NYKTTPPVLDSDGSFFLYSKLTVTKEEWQQGFVFSCSVLHEALHSHYTQKSLS | |
| | LSPGK | |
| 449 | APEAAGGPSVFLFPPKPKDTLMISRTPEVTCVVVDVSHEDPEVKFNWYVDGVE | Clone CH3C.35.23.3 with |
| | VHNAKTKPREEQYNSTYRVVSVLTVLHQDWLNGKEYKCKVSNKALPAPIEKTI | knob, LALA, and M428L and |
| | SKAKGQPREPQVYTLPPSRDELTKNQVSLWCLVKGFYPSDIAVEWESYGTEWV | N434S mutations |
| | NYKTTPPVLDSDGSFFLYSKLTVTKEEWQQGFVFSCSVLHEALHSHYTQKSLS | |
| | LSPGK | |
| 450 | APEAAGGPSVFLFPPKPKDTLMISRTPEVTCVVVDVSHEDPEVKFNWYVDGVE | Clone CH3C.35.23.3 with |
| | VHNAKTKPREEQYNSTYRVVSVLTVLHQDWLNGKEYKCKVSNKALGAPIEKTI | knob, LALAPG, and M428L |
| | SKAKGQPREPQVYTLPPSRDELTKNQVSLWCLVKGFYPSDIAVEWESYGTEWV | and N434S mutations |
| | NYKTTPPVLDSDGSFFLYSKLTVTKEEWQQGFVFSCSVLHEALHSHYTQKSLS | |
| | LSPGK | |
| 451 | APELLGGPSVFLFPPKPKDTLMISRTPEVTCVVVDVSHEDPEVKFNWYVDGVE | Clone CH3C.35.23.3 with |
| | VHNAKTKPREEQYNSTYRVVSVLTVLHQDWLNGKEYKCKVSNKALPAPIEKTI | hole and M428L and N434S |
| | SKAKGQPREPQVYTLPPSRDELTKNQVSLSCAVKGFYPSDIAVEWESYGTEWV | mutations |
| | NYKTTPPVLDSDGSFFLVSKLTVTKEEWQQGFVFSCSVLHEALHSHYTQKSLS | |
| | LSPGK | |
| 452 | APEAAGGPSVFLFPPKPKDTLMISRTPEVTCVVVDVSHEDPEVKFNWYVDGVE | Clone CH3C.35.23.3 with |
| | VHNAKTKPREEQYNSTYRVVSVLTVLHQDWLNGKEYKCKVSNKALPAPIEKTI | hole, LALA, and M428L and |
| | SKAKGQPREPQVYTLPPSRDELTKNQVSLSCAVKGFYPSDIAVEWESYGTEWV | N434S mutations |
| | NYKTTPPVLDSDGSFFLVSKLTVTKEEWQQGFVFSCSVLHEALHSHYTQKSLS | |
| | LSPGK | |
| 453 | APEAAGGPSVFLFPPKPKDTLMISRTPEVTCVVVDVSHEDPEVKFNWYVDGVE | Clone CH3C.35.23.3 with |
| | VHNAKTKPREEQYNSTYRVVSVLTVLHQDWLNGKEYKCKVSNKALGAPIEKTI | hole, LALAPG, and M428L |
| | SKAKGQPREPQVYTLPPSRDELTKNQVSLSCAVKGFYPSDIAVEWESYGTEWV | and N434S mutations |
| | NYKTTPPVLDSDGSFFLVSKLTVTKEEWQQGFVFSCSVLHEALHSHYTQKSLS | |
| | LSPGK | |
| 454 | APELLGGPSVFLFPPKPKDTLMISRTPEVTCVVVDVSHEDPEVKFNWYVDGVE | Clone CH3C.35.23.4 with |
| | VHNAKTKPREEQYNSTYRVVSVLTVLHQDWLNGKEYKCKVSNKALPAPIEKTI | M428L and N434S mutations |
| | SKAKGQPREPQVYTLPPSRDELTKNQVSLTCLVKGFYPSDIAVEWESYGTEWS | |
| | NYKTTPPVLDSDGSFFLYSKLTVSKEEWQQGFVFSCSVLHEALHSHYTQKSLS | |
| | LSPGK | |
| 455 | APELLGGPSVFLFPPKPKDTLMISRTPEVTCVVVDVSHEDPEVKFNWYVDGVE | Clone CH3C.35.23.4 with |
| | VHNAKTKPREEQYNSTYRVVSVLTVLHQDWLNGKEYKCKVSNKALPAPIEKTI | knob and M428L and N434S |
| | SKAKGQPREPQVYTLPPSRDELTKNQVSLWCLVKGFYPSDIAVEWESYGTEWS | mutations |
| | NYKTTPPVLDSDGSFFLYSKLTVSKEEWQQGFVFSCSVLHEALHSHYTQKSLS | |
| | LSPGK | |
| 456 | APEAAGGPSVFLFPPKPKDTLMISRTPEVTCVVVDVSHEDPEVKFNWYVDGVE | Clone CH3C.35.23.4 with |
| | VHNAKTKPREEQYNSTYRVVSVLTVLHQDWLNGKEYKCKVSNKALPAPIEKTI | knob, LALA, and M428L and |
| | SKAKGQPREPQVYTLPPSRDELTKNQVSLWCLVKGFYPSDIAVEWESYGTEWS | N434S mutations |
| | NYKTTPPVLDSDGSFFLYSKLTVSKEEWQQGFVFSCSVLHEALHSHYTQKSLS | |
| | LSPGK | |
| 457 | APEAAGGPSVFLFPPKPKDTLMISRTPEVTCVVVDVSHEDPEVKFNWYVDGVE | Clone CH3C.35.23.4 with |
| | VHNAKTKPREEQYNSTYRVVSVLTVLHQDWLNGKEYKCKVSNKALGAPIEKTI | knob, LALAPG, and M428L |
| | SKAKGQPREPQVYTLPPSRDELTKNQVSLWCLVKGFYPSDIAVEWESYGTEWS | and N434S mutations |
| | NYKTTPPVLDSDGSFFLYSKLTVSKEEWQQGFVFSCSVLHEALHSHYTQKSLS | |
| | LSPGK | |
| 458 | APELLGGPSVFLFPPKPKDTLMISRTPEVTCVVVDVSHEDPEVKFNWYVDGVE | Clone CH3C.35.23.4 with |
| | VHNAKTKPREEQYNSTYRVVSVLTVLHQDWLNGKEYKCKVSNKALPAPIEKTI | hole and M428L and N434S |
| | SKAKGQPREPQVYTLPPSRDELTKNQVSLSCAVKGFYPSDIAVEWESYGTEWS | mutations |
| | NYKTTPPVLDSDGSFFLVSKLTVSKEEWQQGFVFSCSVLHEALHSHYTQKSLS | |
| | LSPGK | |
| 459 | APEAAGGPSVFLFPPKPKDTLMISRTPEVTCVVVDVSHEDPEVKFNWYVDGVE | Clone CH3C.35.23.4 with |
| | VHNAKTKPREEQYNSTYRVVSVLTVLHQDWLNGKEYKCKVSNKALPAPIEKTI | hole, LALA, and M428L and |
| | SKAKGQPREPQVYTLPPSRDELTKNQVSLSCAVKGFYPSDIAVEWESYGTEWS | N434S mutations |
| | NYKTTPPVLDSDGSFFLVSKLTVSKEEWQQGFVFSCSVLHEALHSHYTQKSLS | |
| | LSPGK | |
| 460 | APEAAGGPSVFLFPPKPKDTLMISRTPEVTCVVVDVSHEDPEVKFNWYVDGVE | Clone CH3C.35.23.4 with |
| | VHNAKTKPREEQYNSTYRVVSVLTVLHQDWLNGKEYKCKVSNKALGAPIEKTI | hole, LALAPG, and M428L |
| | SKAKGQPREPQVYTLPPSRDELTKNQVSLSCAVKGFYPSDIAVEWESYGTEWS | and N434S mutations |
| | NYKTTPPVLDSDGSFFLVSKLTVSKEEWQQGFVFSCSVLHEALHSHYTQKSLS | |
| | LSPGK | |
| 461 | APELLGGPSVFLFPPKPKDTLMISRTPEVTCVVVDVSHEDPEVKFNWYVDGVE | Fc sequence with hole and |
| | VHNAKTKPREEQYNSTYRVVSVLTVLHQDWLNGKEYKCKVSNKALPAPIEKTI | M428L and N434S mutations |
| | SKAKGQPREPQVYTLPPSRDELTKNQVSLSCAVKGFYPSDIAVEWESNGQPEN | |
| | NYKTTPPVLDSDGSFFLVSKLTVDKSRWQQGNVFSCSVLHEALHSHYTQKSLS | |
| | LSPGK | |
| 462 | APEAAGGPSVFLFPPKPKDTLMISRTPEVTCVVVDVSHEDPEVKFNWYVDGVE | Fc sequence with hole, |
| | VHNAKTKPREEQYNSTYRVVSVLTVLHQDWLNGKEYKCKVSNKALPAPIEKTI | LALA, and M428L and |
| | SKAKGQPREPQVYTLPPSRDELTKNQVSLSCAVKGFYPSDIAVEWESNGQPEN | N434S mutations |
| | NYKTTPPVLDSDGSFFLVSKLTVDKSRWQQGNVFSCSVLHEALHSHYTQKSLS | |
| | LSPGK | |
| 463 | APELLGGPSVFLFPPKPKDTLMISRTPEVTCVVVDVSHEDPEVKFNWYVDGVE | Fc sequence with knob and |
| | VHNAKTKPREEQYNSTYRVVSVLTVLHQDWLNGKEYKCKVSNKALPAPIEKTI | M428L and N434S mutations |
| | SKAKGQPREPQVYTLPPSRDELTKNQVSLWCLVKGFYPSDIAVEWESNGQPEN | |
| | NYKTTPPVLDSDGSFFLYSKLTVDKSRWQQGNVFSCSVLHEALHSHYTQKSLS | |
| | LSPGK | |
| 464 | APEAAGGPSVFLFPPKPKDTLMISRTPEVTCVVVDVSHEDPEVKFNWYVDGVE | Fc sequence with knob, |
| | VHNAKTKPREEQYNSTYRVVSVLTVLHQDWLNGKEYKCKVSNKALPAPIEKTI | LALA, and M428L and |
| | SKAKGQPREPQVYTLPPSRDELTKNQVSLWCLVKGFYPSDIAVEWESNGQPEN | N434S mutations |
| | NYKTTPPVLDSDGSFFLYSKLTVDKSRWQQGNVFSCSVLHEALHSHYTQKSLS | |
| | LSPGK | |
| 465 | MPALLSLVSLLSVLLMGCVAETGGSGHHHHHHSGTHNTTVFQGVAGQSLQVSC | SS2_NHis_TREM2 |
| | PYDSMKHWGRRKAWCRQLGEKGPCQRVVSTHNLWLLSFLRRWNGSTAITDDTL | |
| | GGTLTITLRNLQPHDAGLYQCQSLHGSEADTLRKVLVEVLADPLDHRDAGDLW | |
| | FPGESESFEDAHVEHSISRSLLEGEIPFPPTSAS | |

It is understood that the examples and embodiments described herein are for illustrative purposes only and that various modifications or changes in light thereof will be suggested to persons skilled in the art and are to be included within the spirit and purview of this application and scope of the appended claims.

What is claimed is:

1. An isolated antibody or antigen-binding portion thereof that specifically binds to a human triggering receptor expressed on myeloid cells 2 (TREM2) protein, wherein the antibody or antigen-binding portion thereof decreases levels of soluble TREM2 protein (sTREM2).

2. The isolated antibody of claim 1, wherein the antibody or antigen-binding portion thereof recognizes an epitope that is the same or substantially the same as the epitope recognized by antibody clone 42E8.H1.

3. The isolated antibody of claim 1, wherein the antibody or antigen-binding portion thereof enhances TREM2 activity.

4. The isolated antibody of claim 3, wherein the antibody or antigen-binding portion thereof induces spleen tyrosine kinase (Syk) phosphorylation.

5. The isolated antibody of claim 3 or 4, wherein the antibody or antigen-binding portion thereof enhances phagocytosis or enhances the migration, differentiation, function, or survival of myeloid cells, microglia, or macrophages.

6. The isolated antibody of claim 5, wherein the antibody or antigen-binding portion thereof enhances microglia function without increasing neuroinflammation.

7. The isolated antibody of any one of claims 3 to 6, wherein the antibody or antigen-binding portion thereof recognizes an epitope that is the same or substantially the same as the epitope recognized by antibody clone 42E8.H1

8. The isolated antibody of claim 3, wherein the antibody increases TREM2 activity in the absence of a TREM2 ligand.

9. The isolated antibody of claim 3, wherein the antibody enhances TREM2 activity without blocking binding of a native TREM2 ligand.

10. The isolated antibody of claim 3, wherein the antibody enhances TREM2 activity that is induced by a TREM2 ligand.

11. The isolated antibody of claim 10, wherein the antibody or antigen-binding portion thereof induces Syk phosphorylation in the presence of a TREM2 ligand.

12. The isolated antibody of claim 3, wherein the antibody selectively enhances activity of a TREM2 ligand.

13. The isolated antibody of claim 3, wherein the antibody prevents activation of TREM2 by a TREM2 ligand.

14. The isolated antibody of claim 3, wherein the antibody blocks binding of a TREM2 ligand to TREM2.

15. The isolated antibody of claim 7, wherein the antibody enhances TREM2 activity that is induced by a TREM2 ligand but does not enhance TREM2 activity in the absence of the TREM2 ligand.

16. The isolated antibody of claim 15, wherein the antibody or antigen-binding portion thereof induces Syk phosphorylation in the presence but not the absence of a TREM2 ligand.

17. The isolated antibody of any one of claims 8 to 16, wherein the TREM2 ligand is selected from the group consisting of 1-palmitoyl-2-(5′-oxo-valeroyl)-sn-glycero-3-phosphocholine (POVPC), 2-Arachidonoylglycerol (2-AG), 7-ketocholesterol (7-KC), 24(S)hydroxycholesterol (240HC), 25(S)hydroxycholesterol (250HC), 27-hydroxycholesterol (270HC), Acyl Carnitine (AC), alkylacylglycerophosphocholine (PAF), α-galactosylceramide (KRN7000), Bis(monoacylglycero)phosphate (BMP), Cardiolipin (CL), Ceramide, Ceramide-1-phosphate (C1P), Cholesteryl ester (CE), Cholesterol phosphate (CP), Diacylglycerol 34:1 (DG 34:1), Diacylglycerol 38:4 (DG 38:4), Diacylglycerol pyrophosphate (DGPP), Dihyrdoceramide (DhCer), Dihydrosphingomyelin (DhSM), Ether phosphatidylcholine (PCe), Free cholesterol (FC), Galactosylceramide (GalCer), Galactosylsphingosine (GalSo), Ganglioside GM1, Ganglioside GM3, Glucosylsphingosine (GlcSo), Hank's Balanced Salt Solution (HBSS), Kdo2-Lipid A (KLA), Lactosylceramide (LacCer), lysoalkylacylglycerophosphocholine (LPAF), Lysophosphatidic acid (LPA), Lysophosphatidylcholine (LPC), Lysophosphatidylethanolamine (LPE), Lysophosphatidylglycerol (LPG), Lysophosphatidylinositol (LPI), Lysosphingomyelin (LSM), Lysophosphatidylserine (LPS), N-Acyl-phosphatidylethanolamine (NAPE), N-Acyl-Serine (NSer), Oxidized phosphatidylcholine (oxPC), Palmitic-acid-9-hydroxy-stearic-acid (PAHSA), Phosphatidylethanolamine (PE), Phosphatidylethanol (PEtOH), Phosphatidic acid (PA), Phosphatidylcholine (PC), Phosphatidylglycerol (PG), Phosphatidylinositol (PI), Phosphatidylserine (PS), Sphinganine, Sphinganine-1-phosphate (Sa1P), Sphingomyelin (SM), Sphingosine, Sphingosine-1-phosphate (So1P), and Sulfatide.

18. The isolated antibody of claim 1, wherein the antibody or antigen-binding portion thereof inhibits TREM2 activity.

19. The isolated antibody of claim 18, wherein the antibody prevents activation of TREM2 by a TREM2 ligand.

20. The isolated antibody of claim 18, wherein the antibody blocks binding of a TREM2 ligand to TREM2.

21. The isolated antibody of claim 19 or 20, wherein the TREM2 ligand is selected from the group consisting of 1-palmitoyl-2-(5′-oxo-valeroyl)-sn-glycero-3-phosphocholine (POVPC), 2-Arachidonoylglycerol (2-AG), 7-ketocholesterol (7-KC), 24(S)hydroxycholesterol (240HC), 25 (S)hydroxycholesterol (25OHC), 27-hydroxycholesterol (270HC), Acyl Carnitine (AC), alkylacylglycerophosphocholine (PAF), α-galactosylceramide (KRN7000), Bis(monoacylglycero)phosphate (BMP), Cardiolipin (CL), Ceramide, Ceramide-1-phosphate (C1P), Cholesteryl ester (CE), Cholesterol phosphate (CP), Diacylglycerol 34:1 (DG 34:1), Diacylglycerol 38:4 (DG 38:4), Diacylglycerol pyrophosphate (DGPP), Dihyrdoceramide (DhCer), Dihydrosphingomyelin (DhSM), Ether phosphatidylcholine (PCe), Free cholesterol (FC), Galactosylceramide (GalCer), Galactosylsphingosine (GalSo), Ganglioside GM1, Ganglioside GM3, Glucosylsphingosine (GlcSo), Hank's Balanced Salt Solution (HBSS), Kdo2-Lipid A (KLA), Lactosylceramide (LacCer), lysoalkylacylglycerophosphocholine (LPAF), Lysophosphatidic acid (LPA), Lysophosphatidylcholine (LPC), Lysophosphatidylethanolamine (LPE), Lysophosphatidylglycerol (LPG), Lysophosphatidylinositol (LPI), Lysosphingomyelin (LSM), Lysophosphatidylserine (LPS), N-Acyl-phosphatidylethanolamine (NAPE), N-Acyl-Serine (NSer), Oxidized phosphatidylcholine (oxPC), Palmitic-acid-9-hydroxy-stearic-acid (PAHSA), Phosphatidylethanolamine (PE), Phosphatidylethanol (PEtOH), Phosphatidic acid (PA), Phosphatidylcholine (PC), Phosphatidylglycerol (PG), Phosphatidylinositol (PI), Phosphatidylserine (PS), Sphinganine, Sphinganine-1-phosphate (Sa1P), Sphingomyelin (SM), Sphingosine, Sphingosine-1-phosphate (So1P), and Sulfatide.

22. The isolated antibody of claim 18, wherein the antibody or antigen-binding portion thereof decreases Syk phosphorylation.

23. An isolated antibody or antigen-binding portion thereof that specifically binds to a human TREM2 protein, wherein the antibody or antigen-binding portion thereof increases levels of sTREM2.

24. The isolated antibody of claim 23, wherein the antibody or antigen-binding portion thereof recognizes an epitope that is the same or substantially the same as the epitope recognized by antibody clone 21D4.D1.

25. The isolated antibody of claim 23, wherein the antibody or antigen-binding portion thereof enhances TREM2 activity.

26. The isolated antibody of claim 25, wherein the antibody or antigen-binding portion thereof induces Syk phosphorylation.

27. The isolated antibody of claim 25 or 26, wherein the antibody or antigen-binding portion thereof enhances phagocytosis or enhances the migration, differentiation, function, or survival of myeloid cells, microglia, or macrophages.

28. The isolated antibody of claim 27, wherein the antibody or antigen-binding portion thereof enhances microglia function without increasing neuroinflammation.

29. The isolated antibody of claim 25, wherein the antibody increases TREM2 activity in the absence of a TREM2 ligand.

30. The isolated antibody of claim 25, wherein the antibody enhances TREM2 activity without blocking binding of a native TREM2 ligand.

31. The isolated antibody of claim 25, wherein the antibody enhances TREM2 activity that is induced by a TREM2 ligand.

32. The isolated antibody of claim 31, wherein the antibody or antigen-binding portion thereof induces Syk phosphorylation in the presence of a TREM2 ligand.

33. The isolated antibody of claim 25, wherein the antibody selectively enhances activity of a TREM2 ligand.

34. The isolated antibody of claim 25, wherein the antibody prevents activation of TREM2 by a TREM2 ligand.

35. The isolated antibody of claim 25, wherein the antibody blocks binding of a TREM2 ligand to TREM2.

36. The isolated antibody of claim 31, wherein the antibody enhances TREM2 activity that is induced by a TREM2 ligand but does not enhance TREM2 activity in the absence of the TREM2 ligand.

37. The isolated antibody of claim 36, wherein the antibody or antigen-binding portion thereof induces Syk phosphorylation in the presence but not the absence of a TREM2 ligand.

38. The isolated antibody of any one of claims 29 to 37, wherein the TREM2 ligand is selected from the group consisting of 1-palmitoyl-2-(5′-oxo-valeroyl)-sn-glycero-3-phosphocholine (POVPC), 2-Arachidonoylglycerol (2-AG), 7-ketocholesterol (7-KC), 24(S)hydroxycholesterol (240HC), 25(S)hydroxycholesterol (250HC), 27-hydroxycholesterol (270HC), Acyl Carnitine (AC), alkylacylglycerophosphocholine (PAF), α-galactosylceramide (KRN7000), Bis(monoacylglycero)phosphate (BMP), Cardiolipin (CL), Ceramide, Ceramide-1-phosphate (C1P), Cholesteryl ester (CE), Cholesterol phosphate (CP), Diacylglycerol 34:1 (DG 34:1), Diacylglycerol 38:4 (DG 38:4), Diacylglycerol pyrophosphate (DGPP), Dihyrdoceramide (DhCer), Dihydrosphingomyelin (DhSM), Ether phosphatidylcholine (PCe), Free cholesterol (FC), Galactosylceramide (GalCer), Galactosylsphingosine (GalSo), Ganglioside GM1, Ganglioside GM3, Glucosylsphingosine (GlcSo), Hank's Balanced Salt Solution (HBSS), Kdo2-Lipid A (KLA), Lactosylceramide (LacCer), lysoalkylacylglycerophosphocholine (LPAF), Lysophosphatidic acid (LPA), Lysophosphatidylcholine (LPC), Lysophosphatidylethanolamine (LPE), Lysophosphatidylglycerol (LPG), Lysophosphatidylinositol (LPI), Lysosphingomyelin (LSM), Lysophosphatidylserine (LPS), N-Acyl-phosphatidylethanolamine (NAPE), N-Acyl-Serine (NSer), Oxidized phosphatidylcholine (oxPC), Palmitic-acid-9-hydroxy-stearic-acid (PAHSA), Phosphatidylethanolamine (PE), Phosphatidylethanol (PEtOH), Phosphatidic acid (PA), Phosphatidylcholine (PC), Phosphatidylglycerol (PG), Phosphatidylinositol (PI), Phosphatidylserine (PS), Sphinganine, Sphinganine-1-phosphate (Sa1P), Sphingomyelin (SM), Sphingosine, Sphingosine-1-phosphate (So1P), and Sulfatide.

39. The isolated antibody of claim 23, wherein the antibody or antigen-binding portion thereof inhibits TREM2 activity.

40. The isolated antibody of claim 39, wherein the antibody prevents activation of TREM2 by a TREM2 ligand.

41. The isolated antibody of claim 39, wherein the antibody blocks binding of a TREM2 ligand to TREM2.

42. The isolated antibody of claim 40 or 41, wherein the TREM2 ligand is selected from the group consisting of 1-palmitoyl-2-(5′-oxo-valeroyl)-sn-glycero-3-phosphocholine (POVPC), 2-Arachidonoylglycerol (2-AG), 7-ketocholesterol (7-KC), 24(S)hydroxycholesterol (240HC), 25 (S)hydroxycholesterol (25OHC), 27-hydroxycholesterol (270HC), Acyl Carnitine (AC), alkylacylglycerophosphocholine (PAF), α-galactosylceramide (KRN7000), Bis(monoacylglycero)phosphate (BMP), Cardiolipin (CL), Ceramide, Ceramide-1-phosphate (C1P), Cholesteryl ester (CE), Cholesterol phosphate (CP), Diacylglycerol 34:1 (DG 34:1), Diacylglycerol 38:4 (DG 38:4), Diacylglycerol pyrophosphate (DGPP), Dihyrdoceramide (DhCer), Dihydrosphingomyelin (DhSM), Ether phosphatidylcholine (PCe), Free cholesterol (FC), Galactosylceramide (GalCer), Galactosylsphingosine (GalSo), Ganglioside GM1, Ganglioside GM3, Glucosylsphingosine (GlcSo), Hank's Balanced Salt Solution (HBSS), Kdo2-Lipid A (KLA), Lactosylceramide (LacCer), lysoalkylacylglycerophosphocholine (LPAF), Lysophosphatidic acid (LPA), Lysophosphatidylcholine (LPC), Lysophosphatidylethanolamine (LPE), Lysophosphatidylglycerol (LPG), Lysophosphatidylinositol (LPI), Lysosphingomyelin (LSM), Lysophosphatidylserine (LPS), N-Acyl-phosphatidylethanolamine (NAPE), N-Acyl-Serine (NSer), Oxidized phosphatidylcholine (oxPC), Palmitic-acid-9-hydroxy-stearic-acid (PAHSA), Phosphatidylethanolamine (PE), Phosphatidylethanol (PEtOH), Phosphatidic acid (PA), Phosphatidylcholine (PC), Phosphatidylglycerol (PG), Phosphatidylinositol (PI), Phosphatidylserine (PS), Sphinganine, Sphinganine-1-phosphate (Sa1P), Sphingomyelin (SM), Sphingosine, Sphingosine-1-phosphate (So1P), and Sulfatide.

43. The isolated antibody of claim 39 or 40, wherein the antibody or antigen-binding portion thereof recognizes an epitope that is the same or substantially the same as the epitope recognized by antibody clone 21D4.D1.

44. The isolated antibody of claim 39, wherein the antibody or antigen-binding portion thereof decreases Syk phosphorylation.

45. An isolated antibody or antigen-binding portion thereof that specifically binds to a human TREM2 protein, wherein the antibody or antigen-binding portion thereof enhances TREM2 activity.

46. The isolated antibody of claim 45, wherein the antibody or antigen-binding portion thereof induces Syk phosphorylation.

47. The isolated antibody of claim 45 or 46, wherein the antibody or antigen-binding portion thereof enhances phagocytosis or enhances the migration, differentiation, function, or survival of myeloid cells, microglia, or macrophages.

48. The isolated antibody of claim 47, wherein the antibody or antigen-binding portion thereof enhances microglia function without increasing neuroinflammation.

49. The isolated antibody of any one of claims 45 to 48, wherein the antibody or antigen-binding portion thereof recognizes an epitope that is the same or substantially the same as the epitope recognized by antibody clone 2G4.B1, 3D3.A1, 7B10.A2, 13B11.A1, 14H11.A1, 21D6.G2, 22G9.D1, 24B4.A1, 26D2.D1, 26E2.A3, 30A8.A1, 38E9.E5, 39H10.A1, 42E8.H1, 43E9.H1, 44E2.H1, 49H11.B1, 52H9.D1, 53H11.D3, 54C2.A1, 55B9.A1, 57D7.A1, 60A4.B1, RS9.E2, RS9.F6, or RS9.F10.

50. The isolated antibody of claim 49, wherein the antibody or antigen-binding portion thereof recognizes an epitope that is the same or substantially the same as the epitope recognized by antibody clone RS9.F6.

51. The isolated antibody of claim 50, wherein the antibody or antigen-binding portion binds a TREM2 fragment that comprises or consists of amino acid residues 140-148.

52. The isolated antibody of claim 50, wherein the antibody or antigen-binding portion thereof binds to an epitope on human TREM2 that comprises amino acid residues 140-144.

53. The isolated antibody of claim 52, wherein the antibody or antigen-binding portion thereof makes direct contact with one or more of residues Asp140, Leu141, Trp142, Phe143, and Pro144.

54. The isolated antibody of claim 53, wherein the antibody or antigen-binding portion thereof makes direct contact with each of residues Asp140, Leu141, Trp142, Phe143, and Pro144.

55. The isolated antibody of claim 45, wherein the antibody increases TREM2 activity in the absence of a TREM2 ligand.

56. The isolated antibody of claim 45, wherein the antibody enhances TREM2 activity without blocking binding of a native TREM2 ligand.

57. The isolated antibody of claim 45, wherein the antibody enhances TREM2 activity that is induced by a TREM2 ligand.

58. The isolated antibody of claim 57, wherein the antibody or antigen-binding portion thereof induces Syk phosphorylation in the presence of a TREM2 ligand.

59. The isolated antibody of claim 45, wherein the antibody selectively enhances activity of a TREM2 ligand.

60. The isolated antibody of claim 45, wherein the antibody blocks binding of a TREM2 ligand to TREM2.

61. The isolated antibody of claim 45, wherein the antibody or antigen-binding portion thereof enhances TREM2 activity that is induced a TREM2 ligand but does not enhance TREM2 activity in the absence of the TREM2 ligand.

62. The isolated antibody of claim 61, wherein the antibody or antigen-binding portion thereof induces Syk phosphorylation in the presence but not the absence of a TREM2 ligand.

63. The isolated antibody of any one of claims 55 to 62, wherein the TREM2 ligand is selected from the group consisting of 1-palmitoyl-2-(5′-oxo-valeroyl)-sn-glycero-3-phosphocholine (POVPC), 2-Arachidonoylglycerol (2-AG), 7-ketocholesterol (7-KC), 24(S)hydroxycholesterol (240HC), 25(S)hydroxycholesterol (250HC), 27-hydroxycholesterol (270HC), Acyl Carnitine (AC), alkylacylglycerophosphocholine (PAF), α-galactosylceramide (KRN7000), Bis(monoacylglycero)phosphate (BMP), Cardiolipin (CL), Ceramide, Ceramide-1-phosphate (C1P), Cholesteryl ester (CE), Cholesterol phosphate (CP), Diacylglycerol 34:1 (DG 34:1), Diacylglycerol 38:4 (DG 38:4), Diacylglycerol pyrophosphate (DGPP), Dihyrdoceramide (DhCer), Dihydrosphingomyelin (DhSM), Ether phosphatidylcholine (PCe), Free cholesterol (FC), Galactosylceramide (GalCer), Galactosylsphingosine (GalSo), Ganglioside GM1, Ganglioside GM3, Glucosylsphingosine (GlcSo), Hank's Balanced Salt Solution (HBSS), Kdo2-Lipid A (KLA), Lactosylceramide (LacCer), lysoalkylacylglycerophosphocholine (LPAF), Lysophosphatidic acid (LPA), Lysophosphatidylcholine (LPC), Lysophosphatidylethanolamine (LPE), Lysophosphatidylglycerol (LPG), Lysophosphatidylinositol (LPI), Lysosphingomyelin (LSM), Lysophosphatidylserine (LPS), N-Acyl-phosphatidylethanolamine (NAPE), N-Acyl-Serine (NSer), Oxidized phosphatidylcholine (oxPC), Palmitic-acid-9-hydroxy-stearic-acid (PAHSA), Phosphatidylethanolamine (PE), Phosphatidylethanol (PEtOH), Phosphatidic acid (PA), Phosphatidylcholine (PC), Phosphatidylglycerol (PG), Phosphatidylinositol (PI), Phosphatidylserine (PS), Sphinganine, Sphinganine-1-phosphate (Sa1P), Sphingomyelin (SM), Sphingosine, Sphingosine-1-phosphate (So1P), and Sulfatide.

64. The isolated antibody of claim 57, wherein the antibody or antigen-binding portion thereof recognizes an epitope that is the same or substantially the same as the epitope recognized by antibody clone 3D3.A1, 8A11.B1, 14D5.F1, 19F10.F3, 21D6.G2, 22B8.B1, 22G9.D1, 26D11.B1, 26E.2.A3, 30A8.A1, 42E8.H1, 43E9.H1, 44E2.H1, 49H11.B1, 52H9.D1, 53H11.D3, 54C2.A1, 59C6.F1, 60 Å4.B1, RS9.F6, or RS9.F10.

65. An isolated antibody or antigen-binding portion thereof that specifically binds to a human TREM2 protein, wherein the antibody or antigen-binding portion thereof inhibits TREM2 activity.

66. The isolated antibody of claim 65, wherein the antibody or antigen-binding portion thereof recognizes an epitope that is the same or substantially the same as the epitope recognized by antibody clone 21D4.D1.

67. The isolated antibody of claim 65, wherein the antibody prevents activation of TREM2 by a TREM2 ligand.

68. The isolated antibody of claim 65, wherein the antibody blocks binding of a TREM2 ligand to TREM2.

69. The isolated antibody of claim 67 or 68, wherein the TREM2 ligand is selected from the group consisting of 1-palmitoyl-2-(5′-oxo-valeroyl)-sn-glycero-3-phosphocholine (POVPC), 2-Arachidonoylglycerol (2-AG), 7-ketocholesterol (7-KC), 24(S)hydroxycholesterol (240HC), 25 (S)hydroxycholesterol (25OHC), 27-hydroxycholesterol (270HC), Acyl Carnitine (AC), alkylacylglycerophosphocholine (PAF), α-galactosylceramide (KRN7000), Bis(monoacylglycero)phosphate (BMP), Cardiolipin (CL), Ceramide, Ceramide-1-phosphate (C1P), Cholesteryl ester (CE), Cholesterol phosphate (CP), Diacylglycerol 34:1 (DG 34:1), Diacylglycerol 38:4 (DG 38:4), Diacylglycerol pyrophosphate (DGPP), Dihyrdoceramide (DhCer), Dihydrosphingomyelin (DhSM), Ether phosphatidylcholine (PCe), Free cholesterol (FC), Galactosylceramide (GalCer), Galactosylsphingosine (GalSo), Ganglioside GM1, Ganglioside GM3, Glucosylsphingosine (GlcSo), Hank's Balanced Salt Solution (HBSS), Kdo2-Lipid A (KLA), Lactosylceramide (LacCer), lysoalkylacylglycerophosphocholine (LPAF), Lysophosphatidic acid (LPA), Lysophosphatidylcholine (LPC), Lysophosphatidylethanolamine (LPE), Lysophosphatidylglycerol (LPG), Lysophosphatidylinositol (LPI), Lysosphingomyelin (LSM), Lysophosphatidylserine (LPS), N-Acyl-phosphatidylethanolamine (NAPE), N-Acyl-Serine (NSer), Oxidized phosphatidylcholine (oxPC), Palmitic-acid-9-hydroxy-stearic-acid (PAHSA), Phosphatidylethanolamine (PE), Phosphatidylethanol (PEtOH), Phosphatidic acid (PA), Phosphatidylcholine (PC), Phosphatidylglycerol (PG), Phosphatidylinositol (PI), Phosphatidylserine (PS), Sphinganine, Sphinganine-1-phosphate (Sa1P), Sphingomyelin (SM), Sphingosine, Sphingosine-1-phosphate (So1P), and Sulfatide.

70. The isolated antibody of claim 67, wherein the antibody or antigen-binding portion thereof recognizes an epitope that is the same or substantially the same as the epitope recognized by antibody clone 2G4.B1, 13B11.A, 14H11.A1, 21D4.D1, 21D11.B1, 24B4.A1, 26D2.D1, 26D5.A1, 30A8.A1, 30F2.A2, 38E9.E5, 39H10.A1, 40H3.A4, 44E3.B1, 51D4.A1, 55B9.A1, 57D7.A1, or RS9.E2.

71. The isolated antibody of claim 65, wherein the antibody or antigen-binding portion thereof decreases Syk phosphorylation.

72. An isolated antibody or antigen-binding portion thereof that specifically binds to a human TREM2 protein, wherein the antibody or antigen-binding portion thereof recognizes an epitope that is the same or substantially the same as the epitope recognized by antibody clone 2G4.B1, 3D3.A1, 7B10.A2, 13B11.A1, 14D5.F1, 14H11.A1, 21D4.D1, 21D6.G2, 21D11, 22B8.B1, 22G9.D1, 24B4.A1, 26D2.D1, 26D5.A1, 26D11.B1, 26E2.A3, 30A8.A1, 30F2.A2, 38E9.E5, 39H10.A1, 40H3.A4, 42E8.H1, 43E9.H1, 44E2.H1, 44E3.B1, 49H11.B1, 51D4, 52H9.D1, 53H11.D3, 54C2.A1, 55B9.A1, 57D7.A1, 59C6.F1, 60A4.B1, RS9.E2, RS9.F6, RS9.F10, RS11.1F5, RS11.1G6, RS11.1A10, RS11.1D11, RS11.4A5, RS11.4H6, RS11.4D7, RS11.4D9, RS11.4F11, RS12.1C6, RS12.1C10, RS12.2D1, RS12.2D4, RS12.2E1, RS12.2F2, RS12.2G1, RS12.2H1, or RS12.3C10.

73. The isolated antibody of claim 72, wherein the antibody or antigen-binding portion thereof has at least 50% overlap with the epitope recognized by an antibody clone selected from the group consisting of 2G4.B1, 3D3.A1, 7B10.A2, 13B11.A1, 14D5.F1, 14H11.A1, 21D4.D1, 21D6.G2, 21D11, 22B8.B1, 22G9.D1, 24B4.A1, 26D2.D1, 26D5.A1, 26D11.B1, 26E2.A3, 30A8.A1, 30F2.A2, 38E9.E5, 39H10.A1, 40H3.A4, 42E8.H1, 43E9.H1, 44E2.H1, 44E3.B1, 49H11.B1, 51D4, 52H9.D1, 53H11.D3, 54C2.A1, 55B9.A1, 57D7.A1, 59C6.F1, 60A4.B1, RS9.E2, RS9.F6, RS9.F10, RS11.1F5, RS11.1G6, RS11.1A10, RS11.1D11, RS11.4A5, RS11.4H6, RS11.4D7, RS11.4D9, RS11.4F11, RS12.1C6, RS12.1C10, RS12.2D1, RS12.2D4, RS12.2E1, RS12.2F2, RS12.2G1, RS12.2H1, and RS12.3C10.

74. The isolated antibody of claim 72, wherein the antibody or antigen-binding portion recognizes an epitope that is the same or substantially the same as the epitope recognized by antibody clone RS9.F6.

75. The isolated antibody of claim 74, wherein the antibody or antigen-binding portion binds to an epitope on human TREM2 that comprises amino acid residues 140-144.

76. The isolated antibody of claim 75, wherein the antibody or antigen-binding portion makes direct contact with one or more of Asp140, Leu141, Trp142, Phe143, and Pro144.

77. The isolated antibody of claim 76, wherein the antibody or antigen-binding portion makes direct contact with each of Asp140, Leu141, Trp142, Phe143, and Pro144.

78. The isolated antibody of claim 74, wherein the antibody or antigen-binding portion binds a TREM2 fragment that comprises or consists of amino acid residues 140-148.

79. The isolated antibody of any one of claims 1 to 78, wherein the antibody or antigen-binding portion thereof comprises:
one or more complementarity determining regions (CDRs) having at least 90% sequence identity to a CDR of antibody clone 2G4.B1, 3D3.A1, 7B10.A2, 13B11.A1, 14D5.F1, 14H11.A1, 21D4.D1, 21D6.G2, 21D11, 22B8.B1, 22G9.D1, 24B4.A1, 26D2.D1, 26D5.A1, 26D11.B1, 26E2.A3, 30A8.A1, 30F2.A2, 38E9.E5, 39H10.A1, 40H3.A4, 42E8.H1, 43E9.H1, 44E2.H1, 44E3.B1, 49H11.B1, 51D4, 52H9.D1, 53H11.D3, 54C2.A1, 55B9.A1, 57D7.A1, 59C6.F1, 60A4.B1, RS9.E2, RS9.F6, RS9.F10, RS11.1F5, RS11.1G6, RS11.1A10, RS11.1D11, RS11.4A5, RS11.4H6, RS11.4D7, RS11.4D9, RS11.4F11, RS12.1C6, RS12.1C10, RS12.2D1, RS12.2D4, RS12.2E1, RS12.2F2, RS12.2G1, RS12.2H1, or RS12.3C10; or
one or more CDRs that has up to two amino acid substitutions relative to a CDR of antibody clone 2G4.B1, 3D3.A1, 7B10.A2, 13B11.A1, 14D5.F1, 14H11.A1, 21D4.D1, 21D6.G2, 21D11, 22B8.B1, 22G9.D1, 24B4.A1, 26D2.D1, 26D5.A1, 26D11.B1, 26E2.A3, 30A8.A1, 30F2.A2, 38E9.E5, 39H10.A1, 40H3.A4, 42E8.H1, 43E9.H1, 44E2.H1, 44E3.B1, 49H11.B1, 51D4, 52H9.D1, 53H11.D3, 54C2.A1, 55B9.A1, 57D7.A1, 59C6.F1, 60A4.B1, RS9.E2, RS9.F6, RS9.F10, RS11.1F5, RS11.1G6, RS11.1A10, RS11.1D11, RS11.4A5, RS11.4H6, RS11.4D7, RS11.4D9, RS11.4F11, RS12.1C6, RS12.1C10, RS12.2D1, RS12.2D4, RS12.2E1, RS12.2F2, RS12.2G1, RS12.2H1, or RS12.3C10.

80. An isolated antibody or antigen-binding portion thereof that specifically binds to a human TREM2 protein, wherein the antibody or antigen-binding portion thereof comprises:
one or more complementarity determining regions (CDRs) having at least 90% sequence identity to a CDR of antibody clone 2G4.B1, 3D3.A1, 7B10.A2, 13B11.A1, 14D5.F1, 14H11.A1, 21D4.D1, 21D6.G2, 21D11, 22B8.B1, 22G9.D1, 24B4.A1, 26D2.D1, 26D5.A1, 26D11.B1, 26E2.A3, 30A8.A1, 30F2.A2, 38E9.E5, 39H10.A1, 40H3.A4, 42E8.H1, 43E9.H1, 44E2.H1, 44E3.B1, 49H11.B1, 51D4, 52H9.D1, 53H11.D3, 54C2.A1, 55B9.A1, 57D7.A1, 59C6.F1, 60A4.B1, RS9.E2, RS9.F6, RS9.F10, RS11.1F5, RS11.1G6, RS11.1A10, RS11.1D11, RS11.4A5, RS11.4H6, RS11.4D7, RS11.4D9, RS11.4F11, RS12.1C6, RS12.1C10, RS12.2D1, RS12.2D4, RS12.2E1, RS12.2F2, RS12.2G1, RS12.2H1, or RS12.3C10; or
one or more CDRs that has up to two amino acid substitutions relative to a CDR of antibody clone 2G4.B1, 3D3.A1, 7B10.A2, 13B11.A1, 14D5.F1, 14H11.A1, 21D4.D1, 21D6.G2, 21D11, 22B8.B1, 22G9.D1, 24B4.A1, 26D2.D1, 26D5.A1, 26D11.B1, 26E2.A3, 30A8.A1, 30F2.A2, 38E9.E5, 39H10.A1, 40H3.A4, 42E8.H1, 43E9.H1, 44E2.H1, 44E3.B1, 49H11.B1, 51D4, 52H9.D1, 53H11.D3, 54C2.A1, 55B9.A1, 57D7.A1, 59C6.F1, 60A4.B1, RS9.E2, RS9.F6, RS9.F10, RS11.1F5, RS11.1G6, RS11.1A10, RS11.1D11, RS11.4A5, RS11.4H6, RS11.4D7, RS11.4D9, RS11.4F11, RS12.1C6, RS12.1C10, RS12.2D1, RS12.2D4, RS12.2E1, RS12.2F2, RS12.2G1, RS12.2H1, or RS12.3C10.

81. The isolated antibody of claim 79 or 80, wherein the antibody or antigen-binding portion thereof comprises each of a heavy chain CDR1 (CDR-H1), a heavy chain CDR2 (CDR-H2), a heavy chain CDR3 (CDR-H3), a light chain CDR1 (CDR-L1), a light chain CDR2 (CDR-L2), and a light chain CDR3 (CDR-L3) that is identical to a CDR-H1, CDR-H2, CDR-H3, CDR-L1, CDR-L2, and CDR-L3 of antibody clone 2G4.B1, 3D3.A1, 7B10.A2, 13B11.A1, 14D5.F1, 14H11.A1, 21D4.D1, 21D6.G2, 21D11, 22B8.B1, 22G9.D1, 24B4.A1, 26D2.D1, 26D5.A1, 26D11.B1, 26E2.A3, 30A8.A1, 30F2.A2, 38E9.E5, 39H10.A1, 40H3.A4, 42E8.H1, 43E9.H1, 44E2.H1, 44E3.B1, 49H11.B1, 51D4, 52H9.D1, 53H11.D3, 54C2.A1, 55B9.A1, 57D7.A1, 59C6.F1, 60A4.B1, RS9.E2, RS9.F6, RS9.F10, RS11.1F5, RS11.1G6, RS11.1A10, RS11.1D11, RS11.4A5, RS11.4H6, RS11.4D7, RS11.4D9, RS11.4F11, RS12.1C6, RS12.1C10, RS12.2D1, RS12.2D4, RS12.2E1, RS12.2F2, RS12.2G1, RS12.2H1, or RS12.3C10.

82. The isolated antibody of any one of claims 1 to 81, wherein the antibody or antigen-binding portion thereof comprises:
a heavy chain variable region comprising an amino acid sequence that has at least 90% sequence identity to the heavy chain variable region of antibody clone 2G4.B1, 3D3.A1, 7B10.A2, 13B11.A1, 14D5.F1, 14H11.A1, 21D4.D1, 21D6.G2, 21D11, 22B8.B1, 22G9.D1, 24B4.A1, 26D2.D1, 26D5.A1, 26D11.B1, 26E2.A3, 30A8.A1, 30F2.A2, 38E9.E5, 39H10.A1, 40H3.A4, 42E8.H1, 43E9.H1, 44E2.H1, 44E3.B1, 49H11.B1, 51D4, 52H9.D1, 53H11.D3, 54C2.A1, 55B9.A1, 57D7.A1, 59C6.F1, 60A4.B1, RS9.E2, RS9.F6, RS9.F10, RS11.1F5, RS11.1G6, RS11.1A10, RS11.1D11, RS11.4A5, RS11.4H6, RS11.4D7, RS11.4D9, RS11.4F11, RS12.1C6, RS12.1C10, RS12.2D1, RS12.2D4, RS12.2E1, RS12.2F2, RS12.2G1, RS12.2H1, or RS12.3C10; and/or
a light chain variable region comprising an amino acid sequence that has at least 90% sequence identity to the light chain variable region of 2G4.B1, 3D3.A1, 7B10.A2, 13B11.A1, 14D5.F1, 14H11.A1, 21D4.D1, 21D6.G2, 21D11, 22B8.B1, 22G9.D1, 24B4.A1, 26D2.D1, 26D5.A1, 26D11.B1, 26E2.A3, 30A8.A1, 30F2.A2, 38E9.E5, 39H10.A1, 40H3.A4, 42E8.H1, 43E9.H1, 44E2.H1, 44E3.B1, 49H11.B1, 51D4, 52H9.D1, 53H11.D3, 54C2.A1, 55B9.A1, 57D7.A1, 59C6.F1, 60A4.B1, RS9.E2, RS9.F6, RS9.F10, RS11.1F5, RS11.1G6, RS11.1A10, RS11.1D11, RS11.4A5, RS11.4H6, RS11.4D7, RS11.4D9, RS11.4F11, RS12.1C6, RS12.1C10, RS12.2D1, RS12.2D4, RS12.2E1, RS12.2F2, RS12.2G1, RS12.2H1, or RS12.3C10.

83. The isolated antibody of claim 82, wherein the antibody or antigen-binding portion thereof comprises:
a heavy chain variable region comprising (i) an amino acid sequence that has at least 75% sequence identity to the heavy chain variable region of antibody clone 2G4.B1, 3D3.A1, 7B10.A2, 13B11.A1, 14D5.F1, 14H11.A1, 21D4.D1, 21D6.G2, 21D11, 22B8.B1, 22G9.D1, 24B4.A1, 26D2.D1, 26D5.A1, 26D11.B1, 26E2.A3, 30A8.A1, 30F2.A2, 38E9.E5, 39H10.A1, 40H3.A4, 42E8.H1, 43E9.H1, 44E2.H1, 44E3.B1, 49H11.B1, 51D4, 52H9.D1, 53H11.D3, 54C2.A1, 55B9.A1, 57D7.A1, 59C6.F1, 60A4.B1, RS9.E2, RS9.F6, RS9.F10, RS11.1F5, RS11.1G6, RS11.1A10, RS11.1D11, RS11.4A5, RS11.4H6, RS11.4D7, RS11.4D9, RS11.4F11, RS12.1C6, RS12.1C10, RS12.2D1, RS12.2D4, RS12.2E1, RS12.2F2, RS12.2G1, RS12.2H1, or RS12.3C10 and (ii) a CDR-H1, CDR-H2, and CDR-H3 that is identical to the CDR-H1, CDR-H2, and CDR-H3 of the antibody clone; and/or
a light chain variable region comprising (i) an amino acid sequence that has at least 75% sequence identity to the light chain variable region of antibody clone 2G4.B1, 3D3.A1, 7B10.A2, 13B11.A1, 14D5.F1, 14H11.A1, 21D4.D1, 21D6.G2, 21D11, 22B8.B1, 22G9.D1, 24B4.A1, 26D2.D1, 26D5.A1, 26D11.B1, 26E2.A3, 30A8.A1, 30F2.A2, 38E9.E5, 39H10.A1, 40H3.A4, 42E8.H1, 43E9.H1, 44E2.H1, 44E3.B1, 49H11.B1, 51D4, 52H9.D1, 53H11.D3, 54C2.A1, 55B9.A1, 57D7.A1, 59C6.F1, 60A4.B1, RS9.E2, RS9.F6, RS9.F10, RS11.1F5, RS11.1G6, RS11.1A10, RS11.1D11, RS11.4A5, RS11.4H6, RS11.4D7, RS11.4D9, RS11.4F11, RS12.1C6, RS12.1C10, RS12.2D1, RS12.2D4, RS12.2E1, RS12.2F2, RS12.2G1, RS12.2H1, or RS12.3C10 and (ii) a CDR-L1, CDR-L2, and CDR-L3 that is identical to the CDR-L1, CDR-L2, and CDR-L3 of the antibody clone.

84. The isolated antibody of any one of claims 1 to 83, wherein the antibody or antigen-binding portion thereof comprises one or more CDRs selected from the group consisting of:
(a) a heavy chain CDR1 sequence having at least 90% sequence identity to the amino acid sequence of any one of SEQ ID NOs:8, 36, 39, 45, 51, 57, 62, 68, 74, 81, 85, 307, and 315 or having up to two amino acid substitutions relative to the amino acid sequence of any one of SEQ ID NOs:8, 36, 39, 45, 51, 57, 62, 68, 74, 81, 85, 307, and 315;
(b) a heavy chain CDR2 sequence having at least 90% sequence identity to the amino acid sequence of any one of SEQ ID NOs:9, 37, 40, 46, 52, 58, 63, 69, 75, 79, 82, 86, 308, and 316 or having up to two amino acid substitutions relative to the amino acid sequence of any one of SEQ ID NOs:9, 37, 40, 46, 52, 58, 63, 69, 75, 79, 82, 86, 308, and 316;
(c) a heavy chain CDR3 sequence having at least 90% sequence identity to the amino acid sequence of any one of SEQ ID NOs:10, 41, 47, 53, 59, 64, 70, 76, 83, 87, 309, and 317 or having up to two amino acid substitutions relative to the amino acid sequence of any one of SEQ ID NOs:10, 41, 47, 53, 59, 64, 70, 76, 83, 87, 309, and 317;
(d) a light chain CDR1 sequence having at least 90% sequence identity to the amino acid sequence of any one of SEQ ID NOs: 11, 42, 48, 54, 60, 65, 71, 77, 88, and 311 or having up to two amino acid substitutions relative to the amino acid sequence of any one of SEQ ID NOs:11, 42, 48, 54, 60, 65, 71, 77, 88, and 311;
(e) a light chain CDR2 sequence having at least 90% sequence identity to the amino acid sequence of any one of SEQ ID NOs:12, 38, 43, 49, 55, 66, 72, 312, and 319 or having up to two amino acid substitutions relative to the amino acid sequence of any one of SEQ ID NOs: 12, 38, 43, 49, 55, 66, 72, 312, and 319; and
(f) a light chain CDR3 sequence having at least 90% sequence identity to the amino acid sequence of any one of SEQ ID NOs: 13, 44, 50, 56, 61, 67, 73, 78, 80, 84, 89, and 313 or having up to two amino acid substitutions relative to the amino acid sequence of any one of SEQ ID NOs: 13, 44, 50, 56, 61, 67, 73, 78, 80, 84, 89, and 313.

85. The isolated antibody of claim 84, wherein the antibody or antigen-binding portion thereof comprises one or more CDRs selected from the group consisting of:
(a) a heavy chain CDR1 sequence comprising the amino acid sequence of any one of SEQ ID NOs:8, 36, 39, 45, 51, 57, 62, 68, 74, 81, 85, 307, and 315;
(b) a heavy chain CDR2 sequence comprising the amino acid sequence of any one of SEQ ID NOs:9, 37, 40, 46, 52, 58, 63, 69, 75, 79, 82, 86, 308, and 316;
(c) a heavy chain CDR3 sequence comprising the amino acid sequence of any one of SEQ ID NOs: 10, 41, 47, 53, 59, 64, 70, 76, 83, 87, 309, and 317;
(d) a light chain CDR1 sequence comprising the amino acid sequence of any one of SEQ ID NOs:11, 42, 48, 54, 60, 65, 71, 77, 88, and 311;
(e) a light chain CDR2 sequence comprising the amino acid sequence of any one of SEQ ID NOs: 12, 38, 43, 49, 55, 66, 72, 312, and 319; and
(f) a light chain CDR3 sequence comprising the amino acid sequence of any one of SEQ ID NOs: 13, 44, 50, 56, 61, 67, 73, 78, 80, 84, 89, and 313.

86. The isolated antibody of claim 85, comprising:
(a) a heavy chain CDR1 sequence comprising the amino acid sequence of SEQ ID NO:8, a heavy chain CDR2 sequence comprising the amino acid sequence of SEQ ID NO:9, a heavy chain CDR3 sequence comprising the amino acid sequence of SEQ ID NO: 10, a light chain CDR1 sequence comprising the amino acid sequence of SEQ ID NO: 11, a light chain CDR2 sequence comprising the amino acid sequence of SEQ ID NO: 12, and a light chain CDR3 sequence comprising the amino acid sequence of SEQ ID NO: 13; or
(b) a heavy chain CDR1 sequence comprising the amino acid sequence of SEQ ID NO:36, a heavy chain CDR2 sequence comprising the amino acid sequence of SEQ ID NO:37, a heavy chain CDR3 sequence comprising the amino acid sequence of SEQ ID NO: 10, a light chain CDR1 sequence comprising the amino acid sequence of SEQ ID NO: 11, a light chain CDR2 sequence comprising the amino acid sequence of SEQ ID NO:38, and a light chain CDR3 sequence comprising the amino acid sequence of SEQ ID NO: 13; or
(c) a heavy chain CDR1 sequence comprising the amino acid sequence of SEQ ID NO:39, a heavy chain CDR2 sequence comprising the amino acid sequence of SEQ ID NO:40, a heavy chain CDR3 sequence comprising the amino acid sequence of SEQ ID NO:41, a light chain CDR1 sequence comprising the amino acid sequence of SEQ ID NO:42, a light chain CDR2 sequence comprising the amino acid sequence of SEQ ID NO:43, and a light chain CDR3 sequence comprising the amino acid sequence of SEQ ID NO:44; or
(d) a heavy chain CDR1 sequence comprising the amino acid sequence of SEQ ID NO:45, a heavy chain CDR2 sequence comprising the amino acid sequence of SEQ ID NO:46, a heavy chain CDR3 sequence comprising the amino acid sequence of SEQ ID NO:47, a light chain CDR1 sequence comprising the amino acid sequence of SEQ ID NO:48, a light chain CDR2 sequence comprising the amino acid sequence of SEQ ID NO:49, and a light chain CDR3 sequence comprising the amino acid sequence of SEQ ID NO:50; or
(e) a heavy chain CDR1 sequence comprising the amino acid sequence of SEQ ID NO:51, a heavy chain CDR2 sequence comprising the amino acid sequence of SEQ ID NO:52, a heavy chain CDR3 sequence comprising the amino acid sequence of SEQ ID NO:53, a light chain CDR1 sequence comprising the amino acid sequence of SEQ ID NO:54, a light chain CDR2 sequence comprising the amino acid sequence of SEQ ID NO:55, and a light chain CDR3 sequence comprising the amino acid sequence of SEQ ID NO:56; or
(f) a heavy chain CDR1 sequence comprising the amino acid sequence of SEQ ID NO:57, a heavy chain CDR2 sequence comprising the amino acid sequence of SEQ ID NO:58, a heavy chain CDR3 sequence comprising the amino acid sequence of SEQ ID NO:59, a light chain CDR1 sequence comprising the amino acid sequence of SEQ ID NO:60, a light chain CDR2 sequence comprising the amino acid sequence of SEQ ID NO:38, and a light chain CDR3 sequence comprising the amino acid sequence of SEQ ID NO:61; or
(g) a heavy chain CDR1 sequence comprising the amino acid sequence of SEQ ID NO:62, a heavy chain CDR2 sequence comprising the amino acid sequence of SEQ ID NO:63, a heavy chain CDR3 sequence comprising the amino acid sequence of SEQ ID NO:64, a light chain CDR1 sequence comprising the amino acid sequence of SEQ ID NO:65, a light chain CDR2 sequence comprising the amino acid sequence of SEQ ID NO:66, and a light chain CDR3 sequence comprising the amino acid sequence of SEQ ID NO:67; or
(h) a heavy chain CDR1 sequence comprising the amino acid sequence of SEQ ID NO:68, a heavy chain CDR2 sequence comprising the amino acid sequence of SEQ ID NO:69, a heavy chain CDR3 sequence comprising the amino acid sequence of SEQ ID NO:70, a light chain CDR1 sequence comprising the amino acid sequence of SEQ ID NO:71, a light chain CDR2 sequence comprising the amino acid sequence of SEQ ID NO:72, and a light chain CDR3 sequence comprising the amino acid sequence of SEQ ID NO:73; or
(i) a heavy chain CDR1 sequence comprising the amino acid sequence of SEQ ID NO:74, a heavy chain CDR2 sequence comprising the amino acid sequence of SEQ ID NO:75, a heavy chain CDR3 sequence comprising the amino acid sequence of SEQ ID NO:76, a light chain CDR1 sequence comprising the amino acid sequence of SEQ ID NO:77, a light chain CDR2 sequence comprising the amino acid sequence of SEQ ID NO:38, and a light chain CDR3 sequence comprising the amino acid sequence of SEQ ID NO:78; or
(j) a heavy chain CDR1 sequence comprising the amino acid sequence of SEQ ID NO:74, a heavy chain CDR2 sequence comprising the amino acid sequence of SEQ ID NO:79, a heavy chain CDR3 sequence comprising the amino acid sequence of SEQ ID NO:76, a light chain CDR1 sequence comprising the amino acid sequence of SEQ ID NO:77, a light chain CDR2 sequence comprising the amino acid sequence of SEQ ID NO:38, and a light chain CDR3 sequence comprising the amino acid sequence of SEQ ID NO:80; or
(k) a heavy chain CDR1 sequence comprising the amino acid sequence of SEQ ID NO:81, a heavy chain CDR2 sequence comprising the amino acid sequence of SEQ ID NO:82, a heavy chain CDR3 sequence comprising the amino acid sequence of SEQ ID NO:83, a light chain CDR1 sequence comprising the amino acid sequence of SEQ ID NO:60, a light chain CDR2 sequence comprising the amino acid sequence of SEQ ID NO:38, and a light chain CDR3 sequence comprising the amino acid sequence of SEQ ID NO:84; or
(l) a heavy chain CDR1 sequence comprising the amino acid sequence of SEQ ID NO:85, a heavy chain CDR2 sequence comprising the amino acid sequence of SEQ ID NO:86, a heavy chain CDR3 sequence comprising the amino acid sequence of SEQ ID NO:87, a light chain CDR1 sequence comprising the amino acid sequence of SEQ ID NO:88, a light chain CDR2 sequence comprising the amino acid sequence of SEQ ID NO:38, and a light chain CDR3 sequence comprising the amino acid sequence of SEQ ID NO:89; or
(m) a heavy chain CDR1 sequence comprising the amino acid sequence of SEQ ID NO:307, a heavy chain CDR2 sequence comprising the amino acid sequence of SEQ ID NO:308, a heavy chain CDR3 sequence comprising the amino acid sequence of SEQ ID NO:309, a light chain CDR1 sequence comprising the amino acid sequence of SEQ ID NO:311, a light chain CDR2 sequence comprising the amino acid sequence of SEQ ID NO:312, and a light chain CDR3 sequence comprising the amino acid sequence of SEQ ID NO:313; or
(n) a heavy chain CDR1 sequence comprising the amino acid sequence of SEQ ID NO:315, a heavy chain CDR2 sequence comprising the amino acid sequence of SEQ ID NO:316, a heavy chain CDR3 sequence comprising the amino acid sequence of SEQ ID NO:317, a light chain CDR1 sequence comprising the amino acid sequence of SEQ ID NO:48, a light chain CDR2 sequence comprising the amino acid sequence of SEQ ID NO:319, and a light chain CDR3 sequence comprising the amino acid sequence of SEQ ID NO:50.

87. The isolated antibody of claim 85, comprising a heavy chain variable region comprising an amino acid sequence that has at least 90% sequence identity to any one of SEQ ID NOs:6, 14, 15, 16, 17, 18, 19, 20, 21, 22, 23, 24, 306, and 314.

88. The isolated antibody of claim 85, comprising a light chain variable region comprising an amino acid sequence that has at least 90% sequence identity to any one of SEQ ID NOs:7, 25, 26, 27, 28, 29, 30, 31, 32, 33, 34, 35, 310, and 318.

89. The isolated antibody of claim 85, comprising:
(a) a heavy chain variable region comprising an amino acid sequence that has at least 90% sequence identity to SEQ ID NO:6; and a light chain variable region comprising an amino acid sequence that has at least 90% sequence identity to SEQ ID NO:7; or
(b) a heavy chain variable region comprising an amino acid sequence that has at least 90% sequence identity to SEQ ID NO:14; and a light chain variable region comprising an amino acid sequence that has at least 90% sequence identity to SEQ ID NO:25; or
(c) a heavy chain variable region comprising an amino acid sequence that has at least 90% sequence identity to SEQ ID NO:15; and a light chain variable region comprising an amino acid sequence that has at least 90% sequence identity to SEQ ID NO:26; or
(d) a heavy chain variable region comprising an amino acid sequence that has at least 90% sequence identity to SEQ ID NO:16; and a light chain variable region comprising an amino acid sequence that has at least 90% sequence identity to SEQ ID NO:27; or
(e) a heavy chain variable region comprising an amino acid sequence that has at least 90% sequence identity to SEQ ID NO:17; and a light chain variable region comprising an amino acid sequence that has at least 90% sequence identity to SEQ ID NO:28; or
(f) a heavy chain variable region comprising an amino acid sequence that has at least 90% sequence identity to SEQ ID NO:18; and a light chain variable region comprising an amino acid sequence that has at least 90% sequence identity to SEQ ID NO:29; or
(g) a heavy chain variable region comprising an amino acid sequence that has at least 90% sequence identity to SEQ ID NO:19; and a light chain variable region comprising an amino acid sequence that has at least 90% sequence identity to SEQ ID NO:30; or
(h) a heavy chain variable region comprising an amino acid sequence that has at least 90% sequence identity to SEQ ID N020; and a light chain variable region comprising an amino acid sequence that has at least 90% sequence identity to SEQ ID NO:31; or
(i) a heavy chain variable region comprising an amino acid sequence that has at least 90% sequence identity to SEQ ID NO:21; and a light chain variable region comprising an amino acid sequence that has at least 90% sequence identity to SEQ ID NO:32; or
(j) a heavy chain variable region comprising an amino acid sequence that has at least 90% sequence identity to SEQ ID NO:22; and a light chain variable region comprising an amino acid sequence that has at least 90% sequence identity to SEQ ID NO:33; or
(k) a heavy chain variable region comprising an amino acid sequence that has at least 90% sequence identity to SEQ ID NO:23; and a light chain variable region comprising an amino acid sequence that has at least 90% sequence identity to SEQ ID NO:34; or
(l) a heavy chain variable region comprising an amino acid sequence that has at least 90% sequence identity to SEQ ID NO:24; and a light chain variable region comprising an amino acid sequence that has at least 90% sequence identity to SEQ ID NO:35; or
(m) a heavy chain variable region comprising an amino acid sequence that has at least 90% sequence identity to SEQ ID NO:306, and a light chain variable region comprising an amino acid sequence that has at least 90% sequence identity to SEQ ID NO:310; or
(n) a heavy chain variable region comprising an amino acid sequence that has at least 90% sequence identity to SEQ ID NO:314, and a light chain variable region comprising an amino acid sequence that has at least 90% sequence identity to SEQ ID NO:318.

90. An isolated antibody or antigen-binding portion thereof that specifically binds to a human TREM2 protein, wherein the antibody or antigen-binding portion thereof comprises:
(a) a light chain variable region; and
(b) a heavy chain variable region comprising a heavy chain CDR1 (CDR-H1), a heavy chain CDR2 (CDR-H2), and a heavy chain CDR3 (CDR-H3), wherein:
(i) CDR-H1 comprises the formula GX₂X₃X₄X₅X₆X₇XX₉X₁₀X₁₁ (I), wherein: X₂ is Y or F; X₃ is T, N, or S; X₄ is F, L, or I; X₅ is T, S, or K; X₆ is D, S, or E; X₇ is D or absent; X₈ is H, Y, or T; X₉ is A, N, G, V, W, T, or Y; X₁₀ is M, I, or W; and X₁₁ is H, Q, or N;
(ii) CDR-H2 comprises the formula X₁X₂X₃X₄X₅X₆X₇X₈X₉X₁₀YX₁₂X₁₃X₁₄X₁₅X₁₆X₁₇ (V), wherein: X₁ is D, V, Y, R, G, or T; X₂ is I, S, or V; X₃ is L, S, N, D, I, or Y; X₄ is P, T, or absent; X₅ is S, Y, N, T, A, G, or F; X₆ is I, S, N, T, or D; X₇ is G or D; X₈ is G, D, N, R, or S; X₉ is R, T, or A; X₁₀ is I, G, S, K, T, N, or R; X₁₂ is G, N, D, or T; X₁₃ is V, Q, E, or P; X₁₄ is K or S; X₁₅ is F, Y or L; X₁₆ is K, R, Q, or is absent; and X₁₇ is G, T, D, S, or is absent; and
(iii) CDR-H3 comprises an amino acid sequence selected from the group consisting of SEQ ID NO:10, SEQ ID NO:47, SEQ ID NO:53, SEQ ID NO:59, SEQ ID NO:70, SEQ ID NO:76, SEQ ID NO:83, SEQ ID NO:87, SEQ ID NO:319, and SEQ ID NO:317 or comprises the formula ARX₃X₄X₅X₆X₇XX₉X₁₀YAX₁₃DY (VIII), wherein: X₃ is G or N; X₄ is D or G; X₅ is D or I; X₆ is S or T; X₇ is Y or T; X₈ is R or A; X₉ is R or G; X₁₀ is G or Y; and X₁₃ is L or M.

91. The isolated antibody of claim 90, wherein the amino acids of formula I are further defined as follows: X₂ is Y; X₃ is T or S; X₅ is T or S; X₈ is H or Y; and X₉ is A, N, G, V, W, or T.

92. The isolated antibody of claim 90 or 91, wherein in CDR-H1 X₇ is absent.

93. The isolated antibody of any one of claims 90 to 92, wherein in CDR-H1 X₁₀ is W and X11 is N.

94. The isolated antibody of claim 90, wherein the amino acids of formula I are further defined as follows: X₃ is T or N; X₇ is absent; X₁₀ is M or I, and X₁₁ is H or Q.

95. The isolated antibody of claim 91 or 92, wherein CDR-H2 has the formula GYTX₄X₅X₆X₈X₉X₁₀X₁₁ (IV), wherein: X₄ is F or L; X₅ is T or S; X₆ is D, S, or E; X₈ is H or Y; X₉ is A, N, G, V, W, or T; X₁₀ is M or I, and X₁₁ is H or Q.

96. The isolated antibody of any one of claims 90 to 95, wherein X₄ of CDR-H1 is F.

97. The isolated antibody of any one of claims 90 to 96, wherein X₅ of CDR-H1 is T.

98. The isolated antibody of claim 96 or 97, wherein X₄ and X₅ of CDR-H1 are F and T, respectively.

99. The isolated antibody of any one of claims 90 to 98, wherein X₆ of CDR-H1 is D or S.

100. The isolated antibody of claim 99, wherein X₆ of CDR-H1 is D.

101. The isolated antibody of claim 99, wherein X₆ of CDR-H1 is S.

102. The isolated antibody of any one of claims 90 to 101, wherein X₈ of CDR-H1 is Y.

103. The isolated antibody of any one of claims 90 to 102, wherein X₁₀ of CDR-H1 is M.

104. The isolated antibody of any one of claims 90 to 103, wherein X₁₁ of CDR-H1 is H.

105. The isolated antibody of claim 103 or 104, wherein X₁₀ and X₁₁ of CDR-H1 are M and H, respectively.

106. The isolated antibody of any one of claims 90 to 102, wherein X₁₀ and X₁₁ of CDR-H1 are I and Q, respectively.

107. The isolated antibody of any one of claims 90 to 106, wherein the amino acids of formula V are further defined as follows: X₁ is V, Y, R, G, or T; X₃ is S, N, D, I, or Y; X₅ is Y, N, T, A, G, or F; X₆ is S, N, T, or D; X₉ is T or A; X₁₂ is N, D, or T; X₁₃ is Q, E, or P; X₁₆ is K, R, or Q; X₁₇ is G, T, D, or S.

108. The isolated antibody of claim 107, wherein the amino acids of formula V are further defined as follows: X₄ is P or T; X₅ is Y, N, T, A, or G; X₈ is G, D, or N; X₁₀ is G, S, K, T, N, or R; X₁₄ is K; X₁₅ is F or Y; and X₁₇ is G, T, or D.

109. The isolated antibody of any one of claims 90 to 108, wherein the antibody comprises a light chain variable region comprising a light chain CDR1 (CDR-L1), a light chain CDR-2 (CDR-L2), and a light chain CDR3 (CDR-L3), wherein:
(i) CDR-L1 comprises the formula X₁SSX₄SLX₇X₈X₉X₁₀X₁₁X₁₂X₁₃X₁₄X₁₅LX₁₇ (IX), wherein: X₁ is R or K; X₄ is Q or K; X₇ is V or L; X₈ is H, D, or Y; X₉ is I, N, or S; X₁₀ is S or absent; X₁₁ is D or N; X₁₂ is G or Q; X₁₃ is N, I, or K; X₁₄ is T or S; X₁₅ is Y or F; and X₁₇ is Q, H, Y, N, or A; or CDR-L1 comprises the formula X₁ASX₄X₅IX₇X₈X₉LX₁₁ (X), wherein: X₁ is R, K, or S; X₄ is E or Q; X₅ is N, D, or G; X₇ is Y or S; X₈ is S or N; X₉ is N, R, or Y; and X₁₁ is A or N;
(ii) CDR-L2 comprises the formula X₁X₂SX₄X₅X₆S (XI), wherein: X₁ is K, Q, Y, V, or L; X₂ is V, M, or T; X₄ is N, K, or Y; X₅ is R or L; and X₆ is F, A, H, or D; or CDR-L2 comprises the amino acid sequence of SEQ ID NO:43, SEQ ID NO:55, or SEQ ID NO:66; and
(iii) CDR-L3 comprises the formula X₁X₂X₃X₄X₅X₆X₇X₈T (XII), wherein: X₁ is S, W, or Q; X₂ is Q or H; X₃ is S, T, G, Y, or F; X₄ is T, F, W, or S; X₅ is H, S, G, or N; X₆ is V, A, F, Y, T, or L; X₇ is P, T, or L; and X₈ is Y, F, P, or W; or CDR-L3 comprises the amino acid sequence of SEQ ID NO:73.

110. The isolated antibody of claim 109, wherein X₄ of formula IX is Q.

111. The isolated antibody of claim 109 or 110, wherein X₈ of formula IX is H.

112. The isolated antibody of any one of claims 109 to 111, wherein X₉ of formula IX is I or S.

113. The isolated antibody of any one of claims 109 to 112, wherein X₁₀ of formula IX is absent.

114. The isolated antibody of any one of claims 109 to 113, wherein X₁₁ of formula IX is N.

115. The isolated antibody of any one of claims 109 to 114, wherein X₁₂ of formula IX is G.

116. The isolated antibody of any one of claims 109 to 115, wherein X₁₃ of formula IX is N or K.

117. The isolated antibody of any one of claims 109 to 116, wherein X₁₄ of formula IX is T.

118. The isolated antibody of any one of claims 109 to 111, wherein X₁₅ of formula IX is Y.

119. The isolated antibody of any one of claims 109 to 118, wherein X₂ of formula XI is V.

120. The isolated antibody of any one of claims 109 to 119, wherein X₄ of formula XI is N.

121. The isolated antibody of any one of claims 109 to 120, wherein X₅ of formula XI is R.

122. The isolated antibody of any one of claims 109 to 121, wherein X₅ of formula XI is L.

123. The isolated antibody of any one of claims 109 to 122, wherein the amino acids of formula XII are further defined as follows: X₁ is Q; X₃ is Y or F; X₄ is F, W, or S; X₅ is S, G, or N; X₆ is Y, T, or L; X₇ is P; X₈ is P, Y, or W.

124. The isolated antibody of any one of claims 109 to 123, wherein X₂ of formula XII is Q.

125. The isolated antibody of any one of claims 109 to 124, wherein X₄ of formula XII is T.

126. The isolated antibody of any one of claims 109 to 125, wherein X₅ of formula XII is H.

127. The isolated antibody of any one of claims 109 to 122, wherein the amino acids of formula XII are further defined as follows: X₁ is S or W; X₂ is Q; X₃ is S, T, or G; X₄ is T; X₅ is H; X₆ is V, A, or F; X₇ is P, T, or L; and X₈ is Y, F, or P.

128. The isolated antibody of claim 127, wherein X₆ of formula XII is V or F.

129. An isolated antibody or antigen-binding portion thereof that specifically binds to a human TREM2 protein, wherein the antibody or antigen-binding portion thereof comprises one or more CDRs selected from the group consisting of:
(a) a heavy chain CDR1 sequence having at least 90% sequence identity to the amino acid sequence of any one of SEQ ID NOs:8, 36, 39, 45, 51, 57, 62, 68, 74, 81, 85, 307, and 315 or having up to two amino acid substitutions relative to the amino acid sequence of any one of SEQ ID NOs:8, 36, 39, 45, 51, 57, 62, 68, 74, 81, 85, 307, and 315;
(b) a heavy chain CDR2 sequence having at least 90% sequence identity to the amino acid sequence of any one of SEQ ID NOs:9, 37, 40, 46, 52, 58, 63, 69, 75, 79, 82, 86, 308, and 316 or having up to two amino acid substitutions relative to the amino acid sequence of any one of SEQ ID NOs:9, 37, 40, 46, 52, 58, 63, 69, 75, 79, 82, 86, 308, and 316;
(c) a heavy chain CDR3 sequence having at least 90% sequence identity to the amino acid sequence of any one of SEQ ID NOs:10, 41, 47, 53, 59, 64, 70, 76, 83, 87, 319, and 317 or having up to two amino acid substitutions relative to the amino acid sequence of any one of SEQ ID NOs:10, 41, 47, 53, 59, 64, 70, 76, 83, 87, 319, and 317;
(d) a light chain CDR1 sequence having at least 90% sequence identity to the amino acid sequence of any one of SEQ ID NOs: 11, 42, 48, 54, 60, 65, 71, 77, 88, and 311 or having up to two amino acid substitutions relative to the amino acid sequence of any one of SEQ ID NOs:11, 42, 48, 54, 60, 65, 71, 77, 88, and 311;
(e) a light chain CDR2 sequence having at least 90% sequence identity to the amino acid sequence of any one of SEQ ID NOs:12, 38, 43, 49, 55, 66, 72, 312, and 319 or having up to two amino acid substitutions relative to the amino acid sequence of any one of SEQ ID NOs: 12, 38, 43, 49, 55, 66, 72, 312, and 319; and
(f) a light chain CDR3 sequence having at least 90% sequence identity to the amino acid sequence of any one of SEQ ID NOs: 13, 44, 50, 56, 61, 67, 73, 78, 80, 84, 89, and 313 or having up to two amino acid substitutions relative to the amino acid sequence of any one of SEQ ID NOs: 13, 44, 50, 56, 61, 67, 73, 78, 80, 84, 89, and 313.

130. The isolated antibody of claim 129, comprising one or more CDRs selected from the group consisting of:
(a) a heavy chain CDR1 comprising the amino acid sequence of any one of SEQ ID NOs:8, 36, 39, 45, 51, 57, 62, 68, 74, 81, 85, 307, and 315;
(b) a heavy chain CDR2 sequence comprising the amino acid sequence of any one of SEQ ID NOs:9, 37, 40, 46, 52, 58, 63, 69, 75, 79, 82, 86, 308, and 316;
(c) a heavy chain CDR3 sequence comprising the amino acid sequence of any one of SEQ ID NOs:10, 41, 47, 53, 59, 64, 70, 76, 83, 87, 319, and 317;
(d) a light chain CDR1 sequence comprising the amino acid sequence of S any one of SEQ ID NOs:11, 42, 48, 54, 60, 65, 71, 77, 88, and 311;
(e) a light chain CDR2 sequence comprising the amino acid sequence of any one of SEQ ID NOs: 12, 38, 43, 49, 55, 66, 72, 312, and 319; and
(f) a light chain CDR3 sequence comprising the amino acid sequence of any one of SEQ ID NOs: 13, 44, 50, 56, 61, 67, 73, 78, 80, 84, 89, and 313.

131. The isolated antibody of claim 130, comprising:
(a) a heavy chain CDR1 sequence comprising the amino acid sequence of SEQ ID NO:8, a heavy chain CDR2 sequence comprising the amino acid sequence of SEQ ID NO:9, a heavy chain CDR3 sequence comprising the amino acid sequence of SEQ ID NO: 10, a light chain CDR1 sequence comprising the amino acid sequence of SEQ ID NO: 11, a light chain CDR2 sequence comprising the amino acid sequence of SEQ ID NO: 12, and a light chain CDR3 sequence comprising the amino acid sequence of SEQ ID NO: 13; or
(b) a heavy chain CDR1 sequence comprising the amino acid sequence of SEQ ID NO:36, a heavy chain CDR2 sequence comprising the amino acid sequence of SEQ ID NO:37, a heavy chain CDR3 sequence comprising the amino acid sequence of SEQ ID NO: 10, a light chain CDR1 sequence comprising the amino acid sequence of SEQ ID NO: 11, a light chain CDR2 sequence comprising the amino acid sequence of SEQ ID NO:38, and a light chain CDR3 sequence comprising the amino acid sequence of SEQ ID NO: 13; or
(c) a heavy chain CDR1 sequence comprising the amino acid sequence of SEQ ID NO:39, a heavy chain CDR2 sequence comprising the amino acid sequence of SEQ ID NO:40, a heavy chain CDR3 sequence comprising the amino acid sequence of SEQ ID NO:41, a light chain CDR1 sequence comprising the amino acid sequence of SEQ ID NO:42, a light chain CDR2 sequence comprising the amino acid sequence of SEQ ID NO:43, and a light chain CDR3 sequence comprising the amino acid sequence of SEQ ID NO:44; or
(d) a heavy chain CDR1 sequence comprising the amino acid sequence of SEQ ID NO:45, a heavy chain CDR2 sequence comprising the amino acid sequence of SEQ ID NO:46, a heavy chain CDR3 sequence comprising the amino acid sequence of SEQ ID NO:47, a light chain CDR1 sequence comprising the amino acid sequence of SEQ ID NO:48, a light chain CDR2 sequence comprising the amino acid sequence of SEQ ID NO:49, and a light chain CDR3 sequence comprising the amino acid sequence of SEQ ID NO:50; or
(e) a heavy chain CDR1 sequence comprising the amino acid sequence of SEQ ID NO:51, a heavy chain CDR2 sequence comprising the amino acid sequence of SEQ ID NO:52, a heavy chain CDR3 sequence comprising the amino acid sequence of SEQ ID NO:53, a light chain CDR1 sequence comprising the amino acid sequence of SEQ ID NO:54, a light chain CDR2 sequence comprising the amino acid sequence of SEQ ID NO:55, and a light chain CDR3 sequence comprising the amino acid sequence of SEQ ID NO:56; or
(f) a heavy chain CDR1 sequence comprising the amino acid sequence of SEQ ID NO:57, a heavy chain CDR2 sequence comprising the amino acid sequence of SEQ ID NO:58, a heavy chain CDR3 sequence comprising the amino acid sequence of SEQ ID NO:59, a light chain CDR1 sequence comprising the amino acid sequence of SEQ ID NO:60, a light chain CDR2 sequence comprising the amino acid sequence of SEQ ID NO:38, and a light chain CDR3 sequence comprising the amino acid sequence of SEQ ID NO:61; or
(g) a heavy chain CDR1 sequence comprising the amino acid sequence of SEQ ID NO:62, a heavy chain CDR2 sequence comprising the amino acid sequence of SEQ ID NO:63, a heavy chain CDR3 sequence comprising the amino acid sequence of SEQ ID NO:64, a light chain CDR1 sequence comprising the amino acid sequence of SEQ ID NO:65, a light chain CDR2 sequence comprising the amino acid sequence of SEQ ID NO:66, and a light chain CDR3 sequence comprising the amino acid sequence of SEQ ID NO:67; or
(h) a heavy chain CDR1 sequence comprising the amino acid sequence of SEQ ID NO:68, a heavy chain CDR2 sequence comprising the amino acid sequence of SEQ ID NO:69, a heavy chain CDR3 sequence comprising the amino acid sequence of SEQ ID NO:70, a light chain CDR1 sequence comprising the amino acid sequence of SEQ ID NO:71, a light chain CDR2 sequence comprising the amino acid sequence of SEQ ID NO:72, and a light chain CDR3 sequence comprising the amino acid sequence of SEQ ID NO:73; or
(i) a heavy chain CDR1 sequence comprising the amino acid sequence of SEQ ID NO:74, a heavy chain CDR2 sequence comprising the amino acid sequence of SEQ ID NO:75, a heavy chain CDR3 sequence comprising the amino acid sequence of SEQ ID NO:76, a light chain CDR1 sequence comprising the amino acid sequence of SEQ ID NO:77, a light chain CDR2 sequence comprising the amino acid sequence of SEQ ID NO:38, and a light chain CDR3 sequence comprising the amino acid sequence of SEQ ID NO:78; or
(j) a heavy chain CDR1 sequence comprising the amino acid sequence of SEQ ID NO:74, a heavy chain CDR2 sequence comprising the amino acid sequence of SEQ ID NO:79, a heavy chain CDR3 sequence comprising the amino acid sequence of SEQ ID NO:76, a light chain CDR1 sequence comprising the amino acid sequence of SEQ ID NO:77, a light chain CDR2 sequence comprising the amino acid sequence of SEQ ID NO:38, and a light chain CDR3 sequence comprising the amino acid sequence of SEQ ID NO:80; or
(k) a heavy chain CDR1 sequence comprising the amino acid sequence of SEQ ID NO:81, a heavy chain CDR2 sequence comprising the amino acid sequence of SEQ ID NO:82, a heavy chain CDR3 sequence comprising the amino acid sequence of SEQ ID NO:83, a light chain CDR1 sequence comprising the amino acid sequence of SEQ ID NO:60, a light chain CDR2 sequence comprising the amino acid sequence of SEQ ID NO:38, and a light chain CDR3 sequence comprising the amino acid sequence of SEQ ID NO:84; or
(l) a heavy chain CDR1 sequence comprising the amino acid sequence of SEQ ID NO:85, a heavy chain CDR2 sequence comprising the amino acid sequence of SEQ ID NO:86, a heavy chain CDR3 sequence comprising the amino acid sequence of SEQ ID NO:87, a light chain CDR1 sequence comprising the amino acid sequence of SEQ ID NO:88, a light chain CDR2 sequence comprising the amino acid sequence of SEQ ID NO:38, and a light chain CDR3 sequence comprising the amino acid sequence of SEQ ID NO:89; or
(m) a heavy chain CDR1 sequence comprising the amino acid sequence of SEQ ID NO:307, a heavy chain CDR2 sequence comprising the amino acid sequence of SEQ ID NO:308, a heavy chain CDR3 sequence comprising the amino acid sequence of SEQ ID NO:309, a light chain CDR1 sequence comprising the amino acid sequence of SEQ ID NO:311, a light chain CDR2 sequence comprising the amino acid sequence of SEQ ID NO:312, and a light chain CDR3 sequence comprising the amino acid sequence of SEQ ID NO:313; or
(n) a heavy chain CDR1 sequence comprising the amino acid sequence of SEQ ID NO:315, a heavy chain CDR2 sequence comprising the amino acid sequence of SEQ ID NO:316, a heavy chain CDR3 sequence comprising the amino acid sequence of SEQ ID NO:317, a light chain CDR1 sequence comprising the amino acid sequence of SEQ ID NO:48, a light chain CDR2 sequence comprising the amino acid sequence of SEQ ID NO:319, and a light chain CDR3 sequence comprising the amino acid sequence of SEQ ID NO:50.

132. The isolated antibody of claim 130 or 131, comprising a heavy chain variable region comprising an amino acid sequence that has at least 90% sequence identity to any one of SEQ ID NOs:6, 14, 15, 16, 17, 18, 19, 20, 21, 22, 23, 24, 306, and 314.

133. The isolated antibody of claim 130 or 131, comprising a light chain variable region comprising an amino acid sequence that has at least 90% sequence identity to any one of SEQ ID NOs:7, 25, 26, 27, 28, 29, 30, 31, 32, 33, 34, 35, 310, and 318.

134. The isolated antibody of any one of claims 129 to 131, comprising:
(a) a heavy chain variable region comprising an amino acid sequence that has at least 90% sequence identity to SEQ ID NO:6; and a light chain variable region comprising an amino acid sequence that has at least 90% sequence identity to SEQ ID NO:7; or
(b) a heavy chain variable region comprising an amino acid sequence that has at least 90% sequence identity to SEQ ID NO:14; and a light chain variable region comprising an amino acid sequence that has at least 90% sequence identity to SEQ ID NO:25; or
(c) a heavy chain variable region comprising an amino acid sequence that has at least 90% sequence identity to SEQ ID NO:15; and a light chain variable region comprising an amino acid sequence that has at least 90% sequence identity to SEQ ID NO:26; or
(d) a heavy chain variable region comprising an amino acid sequence that has at least 90% sequence identity to SEQ ID NO:16; and a light chain variable region comprising an amino acid sequence that has at least 90% sequence identity to SEQ ID NO:27; or
(e) a heavy chain variable region comprising an amino acid sequence that has at least 90% sequence identity to SEQ ID NO:17; and a light chain variable region comprising an amino acid sequence that has at least 90% sequence identity to SEQ ID NO:28; or
(f) a heavy chain variable region comprising an amino acid sequence that has at least 90% sequence identity to SEQ ID NO:18; and a light chain variable region comprising an amino acid sequence that has at least 90% sequence identity to SEQ ID NO:29; or
(g) a heavy chain variable region comprising an amino acid sequence that has at least 90% sequence identity to SEQ ID NO:19; and a light chain variable region comprising an amino acid sequence that has at least 90% sequence identity to SEQ ID NO:30; or
(h) a heavy chain variable region comprising an amino acid sequence that has at least 90% sequence identity to SEQ ID N020; and a light chain variable region comprising an amino acid sequence that has at least 90% sequence identity to SEQ ID NO:31; or
(i) a heavy chain variable region comprising an amino acid sequence that has at least 90% sequence identity to SEQ ID NO:21; and a light chain variable region comprising an amino acid sequence that has at least 90% sequence identity to SEQ ID NO:32; or
(j) a heavy chain variable region comprising an amino acid sequence that has at least 90% sequence identity to SEQ ID NO:22; and a light chain variable region comprising an amino acid sequence that has at least 90% sequence identity to SEQ ID NO:33; or
(k) a heavy chain variable region comprising an amino acid sequence that has at least 90% sequence identity to SEQ ID NO:23; and a light chain variable region comprising an amino acid sequence that has at least 90% sequence identity to SEQ ID NO:34; or
(l) a heavy chain variable region comprising an amino acid sequence that has at least 90% sequence identity to SEQ ID NO:24; and a light chain variable region comprising an amino acid sequence that has at least 90% sequence identity to SEQ ID NO:35; or
(m) a heavy chain variable region comprising an amino acid sequence that has at least 90% sequence identity to SEQ ID NO:306, and a light chain variable region comprising an amino acid sequence that has at least 90% sequence identity to SEQ ID NO:310; or
(n) a heavy chain variable region comprising an amino acid sequence that has at least 90% sequence identity to SEQ ID NO:314, and a light chain variable region comprising an amino acid sequence that has at least 90% sequence identity to SEQ ID NO:318.

135. The isolated antibody of any one of claims 1 to 134, wherein the antibody comprises a first Fc polypeptide and optionally a second Fc polypeptide.

136. The isolated antibody of claim 135, wherein the antibody comprises the first Fc polypeptide and the second Fc polypeptide.

137. The isolated antibody of claim 135 or 136, wherein the first Fc polypeptide is a modified Fc polypeptide and/or the second Fc polypeptide is a modified Fc polypeptide.

138. The isolated antibody of any one of claims 1 to 137, wherein the antibody comprises:
(a) a first antigen-binding portion comprising a first variable region that specifically binds to the human TREM2 protein, wherein the first antigen-binding portion comprises (i) a first heavy chain comprising a first Fc polypeptide and (ii) a first light chain; and
(b) a second antigen-binding portion comprising a second variable region that specifically binds to the human TREM2 protein, wherein the second antigen-binding portion comprises (i) a second heavy chain comprising a first Fc polypeptide and (ii) a second light chain;
wherein the first Fc polypeptide and the second Fc polypeptide form an Fc dimer.

139. The isolated antibody of claim 138, wherein the first Fc polypeptide is a modified Fc polypeptide and/or the second Fc polypeptide is a modified Fc polypeptide.

140. The isolated antibody of claim 138 or 139, wherein the first variable region and the second variable region recognize the same epitope in the human TREM2 protein.

141. The isolated antibody of claim 138 or 139, wherein the first variable region and the second variable region recognize different epitopes in the human TREM2 protein.

142. The isolated antibody of any one of claims 137 to 141, wherein the first Fc polypeptide and the second Fc polypeptide each contain modifications that promote heterodimerization.

143. The isolated antibody of claim 142, wherein one of the Fc polypeptides has a T366W substitution and the other Fc polypeptide has T366S, L368A, and Y407V substitutions, according to EU numbering.

144. The isolated antibody of any one of claims 137 to 143, wherein the first Fc polypeptide and/or the second Fc polypeptide comprises a native FcRn binding site.

145. The isolated antibody of any one of claims 137 to 143, wherein the first Fc polypeptide and/or the second Fc polypeptide comprises a modification that alters FcRn binding.

146. The isolated antibody of any one of claims 137 to 145, wherein the first Fc polypeptide and the second Fc polypeptide do not have effector function.

147. The isolated antibody of any one of claims 137 to 145, wherein the first Fc polypeptide and/or the second Fc polypeptide comprises a modification that reduces effector function.

148. The isolated antibody of claim 147, wherein the modification that reduces effector function comprises substitutions of Ala at position 234 and Ala at position 235, according to EU numbering.

149. The isolated antibody of any one of claims 137 to 148, wherein the first Fc polypeptide and/or the second Fc polypeptide comprises amino acid changes relative to the native Fc sequence that extend serum half-life.

150. The isolated antibody of claim 149, wherein the amino acid changes comprise substitutions of Tyr at position 252, Thr at position 254, and Glu at position 256, according to EU numbering.

151. The isolated antibody of claim 149, wherein the amino acid changes comprise substitutions of Leu at position 428 and Ser at position 434, according to EU numbering.

152. The isolated antibody of claim 149, wherein the amino acid changes comprise a substitution of Ser or Ala at position 434, according to EU numbering.

153. The isolated antibody of any one of claims 137 to 152, wherein the first Fc polypeptide and/or the second Fc polypeptide specifically binds to a transferrin receptor.

154. The isolated antibody of claim 153, wherein the first Fc polypeptide and/or the second Fc polypeptide comprises at least two substitutions at positions selected from the group consisting of 384, 386, 387, 388, 389, 390, 413, 416, and 421, according to EU numbering.

155. The isolated antibody of claim 154, wherein the first Fc polypeptide and/or the second Fc polypeptide comprises substitutions at least three, four, five, six, seven, eight, or nine of the positions.

156. The isolated antibody of claim 154 or 155, wherein the first Fc polypeptide and/or the second Fc polypeptide further comprises one, two, three, or four substitutions at positions comprising 380, 391, 392, and 415, according to EU numbering.

157. The isolated antibody of any one of claims 154 to 156, wherein the first Fc polypeptide and/or the second Fc polypeptide further comprises one, two, or three substitutions at positions comprising 414, 424, and 426, according to EU numbering.

158. The isolated antibody of any one of claims 154 to 157, wherein the first Fc polypeptide and/or the second Fc polypeptide comprises Trp at position 388.

159. The isolated antibody of any one of claims 154 to 158, wherein the first Fc polypeptide and/or the second Fc polypeptide comprises an aromatic amino acid at position 421.

160. The isolated antibody of claim 159, wherein the aromatic amino acid at position 421 is Trp or Phe.

161. The isolated antibody of any one of claims 154 to 160, wherein the first Fc polypeptide and/or the second Fc polypeptide comprises at least one position selected from the following: position 380 is Trp, Leu, or Glu; position 384 is Tyr or Phe; position 386 is Thr; position 387 is Glu; position 388 is Trp; position 389 is Ser, Ala, Val, or Asn; position 390 is Ser or Asn; position 413 is Thr or Ser; position 415 is Glu or Ser; position 416 is Glu; and position 421 is Phe.

162. The isolated antibody of claim 161, wherein the first Fc polypeptide and/or the second Fc polypeptide comprises 2, 3, 4, 5, 6, 7, 8, 9, 10, or 11 positions selected from the following: position 380 is Trp, Leu, or Glu; position 384 is Tyr or Phe; position 386 is Thr; position 387 is Glu; position 388 is Trp; position 389 is Ser, Ala, Val, or Asn; position 390 is Ser or Asn; position 413 is Thr or Ser; position 415 is Glu or Ser; position 416 is Glu; and position 421 is Phe.

163. The isolated antibody of claim 162, wherein the first Fc polypeptide and/or the second Fc polypeptide comprises 11 positions as follows: position 380 is Trp, Leu, or Glu; position 384 is Tyr or Phe; position 386 is Thr; position 387 is Glu; position 388 is Trp; position 389 is Ser, Ala, Val, or Asn; position 390 is Ser or Asn; position 413 is Thr or Ser; position 415 is Glu or Ser; position 416 is Glu; and position 421 is Phe.

164. The isolated antibody of claim 162 or 163, wherein the first Fc polypeptide and/or the second Fc polypeptide has a CH3 domain with at least 85% identity, at least 90% identity, or at least 95% identity to amino acids 111-217 of any one of SEQ ID NOs:100-185, 219-298, and 337-460.

165. The isolated antibody of claim 162 or 163, wherein the first Fc polypeptide and/or the second Fc polypeptide comprises the amino acid sequence of any one of SEQ ID NOs:100-185, 219-298, and 337-460.

166. The isolated antibody of claim 164 or 165, wherein the residues for at least 5, 6, 7, 8, 9, 10, 11, 12, 13, 14, 15, or 16 of the positions corresponding to EU index positions 380, 384, 386, 387, 388, 389, 390, 391, 392, 413, 414, 415, 416, 421, 424 and 426 of any one of SEQ ID NOs:100-185, 219-298, and 337-460 are not deleted or substituted.

167. The isolated antibody of any one of claims 153 to 166, wherein the first Fc polypeptide and/or the second Fc polypeptide binds to the apical domain of the transferrin receptor.

168. The isolated antibody of claim 167, wherein the binding of the antibody or antigen-binding portion thereof to the transferrin receptor does not substantially inhibit binding of transferrin to the transferrin receptor.

169. The isolated antibody of any one of claims 135 to 168, wherein the first Fc polypeptide and/or the second Fc polypeptide has an amino acid sequence identity of at least 75%, or at least 80%, 90%, 92%, or 95%, as compared to the corresponding wild-type Fc polypeptide.

170. The isolated antibody of claim 169, wherein the corresponding wild-type Fc polypeptide is a human IgG1, IgG2, IgG3, or IgG4 Fc polypeptide.

171. The isolated antibody of any one of claims 153 to 121, wherein uptake into the brain of the antibody or antigen-binding portion thereof is at least ten-fold greater as compared to the uptake of the antibody or antigen-binding portion thereof without the modifications in the first Fc polypeptide and/or the second Fc polypeptide that result in transferrin receptor binding.

172. The isolated antibody of any one of claims 135 to 171, wherein one of the Fc polypeptides is not modified to bind to a blood-brain barrier receptor and the other Fc polypeptide is modified to specifically bind to a transferrin receptor.

173. The isolated antibody of any one of claims 1 to 172, wherein the antibody or antigen-binding portion thereof exhibits cross-reactivity with a mouse TREM2 protein.

174. The isolated antibody of any one of claims 1 to 173, wherein the antibody is a monoclonal antibody.

175. The isolated antibody of any one of claims 1 to 173, wherein the antibody is a chimeric antibody.

176. The isolated antibody of any one of claims 1 to 173, wherein the antibody is a humanized antibody.

177. The isolated antibody of any one of claims 1 to 173, wherein the antibody is a fully human antibody.

178. The isolated antibody of any one of claims 1 to 173, wherein the antigen-binding portion is a Fab, a F(ab′)₂, a scFv, or a bivalent scFv.

179. The isolated antibody of any one of claims 1 to 178, wherein the antibody is a multispecific antibody.

180. The isolated antibody of claim 179, wherein the multispecific antibody is a bispecific antibody.

181. The isolated antibody of claim 180, wherein the bispecific antibody recognizes two different TREM2 epitopes.

182. The isolated antibody of claim 180 or 181, wherein the bispecific antibody is capable of inducing TREM2 clustering at the surface of a cell.

183. The isolated antibody of any one of claims 180 to 182, wherein the bispecific antibody has an EC5o that is at least 2-fold lower than a bivalent monospecific antibody comprising the same sequence as a single arm of the bispecific antibody.

184. The isolated antibody of any one of claims 180 to 183, wherein each of the two arms of the bispecific antibody is selected from an antibody clone selected from the group consisting of 2G4.B1, 3D3.A1, 7B10.A2, 13B11.A1, 14D5.F1, 14H11.A1, 21D4.D1, 21D6.G2, 21D11, 22B8.B1, 22G9.D1, 24B4.A1, 26D2.D1, 26D5.A1, 26D11.B1, 26E2.A3, 30A8.A1, 30F2.A2, 38E9.E5, 39H10.A1, 40H3.A4, 42E8.H1, 43E9.H1, 44E2.H1, 44E3.B1, 49H11.B1, 51D4, 52H9.D1, 53H11.D3, 54C2.A1, 55B9.A1, 57D7.A1, 59C6.F1, 60A4.B1, RS9.E2, RS9.F6, and RS9.F10, wherein the two arms are in different epitope bins.

185. A pharmaceutical composition comprising the isolated antibody of any one of claims 1 to 184 and a pharmaceutically acceptable carrier.

186. An antibody that competes with the isolated antibody of any one of claims 1 to 184 for binding to the human TREM2 protein.

187. A kit comprising:
the isolated antibody of any one of claims 1 to 184 or the pharmaceutical composition of claim 185; and
instructions for use thereof.

188. A method of treating a neurodegenerative disease, the method comprising administering to a subject having a neurodegenerative disease the isolated antibody of any one of claims 1 to 184 or the pharmaceutical composition of claim 185.

189. The method of claim 188, wherein the neurodegenerative disease is selected from the group consisting of Alzheimer's disease, primary age-related tauopathy, progressive supranuclear palsy (PSP), frontotemporal dementia, frontotemporal dementia with parkinsonism linked to chromosome 17, argyrophilic grain dementia, amyotrophic lateral sclerosis, amyotrophic lateral sclerosis/parkinsonism-dementia complex of Guam (ALS-PDC), corticobasal degeneration, chronic traumatic encephalopathy, Creutzfeldt-Jakob disease, dementia pugilistica, diffuse neurofibrillary tangles with calcification, Down's syndrome, familial British dementia, familial Danish dementia, Gerstmann-Straussler-Scheinker disease, globular glial tauopathy, Guadeloupean parkinsonism with dementia, Guadelopean PSP, Hallevorden-Spatz disease, hereditary diffuse leukoencephalopathy with spheroids (HDLS), Huntington's disease, inclusion-body myositis, multiple system atrophy, myotonic dystrophy, Nasu-Hakola disease, neurofibrillary tangle-predominant dementia, Niemann-Pick disease type C, pallido-ponto-nigral degeneration, Parkinson's disease, Pick's disease, postencephalitic parkinsonism, prion protein cerebral amyloid angiopathy, progressive subcortical gliosis, subacute sclerosing panencephalitis, and tangle only dementia.

190. The method of claim 189, wherein the neurodegenerative disease is Alzheimer's disease.

191. A method of decreasing levels of sTREM2 in a subject having a neurodegenerative disease, the method comprising administering to the subject the isolated antibody of any one of claims 1 to 22 or 72 to 184.

192. A method of increasing levels of sTREM2 in a subject having a neurodegenerative disease, the method comprising administering to the subject the isolated antibody of any one of claims 23 to 44 or 72 to 184.

193. A method of enhancing TREM2 activity in a subject having a neurodegenerative disease, the method comprising administering to the subject the isolated antibody of any one of claims 45 to 64 or 72 to 184.

194. The method of claim 193, wherein the isolated antibody or antigen-binding portion thereof is an antibody that enhances TREM2 activity that is induced by a ligand.

195. The method of claim 193, wherein the isolated antibody or antigen-binding portion thereof is an antibody that selectively enhances TREM2 activity.

196. The method of claim 193, wherein the isolated antibody or antigen-binding portion thereof is an antibody that enhances TREM2 activity without blocking binding of a native TREM2 ligand.

197. A method of inhibiting TREM2 activity in a subject having a neurodegenerative disease, the method comprising administering to the subject an isolated antibody of any one of claims 65 to 184.

198. The method of any one of claims 193 to 197, wherein the isolated antibody or antigen-binding portion thereof is an antibody that decreases levels of sTREM2.

199. The method of any one of claims 193 to 197, wherein the isolated antibody or antigen-binding portion thereof is an antibody that increases levels of sTREM2.

200. A method of identifying a subject having a neurodegenerative disease as a candidate for treatment with an anti-TREM2 antibody, the method comprising:
measuring the level of sTREM2 in a sample from the subject;
comparing the level of sTREM2 in the sample from the subject to a control value, wherein a level of sTREM2 in the sample from the subject that is elevated relative to the control value identifies the subject as a candidate for treatment; and
for a subject identified as a candidate for treatment, administering to the subject an isolated antibody or an antigen-binding portion thereof that specifically binds to a human TREM2 protein, wherein the isolated antibody or antigen-binding portion thereof decreases levels of sTREM2.

201. A method of treating a subject having a neurodegenerative disease that has been identified as a candidate for treatment with an anti-TREM2 antibody, wherein the subject been identified as having an elevated level of sTREM2, relative to a control value, the method comprising administering to the subject an isolated antibody or an antigen-binding portion thereof that specifically binds to a human TREM2 protein, wherein the isolated antibody or antigen-binding portion thereof decreases levels of sTREM2.

202. The method of claim 200 or 201, wherein the isolated antibody or antigen-binding portion thereof is the antibody of any one of claims 1 to 22 or 72 to 184.

203. A method of identifying a subject having a neurodegenerative disease as a candidate for treatment with an anti-TREM2 antibody, the method comprising:
measuring the level of sTREM2 in a sample from the subject;
comparing the level of sTREM2 in the sample from the subject to a control value, wherein a level of sTREM2 in the sample from the subject that is reduced relative to the control value identifies the subject as a candidate for treatment; and
for a subject identified as a candidate for treatment, administering to the subject an isolated antibody or an antigen-binding portion thereof that specifically binds to a human TREM2 protein, wherein the isolated antibody or antigen-binding portion thereof increases levels of sTREM2.

204. A method of treating a subject having a neurodegenerative disease that has been identified as a candidate for treatment with an anti-TREM2 antibody, wherein the subject has been identified as having a reduced level of sTREM2, relative to a control value, the method comprising administering to the subject an isolated antibody or an antigen-binding portion thereof that specifically binds to a human TREM2 protein, wherein the isolated antibody or antigen-binding portion thereof increases levels of sTREM2.

205. The method of claim 203 or 204, wherein the isolated antibody or antigen-binding portion thereof is the antibody of any one of claims 23 to 44 or 72 to 184.

206. A method of monitoring the efficacy of treatment with an anti-TREM2 antibody for a subject having a neurodegenerative disease, the method comprising:
measuring the level of sTREM2 in a first sample from the subject taken prior to an administration of an anti-TREM2 antibody;
treating the subject with an isolated antibody or an antigen-binding portion thereof that specifically binds to a human TREM2 protein; and
measuring the level of sTREM2 in a second sample from the subject taken subsequent to the administration of the anti-TREM2 antibody;
wherein a change in sTREM2 level in the second sample from the subject, as compared to the first sample from the subject, indicates that the subject is responding to treatment with the anti-TREM2 antibody.

207. The method of claim 71, wherein the isolated antibody or an antigen-binding portion thereof is the antibody of any one of claims 1 to 184.

208. The method of claim 206, wherein a decrease in sTREM2 level in the second sample from the subject, as compared to the first sample from the subject, indicates that the subject is responding to treatment with the anti-TREM2 antibody.

209. The method of claim 206, wherein an increase in sTREM2 level in the second sample from the subject, as compared to the first sample from the subject, indicates that the subject is responding to treatment with the anti-TREM2 antibody.

210. The method of any one of claims 191 to 209, wherein the neurodegenerative disease is selected from the group consisting of Alzheimer's disease, primary age-related tauopathy, progressive supranuclear palsy (PSP), frontotemporal dementia, frontotemporal dementia with parkinsonism linked to chromosome 17, argyrophilic grain dementia, amyotrophic lateral sclerosis, amyotrophic lateral sclerosis/parkinsonism-dementia complex of Guam (ALS-PDC), corticobasal degeneration, chronic traumatic encephalopathy, Creutzfeldt-Jakob disease, dementia pugilistica, diffuse neurofibrillary tangles with calcification, Down's syndrome, familial British dementia, familial Danish dementia, Gerstmann-Straussler-Scheinker disease, globular glial tauopathy, Guadeloupean parkinsonism with dementia, Guadelopean PSP, Hallevorden-Spatz disease, hereditary diffuse leukoencephalopathy with spheroids (HDLS), Huntington's disease, inclusion-body myositis, multiple system atrophy, myotonic dystrophy, Nasu-Hakola disease, neurofibrillary tangle-predominant dementia, Niemann-Pick disease type C, pallido-ponto-nigral degeneration, Parkinson's disease, Pick's disease, postencephalitic parkinsonism, prion protein cerebral amyloid angiopathy, progressive subcortical gliosis, subacute sclerosing panencephalitis, and tangle only dementia.

211. The method of claim 210, wherein the neurodegenerative disease is Alzheimer's disease.
